SEQUENCE LISTING (350 sequences)

SEQ ID NO: 1 - protein, 602 aa, Homo sapiens
MNGGAERAMRSLPSLGGLALLCCAAAAAAAAVASAASAGNVTGGGGAAGQVDASPGPGLRGEPSHPFPRATAPTAQAPRTGPPRATVHRPLAATSPAQSPETTPLWATAGPSSTTFQAPLGPSPTTPPAAERTSTTSQAPTRPAPTTLSTTTGPAPTTPVATTVPAPTTPRTPTPDLPSSSNSSVLPTPPATEAPSSPPPEYVCNCSVVGSLNVNRCNQTTGQCECRPGYQGLHCETCKEGFYLNYTSGLCQPCDCSPHGALSIPCNSSGKCQCKVGVIGSICDRCQDGYYGFSKNGCLPCQCNNRSASCDALTGACLNCQENSKGNHCEECKEGFYQSPDATKECLRCPCSAVTSTGSCSIKSSELEPECDQCKDGYIGPNCNKCENGYYNFDSICRKCQCHGHVDPVKTPKICKPESGECINCLHNTTGFWCENCLEGYVHDLEGNCIKKEVILPTPEGSTILVSNASLTTSVPTPVINSTFTPTTLQTIFSVSTSENSTSALADVSWTQFNIIILTVIIIVVVLLMGFVGAVYMYREYQNRKLNAPFWTIELKEDNISFSSYHDSIPNADVSGLLEDDGNEVAPNGQLTLTTPIHNYKA

SEQ ID NO: 2 - protein, 572 aa, Homo sapiens
AVASAASAGNVTGGGGAAGQVDASPGPGLRGEPSHPFPRATAPTAQAPRTGPPRATVHRPLAATSPAQSPETTPLWATAGPSSTTFQAPLGPSPTTPPAAERTSTTSQAPTRPAPTTLSTTTGPAPTTPVATTVPAPTTPRTPTPDLPSSSNSSVLPTPPATEAPSSPPPEYVCNCSVVGSLNVNRCNQTTGQCECRPGYQGLHCETCKEGFYLNYTSGLCQPCDCSPHGALSIPCNSSGKCQCKVGVIGSICDRCQDGYYGFSKNGCLPCQCNNRSASCDALTGACLNCQENSKGNHCEECKEGFYQSPDATKECLRCPCSAVTSTGSCSIKSSELEPECDQCKDGYIGPNCNKCENGYYNFDSICRKCQCHGHVDPVKTPKICKPESGECINCLHNTTGFWCENCLEGYVHDLEGNCIKKEVILPTPEGSTILVSNASLTTSVPTPVINSTFTPTTLQTIFSVSTSENSTSALADVSWTQFNIIILTVIIIVVVLLMGFVGAVYMYREYQNRKLNAPFWTIELKEDNISFSSYHDSIPNADVSGLLEDDGNEVAPNGQLTLTTPIHNYKA

SEQ ID NO: 3 - protein, 484 aa, Homo sapiens
AVASAASAGNVTGGGGAAGQVDASPGPGLRGEPSHPFPRATAPTAQAPRTGPPRATVHRPLAATSPAQSPETTPLWATAGPSSTTFQAPLGPSPTTPPAAERTSTTSQAPTRPAPTTLSTTTGPAPTTPVATTVPAPTTPRTPTPDLPSSSNSSVLPTPPATEAPSSPPPEYVCNCSVVGSLNVNRCNQTTGQCECRPGYQGLHCETCKEGFYLNYTSGLCQPCDCSPHGALSIPCNSSGKCQCKVGVIGSICDRCQDGYYGFSKNGCLPCQCNNRSASCDALTGACLNCQENSKGNHCEECKEGFYQSPDATKECLRCPCSAVTSTGSCSIKSSELEPECDQCKDGYIGPNCNKCENGYYNFDSICRKCQCHGHVDPVKTPKICKPESGECINCLHNTTGFWCENCLEGYVHDLEGNCIKKEVILPTPEGSTILVSNASLTTSVPTPVINSTFTPTTLQTIFSVSTSENSTSALADVSWTQFN

SEQ ID NO: 4 - protein, 11 aa, Homo sapiens
HGHVDPVKTPK

SEQ ID NO: 5 - protein, 842 aa, Homo sapiens
MAVRPGLWPALLGIVLAAWLRGSGAQQSATVANPVPGANPDLLPHFLVEPEDVYIVKNKPVLLVCKAVPATQIFFKCNGEWVRQVDHVIERSTDGSSGLPTMEVRINVSRQQVEKVFGLEEYWCQCVAWSSSGTTKSQKAYIRIAYLRKNFEQEPLAKEVSLEQGIVLPCRPPEGIPPAEVEWLRNEDLVDPSLDPNVYITREHSLVVRQARLADTANYTCVAKNIVARRRSASAAVIVYVDGSWSPWSKWSACGLDCTHWRSRECSDPAPRNGGEECQGTDLDTRNCTSDLCVHTASGPEDVALYVGLIAVAVCLVLLLLVLILVYCRKKEGLDSDVADSSILTSGFQPVSIKPSKADNPHLLTIQPDLSTTTTTYQGSLCPRQDGPSPKFQLTNGHLLSPLGGGRHTLHHSSPTSEAEEFVSRLSTQNYFRSLPRGTSNMTYGTFNFLGGRLMIPNTGISLLIPPDAIPRGKIYEIYLTLHKPEDVRLPLAGCQTLLSPIVSCGPPGVLLTRPVILAMDHCGEPSPDSWSLRLKKQSCEGSWEDVLHLGEEAPSHLYYCQLEASACYVFTEQLGRFALVGEALSVAAAKRLKLLLFAPVACTSLEYNIRVYCLHDTHDALKEVVQLEKQLGGQLIQEPRVLHFKDSYHNLRLSIHDVPSSLWKSKLLVSYQEIPFYHIWNGTQRYLHCTFTLERVSPSTSDLACKLWVWQVEGDGQSFSINFNITKDTRFAELLALESEAGVPALVGPSAFKIPFLIRQKIISSLDPPCRRGADWRTLAQKLHLDSHLSFFASKPSPTAMILNLWEARHFPNGNLSQLAAAVAGLGQPDAGLFTVSEAEC

SEQ ID NO: 6 - protein, 817 aa, Homo sapiens
QQSATVANPVPGANPDLLPHFLVEPEDVYIVKNKPVLLVCKAVPATQIFFKCNGEWVRQVDHVIERSTDGSSGLPTMEVRINVSRQQVEKVFGLEEYWCQCVAWSSSGTTKSQKAYIRIAYLRKNFEQEPLAKEVSLEQGIVLPCRPPEGIPPAEVEWLRNEDLVDPSLDPNVYITREHSLVVRQARLADTANYTCVAKNIVARRRSASAAVIVYVDGSWSPWSKWSACGLDCTHWRSRECSDPAPRNGGEECQGTDLDTRNCTSDLCVHTASGPEDVALYVGLIAVAVCLVLLLLVLILVYCRKKEGLDSDVADSSILTSGFQPVSIKPSKADNPHLLTIQPDLSTTTTTYQGSLCPRQDGPSPKFQLTNGHLLSPLGGGRHTLHHSSPTSEAEEFVSRLSTQNYFRSLPRGTSNMTYGTFNFLGGRLMIPNTGISLLIPPDAIPRGKIYEIYLTLHKPEDVRLPLAGCQTLLSPIVSCGPPGVLLTRPVILAMDHCGEPSPDSWSLRLKKQSCEGSWEDVLHLGEEAPSHLYYCQLEASACYVFTEQLGRFALVGEALSVAAAKRLKLLLFAPVACTSLEYNIRVYCLHDTHDALKEVVQLEKQLGGQLIQEPRVLHFKDSYHNLRLSIHDVPSSLWKSKLLVSYQEIPFYHIWNGTQRYLHCTFTLERVSPSTSDLACKLWVWQVEGDGQSFSINFNITKDTRFAELLALESEAGVPALVGPSAFKIPFLIRQKIISSLDPPCRRGADWRTLAQKLHLDSHLSFFASKPSPTAMILNLWEARHFPNGNLSQLAAAVAGLGQPDAGLFTVSEAEC

SEQ ID NO: 7 - protein, 279 aa, Homo sapiens
IVARRRSASAAVIVYVDGSWSPWSKWSACGLDCTHWRSRECSDPAPRNGGEECQGTDLDTRNCTSDLCVHTASGPEDVALYVGLIAVAVCLVLLLLVLILVYCRKKEGLDSDVADSSILTSGFQPVSIKPSKADNPHLLTIQPDLSTTTTTYQGSLCPRQDGPSPKFQLTNGHLLSPLGGGRHTLHHSSPTSEAEEFVSRLSTQNYFRSLPRGTSNMTYGTFNFLGGRLMIPNTGISLLIPPDAIPRGKIYEIYLTLHKPEDVRLPLAGCQTLLSPIVS

SEQ ID NO: 8 - protein, 50 aa, Homo sapiens
VYCRKKEGLDSDVADSSILTSGFQPVSIKPSKADNPHLLTIQPDLSTTTT

SEQ ID NO: 9 - protein, 324 aa, Homo sapiens
MAPLLLQLAVLGAALAAAALVLISIVAFTTATKMPALHRHEEEKFFLNAKGQKETLPSIWDSPTKQLSVVVPSYNEEKRLPVMMDEALSYLEKRQKRDPAFTYEVIVVDDGSKDQTSKVAFKYCQKYGSDKVRVITLVKNRGKGGAIRMGIFSSRGEKILMADADGATKFPDVEKLEKGLNDLQPWPNQMAIACGSRAHLEKESIAQRSYFRTLLMYGFHFLVWFLCVKGIRDTQCGFKLFTREAASRTFSSLHVERWAFDVELLYIAQFFKIPIAEIAVNWTEIEGSKLVPFWSWLQMGKDLLFIRLRYLTGAWRLEQTRKMN

SEQ ID NO: 10 - protein, 20 aa, Homo sapiens
DGIWKASFTTFTVTKYWFYR

SEQ ID NO: 11 - protein, 7 aa, Homo sapiens
FTTFTVT

SEQ ID NO: 12 - protein, 8 aa, synthetic construct
SFTTFTVT

SEQ ID NO: 13 - protein, 9 aa, synthetic construct
SFTTFTVTK

SEQ ID NO: 14 - protein, 10 aa, synthetic construct
FTTFTVTKYW

SEQ ID NO: 15 - protein, 10 aa, synthetic construct
ASFTTFTVTK

SEQ ID NO: 16 - protein, 11 aa, synthetic construct
FTTFTVTKYWF

SEQ ID NO: 17 - protein, 11 aa, synthetic construct
ASFTTFTVTKY

SEQ ID NO: 18 - protein, 11 aa, synthetic construct
WKASFTTFTVT

SEQ ID NO: 19 - protein, 12 aa, synthetic construct
SFTTFTVTKYWF

SEQ ID NO: 20 - protein, 12 aa, synthetic construct
KASFTTFTVTKY

SEQ ID NO: 21 - protein, 12 aa, synthetic construct
IWKASFTTFTVT

SEQ ID NO: 22 - protein, 13 aa, synthetic construct
SFTTFTVTKYWFY

SEQ ID NO: 23 - protein, 13 aa, synthetic construct
KASFTTFTVTKYW

SEQ ID NO: 24 - protein, 13 aa, synthetic construct
IWKASFTTFTVTK

SEQ ID NO: 25 - protein, 14 aa, synthetic construct
FTTFTVTKYWFYRL

SEQ ID NO: 26 - protein, 14 aa, synthetic construct
ASFTTFTVTKYWFY

SEQ ID NO: 27 - protein, 14 aa, synthetic construct
WKASFTTFTVTKYW

SEQ ID NO: 28 - protein, 14 aa, synthetic construct
GIWKASFTTFTVTK

SEQ ID NO: 29 - protein, 15 aa, synthetic construct
FTTFTVTKYWFYRLL

SEQ ID NO: 30 - protein, 15 aa, synthetic construct
ASFTTFTVTKYWFYR

SEQ ID NO: 31 - protein, 15 aa, synthetic construct
WKASFTTFTVTKYWF

SEQ ID NO: 32 - protein, 15 aa, synthetic construct
GIWKASFTTFTVTKY

SEQ ID NO: 33 - protein, 15 aa, synthetic construct
FDGIWKASFTTFTVT

SEQ ID NO: 34 - protein, 16 aa, synthetic construct
SFTTFTVTKYWFYRLL

SEQ ID NO: 35 - protein, 16 aa, synthetic construct
KASFTTFTVTKYWFYR

SEQ ID NO: 36 - protein, 16 aa, synthetic construct
IWKASFTTFTVTKYWF

SEQ ID NO: 37 - protein, 16 aa, synthetic construct
DGIWKASFTTFTVTKY

SEQ ID NO: 38 - protein, 16 aa, synthetic construct
SFDGIWKASFTTFTVT

SEQ ID NO: 39 - protein, 17 aa, synthetic construct
SFTTFTVTKYWFYRLLS

SEQ ID NO: 40 - protein, 17 aa, synthetic construct
KASFTTFTVTKYWFYRL

SEQ ID NO: 41 - protein, 17 aa, synthetic construct
IWKASFTTFTVTKYWFY

SEQ ID NO: 42 - protein, 17 aa, synthetic construct
DGIWKASFTTFTVTKYW

SEQ ID NO: 43 - protein, 17 aa, synthetic construct
SFDGIWKASFTTFTVTK

SEQ ID NO: 44 - protein, 18 aa, synthetic construct
FTTFTVTKYWFYRLLSAL

SEQ ID NO: 45 - protein, 18 aa, synthetic construct
ASFTTFTVTKYWFYRLLS

SEQ ID NO: 46 - protein, 18 aa, synthetic construct
WKASFTTFTVTKYWFYRL

SEQ ID NO: 47 - protein, 18 aa, synthetic construct
GIWKASFTTFTVTKYWFY

SEQ ID NO: 48 - protein, 18 aa, synthetic construct
FDGIWKASFTTFTVTKYW

SEQ ID NO: 49 - protein, 18 aa, synthetic construct
HSFDGIWKASFTTFTVTK

SEQ ID NO: 50 - protein, 19 aa, synthetic construct
FTTFTVTKYWFYRLLSALF

SEQ ID NO: 51 - protein, 19 aa, synthetic construct
ASFTTFTVTKYWFYRLLSA

SEQ ID NO: 52 - protein, 19 aa, synthetic construct
WKASFTTFTVTKYWFYRLL

SEQ ID NO: 53 - protein, 19 aa, synthetic construct
GIWKASFTTFTVTKYWFYR

SEQ ID NO: 54 - protein, 19 aa, synthetic construct
FDGIWKASFTTFTVTKYWF

SEQ ID NO: 55 - protein, 19 aa, synthetic construct
HSFDGIWKASFTTFTVTKY

SEQ ID NO: 56 - protein, 19 aa, synthetic construct
GTHSFDGIWKASFTTFTVT

SEQ ID NO: 57 - protein, 8 aa, synthetic construct
FTTFTVTK

SEQ ID NO: 58 - protein, 9 aa, synthetic construct
FTTFTVTKY

SEQ ID NO: 59 - protein, 9 aa, synthetic construct
ASFTTFTVT

SEQ ID NO: 60 - protein, 10 aa, synthetic construct
SFTTFTVTKY

SEQ ID NO: 61 - protein, 10 aa, synthetic construct
KASFTTFTVT

SEQ ID NO: 62 - protein, 11 aa, synthetic construct
SFTTFTVTKYW

SEQ ID NO: 63 - protein, 11 aa, synthetic construct
KASFTTFTVTK

SEQ ID NO: 64 - protein, 12 aa, synthetic construct
FTTFTVTKYWFY

SEQ ID NO: 65 - protein, 12 aa, synthetic construct
ASFTTFTVTKYW

SEQ ID NO: 66 - protein, 12 aa, synthetic construct
WKASFTTFTVTK

SEQ ID NO: 67 - protein, 13 aa, synthetic construct
FTTFTVTKYWFYR

SEQ ID NO: 68 - protein, 13 aa, synthetic construct
ASFTTFTVTKYWF

SEQ ID NO: 69 - protein, 13 aa, synthetic construct
WKASFTTFTVTKY

SEQ ID NO: 70 - protein, 13 aa, synthetic construct
GIWKASFTTFTVT

SEQ ID NO: 71 - protein, 14 aa, synthetic construct
SFTTFTVTKYWFYR

SEQ ID NO: 72 - protein, 14 aa, synthetic construct
KASFTTFTVTKYWF

SEQ ID NO: 73 - protein, 14 aa, synthetic construct
IWKASFTTFTVTKY

SEQ ID NO: 74 - protein, 14 aa, synthetic construct
DGIWKASFTTFTVT

SEQ ID NO: 75 - protein, 15 aa, synthetic construct
SFTTFTVTKYWFYRL

SEQ ID NO: 76 - protein, 15 aa, synthetic construct
KASFTTFTVTKYWFY

SEQ ID NO: 77 - protein, 15 aa, synthetic construct
IWKASFTTFTVTKYW

SEQ ID NO: 78 - protein, 15 aa, synthetic construct
DGIWKASFTTFTVTK

SEQ ID NO: 79 - protein, 16 aa, synthetic construct
FTTFTVTKYWFYRLLS

SEQ ID NO: 80 - protein, 16 aa, synthetic construct
ASFTTFTVTKYWFYRL

SEQ ID NO: 81 - protein, 16 aa, synthetic construct
WKASFTTFTVTKYWFY

SEQ ID NO: 82 - protein, 16 aa, synthetic construct
GIWKASFTTFTVTKYW

SEQ ID NO: 83 - protein, 16 aa, synthetic construct
FDGIWKASFTTFTVTK

SEQ ID NO: 84 - protein, 17 aa, synthetic construct
FTTFTVTKYWFYRLLSA

SEQ ID NO: 85 - protein, 17 aa, synthetic construct
ASFTTFTVTKYWFYRLL

SEQ ID NO: 86 - protein, 17 aa, synthetic construct
WKASFTTFTVTKYWFYR

SEQ ID NO: 87 - protein, 17 aa, synthetic construct
GIWKASFTTFTVTKYWF

SEQ ID NO: 88 - protein, 17 aa, synthetic construct
FDGIWKASFTTFTVTKY

SEQ ID NO: 89 - protein, 17 aa, synthetic construct
HSFDGIWKASFTTFTVT

SEQ ID NO: 90 - protein, 18 aa, synthetic construct
SFTTFTVTKYWFYRLLSA

SEQ ID NO: 91 - protein, 18 aa, synthetic construct
KASFTTFTVTKYWFYRLL

SEQ ID NO: 92 - protein, 18 aa, synthetic construct
IWKASFTTFTVTKYWFYR

SEQ ID NO: 93 - protein, 18 aa, synthetic construct
DGIWKASFTTFTVTKYWF

SEQ ID NO: 94 - protein, 18 aa, synthetic construct
SFDGIWKASFTTFTVTKY

SEQ ID NO: 95 - protein, 18 aa, synthetic construct
THSFDGIWKASFTTFTVT

SEQ ID NO: 96 - protein, 19 aa, synthetic construct
SFTTFTVTKYWFYRLLSAL

SEQ ID NO: 97 - protein, 19 aa, synthetic construct
KASFTTFTVTKYWFYRLLS

SEQ ID NO: 98 - protein, 19 aa, synthetic construct
IWKASFTTFTVTKYWFYRL

SEQ ID NO: 99 - protein, 19 aa, synthetic construct
DGIWKASFTTFTVTKYWFY

SEQ ID NO: 100 - protein, 19 aa, synthetic construct
SFDGIWKASFTTFTVTKYW

SEQ ID NO: 101 - protein, 19 aa, synthetic construct
THSFDGIWKASFTTFTVTK

SEQ ID NO: 102 - protein, 549 aa, Homo sapiens
MGWSCIILFLVATATGVHSAAVASAASAGNVTGGGGAAGQVDASPGPGLRGEPSHPFPRATAPTAQAPRTGPPRATVHRPLAATSPAQSPETTPLWATAGPSSTTFQAPLGPSPTTPPAAERTSTTSQAPTRPAPTTLSTTTGPAPTTPVATTVPAPTTPRTPTPDLPSSSNSSVLPTPPATEAPSSPPPGHQWPVAKMPQKYLGKYACESNLKSKYLPLTQPVMNLRVSEAVKTEYVCNCSVVGSLNVNRCNQTTGQCECRPGYQGLHCETCKEGFYLNYTSGLCQPCDCSPHGALSIPCNSSGKCQCKVGVIGSICDRCQDGYYGFSKNGCLPCQCNNRSASCDALTGACLNCQENSKGNHCEECKEGFYQSPDATKECLRCPCSAVTSTGSCSIKSSELEPECDQCKDGYIGPNCNKCENGYYNFDSICRKCQCHGHVDPVKTPKICKPESGECINCLHNTTGFWCENCLEGYVHDLEGNCIKKEVILPTPEGSTILVSNASLTTSVPTPVINSTFTPTTLQTIFSVSTSENSTSALADVSWTQFN

SEQ ID NO: 103 - protein, 306 aa, synthetic construct
MAVRPGLWPALLGIVLAAWLRGSGAQQSATVANPVPGANPDLLPHFLVEPEDVYIVKNKPVLLVCKAVPATQIFFKCNGEWVRQVDHVIERSTDGSSGLPTMEVRINVSRQQVEKVFGLEEYWCQCVAWSSSGTTKSQKAYIRIAYLRKNFEQEPLAKEVSLEQGIVLPCRPPEGIPPAEVEWLRNEDLVDPSLDPNVYITREHSLVVRQAALADTANYTCVAKNIVARRRSASAAVIVYVDGSWSPWSKWSACGLDCTHWRSRECSDPAPRNGGEECQGTDLDTRNCTSDLCVHTASGPEDVALY

SEQ ID NO: 104 - protein, 297 aa, Homo sapiens
MTTATKMPALHRHEEEKFFLNAKGQKETLPSIWDSPTKQLSVVVPSYNEEKRLPVMMDEALSYLEKRQKADPAFTYEVIVVDDGSKDQTSKVAFKYCQKYGSDKVRVITLVKNRGKGGAIRMGIFSSRGEKILMADADGATKFPDVEKLEKGLNDLQPWPNQMAIACGSRAHLEKESIAQRSYFRTLLMYGFHFLVWFLCVKGIRDTQCGFKLFTREAASRTFSSLHVERWAFDVELLYIAQFFKIPIAEIAVNWTEIEGSKLVPFWSWLQMGKDLLFIRLRYLTGAWRLEQTRKMN

SEQ ID NO: 105 - protein, 467 aa, synthetic construct
MNFGLSLIFLVLILKGVQSEVKLVESGGGLVKPGGSLKLSCAASGFIFSRYGMSWVRQTPEKRLEWVATISSGGGYTSYPDSVKGRFTMSRDNANNNLFLQMSSLRSEDTALYYCARQGGTYYRHDDAPMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 106 - protein, 492 aa, synthetic construct
EVKLVESGGGLVKPGGSLKLSCAASGFIFSRYGMSWVRQTPEKREVKLVESGGGLVKPGGSLKLSCAASGFIFSRYGMSWVRQTPEKRLEWVATISSGGGYTSYPDSVKGRFTMSRDNANNNLFLQMSSLRSEDTALYYCARQGGTYYRHDDAPMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 107 - protein, 124 aa, synthetic construct
EVKLVESGGGLVKPGGSLKLSCAASGFIFSRYGMSWVRQTPEKRLEWVATISSGGGYTSYPDSVKGRFTMSRDNANNNLFLQMSSLRSEDTALYYCARQGGTYYRHDDAPMDYWGQGTSVTVSS

SEQ ID NO: 108 - protein, 5 aa, synthetic construct
RYGMS

SEQ ID NO: 109 - protein, 17 aa, synthetic construct
TISSGGGYTSYPDSVKG

SEQ ID NO: 110 - protein, 15 aa, synthetic construct
QGGTYYRHDDAPMDY

SEQ ID NO: 111 - protein, 1404 aa, synthetic construct
ATGAACTTCGGACTCAGCTTGATTTTCCTTGTCCTAATTTTAAAAGGTGTCCAGTCTGAAGTAAAGCTGGTGGAGTCTGGGGGAGGCTTAGTGAAGCCTGGAGGGTCCCTGAAACTCTCCTGTGCAGCCTCTGGATTCATTTTCAGTAGGTATGGCATGTCTTGGGTTCGCCAGACTCCGGAGAAGAGGCTGGAGTGGGTCGCAACCATTAGTAGTGGTGGTGGTTACACCTCCTATCCAGACAGTGTGAAGGGGCGATTCACCATGTCCAGAGACAATGCCAACAACAACCTGTTCCTGCAAATGAGCAGTCTGAGGTCTGAGGACACGGCCTTATATTACTGTGCAAGACAGGGGGGGACCTACTATAGGCACGACGACGCTCCTATGGACTACTGGGGTCAAGGAACCTCAGTCACCGTCTCCTCAGCCAAAACGACACCCCCATCTGTCTATCCACTGGCCCCTGGATCTGCTGCCCAAACTAACTCCATGGTGACCCTGGGATGCCTGGTCAAGGGCTATTTCCCTGAGCCAGTGACAGTGACCTGGAACTCTGGATCCCTGTCCAGCGGTGTGCACACCTTCCCAGCTGTCCTGCAGTCTGACCTCTACACTCTGAGCAGCTCAGTGACTGTCCCCTCCAGCACCTGGCCCAGCGAGACCGTCACCTGCAACGTTGCCCACCCGGCCAGCAGCACCAAGGTGGACAAGAAAATTGTGCCCAGGGATTGTGGTTGTAAGCCTTGCATATGTACAGTCCCAGAAGTATCATCTGTCTTCATCTTCCCCCCAAAGCCCAAGGATGTGCTCACCATTACTCTGACTCCTAAGGTCACGTGTGTTGTGGTAGACATCAGCAAGGATGATCCCGAGGTCCAGTTCAGCTGGTTTGTAGATGATGTGGAGGTGCACACAGCTCAGACGCAACCCCGGGAGGAGCAGTTCAACAGCACTTTCCGCTCAGTCAGTGAACTTCCCATCATGCACCAGGACTGGCTCAATGGCAAGGAGTTCAAATGCAGGGTCAACAGTGCAGCTTTCCCTGCCCCCATCGAGAAAACCATCTCCAAAACCAAAGGCAGACCGAAGGCTCCACAGGTGTACACCATTCCACCTCCCAAGGAGCAGATGGCCAAGGATAAAGTCAGTCTGACCTGCATGATAACAGACTTCTTCCCTGAAGACATTACTGTGGAGTGGCAGTGGAATGGGCAGCCAGCGGAGAACTACAAGAACACTCAGCCCATCATGGACACAGATGGCTCTTACTTCGTCTACAGCAAGCTCAATGTGCAGAAGAGCAACTGGGAGGCAGGAAATACTTTCACCTGCTCTGTGTTACATGAGGGCCTGCACAACCACCATACTGAGAAGAGCCTCTCCCACTCTCCTGGTAAATGA

SEQ ID NO: 112 - protein, 238 aa, synthetic construct
MESDTLLLWVLLLWVPGSTGDIILSQSPASVAVSLGQRATISCRASESVEYYGRSLMQWYQQKPGQPPKLLIYVASNVESGVPARFSGSGSGTDFSLNIHPVEEDDVAMYFCQQSRKVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 113 - protein, 218 aa, synthetic construct
DIILSQSPASVAVSLGQRATISCRASESVEYYGRSLMQWYQQKPGQPPKLLIYVASNVESGVPARFSGSGSGTDFSLNIHPVEEDDVAMYFCQQSRKVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 114 - protein, 111 aa, synthetic construct
DIILSQSPASVAVSLGQRATISCRASESVEYYGRSLMQWYQQKPGQPPKLLIYVASNVESGVPARFSGSGSGTDFSLNIHPVEEDDVAMYFCQQSRKVPWTFGGGTKLEIK

SEQ ID NO: 115 - protein, 15 aa, synthetic construct
RASESVEYYGRSLMQ

SEQ ID NO: 116 - protein, 7 aa, synthetic construct
VASNVES

SEQ ID NO: 117 - protein, 9 aa, synthetic construct
QQSRKVPWT

SEQ ID NO: 118 - protein, 717 aa, synthetic construct
ATGGAGTCAGACACACTCCTGCTATGGGTGCTGCTGCTCTGGGTTCCAGGCTCCACTGGTGACATTATACTCTCCCAATCTCCAGCTTCTGTGGCTGTGTCTCTGGGGCAGAGAGCCACCATCTCCTGCAGAGCCAGTGAAAGTGTTGAATATTATGGCAGAAGTTTAATGCAGTGGTACCAACAGAAACCAGGACAGCCACCCAAACTCCTCATCTATGTTGCATCCAACGTAGAATCTGGGGTCCCTGCCAGGTTTAGTGGCAGTGGGTCTGGGACAGACTTCAGCCTCAACATCCATCCTGTGGAGGAGGATGATGTTGCAATGTATTTCTGTCAGCAAAGTAGGAAGGTTCCTTGGACGTTCGGTGGAGGCACCAAGCTGGAAATCAAACGGGCTGATGCTGCACCAACTGTATCCATCTTCCCACCATCCAGTGAGCAGTTAACATCTGGAGGTGCCTCAGTCGTGTGCTTCTTGAACAACTTCTACCCCAAAGACATCAATGTCAAGTGGAAGATTGATGGCAGTGAACGACAAAATGGCGTCCTGAACAGTTGGACTGATCAGGACAGCAAAGACAGCACCTACAGCATGAGCAGCACCCTCACGTTGACCAAGGACGAGTATGAACGACATAACAGCTATACCTGTGAGGCCACTCACAAGACATCAACTTCACCCATTGTCAAGAGCTTCAACAGGAATGAGTGTTAG

SEQ ID NO: 119 - protein, 457 aa, synthetic construct
MEWTWVFLFLLSVTAGVHSQFQLQQSGGELMKPGASVKISCKATGYTFSNYWIEWMKQRPGHGLEWIGEILPGSGITKYNEKFKGKATFTADPSSNTAYMQLSSLTSEDSAVYYCSRGGWDGYWGQGTTLVASAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 120 - protein, 438 aa, synthetic construct
QFQLQQSGGELMKPGASVKISCKATGYTFSNYWIEWMKQRPGHGLEWIGEILPGSGITKYNEKFKGKATFTADPSSNTAYMQLSSLTSEDSAVYYCSRGGWDGYWGQGTTLVASAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 121 - protein, 114 aa, synthetic construct
QFQLQQSGGELMKPGASVKISCKATGYTFSNYWIEWMKQRPGHGLEWIGEILPGSGITKYNEKFKGKATFTADPSSNTAYMQLSSLTSEDSAVYYCSRGGWDGYWGQGTTLVAS

SEQ ID NO: 122 - protein, 5 aa, synthetic construct
NYWIE

SEQ ID NO: 123 - protein, 17 aa, synthetic construct
EILPGSGITKYNEKFKG

SEQ ID NO: 124 - protein, 6 aa, synthetic construct
GGWDGY

SEQ ID NO: 125 - protein, 1377 aa, synthetic construct
ATGGAATGGACCTGGGTCTTTCTCTTCCTCCTGTCAGTAACTGCAGGTGTCCACTCCCAGTTTCAGCTGCAGCAGTCTGGAGGTGAGCTGATGAAGCCTGGGGCCTCAGTGAAGATATCCTGCAAGGCTACTGGCTACACATTCAGTAACTACTGGATAGAGTGGATGAAGCAGAGGCCTGGACATGGCCTTGAGTGGATTGGAGAGATTTTACCTGGAAGTGGTATTACTAAATACAATGAGAAGTTCAAGGGCAAGGCCACATTCACTGCAGATCCATCCTCCAACACAGCCTACATGCAACTCAGTAGCCTGACATCTGAGGACTCTGCCGTCTATTACTGTTCAAGAGGGGGCTGGGACGGCTACTGGGGCCAAGGCACCACTCTCACAGTCGCCTCAGCCAAAACGACACCCCCATCTGTCTATCCACTGGCCCCTGGATCTGCTGCCCAAACTAACTCCATGGTGACCCTGGGATGCCTGGTCAAGGGCTATTTCCCTGAGCCAGTGACAGTGACCTGGAACTCTGGATCCCTGTCCAGCGGTGTGCACACCTTCCCAGCTGTCCTGCAGTCTGACCTCTACACTCTGAGCAGCTCAGTGACTGTCCCCTCCAGCACCTGGCCCAGCGAGACCGTCACCTGCAACGTTGCCCACCCGGCCAGCAGCACCAAGGTGGACAAGAAAATTGTGCCCAGGGATTGTGGTTGTAAGCCTTGCATATGTACAGTCCCAGAAGTATCATCTGTCTTCATCTTCCCCCCAAAGCCCAAGGATGTGCTCACCATTACTCTGACTCCTAAGGTCACGTGTGTTGTGGTAGACATCAGCAAGGATGATCCCGAGGTCCAGTTCAGCTGGTTTGTAGATGATGTGGAGGTGCACACAGCTCAGACGCAACCCCGGGAGGAGCAGTTCAACAGCACTTTCCGCTCAGTCAGTGAACTTCCCATCATGCACCAGGACTGGCTCAATGGCAAGGAGTTCAAATGCAGGGTCAACAGTGCAGCTTTCCCTGCCCCCATCGAGAAAACCATCTCCAAAACCAAAGGCAGACCGAAGGCTCCACAGGTGTACACCATTCCACCTCCCAAGGAGCAGATGGCCAAGGATAAAGTCAGTCTGACCTGCATGATAACAGACTTCTTCCCTGAAGACATTACTGTGGAGTGGCAGTGGAATGGGCAGCCAGCGGAGAACTACAAGAACACTCAGCCCATCATGGACACAGATGGCTCTTACTTCGTCTACAGCAAGCTCAATGTGCAGAAGAGCAACTGGGAGGCAGGAAATACTTTCACCTGCTCTGTGTTACATGAGGGCCTGCACAACCACCATACTGAGAAGAGCCTCTCCCACTCTCCTGGTAAATGA

SEQ ID NO: 126 - protein, 233 aa, synthetic construct
MVLAQFLAFLLLWFPGVRCDILMTQSPSSMSVSLGDTVSITCHANQDISSNIGWLQQKPGKSFQGLIYHGTNLEDGVSSRFSGSGSGTDYSLTISSLESEDFADYYCVQYAQFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 127 - protein, 214 aa, synthetic construct
DILMTQSPSSMSVSLGDTVSITCHANQDISSNIGWLQQKPGKSFQGLIYHGTNLEDGVSSRFSGSGSGTDYSLTISSLESEDFADYYCVQYAQFPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 128 - protein, 107 aa, synthetic construct
DILMTQSPSSMSVSLGDTVSITCHANQDISSNIGWLQQKPGKSFQGLIYHGTNLEDGVSSRFSGSGSGTDYSLTISSLESEDFADYYCVQYAQFPYTFGGGTKLEIK

SEQ ID NO: 129 - protein, 11 aa, synthetic construct
HANQDISSNIG

SEQ ID NO: 130 - protein, 7 aa, synthetic construct
HGTNLED

SEQ ID NO: 131 - protein, 9 aa, synthetic construct
VQYAQFPYT

SEQ ID NO: 132 - protein, 702 aa, synthetic construct
ATGGTCCTTGCTCAGTTTCTTGCATTCTTGTTGCTTTGGTTTCCAGGTGTAAGATGTGACATCCTGATGACCCAATCTCCTTCCTCCATGTCTGTATCTCTGGGAGACACAGTCAGCATCACTTGCCATGCAAATCAGGACATTAGTAGTAATATAGGGTGGTTGCAGCAGAAACCAGGGAAATCATTTCAGGGCCTGATCTATCATGGAACCAACTTGGAAGATGGAGTTTCATCAAGGTTCAGTGGCAGTGGATCTGGAACAGATTATTCTCTCACCATCAGCAGTCTGGAATCTGAAGATTTTGCAGACTATTATTGTGTACAGTATGCTCAGTTTCCGTACACGTTCGGAGGGGGGACCAAGTTGGAAATAAAACGGGCTGATGCTGCACCAACTGTATCCATCTTCCCACCATCCAGTGAGCAGTTAACATCTGGAGGTGCCTCAGTCGTGTGCTTCTTGAACAACTTCTACCCCAAAGACATCAATGTCAAGTGGAAGATTGATGGCAGTGAACGACAAAATGGCGTCCTGAACAGTTGGACTGATCAGGACAGCAAAGACAGCACCTACAGCATGAGCAGCACCCTCACGTTGACCAAGGACGAGTATGAACGACATAACAGCTATACCTGTGAGGCCACTCACAAGACATCAACTTCACCCATTGTCAAGAGCTTCAACAGGAATGAGTGTTAG

SEQ ID NO: 133 - protein, 464 aa, synthetic construct
MDWLWNLLFLMAAAQSAQAQIQLVQSGPELKKPGETVKISCKASGYTFTKFGLNWVKQAPGEGLKWMGWLNTYTGEPTYAVDFRGRFAFSLETSASTAYLQIDNLKNEDMATYFCATGGTTVVANPFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 134 - protein, 445 aa, synthetic construct
QIQLVQSGPELKKPGETVKISCKASGYTFTKFGLNWVKQAPGEGLKWMGWLNTYTGEPTYAVDFRGRFAFSLETSASTAYLQIDNLKNEDMATYFCATGGTTVVANPFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 135 - protein, 121 aa, synthetic construct
QIQLVQSGPELKKPGETVKISCKASGYTFTKFGLNWVKQAPGEGLKWMGWLNTYTGEPTYAVDFRGRFAFSLETSASTAYLQIDNLKNEDMATYFCATGGTTVVANPFAYWGQGTLVTVSA

SEQ ID NO: 136 - protein, 5 aa, synthetic construct
KFGLN

SEQ ID NO: 137 - protein, 17 aa, synthetic construct
WLNTYTGEPTYAVDFRG

SEQ ID NO: 138 - protein, 12 aa, synthetic construct
GGTTVVANPFAY

SEQ ID NO: 139 - DNA, 1395 bp, synthetic construct
  source 1..1395: mol_type = other DNA
atggattggctgtggaacttgctattcctgatggcagctgcccaaagtgcccaagcacagatccagttggtgcagtctggacctgaactgaagaagcctggagagacagtcaagatctcctgcaaggcttctgggtataccttcacaaagtttggactgaactgggtgaagcaggctccaggagagggtttaaagtggatgggctggctaaacacctacactggagagccaacatatgctgttgacttcaggggacgatttgccttctctttggaaacctctgccagcactgcctatttgcagatcgacaacctcaaaaatgaggacatggctacatatttctgtgcgaccggaggaactacggtagtagccaatccgtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 140 - protein, 234 aa, synthetic construct
MRFQVQVLGLLLLWISGAQCDVQITQSPSSLAASPGETITINCRASKSISKYLAWYQEKPGKTNKLLIYSGSTLRSGVPSRFSGSGSGTDFTLTISSLEPEDFAMYYCQQHDEYPITFGAGTILELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 141 - protein, 214 aa, synthetic construct
DVQITQSPSSLAASPGETITINCRASKSISKYLAWYQEKPGKTNKLLIYSGSTLRSGVPSRFSGSGSGTDFTLTISSLEPEDFAMYYCQQHDEYPITFGAGTILELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 142 - protein, 107 aa, synthetic construct
DVQITQSPSSLAASPGETITINCRASKSISKYLAWYQEKPGKTNKLLIYSGSTLRSGVPSRFSGSGSGTDFTLTISSLEPEDFAMYYCQQHDEYPITFGAGTILELK

SEQ ID NO: 143 - protein, 11 aa, synthetic construct
RASKSISKYLA

SEQ ID NO: 144 - protein, 7 aa, synthetic construct
SGSTLRS

SEQ ID NO: 145 - protein, 9 aa, synthetic construct
QQHDEYPIT

SEQ ID NO: 146 - DNA, 705 bp, synthetic construct
  source 1..705: mol_type = other DNA
atgaggttccaggttcaggttctggggctccttctgctctggatatcaggtgcccagtgtgatgtccagataacccagtctccatcttctcttgctgcatctcctggagaaaccattactattaattgcagggcaagtaagagcattagcaaatatttagcctggtatcaagagaaacctgggaaaactaataaacttcttatctactctggatccactttgcgatctggagttccatcaaggttcagtggcagtggatctggtacagatttcactctcaccatcagtagcctggagcctgaagattttgcaatgtattactgtcaacagcatgatgaatacccgatcacgttcggtgctgggaccattctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 147 - protein, 469 aa, synthetic construct
MEWSWVFLFFLSVTTGVHSQVQLQQSDAELVKPGASVKISCKASGYTFTDHAIHWVKQKPEQGLEWIGYISPGNGDIKYNEKFEGKATLTADKSSSTAYIQLNSLTSEDCAVYFCIEFYMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 148 - protein, 450 aa, synthetic construct
QVQLQQSDAELVKPGASVKISCKASGYTFTDHAIHWVKQKPEQGLEWIGYISPGNGDIKYNEKFEGKATLTADKSSSTAYIQLNSLTSEDCAVYFCIEFYMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 149 - protein, 114 aa, synthetic construct
QVQLQQSDAELVKPGASVKISCKASGYTFTDHAIHWVKQKPEQGLEWIGYISPGNGDIKYNEKFEGKATLTADKSSSTAYIQLNSLTSEDCAVYFCIEFYMDYWGQGTSVTVSS

SEQ ID NO: 150 - protein, 5 aa, synthetic construct
DHAIH

SEQ ID NO: 151 - protein, 17 aa, synthetic construct
YISPGNGDIKYNEKFEG

SEQ ID NO: 152 - protein, 5 aa, synthetic construct
FYMDY

SEQ ID NO: 153 - DNA, 1410 bp, synthetic construct
  source 1..1410: mol_type = other DNA
atggaatggagctgggtctttctcttcttcctgtcagtaactacaggtgtccactcccaggttcagctgcagcagtctgacgctgagttggtgaaacctggggcttcagtgaagatatcctgcaaggcttctggctacaccttcactgaccatgctatccactgggtaaagcagaagcctgaacagggcctggaatggattggatacatttctcccggaaatggtgatattaagtataatgagaagttcgagggcaaggccacactgactgcagacaaatcctccagcactgcctacatacagctcaacagcctgacatctgaggattgtgcagtgtatttctgtattgaattttatatggactactggggtcaaggaacctcagtcaccgtctcctcagccaaaacaacacccccatcagtctatccactggcccctgggtgtggagatacaactggttcctccgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagaggattacaacagtactatccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatatagcaagctcaatatgaaaacaagcaagtgggagaaaacagattccttctcatgcaacgtgagacacgagggtctgaaaaattactacctgaagaagaccatctcccggtctccgggtaaatga

SEQ ID NO: 154 - protein, 238 aa, synthetic construct
MSPAQFLFLLVLWIRETNGDVVMTQIPLTLSVTIGQPASISCKSSQSLLYSDGQTYLNWVLQRPGQSPKRLIYVVSKVDSGVPDRFTGSGSGTDFTLKISRVEAEDLGVYYCWQGTHFPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 155 - protein, 219 aa, synthetic construct
DVVMTQIPLTLSVTIGQPASISCKSSQSLLYSDGQTYLNWVLQRPGQSPKRLIYVVSKVDSGVPDRFTGSGSGTDFTLKISRVEAEDLGVYYCWQGTHFPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 156 - protein, 112 aa, synthetic construct
DVVMTQIPLTLSVTIGQPASISCKSSQSLLYSDGQTYLNWVLQRPGQSPKRLIYVVSKVDSGVPDRFTGSGSGTDFTLKISRVEAEDLGVYYCWQGTHFPFTFGSGTKLEIK

SEQ ID NO: 157 - protein, 16 aa, synthetic construct
KSSQSLLYSDGQTYLN

SEQ ID NO: 158 - protein, 7 aa, synthetic construct
VVSKVDS

SEQ ID NO: 159 - protein, 9 aa, synthetic construct
WQGTHFPFT

SEQ ID NO: 160 - DNA, 717 bp, synthetic construct
  source 1..717: mol_type = other DNA
atgagtcctgcccagttcctgtttctgttagtgctctggattcgggaaaccaacggtgatgttgtgatgacccagattccactcactttgtcggttaccattggacaaccagcctccatctcttgcaagtcaagtcagagcctcttatatagtgatggtcagacatatttgaactgggtgttacagaggccaggccagtctccaaagcgcctaatctatgtggtgtctaaagtggactctggagtccctgacaggttcactggcagtggatcagggacagatttcacactgaaaatcagcagagtggaggctgaggatttgggagtttattattgctggcaaggtacacattttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 161 - protein, 460 aa, synthetic construct
MRVLILLCLFTAFPGILSVELQESGPDLVKPSQSLSLTCTVTGWSIASGYSWHWIRQFPGNRLEWMAYIHYSGSINYNPSLKSRISITRDTSKNQFFLQLNSVTTEDTATYYCARNYDYPYFFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 162 - protein, 442 aa, synthetic construct
VELQESGPDLVKPSQSLSLTCTVTGWSIASGYSWHWIRQFPGNRLEWMAYIHYSGSINYNPSLKSRISITRDTSKNQFFLQLNSVTTEDTATYYCARNYDYPYFFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 163 - protein, 118 aa, synthetic construct
VELQESGPDLVKPSQSLSLTCTVTGWSIASGYSWHWIRQFPGNRLEWMAYIHYSGSINYNPSLKSRISITRDTSKNQFFLQLNSVTTEDTATYYCARNYDYPYFFDYWGQGTTLTVSS

SEQ ID NO: 164 - protein, 6 aa, synthetic construct
SGYSWH

SEQ ID NO: 165 - protein, 16 aa, synthetic construct
YIHYSGSINYNPSLKS

SEQ ID NO: 166 - protein, 10 aa, synthetic construct
NYDYPYFFDY

SEQ ID NO: 167 - DNA, 1386 bp, synthetic construct
  source 1..1386: mol_type = other DNA
atgagagtgctgattcttttgtgcctgttcacagcctttcctggtatcctgtcttatgtggaacttcaggagtcaggacctgacctggtgaaaccttctcagtcactttcactcacctgcactgtcactggctggtccatcgccagtggttatagctggcactggatccggcagtttccaggaaacagactggaatggatggcctacatacactacagtggtagtattaattacaacccatctctcaaaagtcgcatctctatcactcgagacacatccaagaaccagttcttcctgcaattgaattctgtgactactgaggacacagccacatattactgtgcacgaaactatgattacccgtacttctttgactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 168 - protein, 234 aa, synthetic construct
MESQIQVFVFVFLWLSGVDGDIVMTQSHKFMSTSEGDRVSLTCRASQNVNNAVAWYQQKPGQSPKLLIYSTSYRYSGVPDRFTGSGSGTDFTFTISSVQAEDLAVYYCQQHYNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 169 - protein, 214 aa, synthetic construct
DIVMTQSHKFMSTSEGDRVSLTCRASQNVNNAVAWYQQKPGQSPKLLIYSTSYRYSGVPDRFTGSGSGTDFTFTISSVQAEDLAVYYCQQHYNYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 170 - protein, 107 aa, synthetic construct
DIVMTQSHKFMSTSEGDRVSLTCRASQNVNNAVAWYQQKPGQSPKLLIYSTSYRYSGVPDRFTGSGSGTDFTFTISSVQAEDLAVYYCQQHYNYPLTFGAGTKLELK

SEQ ID NO: 171 - protein, 11 aa, synthetic construct
RASQNVNNAVA

SEQ ID NO: 172 - protein, 7 aa, synthetic construct
STSYRYS

SEQ ID NO: 173 - protein, 9 aa, synthetic construct
QQHYNYPLT

SEQ ID NO: 174 - DNA, 705 bp, synthetic construct
  source 1..705: mol_type = other DNA
atggagtcacagattcaggtctttgtattcgtgtttctctggttgtctggtgttgacggagacattgtgatgacccagtctcacaaattcatgtccacttcagaaggagacagggtcagcctcacctgcagggccagtcaaaatgtgaataatgctgtagcctggtatcaacagaaaccaggacaatctcctaaattgctgatttattcgacatcctaccggtactctggagtccctgatcgcttcactggcagtggatctgggacggatttcactttcaccatcagcagtgtgcaggctgaagacctggcagtctattactgtcagcaacattataattatccgctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 175 - protein, 458 aa, synthetic construct
MEWTWVFLFLLSVTAGVHSQFQLQQSGGELMKPGASVKISCKATGYTFSNYWIEWMKQRPGHGLEWIGEILPGSGITKYNEKFKGKATFTADPSSNTAYMQLSSLTSEDSAVYYCSRGGWDGYWGQGTTLTVASAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 176 - protein, 439 aa, synthetic construct
QFQLQQSGGELMKPGASVKISCKATGYTFSNYWIEWMKQRPGHGLEWIGEILPGSGITKYNEKFKGKATFTADPSSNTAYMQLSSLTSEDSAVYYCSRGGWDGYWGQGTTLTVASAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 177 - protein, 115 aa, synthetic construct
QFQLQQSGGELMKPGASVKISCKATGYTFSNYWIEWMKQRPGHGLEWIGEILPGSGITKYNEKFKGKATFTADPSSNTAYMQLSSLTSEDSAVYYCSRGGWDGYWGQGTTLTVAS

SEQ ID NO: 178 - protein, 5 aa, synthetic construct
NYWIE

SEQ ID NO: 179 - protein, 17 aa, synthetic construct
EILPGSGITKYNEKFKG

SEQ ID NO: 180 - protein, 6 aa, synthetic construct
GGWDGY

SEQ ID NO: 181 - DNA, 1377 bp, synthetic construct
  source 1..1377: mol_type = other DNA
atggaatggacctgggtctttctcttcctcctgtcagtaactgcaggtgtccactcccagtttcagctgcagcagtctggaggtgagctgatgaagcctggggcctcagtgaagatatcctgcaaggctactggctacacattcagtaactactggatagagtggatgaagcagaggcctggacatggccttgagtggattggagagattttacctggaagtggtattactaaatacaatgagaagttcaagggcaaggccacattcactgcagatccatcctccaacacagcctacatgcaactcagtagcctgacatctgaggactctgccgtctattactgttcaagagggggctgggacggctactggggccaaggcaccactctcacagtcgcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 182 - protein, 233 aa, synthetic construct; identical to SEQ ID NO: 126

SEQ ID NO: 183 - protein, 214 aa, synthetic construct; identical to SEQ ID NO: 127

SEQ ID NO: 184 - protein, 107 aa, synthetic construct; identical to SEQ ID NO: 128

SEQ ID NO: 185 - protein, 10 aa, synthetic construct
HANQDISSNI

SEQ ID NO: 186 - protein, 7 aa, synthetic construct
HGTNLED

SEQ ID NO: 187 - protein, 9 aa, synthetic construct
VQYAQFPYT

SEQ ID NO: 188 - DNA, 702 bp, synthetic construct
  source 1..702: mol_type = other DNA
atggtccttgctcagtttcttgcattcttgttgctttggtttccaggtgtaagatgtgacatcctgatgacccaatctccttcctccatgtctgtatctctgggagacacagtcagcatcacttgccatgcaaatcaggacattagtagtaatatagggtggttgcagcagaaaccagggaaatcatttcagggcctgatctatcatggaaccaacttggaagatggagtttcatcaaggttcagtggcagtggatctggaacagattattctctcaccatcagcagtctggaatctgaagattttgcagactattattgtgtacagtatgctcagtttccgtacacgttcggaggggggaccaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 189 - protein, 457 aa, synthetic construct
MEWSWVSLFFLSVTTGVHSQVQLQQSDAELVKPGASVKISCKVSGYTFSDHTLHWMKQRPEQGLEWIGYIYLRNGSVNYNERFKGKATLTADRSSTTAYMQLNSLTSEDSAVFFCARGNFAVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 190 - protein, 438 aa, synthetic construct
QVQLQQSDAELVKPGASVKISCKVSGYTFSDHTLHWMKQRPEQGLEWIGYIYLRNGSVNYNERFKGKATLTADRSSTTAYMQLNSLTSEDSAVFFCARGNFAVWGTGTTVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 191 - protein, 114 aa, synthetic construct
QVQLQQSDAELVKPGASVKISCKVSGYTFSDHTLHWMKQRPEQGLEWIGYIYLRNGSVNYNERFKGKATLTADRSSTTAYMQLNSLTSEDSAVFFCARGNFAVWGTGTTVTVSS

SEQ ID NO: 192 - protein, 5 aa, synthetic construct
DHTLH

SEQ ID NO: 193 - protein, 17 aa, synthetic construct
YIYLRNGSVNYNERFKG

SEQ ID NO: 194 - protein, 5 aa, synthetic construct
GNFAV

SEQ ID NO: 195 - DNA, 1374 bp, synthetic construct
  source 1..1374: mol_type = other DNA
atggaatggagctgggtctctctcttcttcctgtcagtaactacaggtgtccactcccaggttcagctgcaacagtcagacgctgagttggtgaaacctggagcttcagtgaagatatcctgcaaggtttctggctacaccttctctgaccatactcttcactggatgaagcagaggcctgaacagggcctggaatggattggatatatttatcttagaaatggtagtgttaactacaatgagaggttcaagggcaaggccacgttgactgcagacagatcctccaccacagcctacatgcagctcaacagcctgacatctgaggactctgcagtctttttctgtgcaagagggaacttcgctgtctggggcacagggaccacggtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 196 - protein, 238 aa, synthetic construct
MKLPVRLLVLMFWIPASSSDVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 197 - protein, 219 aa, synthetic construct
DVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 198 - protein, 112 aa, synthetic construct
DVVMTQIPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPFTFGSGTKLEIK

SEQ ID NO: 199 - protein, 16 aa, synthetic construct
RSSQSLVHSNGNTYLH

SEQ ID NO: 200 - protein, 7 aa, synthetic construct
KVSNRFS

SEQ ID NO: 201 - protein, 9 aa, synthetic construct
SQSTHVPFT

SEQ ID NO: 202 - DNA, 717 bp, synthetic construct
  source 1..717: mol_type = other DNA
atgaagttgcctgttaggctgttggtgctgatgttctggattcctgcttccagcagtgatgttgtgatgacccaaattccactctccctgcctgtcagtcttggagatcaagcctccatctcttgcagatctagtcagagccttgtacacagtaatggaaacacctatttacattggtacctgcagaagccaggccagtctccaaagctcctgatctataaagtttccaaccgattttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 203 - protein, 464 aa, synthetic construct
MGWSCIILILVAAATGVHSQVQLQQPGAELVKPGASVKMSCKASGYTFTSYWITWVKQRPGQGLEWIGDIYPGSGSTTYNEKFKSKATLTVDTSSSTAYMQLSSLTSEDSAVYYCARRHYGSSSAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 204 - protein, 445 aa, synthetic construct
QVQLQQPGAELVKPGASVKMSCKASGYTFTSYWITWVKQRPGQGLEWIGDIYPGSGSTTYNEKFKSKATLTVDTSSSTAYMQLSSLTSEDSAVYYCARRHYGSSSAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 205 - protein, 121 aa, synthetic construct
QVQLQQPGAELVKPGASVKMSCKASGYTFTSYWITWVKQRPGQGLEWIGDIYPGSGSTTYNEKFKSKATLTVDTSSSTAYMQLSSLTSEDSAVYYCARRHYGSSSAWFAYWGQGTLVTVSA

SEQ ID NO: 206 - protein, 5 aa, synthetic construct
SYWIT

SEQ ID NO: 207 - protein, 17 aa, synthetic construct
DIYPGSGSTTYNEKFKS

SEQ ID NO: 208 - protein, 12 aa, synthetic construct
RHYGSSSAWFAY

SEQ ID NO: 209 - DNA, 1395 bp, synthetic construct
  source 1..1395: mol_type = other DNA
atgggatggagctgtatcatcctcattttggtagcagcagctacaggtgtccactcccaggtccaactgcagcagcctggggctgagcttgtgaagcctggggcttcagtgaagatgtcctgcaaggcttctggctacaccttcaccagctactggataacctgggtgaagcagaggcctggacaaggccttgagtggattggagatatttatcctggtagtggtagtactacctacaatgagaagttcaagagcaaggccacactgactgttgacacatcctccagcacagcctacatgcagctcagcagcctgacatctgaggactctgcggtctattactgtgcaagaaggcactacggtagtagctcggcctggtttgcttactggggccaagggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 210 - protein, 233 aa, synthetic construct
MSSAQFLGLLLLCFQGTRCDIQMTQTTSSLSASLGDRVTISCSASQGIRNYLNWYQQKPDGTVKLLIYYTSSLHSGVPSRFSGSGSGTDYSLTISNLEPEDIATYYCQQYSKLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 211 - protein, 214 aa, synthetic construct
DIQMTQTTSSLSASLGDRVTISCSASQGIRNYLNWYQQKPDGTVKLLIYYTSSLHSGVPSRFSGSGSGTDYSLTISNLEPEDIATYYCQQYSKLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 212 - protein, 107 aa, synthetic construct
DIQMTQTTSSLSASLGDRVTISCSASQGIRNYLNWYQQKPDGTVKLLIYYTSSLHSGVPSRFSGSGSGTDYSLTISNLEPEDIATYYCQQYSKLPFTFGSGTKLEIK

SEQ ID NO: 213 - protein, 11 aa, synthetic construct
SASQGIRNYLN

SEQ ID NO: 214 - protein, 7 aa, synthetic construct
YTSSLHS

SEQ ID NO: 215 - protein, 9 aa, synthetic construct
QQYSKLPFT

SEQ ID NO: 216 - DNA, 702 bp, synthetic construct
  source 1..702: mol_type = other DNA
atgtcctctgctcagttccttggtctcctgttgctctgttttcaaggtaccagatgtgatatccagatgacacagactacatcctccctgtctgcctctctgggagacagagtcaccatcagttgcagtgcaagtcagggcattaggaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttactattgtcagcagtatagtaagcttccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 217 - protein, 464 aa, synthetic construct
MGWSCIILILVAAATGVHSQVQLQQPGAELVKPGASVKMSCKASGYTFTSYWITWVKQRPGQGLEWIGDIYPGSGSTTYNEKFKSKATLTVDTSSSTAYMQFSSLTSEDSAVYYCARRHYGSTSAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 218 - protein, 445 aa, synthetic construct
QVQLQQPGAELVKPGASVKMSCKASGYTFTSYWITWVKQRPGQGLEWIGDIYPGSGSTTYNEKFKSKATLTVDTSSSTAYMQFSSLTSEDSAVYYCARRHYGSTSAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 219 - protein, 121 aa, synthetic construct
QVQLQQPGAELVKPGASVKMSCKASGYTFTSYWITWVKQRPGQGLEWIGDIYPGSGSTTYNEKFKSKATLTVDTSSSTAYMQFSSLTSEDSAVYYCARRHYGSTSAWFAYWGQGTLVTVSA

SEQ ID NO: 220 - protein, 5 aa, synthetic construct
SYWIT

SEQ ID NO: 221 - protein, 17 aa, synthetic construct
DIYPGSGSTTYNEKFKS

SEQ ID NO: 222 - protein, 12 aa, synthetic construct
RHYGSTSAWFAY

SEQ ID NO: 223 - DNA, 1395 bp, synthetic construct
  source 1..1395: mol_type = other DNA
atgggatggagctgtatcatcctcattttggtagcagcagctacaggtgtccactcccaggtccaactgcagcagcctggggctgagcttgtgaagcctggggcttcagtgaagatgtcctgcaaggcttctggctacaccttcaccagctactggataacctgggtgaagcagaggcctggacaaggccttgagtggattggagatatttatcctggtagtggtagtactacctacaatgagaagttcaagagcaaggccacactgactgttgacacatcctccagcacagcctacatgcagttcagcagcctgacatctgaggactctgcggtctattactgtgcaagaaggcattacggtagtacctcggcctggtttgcttactggggccaggggactctggtcactgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 224 - protein, 233 aa, synthetic construct
MSSAQFLGLLLLCFQGTRCDIQMTQTTSSLSASLGDRVTISCSASQGIRNYLNWYQQKPDGTVKLLIYYTSSLHSGVPSRFSGSGSGTDYSLTISNLEPEDIATYFCQQYSKLPFTFGSGTQLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 225 - protein, 214 aa, synthetic construct
DIQMTQTTSSLSASLGDRVTISCSASQGIRNYLNWYQQKPDGTVKLLIYYTSSLHSGVPSRFSGSGSGTDYSLTISNLEPEDIATYFCQQYSKLPFTFGSGTQLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 226 - protein, 107 aa, synthetic construct
DIQMTQTTSSLSASLGDRVTISCSASQGIRNYLNWYQQKPDGTVKLLIYYTSSLHSGVPSRFSGSGSGTDYSLTISNLEPEDIATYFCQQYSKLPFTFGSGTQLEIK

SEQ ID NO: 227 - protein, 11 aa, synthetic construct
SASQGIRNYLN

SEQ ID NO: 228 - protein, 7 aa, synthetic construct
YTSSLHS

SEQ ID NO: 229 - protein, 9 aa, synthetic construct
QQYSKLPFT

SEQ ID NO: 230 - DNA, 702 bp, synthetic construct
  source 1..702: mol_type = other DNA
atgtcctctgctcagttccttggtctcctgttgctctgttttcaaggtaccagatgtgatatccagatgacacagactacatcctccctgtctgcctctctgggagacagagtcaccatcagttgcagtgcaagtcagggcattaggaattatttaaactggtatcagcagaaaccagatggaactgttaaactcctgatctattacacatcaagtttacactcaggagtcccatcaaggttcagtggcagtgggtctgggacagattattctctcaccatcagcaacctggaacctgaagatattgccacttacttttgtcagcaatatagtaagcttccattcacgttcggctcggggacacagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 231 - protein, 457 aa, synthetic construct
MEWSWVSLFFLSLTTGVHSQVQLQQSDAELVKPGASVKISCKVSGYTFTDHTIHWMKQWPEQGLEWIGYIYPRDGGTKYSEKFKGKATLTADKSSSTASMQLNRLTSEDSAVYFCARGQLDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 232 - protein, 438 aa, synthetic construct
QVQLQQSDAELVKPGASVKISCKVSGYTFTDHTIHWMKQWPEQGLEWIGYIYPRDGGTKYSEKFKGKATLTADKSSSTASMQLNRLTSEDSAVYFCARGQLDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 233 - protein, 114 aa, synthetic construct
QVQLQQSDAELVKPGASVKISCKVSGYTFTDHTIHWMKQWPEQGLEWIGYIYPRDGGTKYSEKFKGKATLTADKSSSTASMQLNRLTSEDSAVYFCARGQLDYWGQGTTLTVSS

SEQ ID NO: 234 - protein, 5 aa, synthetic construct
DHTIH

SEQ ID NO: 235 - protein, 17 aa, synthetic construct
YIYPRDGGTKYSEKFKG

SEQ ID NO: 236 - protein, 5 aa, synthetic construct
GQLDY

SEQ ID NO: 237 - DNA, 1374 bp, synthetic construct
  source 1..1374: mol_type = other DNA
atggaatggagctgggtctctctcttcttcctgtcactaactacaggtgtccactcccaggttcagctgcaacagtctgacgctgagttggtgaaacctggagcttcagtgaagatatcctgcaaggtttctggctacaccttcactgaccatactattcactggatgaaacagtggcctgaacagggcctggaatggattggatatatttatcctagagatggtggtactaagtacagtgagaagttcaagggcaaggccacattgactgcagacaaatcctccagcacagcctccatgcagctcaacaggctgacatctgaggactctgcagtctatttctgtgcaagaggtcaactagactactggggccaaggcaccactctcacagtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 238 - protein, 238 aa, synthetic construct
MKLPVRLLVLMFWIPASSSDVVMTQTPLSLPASLGDQASISCRSSQSLVHSSGNTYLYWYLQKPGQSPKLLIYRVSNRFSGVPDRFSGSGSGTDFTLKISRVEADDLGVYFCSQSTHVPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 239 - protein, 219 aa, synthetic construct
DVVMTQTPLSLPASLGDQASISCRSSQSLVHSSGNTYLYWYLQKPGQSPKLLIYRVSNRFSGVPDRFSGSGSGTDFTLKISRVEADDLGVYFCSQSTHVPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 240 - protein, 112 aa, synthetic construct
DVVMTQTPLSLPASLGDQASISCRSSQSLVHSSGNTYLYWYLQKPGQSPKLLIYRVSNRFSGVPDRFSGSGSGTDFTLKISRVEADDLGVYFCSQSTHVPFTFGGGTKLEIK

SEQ ID NO: 241 - protein, 16 aa, synthetic construct
RSSQSLVHSSGNTYLY

SEQ ID NO: 242 - protein, 7 aa, synthetic construct
RVSNRFS

SEQ ID NO: 243 - protein, 9 aa, synthetic construct
SQSTHVPFT

SEQ ID NO: 244 - DNA, 717 bp, synthetic construct
  source 1..717: mol_type = other DNA
atgaagttgcctgttaggctgttggtgctgatgttctggattcctgcttccagcagtgatgttgtgatgacccaaactccactctccctgcctgccagtcttggagatcaagcctccatctcttgcagatctagtcagagccttgtacacagtagtggaaacacctatttatattggtatctgcagaagccaggccagtctccaaagctcctgatctacagagtttccaaccgattttctggggtcccagacaggttcagtggcagtggatcagggacagatttcacactcaagatcagcagagtggaggctgacgatctgggagtttatttctgctctcaaagtacacatgttccgttcacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 245 - protein, 465 aa, synthetic construct
MELCWVFLFLLSVTAGVHSQVQLQQSGAELVKPGASVKLSCKSSGNSFNEYIINWVKQRPGQGLEWIGWFYPGSGTKKCNEKFKAKATLTADKSSSTVYMELSGLTSEDSAVYFCTRHEYYNGSSLGFAYWGQGTLVSVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 246 - protein, 446 aa, synthetic construct
QVQLQQSGAELVKPGASVKLSCKSSGNSFNEYIINWVKQRPGQGLEWIGWFYPGSGTKKCNEKFKAKATLTADKSSSTVYMELSGLTSEDSAVYFCTRHEYYNGSSLGFAYWGQGTLVSVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTKPREEQINSTFRSVSELPIMHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 247 - protein, 122 aa, synthetic construct
QVQLQQSGAELVKPGASVKLSCKSSGNSFNEYIINWVKQRPGQGLEWIGWFYPGSGTKKCNEKFKAKATLTADKSSSTVYMELSGLTSEDSAVYFCTRHEYYNGSSLGFAYWGQGTLVSVSA

SEQ ID NO: 248 - protein, 5 aa, synthetic construct
EYIIN

SEQ ID NO: 249 - protein, 17 aa, synthetic construct
WFYPGSGTKKCNEKFKA

SEQ ID NO: 250 - protein, 13 aa, synthetic construct
HEYYNGSSLGFAY

SEQ ID NO: 251 - DNA, 1398 bp, synthetic construct
  source 1..1398: mol_type = other DNA
atggaattgtgctgggtctttctcttcctcctgtcagtaactgcaggtgtccactcccaggtccagcttcagcagtctggagctgaactggtgaaacccggggcatcagtgaagctgtcctgcaagtcttctggcaactccttcaatgagtatattataaattgggttaaacagaggcctggacagggtcttgagtggatcgggtggttttaccctggaagcggtactaaaaagtgcaatgagaaatttaaggccaaggccacattgactgcggacaaatcctccagtacagtctacatggaactaagtggattgacatctgaagactctgcggtctatttctgtacaagacacgaatattataacggaagtagtctagggtttgcttactggggccaagggacactggtcagtgtctctgcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagccagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgaaaccccgggaggagcagatcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacaaacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 252 - protein, 239 aa, synthetic construct
MRCLAEFLGLLVLWIPGAIGDIVMTQAAPSVTVTPGESVTISCRSSKSLLHSYGSTYLYWFLQRPGQSPQLLIYRMSNLASGVPDRFSGSGSGTAFTLRISRVEAEDVGVYYCMQHLEYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 253 - protein, 219 aa, synthetic construct
DIVMTQAAPSVTVTPGESVTISCRSSKSLLHSYGSTYLYWFLQRPGQSPQLLIYRMSNLASGVPDRFSGSGSGTAFTLRISRVEAEDVGVYYCMQHLEYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 254 - protein, 112 aa, synthetic construct
DIVMTQAAPSVTVTPGESVTISCRSSKSLLHSYGSTYLYWFLQRPGQSPQLLIYRMSNLASGVPDRFSGSGSGTAFTLRISRVEAEDVGVYYCMQHLEYPLTFGAGTKLELK

SEQ ID NO: 255 - protein, 16 aa, synthetic construct
RSSKSLLHSYGSTYLY

SEQ ID NO: 256 - protein, 7 aa, synthetic construct
RMSNLAS

SEQ ID NO: 257 - protein, 9 aa, synthetic construct
MQHLEYPLT

SEQ ID NO: 258 - DNA, 720 bp, synthetic construct
  source 1..720: mol_type = other DNA
atgaggtgcctagctgagttcctggggctgcttgtgctctggatccctggagccattggggatattgtgatgactcaggctgcaccctctgtaactgtcactcctggagagtcagtaaccatctcctgcaggtctagtaagagtctcctccatagttatggcagcacttacttgtattggttcctacagaggccaggccagtctcctcaactcctgatatatcggatgtccaaccttgcctcaggagtcccagacaggttcagtggcagtgggtcaggaactgctttcacactgagaatcagcagagtggaggctgaggatgtgggtgtttattactgtatgcaacatctagaatatccgctcacgttcggtgctgggaccaagctggagttgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 259 - protein, 478 aa, synthetic construct
MYFRLSSVFLVLILKGVQCEVKLVESEGGLVQPGSSMKLSCTASGFTFSDYYMAWVRQVPEKGLEWVANINYDGSSTYYLDSLKSRFIISRDNAKNILYLQMSSLKSEDTATYYCARDSYYYGRSYGYFDVWGTGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 260 - protein, 459 aa, synthetic construct
EVKLVESEGGLVQPGSSMKLSCTASGFTFSDYYMAWVRQVPEKGLEWVANINYDGSSTYYLDSLKSRFIISRDNAKNILYLQMSSLKSEDTATYYCARDSYYYGRSYGYFDVWGTGTTVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 261 - protein, 123 aa, synthetic construct
EVKLVESEGGLVQPGSSMKLSCTASGFTFSDYYMAWVRQVPEKGLEWVANINYDGSSTYYLDSLKSRFIISRDNAKNILYLQMSSLKSEDTATYYCARDSYYYGRSYGYFDVWGTGTTVTVSS

SEQ ID NO: 262 - protein, 5 aa, synthetic construct
DYYMA

SEQ ID NO: 263 - protein, 17 aa, synthetic construct
NINYDGSSTYYLDSLKS

SEQ ID NO: 264 - protein, 14 aa, synthetic construct
DSYYYGRSYGYFDV

SEQ ID NO: 265 - DNA, 1437 bp, synthetic construct
  source 1..1437: mol_type = other DNA
atgtacttcaggctcagctcagtttttcttgttcttattttaaaaggagtccagtgtgaagtgaagctggtggagtctgagggaggcttagtgcagcctggaagttccatgaaactctcctgcacagcctctggattcactttcagtgactattacatggcttgggtccgccaggttccagaaaagggtcttgaatgggttgcaaacattaattatgatggtagtagtacctactatctggactccttgaagagccgtttcatcatctcgagagacaatgcaaagaacattctatacctgcaaatgagcagtctgaagtctgaggacacagccacgtattactgtgcaagagattcctattactacggtcgtagctacgggtacttcgatgtctggggcacagggaccacggtcaccgtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 266 - protein, 239 aa, synthetic construct
MDSQAQVLMLLLLWVSGTCGDIVMSQSPSSLAVSVGEKVTMSCKSSQRLLYSSNQKNYLAWYQQKPGQSPKLLIYWASTRESGVPDRFTGSGSGTDFTLTISSVKAEDLAVYYCQQYYSYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCETHKTSTSPIVKSFNRNEC

SEQ ID NO: 267 - protein, 219 aa, synthetic construct
DIVMSQSPSSLAVSVGEKVTMSCKSSQRLLYSSNQKNYLAWYQQKPGQSPKLLIYWASTRESGVPDRFTGSGSGTDFTLTISSVKAEDLAVYYCQQYYSYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCETHKTSTSPIVKSFNRNEC

SEQ ID NO: 268 - protein, 113 aa, synthetic construct
DIVMSQSPSSLAVSVGEKVTMSCKSSQRLLYSSNQKNYLAWYQQKPGQSPKLLIYWASTRESGVPDRFTGSGSGTDFTLTISSVKAEDLAVYYCQQYYSYPPTFGAGTKLELK

SEQ ID NO: 269 - protein, 17 aa, synthetic construct
KSSQRLLYSSNQKNYLA

SEQ ID NO: 270 - protein, 7 aa, synthetic construct
WASTRES

SEQ ID NO: 271 - protein, 9 aa, synthetic construct
QQYYSYPPT

SEQ ID NO: 272 - DNA, 723 bp, synthetic construct
  source 1..723: mol_type = other DNA
atggattcacaggcccaggttcttatgttactgctgctatgggtatctggtacctgtggggacattgtgatgtcacagtctccatcctccctagctgtgtcagttggagagaaggttactatgagctgcaagtccagtcagagacttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctcccacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 273 - protein, 477 aa, synthetic construct
MYFRLSSVFLVLILKGVQCEVKLVESEGGLVQPGSSMKLSCTASGFTFSDYYMAWVRQVPEKGLEWVANINYDGSSTYYLDSLKSRFIISRDNAKNILYLQMSSLKSDDTATYYCARDSYYYGRFYGYFDVWGTGTTVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 274 - protein, 458 aa, synthetic construct
EVKLVESEGGLVQPGSSMKLSCTASGFTFSDYYMAWVRQVPEKGLEWVANINYDGSSTYYLDSLKSRFIISRDNAKNILYLQMSSLKSDDTATYYCARDSYYYGRFYGYFDVWGTGTTVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 275 - protein, 123 aa, synthetic construct
EVKLVESEGGLVQPGSSMKLSCTASGFTFSDYYMAWVRQVPEKGLEWVANINYDGSSTYYLDSLKSRFIISRDNAKNILYLQMSSLKSDDTATYYCARDSYYYGRFYGYFDVWGTGTTVTVSS

SEQ ID NO: 276 - protein, 5 aa, synthetic construct
DYYMA

SEQ ID NO: 277 - protein, 17 aa, synthetic construct
NINYDGSSTYYLDSLKS

SEQ ID NO: 278 - protein, 14 aa, synthetic construct
DSYYYGRFYGYFDV

SEQ ID NO: 279 - DNA, 1434 bp, synthetic construct
  source 1..1434: mol_type = other DNA
atgtacttcaggctcagctcagtttttcttgttcttattttaaaaggagttcagtgtgaagtgaagctggtggagtctgagggaggcttagtgcagcctggaagttccatgaaactctcctgcacagcctctggattcactttcagtgactattacatggcttgggtccgccaggttccagaaaagggtcttgagtgggttgcaaacattaattatgatggtagtagcacctactatctggactccttgaagagccgtttcatcatctcgagagacaatgcaaagaacattctatacctgcaaatgagcagtctgaagtctgacgacacagccacgtattactgtgcacgagattcctattactacggtcgtttctacgggtacttcgatgtctggggcacagggaccacggtcaccgtctcctcagccaaaacaacagccccatcggtctatccactggcccctgtgtgtggaggtacaactggctcctcggtgactctaggatgcctggtcaagggttatttccctgagccagtgaccttgacctggaactctggatccctgtccagtggtgtgcacaccttcccagctctcctgcagtctggcctctacaccctcagcagctcagtgactgtaacctcgaacacctggcccagccagaccatcacctgcaatgtggcccacccggcaagcagcaccaaagtggacaagaaaattgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 280 - protein, 240 aa, synthetic construct
MDSQAQVLMLLLLWVSGTCGDIVMSQSPSSLAVSVGEKVTMSCKSSQRLLYSTSQKNYLAWYQQKPGQSPKLLIYWASTRESGVPDRFTGSGSGTDFTLTISSVKAEDLAVYYCQQYYSYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 281 - protein, 220 aa, synthetic construct
DIVMSQSPSSLAVSVGEKVTMSCKSSQRLLYSTSQKNYLAWYQQKPGQSPKLLIYWASTRESGVPDRFTGSGSGTDFTLTISSVKAEDLAVYYCQQYYSYPPTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 282 - protein, 113 aa, synthetic construct
DIVMSQSPSSLAVSVGEKVTMSCKSSQRLLYSTSQKNYLAWYQQKPGQSPKLLIYWASTRESGVPDRFTGSGSGTDFTLTISSVKAEDLAVYYCQQYYSYPPTFGAGTKLELK

SEQ ID NO: 283 - protein, 17 aa, synthetic construct
KSSQRLLYSTSQKNYLA

SEQ ID NO: 284 - protein, 7 aa, synthetic construct
WASTRES

SEQ ID NO: 285 - protein, 9 aa, synthetic construct
QQYYSYPPT

SEQ ID NO: 286 - DNA, 723 bp, synthetic construct
  source 1..723: mol_type = other DNA
atggattcacaggcccaggttcttatgttactgctgctatgggtatctggtacctgtggggacattgtgatgtcacagtctccatcctccctagctgtgtcagttggagagaaggttactatgagctgcaagtccagtcagagacttttatatagtaccagtcaaaagaactacttggcctggtaccagcagaagccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctcccacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 287 - protein, 469 aa, synthetic construct
MGWSWIFLFLLSETAGVLSEVQLQQSGPELVKPGASVKMSCKASGYTFTDSNMHWVKQSLGKSLEWIGYINTNNGGTTYNQKFKGTATLTVNKSSSTAYMELRSLTSEDSAVYYCARADGYYWGQGTTLSVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 288 - protein, 450 aa, synthetic construct
EVQLQQSGPELVKPGASVKMSCKASGYTFTDSNMHWVKQSLGKSLEWIGYINTNNGGTTYNQKFKGTATLTVNKSSSTAYMELRSLTSEDSAVYYCARADGYYWGQGTTLSVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 289 - protein, 114 aa, synthetic construct
EVQLQQSGPELVKPGASVKMSCKASGYTFTDSNMHWVKQSLGKSLEWIGYINTNNGGTTYNQKFKGTATLTVNKSSSTAYMELRSLTSEDSAVYYCARADGYYWGQGTTLSVSS

SEQ ID NO: 290 - protein, 5 aa, synthetic construct
DSNMH

SEQ ID NO: 291 - protein, 17 aa, synthetic construct
YINTNNGGTTYNQKFKG

SEQ ID NO: 292 - protein, 5 aa, synthetic construct
ADGYY

SEQ ID NO: 293 - DNA, 1410 bp, synthetic construct
  source 1..1410: mol_type = other DNA
atgggatggagctggatctttctctttctcctgtcagaaactgcaggtgtcctctctgaggtccagctgcaacagtctggacctgagctggtgaagcctggggcttcagtgaagatgtcctgcaaggcttctggatacacattcactgactccaacatgcactgggtgaaacagagccttggaaagagccttgagtggattggttatattaacactaacaacggtggtactacctacaaccagaagttcaagggcacggccacattgactgtaaacaagtcctccagcacagcctacatggagctccgcagcctgacatcggaggattctgcagtctattactgtgcaagagccgatggttactactggggccaaggcaccactctctcagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 294 - protein, 234 aa, synthetic construct
MESQTQVFVYMLLWLSGVDGDIVMTQSQKFMSTSVGDRVSVTCKASQNVGTNVAWYQQKPGQSPKALIYSASSRYSGVPDRLTGSGSGTDFTLTISNVQSEDLAEYFCQQYNSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 295 - protein, 214 aa, synthetic construct
DIVMTQSQKFMSTSVGDRVSVTCKASQNVGTNVAWYQQKPGQSPKALIYSASSRYSGVPDRLTGSGSGTDFTLTISNVQSEDLAEYFCQQYNSFPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 296 - protein, 107 aa, synthetic construct
DIVMTQSQKFMSTSVGDRVSVTCKASQNVGTNVAWYQQKPGQSPKALIYSASSRYSGVPDRLTGSGSGTDFTLTISNVQSEDLAEYFCQQYNSFPLTFGAGTKLELK

SEQ ID NO: 297 - protein, 11 aa, synthetic construct
KASQNVGTNVA

SEQ ID NO: 298 - protein, 7 aa, synthetic construct
SASSRYS

SEQ ID NO: 299 - protein, 9 aa, synthetic construct
QQYNSFPLT

SEQ ID NO: 300 - DNA, 705 bp, synthetic construct
  source 1..705: mol_type = other DNA
atggagtcacagactcaggtctttgtatacatgttgctgtggttgtctggtgttgatggagacattgtgatgacccagtctcaaaaattcatgtccacatcagtaggagacagggtcagcgtcacctgcaaggccagtcagaatgtgggtactaatgtagcctggtatcaacagaaaccagggcaatctcctaaagcactgatttactcggcatcctcccggtacagtggagtccctgatcgcctcacaggcagtggatctgggacagatttcactctcaccatcagcaatgtgcagtctgaggacttggcagagtatttctgtcagcaatataacagctttcctctcacgttcggtgctgggaccaagctggagctgaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 301 - protein, 469 aa, synthetic construct
MGWSWIFLFLLSETAGVLSEVQLQQSGPELVKPGASVKMSCKASGYTFTDYSFHWVKQSLGKSLEWIGYINPNNGGSSYNQKFKGTATLTVNKSSSTAYMELRSLTSEDSAVYYCARADGYYWGQGTILTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 302 - protein, 450 aa, synthetic construct
EVQLQQSGPELVKPGASVKMSCKASGYTFTDYSFHWVKQSLGKSLEWIGYINPNNGGSSYNQKFKGTATLTVNKSSSTAYMELRSLTSEDSAVYYCARADGYYWGQGTILTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 303 - protein, 114 aa, synthetic construct
EVQLQQSGPELVKPGASVKMSCKASGYTFTDYSFHWVKQSLGKSLEWIGYINPNNGGSSYNQKFKGTATLTVNKSSSTAYMELRSLTSEDSAVYYCARADGYYWGQGTILTVSS

SEQ ID NO: 304 - protein, 5 aa, synthetic construct
DYSFH

SEQ ID NO: 305 - protein, 17 aa, synthetic construct
YINPNNGGSSYNQKFKG

SEQ ID NO: 306 - protein, 5 aa, synthetic construct
ADGYY

SEQ ID NO: 307 - DNA, 1410 bp, synthetic construct
  source 1..1410: mol_type = other DNA
atgggatggagctggatctttctctttctcctgtcagaaactgcaggtgtcctctctgaggtccagctgcaacagtctggacctgagctggtgaagcctggggcttcagtgaagatgtcctgcaaggcttctggatacacattcactgactacagcttccactgggtgaagcagagccttggaaagagccttgagtggattggatatattaaccctaataatggtggttcttcctacaaccagaagttcaagggcacggccacattgactgtaaacaagtcctccagcacagcctacatggaactccgcagcctgacatcggaggattctgcagtctattactgtgcaagagccgatggttattactggggccaaggcaccattctcacagtctcctcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 308 - protein, 234 aa, synthetic construct
MESQTQVFVYMLLWLSGVDGDIVMTQSQKFMSTSVGDRVSVTCKASHNVGTNVAWYQQKPGQSPKALIYSASSRFSGVPDRFAGSGSGTDFTLTISSVQSEDLAEYFCQQYNNFPLTFGAGTNLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 309 - protein, 214 aa, synthetic construct
DIVMTQSQKFMSTSVGDRVSVTCKASHNVGTNVAWYQQKPGQSPKALIYSASSRFSGVPDRFAGSGSGTDFTLTISSVQSEDLAEYFCQQYNNFPLTFGAGTNLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 310 - protein, 107 aa, synthetic construct
DIVMTQSQKFMSTSVGDRVSVTCKASHNVGTNVAWYQQKPGQSPKALIYSASSRFSGVPDRFAGSGSGTDFTLTISSVQSEDLAEYFCQQYNNFPLTFGAGTNLELK

SEQ ID NO: 311 - protein, 11 aa, synthetic construct
KASHNVGTNVA

SEQ ID NO: 312 - protein, 7 aa, synthetic construct
SASSRFS

SEQ ID NO: 313 - protein, 9 aa, synthetic construct
QQYNNFPLT

SEQ ID NO: 314 - DNA, 705 bp, synthetic construct
  source 1..705: mol_type = other DNA
atggagtcacagactcaggtctttgtatacatgttgctgtggttgtctggtgttgatggagacattgtgatgacccagtctcaaaaattcatgtccacatcagtaggagacagggtcagcgtcacctgcaaggccagtcacaatgtgggtactaatgtagcctggtatcaacagaagccagggcaatctcctaaagcactgatttactcggcttcctcccggttcagtggagtccctgatcgcttcgcaggcagtggatctgggacagatttcactctcaccatcagcagtgtgcagtctgaagacttggcagagtatttctgtcaacaatataacaactttcctctcacgttcggtgctgggaccaacctggagctgaagcgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 315 - protein, 476 aa, synthetic construct
MGWSCIILILVAAASGVHSQVQLQQPGTELVKPGASVSLSCKTSGYTFTNYWMHWVKQRPGQGLEWIGNINPSNGRSNYIEKFKRKATLTVDTSSSTAYMQLSSLTSEDSAVYFCTRGDYDSDSSWFDYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 316 - protein, 457 aa, synthetic construct
QVQLQQPGTELVKPGASVSLSCKTSGYTFTNYWMHWVKQRPGQGLEWIGNINPSNGRSNYIEKFKRKATLTVDTSSSTAYMQLSSLTSEDSAVYFCTRGDYDSDSSWFDYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 317 - protein, 121 aa, synthetic construct
QVQLQQPGTELVKPGASVSLSCKTSGYTFTNYWMHWVKQRPGQGLEWIGNINPSNGRSNYIEKFKRKATLTVDTSSSTAYMQLSSLTSEDSAVYFCTRGDYDSDSSWFDYWGQGTLVTVSA

SEQ ID NO: 318 - protein, 5 aa, synthetic construct
NYWMH

SEQ ID NO: 319 - protein, 17 aa, synthetic construct
NINPSNGRSNYIEKFKR

SEQ ID NO: 320 - protein, 12 aa, synthetic construct
GDYDSDSSWFDY

SEQ ID NO: 321 - DNA, 1431 bp, synthetic construct
  source 1..1431: mol_type = other DNA
atgggatggagctgtatcatcctcattttggtagcagcagcttcaggtgtccactcccaggtccaactgcagcagcctgggactgaactggtgaagcctggggcttcagtgagcctgtcctgtaagacttctggctacaccttcaccaactactggatgcactgggtgaaacagaggcctggacagggccttgagtggattggaaatattaatccaagcaatggtcgtagtaactacatagagaagttcaagagaaaggccacactgactgtcgacacatcctccagcacagcctacatgcagctcagcagtctgacatctgaggactctgcggtctatttttgtacacgaggggactacgatagtgactcctcctggtttgattactggggccaagggactctggtcactgtctctgcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 322 - protein, 237 aa, synthetic construct
MDFHVQIFSFMLISVTVMLSSGEFVLTQSPAVLAAFPGEKVTITCSVNAFLSSSDLHWYQQRSEASPKPLIYGTSNLASGVPVRFSGSGSGTSFSLTISSMEAEDAATYYCQQWSTYPLTFGAGTKLELKRADAAPTVSIFSPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 323 - protein, 215 aa, synthetic construct
EFVLTQSPAVLAAFPGEKVTITCSVNAFLSSSDLHWYQQRSEASPKPLIYGTSNLASGVPVRFSGSGSGTSFSLTISSMEAEDAATYYCQQWSTYPLTFGAGTKLELKRADAAPTVSIFSPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 324 - protein, 108 aa, synthetic construct
EFVLTQSPAVLAAFPGEKVTITCSVNAFLSSSDLHWYQQRSEASPKPLIYGTSNLASGVPVRFSGSGSGTSFSLTISSMEAEDAATYYCQQWSTYPLTFGAGTKLELK

SEQ ID NO: 325 - protein, 12 aa, synthetic construct
SVNAFLSSSDLH

SEQ ID NO: 326 - protein, 7 aa, synthetic construct
GTSNLAS

SEQ ID NO: 327 - protein, 9 aa, synthetic construct
QQWSTYPLT

SEQ ID NO: 328 - DNA, 714 bp, synthetic construct
  source 1..714: mol_type = other DNA
atggattttcatgtgcagattttcagcttcatgctaatcagtgtcacagtcatgttgtccagtggagaatttgtgctcacccagtctccagcagtcttggctgcatttccgggggagaaggtcaccatcacctgcagtgtcaacgcatttctaagttccagcgacttgcactggtaccagcagaggtcagaagcctcccccaaacccttgatttatggcacatccaacctggcttctggagtccctgttcgcttcagtggcagtggatctgggacctctttctctctcacaatcagcagcatggaggcggaagatgctgccacttattactgtcaacagtggagtacttacccgctcacgttcggtgctgggaccaagctggagctgaaacgggctgatgctgcaccaactgtatccatcttctcaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 329 - protein, 471 aa, synthetic construct
MKCSWIIFFLMAVVTGVNSEVQLQQSGAELVKPGASVKLSCTSSGFNIKDYYIHWVKQRTESGLEWIGRIDPEDVATKYAPKFQGKATMTADTSSNTAYLQLSSLTSEDAAVYYCTFSHYGNYGWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 330 - protein, 452 aa, synthetic construct
EVQLQQSGAELVKPGASVKLSCTSSGFNIKDYYIHWVKQRTESGLEWIGRIDPEDVATKYAPKFQGKATMTADTSSNTAYLQLSSLTSEDAAVYYCTFSHYGNYGWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVRISWFVNNVEVHTAQTQTHREDYNSTIRVVSALPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLDIKTSKWEKTDSFSCNVRHEGLKNYYLKKTISRSPGK

SEQ ID NO: 331 - protein, 116 aa, synthetic construct
EVQLQQSGAELVKPGASVKLSCTSSGFNIKDYYIHWVKQRTESGLEWIGRIDPEDVATKYAPKFQGKATMTADTSSNTAYLQLSSLTSEDAAVYYCTFSHYGNYGWGQGTLVTVSA

SEQ ID NO: 332 - protein, 5 aa, synthetic construct
DYYIH

SEQ ID NO: 333 - protein, 17 aa, synthetic construct
RIDPEDVATKYAPKFQG

SEQ ID NO: 334 - protein, 7 aa, synthetic construct
SHYGNYG

SEQ ID NO: 335 - DNA, 1416 bp, synthetic construct
  source 1..1416: mol_type = other DNA
atgaaatgcagctggatcatcttcttcctgatggcagtggttacaggggtcaattcagaggttcagctgcagcaatctggggcagaactcgtgaagccgggggcctcagtcaagttgtcctgcacatcttctggcttcaacattaaagactactatatacactgggtgaagcagaggactgaatcgggcctggaatggattggaaggattgatcctgaagatgttgcaactaaatatgccccgaaattccagggcaaggccactatgacagcagacacatcctccaacacagcctacctccagctcagcagcctgacatctgaggacgctgccgtctattattgtacttttagtcattatggtaactacgggtggggccaggggactctggtcactgtctctgcagccaaaacaacacccccatcagtctatccactggcccctgggtgtggagatacaactggttcctctgtgactctgggatgcctggtcaagggctacttccctgagtcagtgactgtgacttggaactctggatccctgtccagcagtgtgcacaccttcccagctctcctgcagtctggactctacactatgagcagctcagtgactgtcccctccagcacctggccaagtcagaccgtcacctgcagcgttgctcacccagccagcagcaccacggtggacaaaaaacttgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccggatcagctggtttgtgaacaacgtggaagtacacacagctcagacacaaacccatagagaggattacaacagtactatccgggtggtcagtgccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactacaaggacaccgcaccagtcctggactctgacggttcttacttcatatacagcaagctcgatataaaaacaagcaagtgggagaaaacagattccttctcatgcaacgtgagacacgagggtctgaaaaattactacctgaagaagaccatctcccggtctccgggtaaatga

SEQ ID NO: 336 - protein, 234 aa, synthetic construct
MESQTQVFVYMLLWLSGVDGDTVMTQSQKFMSSSIGDRVSVTCKASKNVGISVAWYQQKPGQSPKALIYSASYRYSGVPDRFTGSGSGTDFTLTINNVQSEDLAEYFCQQYNGYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 337 - protein, 214 aa, synthetic construct
DTVMTQSQKFMSSSIGDRVSVTCKASKNVGISVAWYQQKPGQSPKALIYSASYRYSGVPDRFTGSGSGTDFTLTINNVQSEDLAEYFCQQYNGYPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 338 - protein, 107 aa, synthetic construct
DTVMTQSQKFMSSSIGDRVSVTCKASKNVGISVAWYQQKPGQSPKALIYSASYRYSGVPDRFTGSGSGTDFTLTINNVQSEDLAEYFCQQYNGYPFTFGSGTKLEIK

SEQ ID NO: 339 - protein, 11 aa, synthetic construct
KASKNVGISVA

SEQ ID NO: 340 - protein, 7 aa, synthetic construct
SASYRYS

SEQ ID NO: 341 - protein, 9 aa, synthetic construct
QQYNGYPFT

SEQ ID NO: 342 - DNA, 705 bp, synthetic construct
  source 1..705: mol_type = other DNA
atggagtcacagactcaggtctttgtatacatgttgctgtggttgtctggtgttgatggagacactgtgatgacccagtctcaaaaattcatgtcctcatcaataggagacagggtcagcgtcacctgcaaggccagtaagaatgtgggtattagtgtcgcctggtatcagcagaaaccagggcaatctcctaaagcattgatttactcggcatcctaccggtacagtggagtccctgatcgcttcacaggcagtggatctgggacagatttcactctcaccatcaacaatgtgcagtctgaagacttggcagaatatttctgtcagcaatataacggctatccattcacgttcggctcggggacaaagttggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 343 - protein, 10 aa, synthetic construct
TKIESLKEHG

SEQ ID NO: 344 - protein, 10 aa, synthetic construct
TQIENLKEKG

SEQ ID NO: 345 - protein, 26 aa, synthetic construct
AALEALAEALEALAEALEALAEAAAA

SEQ ID NO: 346 - protein, 25 aa, synthetic construct
GLFEAIEGFIENGWEGMIEGWYGCG

SEQ ID NO: 347 - protein, 16 aa, synthetic construct
RQIKIWFQNRRMKWKK

SEQ ID NO: 348 - protein, 13 aa, synthetic construct
GRKKRRQRRRPPQ

SEQ ID NO: 349 - protein, 26 aa, synthetic construct
GIGAVLKVLTTGLPALISWIKRKRQQ

SEQ ID NO: 350 - protein, 12 aa, synthetic construct
HGHVDPVKTPKI

(19) United States
(12) Patent Application Publication
(10) Pub. No.: US 2026/0335407 A1
(43) Pub. Date: Oct. 29, 2026

(54) COMPOSITIONS AND METHODS FOR TREATING A PATHOGEN-INDUCED INFECTION USING A MEGF9 PROTEIN OR A BIOLOGICALLY ACTIVE FRAGMENT THEREOF

(71) Applicant: BOARD OF REGENTS, THE UNIVERSITY OF TEXAS SYSTEM, Austin, TX (US)

(72) Inventor: Sreerama SHETTY, Tyler, TX (US)

(73) Assignee: BOARD OF REGENTS, THE UNIVERSITY OF TEXAS SYSTEM, Austin, TX (US)

(21) Appl. No.: 19/281,283

(22) Filed: Jul. 25, 2025

Related U.S. Application Data
(63) Continuation-in-part of application No. 18/837,857, filed on Feb. 13, 2023.
(60) Provisional application No. 63/309,728, filed on Feb. 14, 2022.
(60) Provisional application No. 63/479,575, filed on Jan. 12, 2023.

(51) Int. Cl. A61K 38/18; A61K 45/06; A61P 11/00; A61P 31/14; A61P 31/16

(52) U.S. Cl. CPC A61K 38/1808; A61K 45/06; A61P 11/00; A61P 31/14; A61P 31/16

(57) ABSTRACT

Compositions, methods, and processes for making and using a MEGF9 protein or a biologically active fragment of MEGF9 protein for treating a subject in need of therapy for a pathogen-induced infection or a pathogen-induced tissue injury. The disclosure includes treating the influenza virus pathogen-induced lung injury in the subject by administering to the subject a sequence consisting of: HGHVDPVKTPKI (SEQ ID NO: 350).

### CROSS-REFERENCE TO RELATED APPLICATIONS

This application is a continuation-in-part of U.S. application Ser. No. 18/837,857, filed on Aug. 12, 2024, which is a 35 U.S.C. § 371 National Phase Entry Application from PCT/US2023/062474, filed on Feb. 13, 2023, designating the United states, and claims the benefit of U.S. Provisional Application No. 63/309,728, filed Feb. 14, 2022 and U.S. Provisional Application No. 63/479,575, filed Jan. 12, 2023. The entire contents of the above-identified applications are hereby fully incorporated herein by reference.

### STATEMENT REGARDING FEDERALLY SPONSORED RESEARCH

This invention was made with government support under (NIH HL151397) awarded by the National Institutes of Health and under (W81XWH-21-1-0095) awarded by the U.S. Army Medical Research and Materiel, Command, U.S. Department of Defense. The government has certain rights in the invention.

### REFERENCE TO AN ELECTRONIC SEQUENCE LISTING

This application contains a Sequence Listing that has been submitted electronically as an XML file named “4842-111US4.xml”. The XML file, created on Jul. 25, 2025 is 372,736 bytes in size. The content of the electronic sequence listing in the XML file is herein incorporated by reference in its entirety.

### TECHNICAL FIELD

The present disclosure relates generally to the fields of molecular biology and medicine. More particularly, it concerns compositions and methods for treating pathogen-induced diseases with therapeutic proteins, peptides, and/or antibodies.

### BACKGROUND

Pathogens infect and cause disease resulting in millions of deaths annually and countless comorbidities. Outbreaks of coronavirus infections including SARS-CoV-1 in 2002-2003, Middle East respiratory syndrome-related coronavirus (MERS-CoV) identified in 2012, and SARS-CoV-2 identified in 2019 have highlighted the need for new pharmaceuticals and method for battling infections and infection-induced injuries. There is no cure for coronavirus infections and frequent mutations makes vaccination less reliable. Patients that survive coronavirus diseases are likely to develop fibrosis of lungs and other organs due to incomplete repair.

In view of the continuing threat to human health, there is an urgent need for preventive and therapeutic antiviral therapies for infection control as well as therapies for inhibiting apoptosis of injured or damaged lung epithelial cells and for treating acute lung injury.

### SUMMARY OF THE INVENTION

The present disclosure includes molecules, formulations, kits, and methods for treating or preventing a pathogen-induced disease, disorder, symptom, or injury in a subject.

In one aspect, the present disclosure includes a method of treating a subject in need of therapy for a pathogen-induced infection, symptom, disease, disorder, injury, or condition, by administering to the subject:
a) multiple EGF-like-domains-9 (MEGF9) or a biologically active fragment thereof;
b) uncoordinated receptor 5A (UNC5A) or a biologically active fragment thereof;
c) dolichyl-phosphate beta-glucosyltransferase (ALG5) or a biologically active fragment thereof;
d) a combination of two or three of a)-c);
e) an antibody specific for a);
f) an antibody specific for b);
g) an antibody specific for c);
h) a combination of two or three of e)-g); or
i) a combination of at least one of a)-c) and at least one of e)-g).

In one aspect, the present disclosure includes treating a pathogen-induced infection, symptom, disease, disorder, injury, or condition is caused by a viral infection.

In one aspect, the present disclosure includes treating a subject suffering from or at risk of developing pathogen-induced acute lung injury (ALI), acute respiratory distress syndrome (ARDS), pulmonary fibrosis, COPD, asthma, pneumonia, a vascular lung disease, an interstitial lung disease, or a combination thereof. The disclosures of WO2022/217037 are incorporated herein by reference for all purposes.

In one aspect, the present disclosure includes a method or use of a medicament for treating a subject suffering from or at risk of developing an infection by a coronavirus, an influenza virus, a respiratory syncytial virus (RSV), an enterovirus (Picornaviridae), a rhinovirus, parainfluenza, a metapneumovirus, a bocavirus, an adenovirus, or a Coxsackie virus.

In some embodiments, the method comprises administering MEGF9 or a biologically active fragment thereof; and UNC5A or a biologically active fragment thereof. In some embodiments, the method comprises administering UNC5A or a biologically active fragment thereof; and ALG5 or a biologically active fragment thereof. In some embodiments, the method comprises administering MEGF9 or a biologically active fragment thereof; and ALG5 or a biologically active fragment thereof. In some embodiments, the method comprises administering MEGF9 or a biologically active fragment thereof; UNC5A or a biologically active fragment thereof; and ALG5 or a biologically active fragment thereof. In some embodiments, the or biologically active fragment of MEGF9 comprises a sequence with at least 80%, at least 85%, at least 90%, or at least 95% sequence identity to SEQ ID NO: 1. In some embodiments, the UNC5A comprises a sequence with at least 80%, at least 85%, at least 90%, or at least 95% sequence identity to SEQ ID NO: 5. In some embodiments, the ALG5 comprises a sequence with at least 80%, at least 85%, at least 90%, or at least 95% sequence identity to SEQ ID NO: 9. In some embodiments, the ALG5 comprises a sequence with at least 80%, at least 85%, at least 90%, or at least 95% sequence identity to SEQ ID NO: 102. In some embodiments, the biologically active fragment of the ALG5 comprises a sequence of SEQ ID NO: 1 or a one-amino acid or two-amino acid modification thereof. In some embodiments, the biologically active fragment of the ALG5 comprises a sequence of SEQ ID NO: 5 or a one-amino acid or two-amino acid modification thereof. In some embodiments, the biologically active fragment of the ALG5 comprises a sequence of SEQ ID NO: 9 or a one-amino acid or two-amino acid modification thereof. In some embodiments, the biologically active fragment of the ALG5 comprises a sequence of SEQ ID NO: 102 or a one-amino acid or two-amino acid modification thereof. In some embodiments, the biologically active fragment of the MEGF9 comprises a sequence with at least 80%, at least 85%, at least 90%, or at least 95% sequence identity to SEQ ID NO: 3. In some embodiments, the biologically active fragment of the MEGF9 consists of a sequence with at least 80%, at least 85%, at least 90%, or at least 95% sequence identity to SEQ ID NO: 3. In some embodiments, the biologically active fragment of the MEGF9 consists of a sequence with at least 80%, at least 85%, at least 90%, or at least 95% sequence identity to SEQ ID NO: 104. In some embodiments, the biologically active fragment of the MEGF9 comprises a sequence of SEQ ID NO: 4 or a one-amino acid or two-amino acid modification thereof. In some embodiments, the biologically active fragment of the MEGF9 consists of a sequence of SEQ ID NO: 4 or a one-amino acid or two-amino acid modification thereof. In some embodiments, the biologically active fragment of the MEGF9 consists of a sequence of SEQ ID NO: 103 or a one-amino acid or two-amino acid modification thereof. In some embodiments, the biologically active fragment of the UNC5A comprises a sequence with at least 80%, at least 85%, at least 90%, or at least 95% sequence identity to SEQ ID NO: 7. In some embodiments, the biologically active fragment of the UNC5A consists of a sequence with at least 80%, at least 85%, at least 90%, or at least 95% sequence identity to SEQ ID NO: 7. In some embodiments, the biologically active fragment of the UNC5A comprises a sequence with at least 80%, at least 85%, at least 90%, or at least 95% sequence identity to SEQ ID NO: 8. In some embodiments, the biologically active fragment of the UNC5A consists of a sequence with at least 80%, at least 85%, at least 90%, or at least 95% sequence identity to SEQ ID NO: 8. In some embodiments, the biologically active fragment of the UNCSA consists of a sequence with at least 80%, at least 85%, at least 90%, or at least 95% sequence identity to SEQ ID NO: 104. In some embodiments, the biologically active fragment of the UNCSA consists of a sequence of SEQ ID NO: 7 or a one-amino acid or two-amino acid modification thereof. In some embodiments, the biologically active fragment of the UNC5A consists of a sequence of SEQ ID NO: 8 or a one-amino acid or two-amino acid modification thereof. In some embodiments, the method further comprises administering a Cav1 scaffolding domain peptide (CSP). In some embodiments, the CSP comprises a sequence with at least at least 80%, at least 85%, at least 90%, or at least 95% sequence identity to SEQ ID NO: 10. In some embodiments, the biologically active fragment of the CSP comprises a sequence of SEQ ID NO: 11 or a one-amino acid modification thereof. In some embodiments, the biologically active fragment of the CSP consists of a sequence of SEQ ID NO: 11 or a one-amino acid modification thereof. In some embodiments, the pathogen-induced infection, symptom, disease, disorder, injury, or condition is caused by a viral infection. In some embodiments, the subject suffers from pathogen-induced acute lung injury (ALI), acute respiratory distress syndrome (ARDS), pulmonary fibrosis, COPD, asthma, pneumonia, a vascular lung disease, an interstitial lung disease, or a combination thereof. In some embodiments, the viral infection is an infection by an influenza virus, a respiratory syncytial virus (RSV), an enterovirus (Picornaviridae), a rhinovirus, parainfluenza, a metapneumovirus, a bocavirus, an adenovirus, or a Coxsackie virus. In some embodiments, the viral infection is a coronavirus infection. In some embodiments, the coronavirus is MERS-CoV, SARS-CoV-1, or SARS-CoV-2. In some embodiments, the coronavirus is MERS-Cov. In some embodiments, the subject has Middle East respiratory syndrome (MERS). In some embodiments, the coronavirus is SARS-CoV-1. In some embodiments, the coronavirus is SARS-CoV-2. In some embodiments, the subject has severe acute respiratory syndrome (SARS). In some embodiments, the subject has coronavirus disease 2019 (COVID-19). In some embodiments, the antibody is monoclonal antibody (mAb). In some embodiments, the method further comprises administering a), b), c), d), e), f), g), h), or i) to the subject in the form of a pharmaceutical composition that further comprises a pharmaceutically acceptable vehicle or excipient. In some embodiments, the method further administering the pharmaceutical composition via a route selected from the group consisting of intrapulmonary, intravenous, intramuscular, subcutaneous, oral, or in any combination thereof. In some embodiments, the method further administering the pharmaceutical composition via an intrapulmonary route of administration. In some embodiments, the method further administering the pharmaceutical composition via inhalation. In some embodiments, the method further administering the pharmaceutical composition in the form of a dry powder formulation. In some embodiments, the dry powder comprises less than 10% or less than 1% (by weight) of water. In some embodiments, the pharmaceutical composition is formulated for lung delivery, inhalation, or pressurized metered dose inhalation. In some embodiments, the method further comprises administering at least one additional therapeutic agent. In some embodiments, the at least one additional therapeutic agent is chloroquine, hydroxychloroquine, type I interferon, Azithromycin, Tocilizumab, sarilumab, interferon beta, anti-virals, remdesivir, baricitinib, dexamethasone, monoclonal antibodies including bamlanivimab (LY-CoV555), etesevimab, Casirivimab and imdevimab, AZD7442, VIR-7831, bemnifosbuvir (AT-527), lenzilumab, leronlimab, favipiravir, lopinavir, nirmatrelvir, molnupiravir, ritonavir, or a combination thereof.

In one aspect, the present disclosure includes vector including a nucleic acid molecule comprising one or more sequences encoding a) MEGF9 or a biologically active fragment thereof; b) UNC5A or a biologically active fragment thereof; c) ALG5 or a biologically active fragment thereof; or a combination thereof, or a cell including the vector.

In one aspect, the present disclosure includes method of producing a therapeutic protein by culturing a cell in a culture medium under a condition sufficient to produce a therapeutic protein comprising the MEGF9, UNC5A, ALG5, and/or biological fragment thereof and recovering the therapeutic protein. In some embodiments, the method further comprises isolating the therapeutic protein recovered from the cell or the culture medium. In some embodiments, the method further comprises formulating the therapeutic protein into a pharmaceutical composition. In some embodiments, the therapeutic protein is formulated as dry powder. In some embodiments, the dry powder is produced by a milling process, spray-drying process, jet milling, ball milling, or wet milling. In some embodiments, the dry powder is produced by thin film freezing. In some embodiments, the subject is a human.

In one aspect, the present disclosure includes use of an antibody specific for any one or combination of SEQ ID NOs: 1-9 and 102-104 for the treatment of an infection by a coronavirus, an influenza virus, a respiratory syncytial virus (RSV), an enterovirus (Picornaviridae), a rhinovirus, parainfluenza, a metapneumovirus, a bocavirus, an adenovirus, or a Coxsackie virus in a subject.

In one aspect, the present disclosure includes use of an antibody specific for any one of SEQ ID NOs:1-9 and 102-104 for the manufacture of a medicament for treatment of an infection by a coronavirus, an influenza virus, a respiratory syncytial virus (RSV), an enterovirus (Picornaviridae), a rhinovirus, parainfluenza, a metapneumovirus, a bocavirus, an adenovirus, or a Coxsackie virus in a subject.

In some embodiments, the medicament further comprises peptide CSP7. In some embodiments, the antibody is specific for SEQ ID NO: 1. In some embodiments, the antibody is specific for SEQ ID NO: 2. In some embodiments, the antibody is specific for SEQ ID NO: 3. In some embodiments, the antibody is specific for SEQ ID NO: 4. In some embodiments, the antibody is specific for SEQ ID NO: 5. In some embodiments, the antibody is specific for SEQ ID NO: 6. In some embodiments, the antibody is specific for SEQ ID NO: 7. In some embodiments, the antibody is specific for SEQ ID NO: 8. In some embodiments, the antibody is specific for SEQ ID NO: 9. In some embodiments, the antibody is specific for SEQ ID NO: 102. In some embodiments, the antibody is specific for SEQ ID NO: 103. In some embodiments, the antibody is specific for SEQ ID NO: 104. In some embodiments, the antibody is a mAb.

In one aspect, the present disclosure includes an M9-binding protein comprising:
a heavy chain variable region comprising
   a variable heavy chain complementarity determining region 1 (V_{H} CDR1) comprising SEQ ID NO: 108,
   a variable heavy chain complementarity determining region 2 (V_{H} CDR2) comprising SEQ ID NO: 109, and
   a variable heavy chain complementarity determining region 3 (V_{H} CDR3) comprising SEQ ID NO: 110; and
a light chain variable region comprising
   a variable light chain complementarity determining region 1 (V_{L} CDR1) comprising SEQ ID NO: 115,
   a variable light chain complementarity determining region 2 (V_{L} CDR2) comprising SEQ ID NO: 116, and
   a variable light chain complementarity determining region 3 (V_{L} CDR3) comprising a SEQ ID NO: 117.

In some embodiments, the heavy chain variable region comprises a sequence at least 80%, 85%, 90%, 95%, 99%, or 100% identical to SEQ ID NO: 107, and the light chain variable region comprises a sequence at least 80%, 85%, 90%, 95%, 99%, or 100% identical to SEQ ID NO: 114.

In one aspect, the present disclosure includes an M9-binding protein comprising:
a heavy chain variable region comprising
   a V_{H} CDR1 comprising SEQ ID NO: 122,
   a V_{H} CDR2 comprising SEQ ID NO: 123, and
   a V_{H} CDR3 comprising SEQ ID NO: 124; and
a light chain variable region comprising
   a V_{L} CDR1 comprising SEQ ID NO: 129,
   a V_{L} CDR2 comprising SEQ ID NO: 130, and
   a V_{L} CDR3 comprising a SEQ ID NO: 131.

In some embodiments, the heavy chain variable region comprises a sequence at least 80%, 85%, 90%, 95%, 99%, or 100% identical to SEQ ID NO: 121, and the light chain variable region comprises a sequence at least 80%, 85%, 90%, 95%, 99%, or 100% identical to SEQ ID NO: 128.

In one aspect, the present disclosure includes an M9-binding protein comprising:
a heavy chain variable region comprising
   a V_{H} CDR1 comprising SEQ ID NO: 136,
   a V_{H} CDR2 comprising SEQ ID NO: 137, and
   a V_{H} CDR3 comprising SEQ ID NO: 138; and
a light chain variable region comprising
   a V_{L} CDR1 comprising SEQ ID NO: 143,
   a V_{L} CDR2 comprising SEQ ID NO: 144, and
   a V_{L} CDR3 comprising a SEQ ID NO: 145.

In some embodiments, the heavy chain variable region comprises a sequence at least 80%, 85%, 90%, 95%, 99%, or 100% identical to SEQ ID NO: 135, and the light chain variable region comprises a sequence at least 80%, 85%, 90%, 95%, 99%, or 100% identical to SEQ ID NO: 142.

In one aspect, the present disclosure includes an M9-binding protein comprising:
a heavy chain variable region comprising
   a V_{H} CDR1 comprising SEQ ID NO: 150,
   a V_{H} CDR2 comprising SEQ ID NO: 151, and
   a V_{H} CDR3 comprising SEQ ID NO: 152; and
a light chain variable region comprising
   a V_{L} CDR1 comprising SEQ ID NO: 157,
   a V_{L} CDR2 comprising SEQ ID NO: 158, and
   a V_{L} CDR3 comprising a SEQ ID NO: 159.

In some embodiments, the heavy chain variable region comprises a sequence at least 80%, 85%, 90%, 95%, 99%, or 100% identical to SEQ ID NO: 149, and the light chain variable region comprises a sequence at least 80%, 85%, 90%, 95%, 99%, or 100% identical to SEQ ID NO: 156.

In one aspect, the present disclosure includes an M9-binding protein comprising:
a heavy chain variable region comprising
   a V_{H} CDR1 comprising SEQ ID NO: 164,
   a V_{H} CDR2 comprising SEQ ID NO: 165, and
   a V_{H} CDR3 comprising SEQ ID NO: 166; and
a light chain variable region comprising
   a V_{L} CDR1 comprising SEQ ID NO: 171,
   a V_{L} CDR2 comprising SEQ ID NO: 172, and
   a V_{L} CDR3 comprising a SEQ ID NO: 173.

In some embodiments, the heavy chain variable region comprises a sequence at least 80%, 85%, 90%, 95%, 99%, or 100% identical to SEQ ID NO: 163, and the light chain variable region comprises a sequence at least 80%, 85%, 90%, 95%, 99%, or 100% identical to SEQ ID NO: 170.

In one aspect, the present disclosure includes an M9-binding protein comprising:
a heavy chain variable region comprising
   a V_{H} CDR1 comprising SEQ ID NO: 178,
   a V_{H} CDR2 comprising SEQ ID NO: 179, and
   a CDR3 comprising SEQ ID NO: 180; and
a light chain variable region comprising
   a V_{L} CDR1 comprising SEQ ID NO: 185,
   a V_{L} CDR2 comprising SEQ ID NO: 186, and
   a V_{L} CDR3 comprising a SEQ ID NO: 187.

In some embodiments, the heavy chain variable region comprises a sequence at least 80%, 85%, 90%, 95%, 99%, or 100% identical to SEQ ID NO: 177, and the light chain variable region comprises a sequence at least 80%, 85%, 90%, 95%, 99%, or 100% identical to SEQ ID NO: 184.

In one aspect, the present disclosure includes an U5A-binding protein comprising:
a heavy chain variable region comprising
   a V_{H} CDR1 comprising SEQ ID NO: 192,
   a V_{H} CDR2 comprising SEQ ID NO: 193, and
   a V_{H} CDR3 comprising SEQ ID NO: 194; and
a light chain variable region comprising
   a V_{L} CDR1 comprising SEQ ID NO: 199,
   a V_{L} CDR2 comprising SEQ ID NO: 200, and
   a V_{L} CDR3 comprising a SEQ ID NO: 201.

In some embodiments, the heavy chain variable region comprises a sequence at least 80%, 85%, 90%, 95%, 99%, or 100% identical to SEQ ID NO: 191, and the light chain variable region comprises a sequence at least 80%, 85%, 90%, 95%, 99%, or 100% identical to SEQ ID NO: 198.

In one aspect, the present disclosure includes an U5A-binding protein comprising:
a heavy chain variable region comprising
   a V_{H} CDR1 comprising SEQ ID NO: 206,
   a V_{H} CDR2 comprising SEQ ID NO: 207, and
   a V_{H} CDR3 comprising SEQ ID NO: 208; and
a light chain variable region comprising
   a V_{L} CDR1 comprising SEQ ID NO: 213,
   a V_{L} CDR2 comprising SEQ ID NO: 214, and
   a V_{L} CDR3 comprising a SEQ ID NO: 215.

In some embodiments, the heavy chain variable region comprises a sequence at least 80%, 85%, 90%, 95%, 99%, or 100% identical to SEQ ID NO: 205, and the light chain variable region comprises a sequence at least 80%, 85%, 90%, 95%, 99%, or 100% identical to SEQ ID NO: 212.

In one aspect, the present disclosure includes an U5A-binding protein comprising:
a heavy chain variable region comprising
   a V_{H} CDR1 comprising SEQ ID NO: 220,
   a V_{H} CDR2 comprising SEQ ID NO: 221, and
   a V_{H} CDR3 comprising SEQ ID NO: 222; and
a light chain variable region comprising
   a V_{L} CDR1 comprising SEQ ID NO: 227,
   a V_{L} CDR2 comprising SEQ ID NO: 228, and
   a V_{L} CDR3 comprising a SEQ ID NO: 229.

In some embodiments, the heavy chain variable region comprises a sequence at least 80%, 85%, 90%, 95%, 99%, or 100% identical to SEQ ID NO: 219, and the light chain variable region comprises a sequence at least 80%, 85%, 90%, 95%, 99%, or 100% identical to SEQ ID NO: 226.

In one aspect, the present disclosure includes a U5A-binding protein comprising:
a heavy chain variable region comprising
   a V_{H} CDR1 comprising SEQ ID NO: 234,
   a V_{H} CDR2 comprising SEQ ID NO: 235, and
   a V_{H} CDR3 comprising SEQ ID NO: 236; and
a light chain variable region comprising
   a V_{L} CDR1 comprising SEQ ID NO: 241,
   a V_{L} CDR2 comprising SEQ ID NO: 242, and
   a V_{L} CDR3 comprising a SEQ ID NO: 243.

In some embodiments, the heavy chain variable region comprises a sequence at least 80%, 85%, 90%, 95%, 99%, or 100% identical to SEQ ID NO: 233, and the light chain variable region comprises a sequence at least 80%, 85%, 90%, 95%, 99%, or 100% identical to SEQ ID NO: 240.

In one aspect, the present disclosure includes a U5A-binding protein comprising:
a heavy chain variable region comprising
   a V_{H} CDR1 comprising SEQ ID NO: 248,
   a V_{H} CDR2 comprising SEQ ID NO: 249, and
   a V_{H} CDR3 comprising SEQ ID NO: 250; and
a light chain variable region comprising
   a V_{L} CDR1 comprising SEQ ID NO: 255,
   a V_{L} CDR2 comprising SEQ ID NO: 256, and
   a V_{L} CDR3 comprising a SEQ ID NO: 257.

In some embodiments, the heavy chain variable region comprises a sequence at least 80%, 85%, 90%, 95%, 99%, or 100% identical to SEQ ID NO: 247, and the light chain variable region comprises a sequence at least 80%, 85%, 90%, 95%, 99%, or 100% identical to SEQ ID NO: 254.

In one aspect, the present disclosure includes an ALG5-binding protein comprising:
a heavy chain variable region comprising
   a V_{H} CDR1 comprising SEQ ID NO: 262,
   a V_{H} CDR2 comprising SEQ ID NO: 263, and
   a V_{H} CDR3 comprising SEQ ID NO: 264; and
a light chain variable region comprising
   a V_{L} CDR1 comprising SEQ ID NO: 269,
   a V_{L} CDR2 comprising SEQ ID NO: 270, and
   a V_{L} CDR3 comprising a SEQ ID NO: 271.

In some embodiments, the heavy chain variable region comprises a sequence at least 80%, 85%, 90%, 95%, 99%, or 100% identical to SEQ ID NO: 261, and the light chain variable region comprises a sequence at least 80%, 85%, 90%, 95%, 99%, or 100% identical to SEQ ID NO: 268.

In one aspect, the present disclosure includes an ALG5-binding protein comprising:
a heavy chain variable region comprising
   a V_{H} CDR1 comprising SEQ ID NO: 276,
   a V_{H} CDR2 comprising SEQ ID NO: 277, and
   a V_{H} CDR3 comprising SEQ ID NO: 278; and
a light chain variable region comprising
   a V_{L} CDR1 comprising SEQ ID NO: 283,
   a V_{L} CDR2 comprising SEQ ID NO: 284, and
   a V_{L} CDR3 comprising a SEQ ID NO: 285.

In some embodiments, the heavy chain variable region comprises a sequence at least 80%, 85%, 90%, 95%, 99%, or 100% identical to SEQ ID NO: 275, and the light chain variable region comprises a sequence at least 80%, 85%, 90%, 95%, 99%, or 100% identical to SEQ ID NO: 282.

In one aspect, the present disclosure includes an ALG5-binding protein comprising:
a heavy chain variable region comprising
   a V_{H} CDR1 comprising SEQ ID NO: 290,
   a V_{H} CDR2 comprising SEQ ID NO: 291, and
   a V_{H} CDR3 comprising SEQ ID NO: 292; and
a light chain variable region comprising
   a V_{L} CDR1 comprising SEQ ID NO: 297,
   a V_{L} CDR2 comprising SEQ ID NO: 298, and
   a V_{L} CDR3 comprising a SEQ ID NO: 299.

In some embodiments, the heavy chain variable region comprises a sequence at least 80%, 85%, 90%, 95%, 99%, or 100% identical to SEQ ID NO: 289, and the light chain variable region comprises a sequence at least 80%, 85%, 90%, 95%, 99%, or 100% identical to SEQ ID NO: 296.

In one aspect, the present disclosure includes an ALG5-binding protein comprising:
a heavy chain variable region comprising
   a V_{H} CDR1 comprising SEQ ID NO: 304,
   a V_{H} CDR2 comprising SEQ ID NO: 305, and
   a V_{H} CDR3 comprising SEQ ID NO: 306; and
a light chain variable region comprising
   a V_{L} CDR1 comprising SEQ ID NO: 311,
   a V_{L} CDR2 comprising SEQ ID NO: 312, and
   a V_{L} CDR3 comprising a SEQ ID NO: 313.

In some embodiments, the heavy chain variable region comprises a sequence at least 80%, 85%, 90%, 95%, 99%, or 100% identical to SEQ ID NO: 303, and the light chain variable region comprises a sequence at least 80%, 85%, 90%, 95%, 99%, or 100% identical to SEQ ID NO: 310.

In one aspect, the present disclosure includes an ALG5-binding protein comprising:
a heavy chain variable region comprising
   a V_{H} CDR1 comprising SEQ ID NO: 318,
   a V_{H} CDR2 comprising SEQ ID NO: 319, and
   a V_{H} CDR3 comprising SEQ ID NO: 320; and
a light chain variable region comprising
   a V_{L} CDR1 comprising SEQ ID NO: 325,
   a V_{L} CDR2 comprising SEQ ID NO: 326, and
   a V_{L} CDR3 comprising a SEQ ID NO: 327.

In some embodiments, the heavy chain variable region comprises a sequence at least 80%, 85%, 90%, 95%, 99%, or 100% identical to SEQ ID NO: 317, and the light chain variable region comprises a sequence at least 80%, 85%, 90%, 95%, 99%, or 100% identical to SEQ ID NO: 324.

In one aspect, the present disclosure includes an ALG5-binding protein comprising:
a heavy chain variable region comprising
   a V_{H} CDR1 comprising SEQ ID NO: 332,
   a V_{H} CDR2 comprising SEQ ID NO: 333, and
   a V_{H} CDR3 comprising SEQ ID NO: 334; and
a light chain variable region comprising
   a V_{L} CDR1 comprising SEQ ID NO: 339,
   a V_{L} CDR2 comprising SEQ ID NO: 340, and
   a V_{L} CDR3 comprising a SEQ ID NO: 341.

In some embodiments, the heavy chain variable region comprises a sequence at least 80%, 85%, 90%, 95%, 99%, or 100% identical to SEQ ID NO: 331, and the light chain variable region comprises a sequence at least 80%, 85%, 90%, 95%, 99%, or 100% identical to SEQ ID NO: 338.

Other features and characteristics of the subject matter of this disclosure, as well as the methods of operation, functions of related elements of structure and the combination of parts, and economies of manufacture, will become more apparent upon consideration of the following description and the appended claims, all of which form a part of this specification.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows an antivirus assay using various concentrations of the drug remdesivir.

FIG. 2 shows an antivirus assay using various concentrations of a combination of full-length human MEGF9, UNC5A, and ALG5.

FIG. 3 shows an antivirus assay using various concentrations of a combination of ECDs of human MEGF9, UNC5A, and ALG5.

FIGS. 4A-4F show PRMNT assay results using SARS-CoV-2 WA-1 strain. FIG. 4A shows PRMNT results with use of recombinant full length MEGF9 (M9^{FL}). FIG. 4B shows PRMNT results with use of recombinant full length UNC5a (U5a^{FL}). FIG. 4C shows PRMNT results with the use of recombinant full length ALG5 (A5^{FL}). FIG. 4D shows PRMNT results with use of combined M9^{FL}, U5a^{FL}, and A5^{FL}. FIG. 4E shows PRMNT results with use of the vehicle (blank). FIG. 4F shows PRMNT results with use of Remdesivir. FIGS. 4G-4K show a repeated set of PRMNT assay results using SARS-CoV-2 WA-1 strain. FIG. 4G shows PRMNT results with use of recombinant full length MEGF9 (M9^{FL}). FIG. 4H shows PRMNT results with use of recombinant full length UNC5a (U5a^{FL}). FIG. 4I shows PRMNT results with the use of recombinant full length ALG5 (A5^{FL}). FIG. 4J shows PRMNT results with use of combined M9^{FL}, U5a^{FL}, and A5^{FL}. FIG. 4K shows PRMNT results with use of Remdesivir.

FIG. 5 shows an exemplary study design of in vivo treatment of virus infection.

FIGS. 6A-6D show results on IVIS Day 2. FIG. 6A shows whole animal Nluc images. FIG. 6B shows lung bright field images. FIG. 6C shows lung Nluc images. FIG. 6D shows lung mCherry images.

FIGS. 7A-7D show results on IVIS Day 4. FIG. 7A shows whole animal Nluc images.

FIG. 7B shows lung bright field images. FIG. 7C shows lung Nluc images. FIG. 7D shows lung mCherry images.

FIGS. 8A-8B show results of virus titrations. FIG. 8A shows virus titration in lungs. FIG. 8B shows virus titration in nasal turbinates.

FIG. 9 shows body weight change of the animals in a virus infection experiment. The difference observed is highly statistically significant. The p value is less than 0.0001 on day 6 between the Mock treated-SARS-CoV2 infected group and the M9^{FL} treated SARS-CoV2 infected group.

FIG. 10 shows survival of the animals in a virus infection experiment.

FIGS. 11A-11D show the induction of U5a expression by TSE-induced lung injury. WT mice were kept in ambient AIR or exposed to tobacco smoke (TSE) for 4 h/day 5 days a week for 20 weeks. Alveolar epithelial type II (ATII) cells were isolated. The cell lysates were immunoblotted for U5a and β-actin (loading control) protein. FIG. 11A shows U5a levels of the control and TSE groups. The total RNA isolated from ATII cell lysates were analyzed for U5a mRNA by pPCR. FIG. 11B shows the mRNA levels of the control and TSE groups. Lung sections from WT mice kept in ambient AIR or exposed to TS for 20 weeks (FIG. 11C) or Human normal lung (nL) section from control donors or from patients with COPD (FIG. 11D) were subjected to immunohistochemical (IHC) analysis using anti-U5a antibody.

FIGS. 12A-12D show results of treating mice with TSE-induced lung injury with U5a proteins and anti-U5a antibody. Total lung RNA were analyzed for MUC5AC (M5Ac) (FIG. 12A), FOXA2 (FIG. 12B) and surfactant protein-C (SP-C) (FIG. 12C) mRNA by qPCR. Lung sections of mice treated as in FIG. 12A were subjected to IHC analysis for M5Ac (FIG. 12D).

FIGS. 13A-13C show percent survival (FIG. 13A) and body weight loss (FIG. 13B) of IAV infected wild-type (WT) mice treated with or without 2.5 mg/kg recombinant full length M9 protein (M9^{FL}). Viral titer is shown by Western Blot analysis of Nucleoprotein (NP) levels from whole lung homogenates of the saline treated control animals and the IAV infected animals (with M9^{FL} treatment and without “none”) (FIG. 13C).

FIGS. 14A-14B show IAV antigen in situ using anti-NP antibody on formalin fixed and paraffin embedded lung sections of IAV infected wild-type (WT) mice treated with or without 2.5 mg/kg recombinant full length M9 protein (M9^{FL}).

FIGS. 15A-15B show Western blotting (FIG. 15A) and density values (FIG. 15B) using anti-M9 antibody to assess changes in M9 expression due to IAV infection in the lungs using total lung homogenates of IAV infected wild-type (WT) mice treated with or without 2.5 mg/kg recombinant full length M9 protein (M9^{FL}).

FIG. 16 shows expression or phosphorylation of molecular factors associated with lung injury (ZBP1, MLKL, phospho-MLKL, cleaved caspase-3 (Cl. Cas-3)) by Western blot analysis of total lung homogenates of WT mice from the saline treated control animals and the IAV infected animals (with M9′ treatment and without “none”).

FIGS. 17A-17E show percent survival (FIG. 17A), body and lung weights of saline treated control WT mice or IAV infected WT mice treated with M9^{P} (1.25 mg/kg) or without (FIGS. 17B-17D). In FIG. 17D, IAV infected mice without M9^{P} treatment are labeled “WT.” Total lung homogenates of the saline treated control animals and the IAV infected animals (with M9^{P} treatment and without “none”) were subjected to Western blotting using anti-NP antibody to assess viral titer (FIG. 17E).

FIG. 18 shows IAV antigen in situ_using anti-NP antibody on formalin fixed and paraffin embedded lung sections of IAV infected wild-type (WT) mice treated with or without 1.25 mg/kg M9^{P}.

FIG. 19 shows Western blotting using anti-M9 antibody to assess changes in M9 expression due to IAV infection in the lungs using total lung homogenates of IAV infected wild-type (WT) mice treated with or without 1.25 mg/kg M9.

FIG. 20 shows expression of molecular factors associated with lung injury (cleaved caspase-3 (Cl. Cas-3) and surfactant protein-C (SP-C)) by Western blot analysis of total lung homogenates of WT mice from the saline treated control animals and the IAV infected animals (with M9^{P} treatment and without “none”).

FIG. 21 shows induction of cleaved caspase-3 (Cl. Cas-3) by Western blot analysis of isolated alveolar macrophages (amQs) of WT mice from the saline treated control animals and the IAV infected animals (with M9^{P} treatment and without “none”).

FIG. 22 shows primary human alveolar epithelial cells (AECs) treated with saline or IAV (with M9^{P} treatment and without “none”).

The figures herein are for illustrative purposes only and are not necessarily drawn to scale.

### DETAILED DESCRIPTION

While aspects of the subject matter of the present disclosure may be embodied in a variety of forms, the following description is merely intended to disclose some of these forms as specific examples of the subject matter encompassed by the present disclosure. Accordingly, the subject matter of this disclosure is not intended to be limited to the forms or embodiments so described.

The present disclosure overcomes challenges associated with current technologies by providing proteins, biologically active peptides thereof, and antibodies specific for these proteins as therapeutic agents for treating infections, infection-induced disorders, infection-induced pathologies, and infection-induced injuries. In one aspect, the proteins, biologically active peptides thereof, and antibodies specific for these proteins are effective against viral infections. In one aspect, the present disclosure includes methods and uses of the proteins, biologically active peptides thereof, and antibodies specific for these proteins for disease treatment and prevention, particularly inhibiting virus growth and related syndromes. In some aspects, pharmaceutical formulations of multiple EGF-like-domains-9 (MEGF9), uncoordinated receptor 5A (UNC5A), dolichyl-phosphate beta-glucosyltransferase (ALG5), and/or biologically active peptides thereof, and antibodies specific for these proteins are provided. Also provided herein is a method, composition, or kit for treating pathogen-induced disease (e.g., lung diseases), by administering to the subject a therapeutically effective amount of one or more of the proteins and/or biologically active fragments thereof.

### I. Definitions

As used herein, “a” or “an” may mean one or more. As used herein in the claim(s), when used in conjunction with the word “comprising,” the words “a” or “an” may mean one or more than one.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component may be detected with standard analytical methods.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of,” as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value. Numerical quantities given are approximate, meaning that the term “around,” “about” or “approximately” can be inferred if not expressly stated.

The term “antibody” is meant to include both intact immunoglobulin (Ig) molecules as well as fragments and derivative thereof, that may be produced by proteolytic cleavage of Ig molecules or engineered genetically or chemically. Fragments include, for example, Fab, Fab′, F(ab′)₂ and Fv, each of which is capable of binding antigen. These fragments lack the Fc fragment of intact Ab and have an additional advantage, if used therapeutically, of clearing more rapidly from the circulation and undergoing less non-specific tissue binding than intact antibodies. Papain treatment of Ig's produces Fab fragments; pepsin treatment produces F(ab′)₂ fragments. These fragments may also be produced by genetic or protein engineering using methods well known in the art. A Fab fragment is a multimeric protein consisting of the portion of an Ig molecule containing the immunologically active portions of an Ig heavy (H) chain and an Ig light (L) chain covalently coupled together and capable of specifically combining with antigen. Fab fragments are typically prepared by proteolytic digestion of substantially intact Ig molecules with papain using methods that are well known in the art. However, a Fab fragment may also be prepared by expressing in a suitable host cell the desired portions of Ig H chain and L chain using methods well known in the art. A (Fab′)₂ fragment is a tetramer that includes a fragment of two H and two L chains. The Fv fragment is a multimeric protein consisting of the immunologically active portions of an Ig H chain variable (V) region (V_{H}) and an Ig L chain V region (V_{L}) covalently coupled together and capable of specifically combining with antigen. Fv fragments are typically prepared by expressing in suitable host cell the desired portions of Ig V_{H} region and V_{L} region using methods well known in the art.

Single-chain antigen-binding protein or single chain Ab, also referred to as “scFv,” is a polypeptide composed of an Ig V_{L} amino acid sequence tethered to an Ig V_{H} amino acid sequence by a peptide that links the C-terminus of the V_{L} sequence to the N-terminus of the V_{H} sequence.

### Chimeric Antibodies

The chimeric antibodies of the invention comprise individual chimeric H and L Ig chains. The chimeric H chain comprises an antigen binding region derived from the H chain of a non-human Ab specific for either M9, or UNC5A or ALG5, which is linked to at least a portion of a human C_{H} region. A chimeric L chain comprises an antigen binding region derived from the L chain of a non-human Ab specific for the target antigen linked to at least a portion of a human C_{L} region. As used herein, the term “antigen binding region” refers to that portion of an Ab molecule which contains the amino acid residues that interact with an antigen and confer on the Ab its specificity and affinity for the antigen. The Ab region includes the “framework” amino acid residues necessary to maintain the proper conformation of the antigen-binding (or “contact”) residues.

As used herein, the term “chimeric antibody” includes monovalent, divalent or polyvalent Igs. A monovalent chimeric Ab is an HL dimer formed by a chimeric H chain associated through disulfide bridges with a chimeric L chain. A divalent chimeric Ab is tetramer H₂L₂ formed by two HL dimers associated through at least one disulfide bridge. A polyvalent chimeric Ab can also be produced, for example, by employing a C_{H} region that aggregates (e.g., from an IgM H chain, termed the p chain).

Antibodies, fragments or derivatives having chimeric H chains and L chains of the same or different V region binding specificity, can be prepared by an appropriate association of the individual polypeptide chains, as taught, for example by Sears et al., Proc. Natl. Acad. Sci. USA 72:353-357 (1975). With this approach, hosts expressing chimeric H chains (or their derivatives) are separately cultured from hosts expressing chimeric L chains (or their derivatives), and the Ig chains are separately recovered and then associated. Alternatively, the hosts can be co-cultured and the chains allowed to associate spontaneously in the culture medium, followed by recovery of the assembled Ig, fragment or derivative.

The antigen binding region of the chimeric Ab (or a human mAb) of the present invention is derived preferably from a non-human Ab specific for the described polypeptides. The non-human Ab producing cell from which the V region of the Ab of the invention is derived may be a B lymphocyte obtained from the blood, spleen, lymph nodes or other tissue of an animal immunized with one of the polypeptides or a peptide or a relevant epitope thereof. The Ab-producing cell contributing the nucleotide sequences encoding the antigen-binding region of the chimeric Ab of the present invention may also be produced by transformation of a non-human, such as a primate, or a human cell. For example, a B lymphocyte (Kozbor et al. Immunol. Today 4:72-79 (1983)). Alternatively, the B lymphocyte may be transformed by providing a transforming gene or transforming gene product, as is well-known in the art. Preferably, the antigen binding region will be of murine origin. In other embodiments, the antigen binding region may be derived from other animal species, in particular, rodents such as rat or hamster.

The murine or chimeric mAb of the present invention may be produced in large quantities by injecting hybridoma or transfectoma cells secreting the Ab into the peritoneal cavity of mice and, after an appropriate time, harvesting the ascites fluid, which contains a high titer of the mAb. Alternatively, the antibodies may be produced by culturing hybridoma (or transfectoma) cells in vitro and isolating secreted mAb from the cell culture medium.

Human genes that encode the constant C regions of the chimeric antibodies of the present invention may be derived from a human fetal liver library or from any human cell including those which express and produce human Igs. The human C_{H} region can be derived from any of the known classes or isotypes of human H chains, including γ, μ, α, δ or ε, and subtypes thereof, such as G1, G2, G3 and G4. Since the H chain isotype is responsible for the various effector functions of an Ab, the choice of C_{H} region will be guided by the desired effector functions. Preferably, the C_{H} region is derived from γ1 (IgG1), γ3 (IgG3), γ4 (IgG4), or μ (IgM). The human C_{L} region can be derived from either human L chain isotype, κ or λ.

Genes encoding human Ig C regions are obtained from human cells by standard cloning techniques (Sambrook, J. et al., Molecular Cloning: A Laboratory Manual, 2nd Edition (or later edition), Cold Spring Harbor Press, Cold Spring Harbor, NY (1989)). Human C region genes are readily available from known clones containing genes representing the two classes of L chains, the five classes of H chains and subclasses thereof. Chimeric Ab fragments, such as F(ab′)₂ and Fab, can be prepared by designing a chimeric H chain gene which is appropriately truncated. For example, a chimeric gene encoding an H chain portion of an F(ab′)₂ fragment would include DNA sequences encoding the CH₁ domain and hinge region of the H chain, followed by a translational stop codon to yield the truncated molecule.

The chimeric Ig coding sequences or genes of the present invention can also be expressed in nonlymphoid mammalian cells or in other eukaryotic cells, such as yeast, or in prokaryotic cells, in particular bacteria. Yeast provides substantial advantages over bacteria for the production of Ig H and L chains. Yeasts carry out post-translational peptide modifications including glycosylation. A number of recombinant DNA strategies now exist which utilize strong promoter sequences and high copy number plasmids, which can be used for the production of the desired proteins in yeast. Yeast recognizes leader sequences of cloned mammalian gene products and secretes peptides bearing leader sequences (i.e., pre-peptides). Yeast gene expression systems can be routinely evaluated for the levels of production, secretion and the stability of chimeric H and L chain proteins and assembled chimeric Abs. Any of a series of yeast gene expression systems incorporating promoter and termination elements from the actively expressed genes coding for glycolytic enzymes produced in large quantities when yeasts are grown in media rich in glucose can be utilized. Bacterial strains may also be utilized as hosts for the production of Ab molecules or Ab fragments described by this invention. Preferred hosts are mammalian cells, grown in vitro or in vivo. Mammalian cells provide post-translational modifications to Ig protein molecules including leader peptide removal, folding and assembly of H and L chains, glycosylation of the Ab molecules, and secretion of functional Ab protein. Mammalian cells which may be useful as hosts for the production of Ab proteins, in addition to the cells of lymphoid origin described above, include cells of fibroblast origin, such as Vero (ATCC CRL 81) or CHO-K1 (ATCC CRL 61). Many vector systems are available for the expression of cloned H and L chain genes in mammalian cells (see Glover, supra). Different approaches can be followed to obtain complete H₂L₂ Abs.

For in vivo use, particularly for injection into humans, it is desirable to decrease the immunogenicity of the mAb by making mouse-human (or rodent-human) chimeric Abs as above, or by humanizing the Abs using methods known in the art. The humanized Ab may be the product of an animal having transgenic human Ig Constant region genes (see for example WO90/10077 and WO90/04036). Alternatively, the Ab of interest may be genetically engineered to substitute the CH₁, CH₂, CH₃, hinge domains, and/or the framework domain with the corresponding human sequence (see WO92/02190).

### Single Chain Antibodies

The Ab of the present invention may be produced as a single chain Ab or scFv instead of the normal multimeric structure. Single chain Abs include the hypervariable regions from an Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; Jost C R et al., J Biol Chem. 1994 269:26267-26273; U.S. Pat. Nos. 4,704,692, 4,853,871, 4,94,6778, 5,260,203, 5,455,030). DNA sequences encoding the V regions of the H chain and the L chain are ligated to a linker encoding at least about 4 amino acids (typically small neutral amino acids). The protein encoded by this fusion allows assembly of a functional variable region that retains the specificity and affinity of the original Ab.

Antibodies can be selected for particular desired properties. In the case of an Ab to be used in vivo, Ab screening procedures can include any of the in vitro or in vivo bioassays that measure binding to MEGF9, UNC5A or ALG5 or a peptide epitope thereof, or to cells expressing the relevant polypeptide or peptide epitope.

A “biologically active fragment” of a protein refers to a peptide whose the amino acid sequence may include less amino acids than the corresponding full-length protein but still enough amino acids to confer the activity or function of the corresponding full-length protein. For example, a biological active fragment of a protein may have at least 20% of the biological or biochemical activity of the corresponding full-length protein (e.g., as measured by an in vitro or an in vivo assay). In some examples, the biological active peptide may have an increase biological or biochemical activity as compared to the corresponding full-length protein.

The term “identity” or “homology” shall be construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions shall be construed as reducing identity or homology. Methods and computer programs for the alignment are well known in the art. Sequence identity may be measured using sequence analysis software. A protein or peptide has a certain percentage (for example, 80%, 85%, 90%, or 95%) of “sequence identity” or “homology” to another sequence means that, when aligned, that percentage of bases (or amino acids) are the same in comparing the two sequences. This alignment and the percent homology or sequence identity may be determined using software programs known in the art, for example those described in Current Protocols In Molecular Biology (F. M. Ausubel et al., eds., 1987) Supplement 30, section 7.7.18, Table 7.7.1.

The term “polypeptide” or “protein” is used in its broadest sense to refer to a compound of two or more subunit amino acids, amino acid analogs, or peptidomimetics. The subunits may be linked by peptide bonds. In some embodiments, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. The term “peptidomimetic” or “peptide mimic” means that a peptide according to the invention is modified in such a way that it includes at least one non-peptidic bond such as, for example, urea bond, carbamate bond, sulfonamide bond, hydrazine bond, or any other covalent bond. A peptide of three or more amino acids is commonly called an oligopeptide if the peptide chain is short. If the peptide chain is long, the peptide is commonly called a polypeptide or a protein.

The terms “subject” and “individual” and “patient” are used interchangeably herein, and refer to an animal, for example a human or non-human animal (e.g., a mammal), to whom treatment, including prophylactic treatment, with a pharmaceutical composition as disclosed herein, is provided. The term “subject” as used herein refers to human and non-human animals. The term “non-human animals” includes all vertebrates, e.g., mammals, such as non-human primates (particularly higher primates), sheep, dogs, rodents (e.g. mouse or rat), guinea pigs, goats, pigs, cats, rabbits, cows, and non-mammals such as chickens, amphibians, reptiles etc. In one embodiment, the subject is human. In another embodiment, the subject is an experimental animal or animal substitute as a disease model. In another embodiment, the subject is a farm animal. Non-human mammals include mammals such as non-human primates (particularly higher primates), sheep, dogs, rodents (e.g. mouse or rat), guinea pigs, goats, pigs, cats, rabbits, horses, and cows. In some aspects, the non-human animal is a companion animal such as a dog or a cat.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease.

By “isolated” it is meant that the polypeptide has been separated from any natural environment, such as a body fluid, e.g., blood, and separated from the components that naturally accompany the peptide.

By isolated and “substantially pure” is meant a polypeptide that has been separated and purified to at least some degree from the components that naturally accompany it. For example, a polypeptide is substantially pure when it is at least about 60%, or at least about 70%, at least about 80%, at least about 90%, at least about 95%, or even at least about 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

The term “variant” as used herein refers to a polypeptide or nucleic acid that differs from the polypeptide or nucleic acid by one or more amino acid or nucleic acid deletions, additions, substitutions or side-chain modifications, yet retains one or more specific functions or biological activities of the naturally occurring molecule. Amino acid substitutions include alterations in which an amino acid is replaced with a different naturally occurring or a non-conventional amino acid residue. Such substitutions may be classified as “conservative,” in which case an amino acid residue contained in a polypeptide is replaced with another naturally occurring amino acid of similar character either in relation to polarity, side chain functionality or size. Such conservative substitutions are well known in the art. Substitutions encompassed by the present invention may also be “non-conservative,” in which an amino acid residue, which is present in a peptide is substituted with an amino acid having different properties, such as naturally-occurring amino acid from a different group (e.g., substituting a charged or hydrophobic amino; acid with alanine), or alternatively, in which a naturally-occurring amino acid is substituted with a non-conventional amino acid. In some embodiments, amino acid substitutions may be conservative. Also encompassed within the term variant when used with reference to a polynucleotide or polypeptide, refers to a polynucleotide or polypeptide that may vary in primary, secondary, or tertiary structure, as compared to a reference polynucleotide or polypeptide, respectively (e.g., as compared to a wild-type polynucleotide or polypeptide).

The term “insertions” or “deletions” are typically in the range of about 1 to 5 amino acids. The variation allowed may be experimentally determined by producing the peptide synthetically while systematically making insertions, deletions, or substitutions of nucleotides in the sequence using recombinant DNA techniques.

The term “substitution” when referring to a peptide, refers to a change in an amino acid for a different entity, for example another amino acid or amino acid moiety. Substitutions may be conservative or non-conservative substitutions.

An “analog” of a molecule such as a peptide refers to a molecule similar in function to either the entire molecule or to a fragment thereof. The term “analog” is also intended to include allelic species and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N- or C-terminal amino acids. Examples of unnatural amino acids are, for example but not limited to; disubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine. Fragments and analogs may be screened for prophylactic or therapeutic efficacy in transgenic animal models as described below.

By “covalently bonded” is meant joined either directly or indirectly (e.g., through a linker) by a covalent chemical bond. In some aspects of all the embodiments of the invention, the fusion peptides are covalently bonded.

The term “fusion protein” as used herein refers to a recombinant protein of two or more proteins or peptides. Fusion proteins may be produced, for example, by a nucleic acid sequence encoding one protein is joined to the nucleic acid encoding another protein such that they constitute a single open-reading frame that may be translated in the cells into a single polypeptide harboring all the intended proteins. The order of arrangement of the proteins may vary. Fusion proteins may include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AU1, AU5, and avidin. Half-life extenders include Fc domain and serum albumin.

The term “airway” refers herein to any portion of the respiratory tract including the upper respiratory tract, the respiratory airway, and the lungs. The upper respiratory tract includes the nose and nasal passages, mouth, and throat. The respiratory airway includes the larynx, trachea, bronchi and bronchioles. The lungs include the respiratory bronchioles, alveolar ducts, alveolar sacs and alveoli.

The terms “nebulizing,” “nebulized” and other grammatical variations, refer herein to the process of converting a liquid into small aerosol droplets. In some embodiments, the aerosol droplets have a median diameter of approximately 2-10 μm. In some embodiments, the aerosol droplets have a median diameter of approximately 2-4 μm.

The term “air jet mill” refers to a device or method for reducing particle size by using a jet of compressed gas to impact particles into one another, thereby pulverizing the particles. An air jet mill may be used to reduce the size of peptide particles. Other mechanical milling devices that perform the same function may also be used interchangeably with the air jet mill. Air jet milling may occur under various environmental parameters such as temperature, pressure, relative/absolution humidity, oxygen content, etc.

The term “ball mill” refers to a device or method for reducing particle size by adding the particle of interest and a grinding medium to the interior of a cylinder and rotating the cylinder. The particles of interest may be broken down as the grinding medium rises and falls along the exterior of the cylinder as it rotates. A ball mill may be used to reduce the size of peptide particles. Other mechanical milling devices that perform the same function may also be used interchangeably with the air jet mill.

The term “wet mill” or “media mill” refers to a device or method for reducing particle size by adding the particle of interest to device with an agitator, containing a media comprising a liquid and a grinding medium. With the addition of the particle of interest, as the agitator rotates, the energy it disperses causes the grinding medium and particles of interest to come into contact and break down the particles of interest. Other mechanical milling devices that perform the same function may also be used interchangeably with the air jet mill.

The term “high pressure homogenization” refers to a method of reducing particle size by adding the particle of interest to a device, which combines both pressure and mechanical forces to break down the particle of interest. Mechanical forces used in high pressure homogenization may include impact, shear, and cavitation, among others. Other mechanical milling devices that perform the same function may also be used interchangeably with the air jet mill.

The term “cryogenic mill” refers to a device or method for reducing particle size by first chilling a particle of interest with dry ice, liquid nitrogen, or other cryogenic liquid, and subsequently milling the particle of interest to reduce the size. Other mechanical milling devices that perform the same function may also be used interchangeably with the air jet mill.

The phrase “effective amount” or “therapeutically effective” means a dosage of a drug or agent sufficient to produce a desired therapeutic result. The desired therapeutic result may be improvement in the condition to be treated by a claimed method, use, or composition.

As used herein, “excipient” refers to pharmaceutically acceptable carriers that are relatively inert substances used to facilitate administration or delivery of an Active Pharmaceutical Ingredient (API) into a subject or used to facilitate processing of an API into drug formulations that may be used pharmaceutically for delivery to the site of action in a subject. Excipients or pharmaceutically acceptable carriers include all of the inactive components of the dosage form except for the active ingredient(s). Non-limiting examples of excipients include carrier agents, bulking agents, stabilizing agents, surfactants, surface modifiers, solubility enhancers, buffers, encapsulating agents, antioxidants, preservatives, nonionic wetting or clarifying agents, viscosity-increasing agents, and absorption-enhancing agents. “Excipient free” refers to the pharmaceutical composition of interest in a formulation free of any excipients.

The phrases “pharmaceutical composition” or “pharmacologically acceptable composition” refers to molecular entities and compositions that do not produce an adverse, allergic, or other untoward reaction when administered to an animal, such as a human, as appropriate. The preparation of a pharmaceutical composition comprising one or more of the proteins and peptides described herein, or additional active ingredients will be known to those of skill in the art in light of the present disclosure. Moreover, for animal (e.g., human) administration, it will be understood that preparations should meet bioburden, sterility, pyrogenicity, general safety, and/or purity standards as required by the FDA or other recognized regulatory authority.

As used herein, “pharmaceutically acceptable carrier” includes any and all excipients, buffers, co-solvents, tonicity agents, processing aids, aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, disintegration agents, lubricants, flavor modifiers (e.g., sweetening agents, flavoring agents), such like materials and combinations thereof, as would be known to one of ordinary skill in the art. The pH and exact concentration of the various components in a pharmaceutical composition may be adjusted according to well-known parameters. In some aspects, the carrier may encapsulate a therapeutic agent, but not itself be consumed or administered to a subject (e.g., a shell capsule encasing a dry powder composition, such as for use in a dry powder inhaler).

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

As used herein, a plurality of compounds, elements, or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Furthermore, certain compositions, elements, excipients, ingredients, disorders, conditions, properties, steps, or the like may be discussed in the context of one specific embodiment or aspect or in a separate paragraph or section of this disclosure. It is understood that this is merely for convenience and brevity, and any such disclosure is equally applicable to and intended to be combined with any other embodiments or aspects found anywhere in the present disclosure and claims, which all form the application and claimed invention at the filing date. For example, a list of method steps, active agents, kits, or compositions described with respect to a formulation or method of treating a certain subject is intended to and does find direct support for embodiments related to compositions, formulations, and methods described in any other part of this disclosure, even if those method steps, active agents, kits, or compositions are not re-listed in the context or section of that embodiment or aspect.

### II. Therapeutic Proteins, Peptides, and Antibodies

In some aspects, the present disclosure provides proteins and peptides (e.g., biologically active fragments of the proteins) that may be used to inhibit virus growth and treat related disease. The proteins and peptides may be synthetic, recombinant, or chemically modified peptides isolated or generated using methods well known in the art. The present disclosure also provides compositions (e.g., pharmaceutical compositions) comprising one or more of the proteins and peptides provided herein.

### A. MEGF9 and Biologically Active Fragments Thereof

In some embodiments, the present disclosure provides a Multiple EGF-like-domains-9 (“MEGF9” or “M9”), and biologically active fragments thereof, and specific antibodies for M9 or biologically active fragments thereof. The MEGF9 may be a human MEGF9. In one example, the MEGF9 may comprise the sequence of SEQ ID NO: 1 (full-length human MEGF9 with signal peptide, Accession Number Q9H1U4, see uniprot.org/uniprot/Q9H1U4). The protein of SEQ ID NO: 1 comprises the following domains: signal peptide (amino acids 1-30 of SEQ ID NO: 1), extracellular domain (ECD) (amino acids 31-514 of SEQ ID NO: 1), Transmembrane (helical) domain (amino acids 515-535 of SEQ ID NO: 1), cytoplasmic domain (amino acids 536-602 of SEQ ID NO: 1), and the polypeptide having SEQ ID NO: 102.

In some examples, the MEGF9 may comprise a sequence with at least 80%, e.g., at least 85%, at least 90%, at least 95%, or at least 99% identity to SEQ ID NO: 1. In some examples, the MEGF may consist of a sequence of SEQ ID NO: 1. In some examples, the MEGF may consist of a sequence with at least 80%, e.g., at least 85%, at least 90%, at least 95%, or at least 99% identity to SEQ ID NO: 1.

In some examples, the MEGF9 may comprise a mature protein without the signal peptide (SEQ ID NO: 2). In one example, the MEGF9 may comprise a sequence with at least 80%, e.g., at least 85%, at least 90%, at least 95%, or at least 99% identity to SEQ ID NO: 2. In some examples, the MEGF9 may consist of a sequence of SEQ ID NO: 2. In some examples, the MEGF9 may consist of a sequence with at least 80%, e.g., at least 85%, at least 90%, at least 95%, or at least 99% identity to SEQ ID NO: 2.

In some embodiments, a biological active fragment of the MEGF9 may comprise a sequence in the ECD of MEGF9. In some examples, the biological active fragment of the MEGF9 may comprise a sequence of SEQ ID NO: 3. In some examples, the biological active fragment of the MEGF9 may comprise a sequence with at least 80%, e.g., at least 85%, at least 90%, at least 95%, or at least 99% identity to SEQ ID NO: 3.

In some examples, the biological active fragment of the MEGF9 may comprise a sequence of SEQ ID NO: 102. In some examples, the biological active fragment of the MEGF9 may comprise a sequence with at least 80%, e.g., at least 85%, at least 90%, at least 95%, or at least 99% identity to SEQ ID NO: 102. In some examples, the biological active fragment of the MEGF9 may consist of a sequence of SEQ ID NO: 102. In some examples, the biological active fragment of the MEGF9 may consist of a sequence with at least 80%, e.g., at least 85%, at least 90%, at least 95%, or at least 99% identity to SEQ ID NO: 102.

In some examples, the biological active fragment of the MEGF9 may consist of a sequence of SEQ ID NO: 3. In some examples, the biological active fragment of the MEGF9 may consist of a sequence with at least 80%, e.g., at least 85%, at least 90%, at least 95%, or at least 99% identity to SEQ ID NO: 3.

In some examples, the biological active fragment of the MEGF9 may comprise or consist of a portion of the ECD. In some examples, the biological active fragment of the MEGF9 may comprise or consist of a sequence of SEQ ID NO: 4. In some examples, the biological active fragment of the MEGF9 may comprise or consist of a sequence with a one-amino acid, two-amino acid, three-amino acid, four-amino acid, five-amino acid, or six-amino acid modification of SEQ ID NO: 4.

The present disclosure also includes antibodies against MEGF9. In one aspect, the present disclosure includes antibodies generated against SEQ ID NOs: 1-4 and 102 and fragments thereof. In one aspect, the present disclosure includes use of:

### Anti-M9 Antibody Products

(1) Mybiosource: Catalog #MBS9605643
Clonality: Polyclonal, Isotype: IgG, Host: Rabbit
(2) Invitrogen: #PA5-106685

### Clonality: Polyclonal, Isotype: IgG, Host: Rabbit

In some embodiments, the present disclosure provides an M9-binding protein, e.g., an M9 antibody or an M9-binding fragment thereof. In some embodiments, the M9-binding protein comprises a heavy chain variable region comprising three variable heavy chain complementarity determining regions (V_{H} CDR1, V_{H} CDR2, and V_{H} CDR3), and a light chain variable region comprising three variable light chain CDRs (V_{L} CDR1, V_{L} CDR2, and V_{L} CDR3).

In some examples, in the M9-binding protein, the V_{H} CDR1 comprises SEQ ID NO: 108, the V_{H} CDR2 comprises SEQ ID NO: 109, the V_{H} CDR3 comprises SEQ ID NO: 110, the V_{L} CDR1 comprises SEQ ID NO: 115, the V_{L} CDR2 comprises SEQ ID NO: 116, and the V_{L} CDR3 comprises SEQ ID NO: 117. In some examples, in the M9-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 107; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 114. In some examples, the M9-binding protein comprises a heavy chain variable region comprising SEQ ID NO: 107 and a light chain variable region comprising SEQ ID NO: 114. In some examples, the M9-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 106; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 113. In some examples, the M9-binding protein comprises a heavy chain comprising SEQ ID NO: 106 and a light chain comprising SEQ ID NO: 113. In some examples, the M9-binding protein comprises a heavy chain comprising SEQ ID NO: 105 and a light chain comprising SEQ ID NO: 112.

In some examples, in the M9-binding protein, the V_{H} CDR1 comprises SEQ ID NO: 122, the V_{H} CDR2 comprises SEQ ID NO: 123, the V_{H} CDR3 comprises SEQ ID NO: 124, the V_{L} CDR1 comprises SEQ ID NO: 129, the V_{L} CDR2 comprises SEQ ID NO: 130, and the V_{L} CDR3 comprises SEQ ID NO: 131. In some examples, in the M9-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 121; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 128. In some examples, the M9-binding protein comprises a heavy chain variable region comprising SEQ ID NO: 121 and a light chain variable region comprising SEQ ID NO: 128. In some examples, the M9-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 120; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 127. In some examples, the M9-binding protein comprises a heavy chain comprising SEQ ID NO: 120 and a light chain comprising SEQ ID NO: 127. In some examples, the M9-binding protein comprises a heavy chain comprising SEQ ID NO: 119 and a light chain comprising SEQ ID NO: 126.

In some examples, in the M9-binding protein, the V_{H} CDR1 comprises SEQ ID NO: 136, the V_{H} CDR2 comprises SEQ ID NO: 137, the V_{H} CDR3 comprises SEQ ID NO: 138, the V_{L} CDR1 comprises SEQ ID NO: 143, the V_{L} CDR2 comprises SEQ ID NO: 144, and the V_{L} CDR3 comprises SEQ ID NO: 145. In some examples, in the M9-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 135; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 142. In some examples, the M9-binding protein comprises a heavy chain variable region comprising SEQ ID NO: 135 and a light chain variable region comprising SEQ ID NO: 142. In some examples, the M9-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 134; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 141. In some examples, the M9-binding protein comprises a heavy chain comprising SEQ ID NO: 134 and a light chain comprising SEQ ID NO: 141. In some examples, the M9-binding protein comprises a heavy chain comprising SEQ ID NO: 133 and a light chain comprising SEQ ID NO: 140.

In some examples, in the M9-binding protein, the V_{H} CDR1 comprises SEQ ID NO: 150, the V_{H} CDR2 comprises SEQ ID NO: 151, the V_{H} CDR3 comprises SEQ ID NO: 152, the V_{L} CDR1 comprises SEQ ID NO: 157, the V_{L} CDR2 comprises SEQ ID NO: 158, and the V_{L} CDR3 comprises SEQ ID NO: 159. In some examples, in the M9-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 149; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 156. In some examples, the M9-binding protein comprises a heavy chain variable region comprising SEQ ID NO: 149 and a light chain variable region comprising SEQ ID NO: 156. In some examples, the M9-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 148; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 155. In some examples, the M9-binding protein comprises a heavy chain comprising SEQ ID NO: 148 and a light chain comprising SEQ ID NO: 155. In some examples, the M9-binding protein comprises a heavy chain comprising SEQ ID NO: 147 and a light chain comprising SEQ ID NO: 154.

In some examples, in the M9-binding protein, the V_{H} CDR1 comprises SEQ ID NO: 164, the V_{H} CDR2 comprises SEQ ID NO: 165, the V_{H} CDR3 comprises SEQ ID NO: 166, the V_{L} CDR1 comprises SEQ ID NO: 171, the V_{L} CDR2 comprises SEQ ID NO: 172, and the V_{L} CDR3 comprises SEQ ID NO: 173. In some examples, in the M9-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 163; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 170. In some examples, the M9-binding protein comprises a heavy chain variable region comprising SEQ ID NO: 163 and a light chain variable region comprising SEQ ID NO: 170. In some examples, the M9-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 162; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 169. In some examples, the M9-binding protein comprises a heavy chain comprising SEQ ID NO: 162 and a light chain comprising SEQ ID NO: 169. In some examples, the M9-binding protein comprises a heavy chain comprising SEQ ID NO: 161 and a light chain comprising SEQ ID NO: 168.

In some examples, in the M9-binding protein, the V_{H} CDR1 comprises SEQ ID NO: 178, the V_{H} CDR2 comprises SEQ ID NO: 189, the V_{H} CDR3 comprises SEQ ID NO: 180, the V_{L} CDR1 comprises SEQ ID NO: 185, the V_{L} CDR2 comprises SEQ ID NO: 186, and the V_{L} CDR3 comprises SEQ ID NO: 187. In some examples, in the M9-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 177; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 184. In some examples, the M9-binding protein comprises a heavy chain variable region comprising SEQ ID NO: 177 and a light chain variable region comprising SEQ ID NO: 184. In some examples, the M9-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 176; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 183. In some examples, the M9-binding protein comprises a heavy chain comprising SEQ ID NO: 176 and a light chain comprising SEQ ID NO: 183. In some examples, the M9-binding protein comprises a heavy chain comprising SEQ ID NO: 175 and a light chain comprising SEQ ID NO: 182.

In some embodiments, the present disclosure provides nucleic acids encoding an M9-binding protein, e.g., SEQ ID NOs: 111, 125, 139, 153, 167, 181, 118, 132, 146, 160, 174, and 188.

In some embodiments, the M9 antibody is a monoclonal antibody.

### B. UNC5A and Biologically Active Fragments Thereof

In some embodiments, the present disclosure provides Uncoordinated receptor 5A (“UNC5A” or “U5A”), biologically active fragments thereof, and specific antibodies for UNC5A or biologically active fragments thereof. The UNC5A may be a human UNC5A. In one example, the UNC5A may comprise the sequence of SEQ ID NO: 5 (full-length human UNC5A with signal peptide, Accession Number Q6ZN44, see uniprot.org/uniprot/Q6ZN44). The protein of SEQ ID NO: 5 comprises the following domains: signal peptide (amino acids 1-25), Ig-like domain (amino acids 44-141 of SEQ ID NO: 5), Ig-like C2-type domain (amino acids 155-234 of SEQ ID NO: 5), TSP type-1 domain (amino acids 242-294 of SEQ ID NO: 5), ZU (interacting with Netrin receptor DCC) (amino acids 441-584 of SEQ ID NO: 5), and death domain (amino acids 761-841 of SEQ ID NO: 5). In some examples, the biological active fragment of the UNC5A may comprise a sequence in the ECD of UNC5A, e.g., amino acids 26-306 of SEQ ID NO: 5). In some aspects, the present disclosure includes the polypeptide having SEQ ID NO: 103.

In some examples, the UNC5A may comprise a sequence with at least 80%, e.g., at least 85%, at least 90%, at least 95%, or at least 99% identity to SEQ ID NO: 5. In some examples, the UNC5A may consist of a sequence of SEQ ID NO: 5. In some examples, the UNC5A may consist of a sequence with at least 80%, e.g., at least 85%, at least 90%, at least 95%, or at least 99% identity to SEQ ID NO: 5.

In some examples, the UNC5A may comprise a mature protein without the signal peptide (SEQ ID NO: 6). In some examples, the UNC5A may comprise a sequence with at least 80%, e.g., at least 85%, at least 90%, at least 95%, or at least 99% identity to SEQ ID NO: 6. In some examples, the UNCSA may consist of a sequence of SEQ ID NO: 6. In some examples, the UNCSA may consist of a sequence with at least 80%, e.g., at least 85%, at least 90%, at least 95%, or at least 99% identity to SEQ ID NO: 6.

In some examples, a biological active fragment of the UNC5A may comprise a sequence of SEQ ID NO: 7. In some examples, the biological active fragment of the UNC5A may comprise a sequence with at least 80%, e.g., at least 85%, at least 90%, at least 95%, or at least 99% identity to SEQ ID NO: 7.

In some examples, the biological active fragment of the UNCSA may consist of a sequence of SEQ ID NO: 7. In some examples, the biological active fragment of the UNCSA may consist of a sequence with at least 80%, e.g., at least 85%, at least 90%, at least 95%, or at least 99% identity to SEQ ID NO: 7.

In some examples, the biological active fragment of the UNCSA may comprise a sequence of SEQ ID NO: 8. In some examples, the biological active fragment of the UNCSA may comprise a sequence with at least 80%, e.g., at least 85%, at least 90%, at least 95%, or at least 99% identity to SEQ ID NO: 8.

In some examples, the biological active fragment of the UNCSA may consist of a sequence of SEQ ID NO: 8. In some examples, the biological active fragment of the UNCSA may consist of a sequence with at least 80%, e.g., at least 85%, at least 90%, at least 95%, or at least 99% identity to SEQ ID NO: 8.

In some examples, the biological active fragment of the UNCSA may comprise a sequence of SEQ ID NO: 103. In some examples, the biological active fragment of the MEGF9 may comprise a sequence with at least 80%, e.g., at least 85%, at least 90%, at least 95%, or at least 99% identity to SEQ ID NO: 103. In some examples, the biological active fragment of the UNC5A may consist of a sequence of SEQ ID NO: 103. In some examples, the biological active fragment of the MEGF9 may consist of a sequence with at least 80%, e.g., at least 85%, at least 90%, at least 95%, or at least 99% identity to SEQ ID NO: 103.

The present disclosure also includes antibodies against UNC5A. In one aspect, the present disclosure includes antibodies generated against SEQ ID NOs: 5-8 and 103 and fragments thereof. In one aspect, the present disclosure includes use of:

### Anti-U5A Antibody Products

1) ProteinTech, #22068-1-AP
   Clonality: Polyclonal, Isotype: IgG, Host: Rabbit
2) Sigma, #SAB2108252, Lot #QC23528
   Clonality: Polyclonal, Isotype: IgG, Host: Rabbit

In some embodiments, the present disclosure provides a U5A-binding protein, e.g., a U5A antibody or an U5A-binding fragment thereof. In some embodiments, the U5A-binding protein comprises a heavy chain variable region comprising three variable heavy chain complementarity determining regions (V_{H} CDR1, V_{H} CDR2, and V_{H} CDR3), and a light chain variable region comprising three variable light chain CDRs (V_{L} CDR1, V_{L} CDR2, and V_{L} CDR3).

In some examples, in the U5A-binding protein, the V_{H} CDR1 comprises SEQ ID NO: 192, the V_{H} CDR2 comprises SEQ ID NO: 193, the V_{H} CDR3 comprises SEQ ID NO: 194, the V_{L} CDR1 comprises SEQ ID NO: 199, the V_{L} CDR2 comprises SEQ ID NO: 200, and the V_{L} CDR3 comprises SEQ ID NO: 201. In some examples, in the U5A-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 191; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 198. In some examples, the U5A-binding protein comprises a heavy chain variable region comprising SEQ ID NO: 191 and a light chain variable region comprising SEQ ID NO: 198. In some examples, the U5A-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 190; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 197. In some examples, the U5A-binding protein comprises a heavy chain comprising SEQ ID NO: 190 and a light chain comprising SEQ ID NO: 197. In some examples, the U5A-binding protein comprises a heavy chain comprising SEQ ID NO: 089 and a light chain comprising SEQ ID NO: 196.

In some examples, in the U5A-binding protein, the V_{H} CDR1 comprises SEQ ID NO: 206, the V_{H} CDR2 comprises SEQ ID NO: 207, the V_{H} CDR3 comprises SEQ ID NO: 208, the V_{L} CDR1 comprises SEQ ID NO: 213, the V_{L} CDR2 comprises SEQ ID NO: 214, and the V_{L} CDR3 comprises SEQ ID NO: 215. In some examples, in the U5A-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 205; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 212. In some examples, the U5A-binding protein comprises a heavy chain variable region comprising SEQ ID NO: 205 and a light chain variable region comprising SEQ ID NO: 212. In some examples, the U5A-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 204; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 211. In some examples, the U5A-binding protein comprises a heavy chain comprising SEQ ID NO: 204 and a light chain comprising SEQ ID NO: 211. In some examples, the U5A-binding protein comprises a heavy chain comprising SEQ ID NO: 203 and a light chain comprising SEQ ID NO: 210.

In some examples, in the U5A-binding protein, the V_{H} CDR1 comprises SEQ ID NO: 220, the V_{H} CDR2 comprises SEQ ID NO: 221, the V_{H} CDR3 comprises SEQ ID NO: 222, the V_{L} CDR1 comprises SEQ ID NO: 227, the V_{L} CDR2 comprises SEQ ID NO: 228, and the V_{L} CDR3 comprises SEQ ID NO: 229. In some examples, in the U5A-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 219; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 226. In some examples, the U5A-binding protein comprises a heavy chain variable region comprising SEQ ID NO: 219 and a light chain variable region comprising SEQ ID NO: 226. In some examples, the U5A-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 218; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 225. In some examples, the U5A-binding protein comprises a heavy chain comprising SEQ ID NO: 218 and a light chain comprising SEQ ID NO: 225. In some examples, the U5A-binding protein comprises a heavy chain comprising SEQ ID NO: 217 and a light chain comprising SEQ ID NO: 224.

In some examples, in the U5A-binding protein, the V_{H} CDR1 comprises SEQ ID NO: 234, the V_{H} CDR2 comprises SEQ ID NO: 235, the V_{H} CDR3 comprises SEQ ID NO: 236, the V_{L} CDR1 comprises SEQ ID NO: 241, the V_{L} CDR2 comprises SEQ ID NO: 242, and the V_{L} CDR3 comprises SEQ ID NO: 243. In some examples, in the U5A-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 233; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 240. In some examples, the U5A-binding protein comprises a heavy chain variable region comprising SEQ ID NO: 233 and a light chain variable region comprising SEQ ID NO: 240. In some examples, the U5A-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 232; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 239. In some examples, the U5A-binding protein comprises a heavy chain comprising SEQ ID NO: 232 and a light chain comprising SEQ ID NO: 239. In some examples, the U5A-binding protein comprises a heavy chain comprising SEQ ID NO: 231 and a light chain comprising SEQ ID NO: 238.

In some examples, in the U5A-binding protein, the V_{H} CDR1 comprises SEQ ID NO: 248, the V_{H} CDR2 comprises SEQ ID NO: 249, the V_{H} CDR3 comprises SEQ ID NO: 250, the V_{L} CDR1 comprises SEQ ID NO: 255, the V_{L} CDR2 comprises SEQ ID NO: 256, and the V_{L} CDR3 comprises SEQ ID NO: 257. In some examples, in the U5A-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 247; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 254. In some examples, the U5A-binding protein comprises a heavy chain variable region comprising SEQ ID NO: 247 and a light chain variable region comprising SEQ ID NO: 254. In some examples, the U5A-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 246; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 253. In some examples, the U5A-binding protein comprises a heavy chain comprising SEQ ID NO: 246 and a light chain comprising SEQ ID NO: 253. In some examples, the U5A-binding protein comprises a heavy chain comprising SEQ ID NO: 245 and a light chain comprising SEQ ID NO: 252.

In some embodiments, the present disclosure provides nucleic acids encoding an U5A-binding protein, e.g., SEQ ID NOs: 195, 209, 223, 237, 251, 202, 216, 230, 244, and 258.

In some embodiments, the U5A antibody is a monoclonal antibody.

### C. ALG5 and Biologically Active Fragments Thereof

In some embodiments, the present disclosure provides ALG5 (“A5”) and biologically active fragments thereof. The ALG5 may be a human ALG5. In one example, the ALG5 may comprise the sequence of SEQ ID NO: 9 (full-length human ALG5, Accession Number Q9Y673, see www.uniprot.org/uniprot/Q9Y673). The protein of SEQ ID NO: 9 comprises the following domains: cytoplasmic domain (amino acids 1-7), transmembrane (helical) domain (amino acids 8-28), and lumenal domain (amino acids 29-324). In some aspects, the present disclosure includes the polypeptide having SEQ ID NO: 104.

In some examples, the ALG5 may comprise a sequence with at least 80%, e.g., at least 85%, at least 90%, at least 95%, or at least 99% identity to SEQ ID NO: 9. In some examples, the ALG5 may consist of a sequence of SEQ ID NO: 9. In some examples, the ALG5 may consist of a sequence with at least 80%, e.g., at least 85%, at least 90%, at least 95%, or at least 99% identity to SEQ ID NO: 9.

In some examples, the biological active fragment of the ALG5 may comprise a sequence of SEQ ID NO: 104. In some examples, the biological active fragment of the ALG5 may comprise a sequence with at least 80%, e.g., at least 85%, at least 90%, at least 95%, or at least 99% identity to SEQ ID NO: 104. In some examples, the ALG5 may consist of a sequence of SEQ ID NO: 104. In some examples, the ALG5 may consist of a sequence with at least 80%, e.g., at least 85%, at least 90%, at least 95%, or at least 99% identity to SEQ ID NO: 104.

The present disclosure also includes antibodies against ALG5. In one aspect, the present disclosure includes antibodies generated against SEQ ID NOs: 9 and 104 and fragments thereof. In one aspect, the present disclosure includes use of:
Anti-ALG5 antibodies are offered by a number of suppliers. This target gene encodes the protein “ALG5 dolichyl-phosphate beta-glucosyltransferase” in humans and may also be known as bA421P11.2. Structurally, the protein is reported to be 36.9 kDa in mass. Based on gene name, yeast, canine, porcine, monkey, mouse and rat orthologs may also be found. For more comprehensive antibody product information (such as immunogen, specificity, applications, and more), visit the supplier page noted below.
novusbio.com/products/alg5-antibody_nbp1-88767
thermofisher.com/antibody/product/ALG5-Antibody-Polyclonal/PA5-52496
ptglab.com/products/ALG5-Antibody-16046-1-AP.htm
thermofisher.com/antibody/product/ALG5-Antibody-Polyclonal/PA5-109380
thermofisher.com/antibody/product/ALG5-Antibody-Polyclonal/PA5-114185
sigmaaldrich.com/catalog/product/sigma/sab1410599?lang=en&region=US
atlasantibodies.com/products/antibodies/primary-antibodies/triple-a-polyclonals/alg5-antibody-hpa007989/

In some embodiments, the present disclosure provides an ALG5-binding protein, e.g., an ALG5 antibody or an ALG5-binding fragment thereof. In some embodiments, the ALG5-binding protein comprises a heavy chain variable region comprising three variable heavy chain complementarity determining regions (V_{H} CDR1, V_{H} CDR2, and V_{H} CDR3), and a light chain variable region comprising three variable light chain CDRs (V_{L} CDR1, V_{L} CDR2, and V_{L} CDR3).

In some examples, in the ALG5-binding protein, the V_{H} CDR1 comprises SEQ ID NO: 262, the V_{H} CDR2 comprises SEQ ID NO: 263, the V_{H} CDR3 comprises SEQ ID NO: 264, the V_{L} CDR1 comprises SEQ ID NO: 269, the V_{L} CDR2 comprises SEQ ID NO: 270, and the V_{L} CDR3 comprises SEQ ID NO: 271. In some examples, in the ALG5-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 261; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 268. In some examples, the ALG5-binding protein comprises a heavy chain variable region comprising SEQ ID NO: 261 and a light chain variable region comprising SEQ ID NO: 268. In some examples, the ALG5-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 260; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 267. In some examples, the ALG5-binding protein comprises a heavy chain comprising SEQ ID NO: 260 and a light chain comprising SEQ ID NO: 267. In some examples, the ALG5-binding protein comprises a heavy chain comprising SEQ ID NO: 259 and a light chain comprising SEQ ID NO: 266.

In some examples, in the ALG5-binding protein, the V_{H} CDR1 comprises SEQ ID NO: 276, the V_{H} CDR2 comprises SEQ ID NO: 277, the V_{H} CDR3 comprises SEQ ID NO: 278, the V_{L} CDR1 comprises SEQ ID NO: 283, the V_{L} CDR2 comprises SEQ ID NO: 284, and the V_{L} CDR3 comprises SEQ ID NO: 285. In some examples, in the ALG5-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 275; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 282. In some examples, the ALG5-binding protein comprises a heavy chain variable region comprising SEQ ID NO: 275 and a light chain variable region comprising SEQ ID NO: 282. In some examples, the ALG5-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 274; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 281. In some examples, the ALG5-binding protein comprises a heavy chain comprising SEQ ID NO: 274 and a light chain comprising SEQ ID NO: 281. In some examples, the ALG5-binding protein comprises a heavy chain comprising SEQ ID NO: 273 and a light chain comprising SEQ ID NO: 280.

In some examples, in the ALG5-binding protein, the V_{H} CDR1 comprises SEQ ID NO: 290, the V_{H} CDR2 comprises SEQ ID NO: 291, the V_{H} CDR3 comprises SEQ ID NO: 292, the V_{L} CDR1 comprises SEQ ID NO: 297, the V_{L} CDR2 comprises SEQ ID NO: 298, and the V_{L} CDR3 comprises SEQ ID NO: 299. In some examples, in the ALG5-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 289; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 296. In some examples, the ALG5-binding protein comprises a heavy chain variable region comprising SEQ ID NO: 289 and a light chain variable region comprising SEQ ID NO: 296. In some examples, the ALG5-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 288; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 295. In some examples, the ALG5-binding protein comprises a heavy chain comprising SEQ ID NO: 288 and a light chain comprising SEQ ID NO: 295. In some examples, the ALG5-binding protein comprises a heavy chain comprising SEQ ID NO: 287 and a light chain comprising SEQ ID NO: 294.

In some examples, in the ALG5-binding protein, the V_{H} CDR1 comprises SEQ ID NO: 304, the V_{H} CDR2 comprises SEQ ID NO: 305, the V_{H} CDR3 comprises SEQ ID NO: 306, the V_{L} CDR1 comprises SEQ ID NO: 311, the V_{L} CDR2 comprises SEQ ID NO: 312, and the V_{L} CDR3 comprises SEQ ID NO: 313. In some examples, in the ALG5-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 303; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 310. In some examples, the ALG5-binding protein comprises a heavy chain variable region comprising SEQ ID NO: 303 and a light chain variable region comprising SEQ ID NO: 310. In some examples, the ALG5-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 302; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 309. In some examples, the ALG5-binding protein comprises a heavy chain comprising SEQ ID NO: 302 and a light chain comprising SEQ ID NO: 309. In some examples, the ALG5-binding protein comprises a heavy chain comprising SEQ ID NO: 301 and a light chain comprising SEQ ID NO: 308.

In some examples, in the ALG5-binding protein, the V_{H} CDR1 comprises SEQ ID NO: 318, the V_{H} CDR2 comprises SEQ ID NO: 319, the V_{H} CDR3 comprises SEQ ID NO: 320, the V_{L} CDR1 comprises SEQ ID NO: 325, the V_{L} CDR2 comprises SEQ ID NO: 326, and the V_{L} CDR3 comprises SEQ ID NO: 327. In some examples, in the ALG5-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 317; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 324. In some examples, the ALG5-binding protein comprises a heavy chain variable region comprising SEQ ID NO: 317 and a light chain variable region comprising SEQ ID NO: 324. In some examples, the ALG5-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 316; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 323. In some examples, the ALG5-binding protein comprises a heavy chain comprising SEQ ID NO: 316 and a light chain comprising SEQ ID NO: 323. In some examples, the ALG5-binding protein comprises a heavy chain comprising SEQ ID NO: 315 and a light chain comprising SEQ ID NO: 322.

In some examples, in the ALG5-binding protein, the V_{H} CDR1 comprises SEQ ID NO: 332, the V_{H} CDR2 comprises SEQ ID NO: 333, the V_{H} CDR3 comprises SEQ ID NO: 334, the V_{L} CDR1 comprises SEQ ID NO: 339, the V_{L} CDR2 comprises SEQ ID NO: 340, and the V_{L} CDR3 comprises SEQ ID NO: 341. In some examples, in the ALG5-binding protein, the heavy chain variable region comprises the V_{H} CDR1, V_{H} CDR2, and V_{H} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 331; and the light chain variable region comprises the V_{L} CDR1, V_{L} CDR2, and V_{L} CDR3 above, and comprises a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 338. In some examples, the ALG5-binding protein comprises a heavy chain variable region comprising SEQ ID NO: 331 and a light chain variable region comprising SEQ ID NO: 338. In some examples, the ALG5-binding protein comprises: a heavy chain comprising the V_{H} CDR1, V_{H} CDR2, and V_{H} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 330; and a light chain comprising the V_{L} CDR1, V_{L} CDR2, and V_{L} CDR3 above, and a sequence that is at least 80%, e.g., at least 85%, 86%, 87%, 88%, 89%, 90%, 91%, 92%, 93%, 94, 95%, 96%, 97%, 98%, or 99% identical to SEQ ID NO: 337. In some examples, the ALG5-binding protein comprises a heavy chain comprising SEQ ID NO: 330 and a light chain comprising SEQ ID NO: 337. In some examples, the ALG5-binding protein comprises a heavy chain comprising SEQ ID NO: 329 and a light chain comprising SEQ ID NO: 336.

In some embodiments, the present disclosure provides nucleic acids encoding an ALG5-binding protein, e.g., SEQ ID NOs: 265, 279, 293, 307, 321, 335, 272, 286, 300, 314, 328, and 342.

In some embodiments, the ALG5 antibody is a monoclonal antibody.

### D. Caveolin-1 Scaffolding Domain Peptides

In some embodiments, the methods for treating pathogen-induced diseases herein may further comprise administering a caveolin-1 (Cav-1) scaffolding domain peptide (CSP). The Caveolin-1 (Cav-1) scaffolding domain or polypeptide interferes with Cav-1 interaction with Src kinases mimics the combined effect of uPA and anti-β1-integrin antibody. In some examples, the CSP may be comprised in the same pharmaceutical composition that comprises the MEGF9, UNC5A, ALG5, and/or biological active fragment thereof. In some examples, the CSP may be comprised in a pharmaceutical composition than the one comprising the MEGF9, UNC5A, ALG5, and/or biological active fragment thereof. In some examples, the CSP may be administered at the same time as the MEGF9, UNC5A, ALG5, and/or biological active fragment thereof. In some examples, the CSP may be administered prior to the administration of the MEGF9, UNC5A, ALG5, and/or biological active fragment thereof. In some examples, the CSP may be administered after the administration of the MEGF9, UNC5A, ALG5, and/or biological active fragment thereof.

In some examples, the CSP may comprise a sequence of SEQ ID NO: 10 (FTTFTVT). In some examples, the CSP may comprise a sequence with at least 80%, e.g., at least 85%, at least 90%, at least 95%, or at least 99% identity to SEQ ID NO: 10.

In some examples, the CSP may consist of a sequence of SEQ ID NO: 10. In some examples, the CSP may consist of a sequence with at least 80%, e.g., at least 85%, at least 90%, at least 95%, or at least 99% identity to SEQ ID NO: 10.

In some examples, the CSP may comprise a sequence of SEQ ID NO: 11. In some examples, the CSP may comprise a sequence with a one-amino acid, two-amino acid, three-amino acid, or four-amino acid modification of SEQ ID NO: 11.

In some examples, the CSP may consist of a sequence of SEQ ID NO: 11. In some examples, the CSP may consist of a sequence with a one-amino acid, two-amino acid, three-amino acid, or four-amino acid modification of SEQ ID NO: 11.

In some examples, the CSP may comprise or consist of any one of sequences shown in Table 1.

TABLE 1
Exemplary Cav-1 peptides

| SEQ ID NO: | Sequences |
|---|---|
| 12 | SFTTFTVT |
| 13 | SFTTFTVTK |
| 14 | FTTFTVTKYW |
| 15 | ASFTTFTVTK |
| 16 | FTTFTVTKYWF |
| 17 | ASFTTFTVTKY |
| 18 | WKASFTTFTVT |
| 19 | SFTTFTVTKYWF |
| 20 | KASFTTFTVTKY |
| 21 | IWKASFTTFTVT |
| 22 | SFTTFTVTKYWFY |
| 23 | KASFTTFTVTKYW |
| 24 | IWKASFTTFTVTK |
| 25 | FTTFTVTKYWFYRL |
| 26 | ASFTTFTVTKYWFY |
| 27 | WKASFTTFTVTKYW |
| 28 | GIWKASFTTFTVTK |
| 29 | FTTFTVTKYWFYRLL |
| 30 | ASFTTFTVTKYWFYR |
| 31 | WKASFTTFTVTKYWF |
| 32 | GIWKASFTTFTVTKY |
| 33 | FDGIWKASFTTFTVT |
| 34 | SFTTFTVTKYWFYRLL |
| 35 | KASFTTFTVTKYWFYR |
| 36 | IWKASFTTFTVTKYWF |
| 37 | DGIWKASFTTFTVTKY |
| 38 | SFDGIWKASFTTFTVT |
| 39 | SFTTFTVTKYWFYRLLS |
| 40 | KASFTTFTVTKYWFYRL |
| 41 | IWKASFTTFTVTKYWFY |
| 42 | DGIWKASFTTFTVTKYW |
| 43 | SFDGIWKASFTTFTVTK |
| 44 | FTTFTVTKYWFYRLLSAL |
| 45 | ASFTTFTVTKYWFYRLLS |
| 46 | WKASFTTFTVTKYWFYRL |
| 47 | GIWKASFTTFTVTKYWFY |
| 48 | FDGIWKASFTTFTVTKYW |
| 49 | HSFDGIWKASFTTFTVTK |
| 50 | FTTFTVTKYWFYRLLSALF |
| 51 | ASFTTFTVTKYWFYRLLSA |
| 52 | WKASFTTFTVTKYWFYRLL |
| 53 | GIWKASFTTFTVTKYWFYR |
| 54 | FDGIWKASFTTFTVTKYWF |
| 55 | HSFDGIWKASFTTFTVTKY |
| 56 | GTHSFDGIWKASFTTFTVT |
| 57 | FTTFTVTK |
| 58 | FTTFTVTKY |
| 59 | ASFTTFTVT |
| 60 | SFTTFTVTKY |
| 61 | KASFTTFTVT |
| 62 | SFTTFTVTKYW |
| 63 | KASFTTFTVTK |
| 64 | FTTFTVTKYWFY |
| 65 | ASFTTFTVTKYW |
| 66 | WKASFTTFTVTK |
| 67 | FTTFTVTKYWFYR |
| 68 | ASFTTFTVTKYWF |
| 69 | WKASFTTFTVTKY |
| 70 | GIWKASFTTFTVT |
| 71 | SFTTFTVTKYWFYR |
| 72 | KASFTTFTVTKYWF |
| 73 | IWKASFTTFTVTKY |
| 74 | DGIWKASFTTFTVT |
| 75 | SFTTFTVTKYWFYRL |
| 76 | KASFTTFTVTKYWFY |
| 77 | IWKASFTTFTVTKYW |
| 78 | DGIWKASFTTFTVTK |
| 79 | FTTFTVTKYWFYRLLS |
| 80 | ASFTTFTVTKYWFYRL |
| 81 | WKASFTTFTVTKYWFY |
| 82 | GIWKASFTTFTVTKYW |
| 83 | FDGIWKASFTTFTVTK |
| 84 | FTTFTVTKYWFYRLLSA |
| 85 | ASFTTFTVTKYWFYRLL |
| 86 | WKASFTTFTVTKYWFYR |
| 87 | GIWKASFTTFTVTKYWF |
| 88 | FDGIWKASFTTFTVTKY |
| 89 | HSFDGIWKASFTTFTVT |
| 90 | SFTTFTVTKYWFYRLLSA |
| 91 | KASFTTFTVTKYWFYRLL |
| 92 | IWKASFTTFTVTKYWFYR |
| 93 | DGIWKASFTTFTVTKYWF |
| 94 | SFDGIWKASFTTFTVTKY |
| 95 | THSFDGIWKASFTTFTVT |
| 96 | SFTTFTVTKYWFYRLLSAL |
| 97 | KASFTTFTVTKYWFYRLLS |
| 98 | IWKASFTTFTVTKYWFYRL |
| 99 | DGIWKASFTTFTVTKYWFY |
| 100 | SFDGIWKASFTTFTVTKYW |
| 101 | THSFDGIWKASFTTFTVTK |

The CSP may have the activity of the native CAV-1 polypeptide in in vitro or in vivo assays of binding or of biological activity. For example, the CSP may inhibit or prevent apoptosis of LECs induced by BLM in vitro or in vivo with activity at least about 20% of the activity of the native CAV-1 polypeptide, or at least about 30%, 40%, 50%, 60%, 65%, 70%, 75%, 80%, 85%, 90%, about 95%, 97%, 99%, and any range derivable therein, such as, for example, from about 70% to about 80%, and more preferably from about 81% to about 90%; or even more preferably, from about 91% to about 99%. The CSP may have 100% or even greater activity than the native Cav-1 polypeptide. Assays for testing biological activity, e.g., anti-fibrotic activity, the ability to affect expression of uPA, uPAR and PAI-1 mRNAs, or inhibit proliferation of lung fibroblasts, are well-known in the art.

### E. Modifications

The proteins and biologically active fragments thereof also include modifications of the exemplary proteins and peptides described herein. Modifications may be made to amino acids on the N-terminus, C-terminus, or internally. The proteins and biologically active fragments thereof may include conservative or non-conservative amino acid changes, as described below. Examples of the modifications include amino acid substitutions, additions, deletions, fusions and truncations in the original proteins or peptide. The proteins and biologically active fragments thereof may also include insertions, deletions or substitutions of amino acids, including insertions and substitutions of amino acids (and other molecules) that do not normally occur in the peptide sequence that may be the basis of the modified variant, for example but not limited to insertion L-amino acids, or non-standard amino acids such as ornithine, which do not normally occur in human proteins.

### 1. Substitutions

In some embodiments, the protein or peptide may comprise one or more substitutions of the sequences described herein. In some examples, the substitution may be a conservative amino acid substitution. Conservative amino acid substitutions result from replacing one amino acid with another having similar structural and/or chemical properties, such as the replacement of a leucine with an isoleucine or valine, an aspartate with a glutamate, or a threonine with a serine. Thus, a conservative substitution of a particular amino acid sequence refers to substitution of those amino acids that are not critical for polypeptide activity or substitution of amino acids with other amino acids having similar properties (e.g., acidic, basic, positively or negatively charged, polar or non-polar, etc.) such that the substitution of even critical amino acids does not reduce the activity of the protein or peptide. Conservative substitution tables providing functionally similar amino acids are well known in the art. For example, the following six groups each contain amino acids that may be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W). In some embodiments, individual substitutions, deletions, or additions that alter, add, or delete a single amino acid or a small percentage of amino acids may also be considered conservative substitutions if the change does not reduce the activity of the protein or peptide. Insertions or deletions may be typically in the range of about 1 to 6 amino acids.

In some embodiments, one may select the amino acid that will substitute an existing amino acid based on the location of the existing amino acid, e.g., its exposure to solvents (i.e. if the amino acid may be exposed to solvents or may be present on the outer surface of the protein or peptide as compared to internally localized amino acids not exposed to solvents). Selection of such conservative amino acid substitutions are well known in the art, for example as disclosed in Dordo et al, J. Mol Biol, 1999, 217, 721-739 and Taylor et al, J. Theor. Biol. 119(1986); 205-218 and S. French and B. Robson, J. Mol. Evol. 19(1983)171. Accordingly, one may select conservative amino acid substitutions suitable for amino acids on the exterior of a protein or peptide (e.g., amino acids exposed to a solvent), for example, but not limited to, the following substitutions may be used: substitution of Y with F, T with S or K, P with A, E with D or Q, N with D or G, R with K, G with N or A, T with S or K, D with N or E, I with L or V, F with Y, S with T or A, R with K, G with N or A, K with R, A with S, K or P.

In some embodiments, one may also select conservative amino acid substitutions encompassing suitable for amino acids on the interior of a protein or peptide, for example one may use suitable conservative substitutions for amino acids may be on the interior of a protein or peptide (i.e. the amino acids are not exposed to a solvent), for example but not limited to, one may use the following conservative substitutions: where Y may be substituted with F, T with A or S, I with L or V, W with Y, M with L, N with D, G with A, T with A or S, D with N, I with L or V, F with Y or L, S with A or T and A with S, G, T or V. In some embodiments, non-conservative amino acid substitutions may be also encompassed within the term of variants.

In some aspects, amino acid substitutions may be made in a protein or peptide at one or more positions wherein the substitution may be for an amino acid having a similar hydrophilicity. The importance of the hydropathic amino acid index in conferring interactive biologic function on a protein may be generally understood in the art. It may be accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus, such conservative substitution may be made in a polypeptide and may only have minor effects on their activity. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4). These values may be used as a guide and thus substitution of amino acids whose hydrophilicity values may be within ±2, within ±1, or within ±0.5. Thus, any of the proteins or peptides described herein may be modified by the substitution of an amino acid, for different, but homologous amino acid with a similar hydrophilicity value. Amino acids with hydrophilicities within +/−1.0 points, or +/−0.5 points, may be considered homologous.

In some aspects, the protein or peptide may comprise non-naturally occurring amino acids. The protein may peptide may comprise a combination of naturally occurring and non-naturally occurring amino acids, or may comprise only non-naturally occurring amino acids. The non-naturally occurring amino acids may include synthetic non-native amino acids, substituted amino acids, or one or more D-amino acids (or other components of the composition, with exception for protease recognition sequences) as desirable in certain situations. D-amino acid-containing peptides exhibit increased stability in vitro or in vivo compared to L-amino acid-containing forms. Thus, the construction of peptides incorporating D-amino acids may be particularly useful when greater in vivo or intracellular stability may be desired or required. More specifically, D-peptides may be resistant to endogenous peptidases and proteases, thereby providing better oral trans-epithelial and transdermal delivery of linked drugs and conjugates, improved bioavailability of membrane-permanent complexes, and prolonged intravascular and interstitial lifetimes when such properties may be desirable. Additionally, D-peptides cannot be processed efficiently for major histocompatibility complex class II-restricted presentation to T helper cells and may be therefore less likely to induce humoral immune responses in the whole organism.

In addition to the 20 “standard” L-amino acids, D-amino acids or non-standard, modified or unusual amino acids which are well-defined in the art may also be contemplated for use in the present disclosure. Phosphorylated amino acids (Ser, Thr, Tyr), glycosylated amino acids (Ser, Thr, Asn), β-amino acids, GABA, ω-amino acids may be further contemplated for use in the present disclosure. These include β-alanine (β-Ala) and other ω-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); norleucine (Nle); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); homoarginine (hArg); N-acetyl lysine (AcLys); 2,4-diaminobutyric acid (Dbu; Dab); p-aminophenylalanine (Phe(pNH₂)); N-methyl valine (MeVal); homocysteine (hCys), homophenylalanine (hPhe), and homoserine (hSer); hydroxyproline (Hyp), homoproline (hPro), N-methylated amino acids, and peptoids (N-substituted glycines).

### F. Derivatives

In some aspects, the protein or peptide may be derivatives of any of SEQ ID NOs: 1-9. The term “derivative” as used herein refers to proteins and peptides that are chemically modified, for example but not limited to by techniques such as acetylation, ubiquitination, labeling, pegylation (derivatization with polyethylene glycol), lipidation, glycosylation, amidation, cyclization, or addition of other molecules. The proteins and peptides may be provided in a cyclic form, e.g., as a cyclic peptide or as a lactam. Alternatively, or in addition, proteins and the peptides may be provided as a branched peptide. A molecule may be also a “derivative” of another molecule when it contains additional chemical moieties not normally a part of the molecule. Such moieties may alter the pH or improve the molecule's solubility, absorption, biological half-life, etc. The moieties may alternatively decrease the toxicity of the molecule, eliminate or attenuate any undesirable side effect of the molecule, etc. Moieties capable of mediating such effects are disclosed in Remington's Pharmaceutical Sciences, 18th edition, A. R. Gennaro, Ed., MackPubl., Easton, PA (1990), incorporated herein, by reference, in its entirety.

The term “functional” when used in conjunction with “derivative” or “variant” refers to a polypeptide of the invention that possesses a biological activity (either functional or structural) that may be substantially similar to a biological activity of the entity or molecule it may be a functional derivative or functional variant thereof. The term functional derivative may be intended to include the fragments, analogues or chemical derivatives of a molecule.

The modified proteins and peptides may comprise co-translational and post-translational (e.g., C-terminal peptide cleavage) modifications, such as, for example, disulfide-bond formation, glycosylation, acetylation, phosphorylation, proteolytic cleavage (e.g., cleavage by furins or metalloproteases), and the like to the extent that such modifications do not affect the function of the peptides.

In yet a further aspect, the protein or peptide may be “retro-inverso peptides.” A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide may contain L-amino acids or D-amino acids, or a mixture of L-amino acids and D-amino acids, up to all of the amino acids being the D-isomer. Partial retro-inverso peptide analogues may be polypeptides in which only part of the sequence may be reversed and replaced with enantiomeric amino acid residues. Since the retro-inverted portion of such an analogue has reversed amino and carboxyl termini, the amino acid residues flanking the retro-inverted portion may be replaced by side-chain-analogous a-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which may be also incorporated herein in its entirety by reference.

### G. Terminal Modifications

The protein and peptide may be modified (at its amino terminus or carboxy terminus. Examples of amino terminal modifications include, e.g., N-glycated, N-alkylated, N-acetylated or N-acylated amino acid. A terminal modification may include a pegylation. An example of a carboxy terminal modification may be a C-terminal amidated amino acid. The proteins and peptides may be cross-linked or have a cross-linking site (for example, the protein or peptide may a cysteinyl residue and thus forms cross-linked dimers in culture or in vivo. One or more peptidyl bonds may be replaced by a non-peptidyl linkage; the N-terminus or the C-terminus may be replaced, and individual amino acid moieties may be modified through treatment with agents capable of reacting with selected side chains or terminal residues, and so forth. Either the C-terminus or the N-terminus of the sequences, or both, may be linked to a carboxylic acid functional groups or an amine functional group, respectively.

Examples of N-terminal protecting groups include acyl groups (—CO—R1) and alkoxy carbonyl or aryloxy carbonyl groups (—CO—O—R1), wherein R1 may be an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—, Adamantan, naphtalen, myristoleyl, toluen, biphenyl, cinnamoyl, nitrobenzoy, toluoyl, furoyl, benzoyl, cyclohexane, norbornane, or Z-caproic. In order to facilitate the N-acylation, one to four glycine residues may be present at the N-terminus of the molecule.

Carboxy terminal modifications include acylation with carboxylic acids: formic, acetic, propionic, fatty acids (myristic, palmitic, stearic), succinic, benzoic, carbobenzoxy (Cbz); acetylation and biotinylation. Amino terminal modifications include: (i) acylation with carboxylic acids: formic, acetic, propionic, fatty acids (myristic, palmitic, stearic, etc) succinic, benzoic, carbobenzoxy (Cbz); (ii) biotinylation; (iii) amidation; (iv) attachment of dyes such as fluorescein (FITC, FAM, etc.), 7-hydroxy-4-methylcoumarin-3-acetic acid, 7-hydroxycoumarin-3-acetic acid, 7-metoxycoumarin-3-acetic acid and other coumarins; rhodamines (5-carboxyrhodamine 110 or 6G, 5(6)-TAMRA, ROX); N-[4-(4-dimethylamino)phenylazo]bezoic acid (Dabcyl), 2,4-dinitrobenzene (Dnp), 5-dimethylaminonaphthalene-1-sulfonic acid (Dansyl) and other dyes; and (v) polyethyleneglycol.

The carboxyl group at the C-terminus of a protein or peptide may be protected, for example, by a group including but not limited to an amide (i.e., the hydroxyl group at the C-terminus may be replaced with —NH2, —NHR2 and —NR2R3) or ester (i.e. the hydroxyl group at the C-terminus may be replaced with —OR2). R2 and R3 may be optionally independently an aliphatic, substituted aliphatic, benzyl, substituted benzyl, aryl or a substituted aryl group. In addition, taken together with the nitrogen atom, R2 and R3 may optionally form a C4 to C8 heterocyclic ring with from about 0-2 additional heteroatoms such as nitrogen, oxygen or sulfur. Non-limiting suitable examples of suitable heterocyclic rings include piperidinyl, pyrrolidinyl, morpholino, thiomorpholino or piperazinyl. Examples of C-terminal protecting groups include —NH2, —NHCH3, —N(CH3)2, —NH(ethyl), —N(ethyl)2, —N(methyl) (ethyl), —NH(benzyl), —N(C1-C4 alkyl)(benzyl), —NH(phenyl), —N(C1-C4 alkyl)(phenyl), —OCH3, —O-(ethyl), —O-(n-propyl), —O-(n-butyl), —O-(iso-propyl), —O-(sec-butyl), —O-(t-butyl), —O-benzyl and —O-phenyl.

### H. Side Chain Modifications

The amino acids of the protein and peptide may optionally be modified according to any one of the following exemplary types of modification. Non-limiting exemplary types of modification include carboxymethylation, acylation, phosphorylation, glycosylation or fatty acylation. Ether bonds may optionally be used to join the serine or threonine hydroxyl to the hydroxyl of a sugar. Amide bonds may optionally be used to join the glutamate or aspartate carboxyl groups to an amino group on a sugar (Gang and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds may also optionally be formed between amino acids and carbohydrates. Fatty acid acyl derivatives may optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a protein or peptide herein, refers to a protein or peptide where at least one of its amino acid residues may be modified either by natural processes, such as processing or other post-translational modifications, or by chemical modification techniques which are well known in the art. Examples of the numerous known modifications typically include acetylation, acylation, amidation, ADP-ribosylation, glycosylation, GPI anchor formation, covalent attachment of a lipid or lipid derivative, methylation, myristylation, pegylation, prenylation, phosphorylation, ubiquitination, or any similar process.

Other types of modifications may optionally include the addition of a cycloalkane moiety to a biological molecule, such as a protein, as described in PCT Application No. WO 2006/050262, hereby incorporated by reference as if fully set forth herein. These moieties may be designed for use with biomolecules and may optionally be used to impart various properties to proteins.

Furthermore, optionally any point on a protein or peptide may be modified. For example, pegylation of a glycosylation moiety on a protein or peptide may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein or peptide through a glycosyl linker.

Covalent modifications of the proteins and peptides are included within the scope of this invention. Other types of covalent modifications of the proteins or peptides may be introduced into the molecule by reacting targeted amino acid residues with an organic derivatizing agent that may be capable of reacting with selected side chains or the N- or C-terminal residues.

Cysteinyl residues may be reacted with α-haloacetates (and corresponding amines), such as chloroacetic acid or chloroacetamide, to give carboxymethyl or carboxyamidomethyl derivatives. Cysteinyl residues also may be derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues may be derivatized by reaction with diethylpyrocarbonate at pH 5.5-7.0 because this agent may be relatively specific for the histidyl side chain. Para-bromophenacyl bromide also may be useful; the reaction may be preferably performed in 0.1M sodium cacodylate at pH 6.0.

Lysinyl and amino-terminal residues may be reacted with succinic or other carboxylic acid anhydrides. Derivatization with these agents may have the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione, and transaminase-catalyzed reaction with glyoxylate.

Arginyl residues may be modified by reaction with one or several conventional reagents, among them phenylglyoxal, 2,3-butanedione, 1,2-cyclohexanedione, and ninhydrin.

Derivatization of arginine residues may need the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. For example, N-acetylimidizole and tetranitromethane may be used to form O-acetyl tyrosyl species and 3-nitro derivatives, respectively. Tyrosyl residues may be iodinated using ¹²⁵I or ¹³¹I to prepare labeled proteins or peptides for use in radioimmunoassay.

Carboxyl side groups (aspartyl or glutamyl) may be selectively modified by reaction with carbodiimides (R—N═C═N—R′), where R and R′ may be different alkyl groups, such as 1-cyclohexyl-3-(2-morpholinyl-4-ethyl)carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Derivatization with bifunctional agents may be useful for crosslinking to a water-insoluble support matrix or surface for use in the method for purifying anti-CHF antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that may be capable of forming crosslinks in the presence of light. Alternatively or additionally, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for protein immobilization.

Glutaminyl and asparaginyl residues may be frequently deamidated to the corresponding glutamyl and aspartyl residues, respectively. These residues may be deamidated under neutral or basic conditions.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, pp. 79-86 [1983]), acetylation of the N-terminal amine, and amidation of any C-terminal carboxyl group.

### I. Capping

The protein and peptide may be capped at the N- and/or C-termini with an acyl (abbreviated “Ac”) and an amido (abbreviated “Am”) group, respectively, for example acetyl (CH₃CO—) at the N-terminus and amido (—NH₂) at the C-terminus. A broad range of N-terminal capping functions, preferably in a linkage to the terminal amino group, is contemplated, for example: formyl;
alkanoyl, having from 1 to 10 carbon atoms, such as acetyl, propionyl, butyryl;
alkenoyl, having from 1 to 10 carbon atoms, such as hex-3-enoyl;
alkynoyl, having from 1 to 10 carbon atoms, such as hex-5-ynoyl;
aroyl, such as benzoyl or 1-naphthoyl;
heteroaroyl, such as 3-pyrroyl or 4-quinoloyl;
alkylsulfonyl, such as methanesulfonyl;
arylsulfonyl, such as benzenesulfonyl or sulfanilyl;
heteroarylsulfonyl, such as pyridine-4-sulfonyl;
substituted alkanoyl, having from 1 to 10 carbon atoms, such as 4-aminobutyryl;
substituted alkenoyl, having from 1 to 10 carbon atoms, such as 6-hydroxy-hex-3-enoyl;
substituted alkynoyl, having from 1 to 10 carbon atoms, such as 3-hydroxy-hex-5-ynoyl;
substituted aroyl, such as 4-chlorobenzoyl or 8-hydroxy-naphth-2-oyl;
substituted heteroaroyl, such as 2,4-dioxo-1,2,3,4-tetrahydro-3-methyl-quinazolin-6-oyl;
substituted alkylsulfonyl, such as 2-aminoethanesulfonyl;
substituted arylsulfonyl, such as 5-dimethylamino-1-naphthalenesulfonyl;
substituted heteroarylsulfonyl, such as 1-methoxy-6-isoquinolinesulfonyl; carbamoyl or thiocarbamoyl;
substituted carbamoyl (R′—NH—CO) or substituted thiocarbamoyl (R′—NH—CS) wherein R′ may be alkyl, alkenyl, alkynyl, aryl, heteroaryl, substituted alkyl, substituted alkenyl, substituted alkynyl, substituted aryl, or substituted heteroaryl;
substituted carbamoyl (R′—NH—CO) and substituted thiocarbamoyl (R′—NH—CS) wherein R′ may be alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

The C-terminal capping function may either be in an amide or ester bond with the terminal carboxyl. Capping functions that provide for an amide bond are designated as NR¹R² wherein R¹ and R² may be independently drawn from the following group: hydrogen;
alkyl, preferably having from 1 to 10 carbon atoms, such as methyl, ethyl, isopropyl;
alkenyl, preferably having from 1 to 10 carbon atoms, such as prop-2-enyl;
alkynyl, preferably having from 1 to 10 carbon atoms, such as prop-2-ynyl;
substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl;
substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl;
substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;
aroylalkyl having up to 10 carbon atoms, such as phenacyl or 2-benzoylethyl;
aryl, such as phenyl or 1-naphthyl;
heteroaryl, such as 4-quinolyl;
alkanoyl having from 1 to 10 carbon atoms, such as acetyl or butyryl;
aroyl, such as benzoyl;
heteroaroyl, such as 3-quinoloyl;
OR′ or NR′R″ where R′ and R″ may be independently hydrogen, alkyl, aryl, heteroaryl, acyl, aroyl, sulfonyl, sulfinyl, or SO₂—R′″ or SO—R′″ where R′″ may be substituted or unsubstituted alkyl, aryl, heteroaryl, alkenyl, or alkynyl.

Capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

Either the N-terminal or the C-terminal capping function, or both, may be of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups may allow the addition of other activities on the protein or peptide. For example, the presence of a sulfhydryl group linked to the N- or C-terminal cap may permit conjugation of the derivatized peptide to other molecules.

### J. Multimerization

In some embodiments, the protein or peptide may be longer molecules built from multimerizing (e.g., repeating) units of a protein or peptide described herein. A protein or peptide multimer may comprise different combinations of proteins or peptides. Such multimeric protein or peptide be made by chemical synthesis or by recombinant DNA techniques as discussed herein. When produced by chemical synthesis, the oligomers may have from 2-5 repeats of a core polypeptide sequence, and the total number of amino acids in the multimer may be less than about 160 residues, e.g., not more than 100 residues (or their equivalents, when including linkers or spacers).

### K. Peptidomimetics

The protein or peptide herein may be a peptidomimetic compound, which mimics the biological effects of the corresponding native protein or peptide. A peptidomimetic agent may be an unnatural peptide or a non-peptide agent that recreates the stereospatial properties of the binding elements of the native protein or peptide such that it has the binding activity and biological activity of the native protein or peptide. Similar to a native protein or peptide, or multimer, a peptidomimetic will have a binding face (which interacts with any ligand to which native protein or peptide binds) and a non-binding face.

In some aspects, the present disclosure also includes compounds that retain partial peptide characteristics. For example, any proteolytically unstable bond within a protein or peptide may be selectively replaced by a non-peptidic element such as an isostere (N-methylation; D-amino acid) or a reduced peptide bond while the rest of the molecule retains its peptidic nature.

The peptidomimetic compounds, either agonists, substrates or inhibitors, included those described for a number of bioactive peptides/polypeptides such as opioid peptides, VIP, thrombin, HIV protease, etc. Methods for designing and preparing peptidomimetic compounds are known in the art (Hruby, V J, Biopolymers 33:1073-1082 (1993); Wiley, R A et al., Med. Res. Rev. 13:327-384 (1993); Moore et al., Adv. in Pharmacol 33:91-141 (1995); Giannis et al., Adv. in Drug Res. 29:1-78 (1997). Certain mimetics that mimic secondary structure are described in Johnson et al., In: Biotechnology and Pharmacy, Pezzuto et al., Chapman and Hall (Eds.), NY, 1993. These methods may be used to make peptidomimetics that possess at least the binding capacity and specificity of the native protein or peptide and may possess the biological activity. Knowledge of peptide chemistry and general organic chemistry available to those skilled in the art may be sufficient, in view of the present disclosure, for designing and synthesizing such compounds.

For example, such peptidomimetics may be identified by inspection of the three-dimensional structure of a protein or peptide either free or bound in complex with a ligand (e.g., soluble uPAR or a fragment thereof). Alternatively, the structure of a protein or peptide bound to its ligand may be gained by the techniques of nuclear magnetic resonance spectroscopy. Greater knowledge of the stereochemistry of the interaction of the protein or peptide with its ligand or receptor may permit the rational design of such peptidomimetic agents. The structure of a protein or peptide in the absence of ligand may also provide a scaffold for the design of mimetic molecules.

### L. PEGylation

The protein or peptide may be conjugated with heterologous polypeptide segments or polymers, such as polyethylene glycol. The protein or peptide may be linked to PEG to increase the hydrodynamic radius of the enzyme and hence increase the serum persistence. The protein or peptide may be conjugated to any targeting agent, such as a ligand having the ability to specifically and stably bind to an external receptor (see e.g., U.S. Patent Publ. 2009/0304666).

In certain aspects, methods and compositions of the embodiments related to PEGylation of the protein or peptide. PEGylation may be the process of covalent attachment of poly(ethylene glycol) polymer chains to another molecule, normally a drug or therapeutic protein. PEGylation may be achieved by incubation of a reactive derivative of PEG with the target macromolecule. The covalent attachment of PEG to a drug or therapeutic protein may “mask” the agent from the host's immune system (reduced immunogenicity and antigenicity) or increase the hydrodynamic size (size in solution) of the agent, which may prolong its circulatory time by reducing renal clearance. PEGylation may also provide water solubility to hydrophobic drugs and proteins.

The first step of the PEGylation may be the suitable functionalization of the PEG polymer at one or both terminals. PEGs that are activated at each terminus with the same reactive moiety are known as “homobifunctional,” whereas if the functional groups present may be different, then the PEG derivative may be referred as “heterobifunctional” or “heterofunctional.” The chemically active or activated derivatives of the PEG polymer may be prepared to attach the PEG to the desired molecule.

The choice of the suitable functional group for the PEG derivative may be based on the type of available reactive group on the molecule that may be coupled to the PEG. For proteins, typical reactive amino acids include lysine, cysteine, histidine, arginine, aspartic acid, glutamic acid, serine, threonine, and tyrosine. The N-terminal amino group and the C-terminal carboxylic acid may also be used.

The techniques used to form first generation PEG derivatives may be generally reacting the PEG polymer with a group that may be reactive with hydroxyl groups, typically anhydrides, acid chlorides, chloroformates, and carbonates. In the second generation PEGylation chemistry more efficient functional groups, such as aldehyde, esters, amides, etc., may be made available for conjugation.

As applications of PEGylation have become more and more advanced and sophisticated, there has been an increase in need for heterobifunctional PEGs for conjugation. These heterobifunctional PEGs may be very useful in linking two entities, where a hydrophilic, flexible, and biocompatible spacer may be needed. Preferred end groups for heterobifunctional PEGs may be maleimide, vinyl sulfones, pyridyl disulfide, amine, carboxylic acids, and NHS esters.

The most common modification agents, or linkers, may be based on methoxy PEG (mPEG) molecules. Their activity depends on adding a protein-modifying group to the alcohol end. In some instances, polyethylene glycol (PEG diol) may be used as the precursor molecule. The diol may be subsequently modified at both ends in order to make a hetero- or homo-dimeric PEG-linked molecule.

Protein or peptide may PEGylated at nucleophilic sites, such as unprotonated thiols (cysteinyl residues) or amino groups. Examples of cysteinyl-specific modification reagents include PEG maleimide, PEG iodoacetate, PEG thiols, and PEG vinylsulfone. All four may be strongly cysteinyl-specific under mild conditions and neutral to slightly alkaline pH but each has some drawbacks. The thioether formed with the maleimides may be somewhat unstable under alkaline conditions so there may be some limitation to formulation options with this linker. The carbamothioate linkage formed with iodo PEGs may be more stable, but free iodine may modify tyrosine residues under some conditions. PEG thiols form disulfide bonds with protein thiols, but this linkage may also be unstable under alkaline conditions. PEG-vinylsulfone reactivity may be relatively slow compared to maleimide and iodo PEG; however, the thioether linkage formed may be quite stable. Its slower reaction rate also may make the PEG-vinylsulfone reaction easier to control.

Site-specific PEGylation at native cysteinyl residues may be carried out. On the other hand, site-directed mutagenesis may be used to incorporate cysteinyl PEGylation sites for thiol-specific linkers. The cysteine mutation may be designed such that it may be accessible to the PEGylation reagent and may be still biologically active after PEGylation.

The proteins and peptides may be modified by amine-specific modification agents, e.g., PEG NHS ester, PEG tresylate, PEG aldehyde, PEG isothiocyanate, and several others. All may react under mild conditions and may be specific for amino groups. The PEG NHS ester may be one of the more reactive agents; however, its high reactivity may make the PEGylation reaction difficult to control on a large scale. PEG aldehyde forms an imine with the amino group, which may be then reduced to a secondary amine with sodium cyanoborohydride. Unlike sodium borohydride, sodium cyanoborohydride may not reduce disulfide bonds. However, this chemical may be highly toxic and may need to be handled cautiously, particularly at lower pH where it becomes volatile.

Because these reagents react with unprotonated amino groups, it may be possible to direct the PEGylation to lower-pK amino groups by performing the reaction at a lower pH. Generally the pK of the alpha-amino group may be 1-2 pH units lower than the epsilon-amino group of lysine residues. By PEGylating the molecule at pH 7 or below, high selectivity for the N-terminus frequently may be attained. This may be if the N-terminal portion of the protein may be not required for biological activity. Still, the pharmacokinetic benefits from PEGylation may result in a product with much greater in vivo bioactivity regardless of PEGylation chemistry.

There may be several parameters to consider when developing a PEGylation procedure. The “design of experiments” approach to optimization of PEGylation conditions may be very useful. For thiol-specific PEGylation reactions, parameters to consider include: protein concentration, PEG-to-protein ratio (on a molar basis), temperature, pH, reaction time, and in some instances, the exclusion of oxygen. For example, oxygen may contribute to intermolecular disulfide formation by the protein, which may reduce the yield of the PEGylated product. The same factors may be considered (with the exception of oxygen) for amine-specific modification except that pH may be even more critical, particularly when targeting the N-terminal amino group.

For both amine- and thiol-specific modifications, the reaction conditions may affect the stability of the protein. This may limit the temperature, protein concentration, and pH. In addition, the reactivity of the PEG linker may need to be known before starting the PEGylation reaction. For example, if the PEGylation agent may be only 70 percent active, the amount of PEG used may ensure that only active PEG molecules may be counted in the protein-to-PEG reaction stoichiometry.

### M. Fusion Proteins

In some embodiments, the protein and peptide may be a fusion protein of multiple proteins or peptides (e.g., fusion proteins of one or more sequences described in SEQ ID NOs: 1-11). The fusion protein may have the protein or peptides of the embodiments linked at the N- or C-terminus to a heterologous peptide or protein. For example, the fusion proteins may also employ leader sequences from other species to permit the recombinant expression of a protein in a heterologous host. Fusion proteins may comprise a half-life extender. Another useful fusion includes the addition of a protein affinity tag, such as a serum albumin affinity tag or six histidine residues, or an immunologically active domain, such as an antibody epitope, preferably cleavable, to facilitate purification of the fusion protein. Examples of affinity tags include polyhistidine, chitin binding protein (CBP), maltose binding protein (MBP), and glutathione-S-transferase (GST).

In some embodiments, the protein or peptide of the embodiments may be linked to a peptide that increases the in vivo half-life, such as an XTEN® polypeptide (Schellenberger et al., 2009), IgG Fc domain, albumin, or albumin binding peptide.

Methods of generating fusion proteins are well known to those of skill in the art. Such proteins may be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that may be spliced between the polypeptides connected in tandem. The linker would be of sufficient length to allow proper folding of the resulting fusion protein.

### N. Linkers

In certain embodiments, the protein or peptide may be chemically conjugated using bifunctional cross-linking reagents or fused at the protein level with peptide linkers. Bifunctional cross-linking reagents may be used for a variety of purposes, including preparation of affinity matrices, modification and stabilization of diverse structures, identification of ligand and receptor binding sites, and structural studies. Suitable peptide linkers may also be used to link the polypeptide of the embodiments, such as Gly-Ser linkers.

Homobifunctional reagents that carry two identical functional groups may provide highly efficient in inducing cross-linking between identical and different macromolecules or subunits of a macromolecule, and linking of polypeptide ligands to their specific binding sites. Heterobifunctional reagents may contain two different functional groups. By taking advantage of the differential reactivities of the two different functional groups, cross-linking may be controlled both selectively and sequentially. The bifunctional cross-linking reagents may be divided according to the specificity of their functional groups, e.g., amino-, sulfhydryl-, guanidine-, indole-, carboxyl-specific groups. Reagents directed to free amino groups may be used because of their commercial availability, ease of synthesis, and the mild reaction conditions under which they may be applied.

Heterobifunctional cross-linking reagents may contain a primary amine-reactive group and a thiol-reactive group. In some examples, heterobifunctional cross-linking reagents and methods of using the cross-linking reagents may be those described in U.S. Pat. No. 5,889,155, incorporated herein by reference in its entirety. The cross-linking reagents may combine a nucleophilic hydrazide residue with an electrophilic maleimide residue, allowing coupling, in one example, of aldehydes to free thiols. The cross-linking reagent may be modified to cross-link various functional groups.

Additionally, any other linking/coupling agents and/or mechanisms known to those of skill in the art may be used to combine polypeptides of the embodiments, such as, for example, antibody-antigen interaction, avidin biotin linkages, amide linkages, ester linkages, thioester linkages, ether linkages, thioether linkages, phosphoester linkages, phosphoramide linkages, anhydride linkages, disulfide linkages, ionic and hydrophobic interactions, bispecific antibodies and antibody fragments, or combinations thereof.

A cross-linker having reasonable stability in blood may be used. Numerous types of disulfide-bond containing linkers are known that may be successfully employed to conjugate targeting and therapeutic/preventative agents. Linkers that contain a disulfide bond that may be sterically hindered may prove to give greater stability in vivo. These linkers may be thus one group of linking agents.

Non-hindered linkers may also be employed in accordance herewith. Other useful cross-linkers, not considered to contain or generate a protected disulfide, include SATA, SPDP, and 2-iminothiolane (Wawrzynczak and Thorpe, 1987). The use of such cross-linkers is well understood in the art. Another embodiment involves the use of flexible linkers.

Once chemically conjugated, the proteins and peptide may be purified to separate the conjugate from unconjugated agents and from other contaminants. A large number of purification techniques are available for use in providing conjugates of a sufficient degree of purity to render them clinically useful. Examples of purification methods include those based upon size separation, such as gel filtration, gel permeation, or high performance liquid chromatography. Other chromatographic techniques, such as Blue-Sepharose separation, may also be used. Conventional methods to purify the fusion proteins from inclusion bodies may be useful, such as using weak detergents, such as sodium N-lauroyl-sarcosine (SLS).

### O. Cell Penetrating and Membrane Translocation Peptides

In some embodiments, the protein and peptide may further comprise a cell-binding domain or cell penetrating peptide (CPP). As used herein the terms “cell penetrating peptide” and “membrane translocation domain” and “protein transduction domain” are used interchangeably and refer to segments of polypeptide sequence that allow a polypeptide to cross the cell membrane (e.g., the plasma membrane in the case a eukaryotic cell). Examples of CPPs include segments derived from HIV-binding peptides, HIV-1 Tat (HIV), Tat-derived peptides, Penetratin, VP22 derived or analog peptides, HSV VP22 (Herpes simplex), protegrin I, MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides (particularly from Drosophila Antennapedia), pAntp, T1 (TKIESLKEHG, SEQ ID NO: 343), T2 (TQIENLKEKG, SEQ ID NO: 344), 26 (AALEALAEALEALAEALEALAEAAAA, SEQ ID NO: 345), INF7 (GLFEAIEGFIENGWEGMIEGWYGCG, SEQ ID NO: 346) pIsl, FGF, Lactoferrin, Transportan, Buforin-2, Bac715-24, SynB, SynB(1), pVEC, hCT-derived peptides, SAP, or histones.

Cell penetrating peptides may have an amino acid composition that either contains a high relative abundance of positively charged amino acids such as lysine or arginine or have a sequence that contains an alternating pattern of polar/charged amino acids and non-polar, hydrophobic amino acids. These two types of structures are referred to as polycationic or amphipathic, respectively. In some examples, cell penetrating peptides (CPPs) may be peptides of 8 to 50 residues that have the ability to cross the cell membrane and enter into most cell types. In some examples, CPPs also include protein transduction domain (PTDs) reflecting their origin as occurring in natural proteins. Frankel and Pabo simultaneously to Green and Lowenstein described the ability of the trans-activating transcriptional activator from the human immunodeficiency virus 1 (HIV-TAT) to penetrate into cells (Frankel, A. D. and C. O. Pabo, Cellular uptake of the tat protein from human immunodeficiency virus. Cell, 1988. 55(6): p. 1189-93). In 1991, transduction into neural cells of the Antennapedia homeodomain (DNA-binding domain) from Drosophila melanogaster was described (Joliot, A., et al., Antennapedia homeobox peptide regulates neural morphogenesis. Proc Natl Acad Sci USA, 1991. 88(5): p. 1864-8). In 1994, the first 16-mer peptide CPP called Penetratin (RQIKIWFQNRRMKWKK, SEQ ID NO: 347) was characterized from the third helix of the homeodomain of Drosophila Antennapedia homeobox gene product (Derossi, D., et al., The third helix of the Antennapedia homeodomain translocates through biological membranes. J Biol Chem, 1994. 269(14): p. 10444-50), followed in 1998 by the identification of the minimal domain of TAT required for protein transduction (e.g., GRKKRRQRRRPPQ, SEQ ID NO: 348) (Vives, E., P. Brodin, and B. Lebleu, A truncated HIV-1 Tat protein basic domain rapidly translocates through the plasma membrane and accumulates in the cell nucleus. J Biol Chem, 1997. 272(25): p. 16010-7). Over the past two decades, dozens of peptides were described from different origins including viral proteins, e.g. herpes virus VP22 (Elliott, G. and P. O'Hare, Intercellular trafficking and protein delivery by a herpesvirus structural protein. Cell, 1997. 88(2): p. 223-33), or from venoms, e.g. melittin (GIGAVLKVLTTGLPALISWIKRKRQQ, SEQ ID NO: 349) (Dempsey, C. E., The actions of melittin on membranes. Biochim Biophys Acta, 1990. 1031 (2): p. 143-61), mastoporan (Konno, K., et ah, Structure and biological activities of eumenine mastoparan-AF (EMP-AF), a new mast cell degranulating peptide in the venom of the solitary wasp (Anterhynchium flavomarginatum micado). Toxicon, 2000. 38(11):1505-15), maurocalcin (Esteve, E., et al., Transduction of the scorpion toxin maurocalcine into cells. Evidence that the toxin crosses the plasma membrane. J Biol Chem, 2005. 280(13): p. 12833-9), crotamine (Nascimento, F. D., et al., Crotamine mediates gene delivery into cells through the binding to heparan sulfate proteoglycans. J Biol Chem, 2007. 282(29): p. 21 349-60) or buforin (Kobayashi, S., et al., Membrane translocation mechanism of the antimicrobial peptide buforin 2. Biochemistry, 2004. 43(49): p. 15610-6). Synthetic CPPs were also designed including the poly-arginine (R8, R9, R10 and R12) (Futaki, S., et al., Arginine-rich peptides. An abundant source of membrane-permeable peptides having potential as carriers for intracellular protein delivery. J Biol Chem, 2001. 276(8): p. 5836-40) or transportan (Pooga, M., et al., Cell penetration by transportan. FASEB J, 1998. 12(1): p. 67-77). Any of the above described CPPs may be used as cell penetrating peptide in the protein or peptide according to the present invention. Various CPPs, which may be used as cell penetrating peptide in the protein or peptide herein include those disclosed in the review: Milletti, F., Cell-penetrating peptides: classes, origin, and current landscape. Drug Discov Today 17 (15-16): 850-60, 2012.

### III. Methods of Use

In another aspect, the present disclosure provides a method of using the protein and peptide described herein and mutants, variants, analogs or derivatives thereof. The method may relate to administering a composition (e.g., pharmaceutical composition) comprising one or more of the proteins and/or peptides described herein or their pharmaceutically acceptable modifications in a pharmaceutically acceptable carrier to a subject, for treating or preventing a disease, injury, or infection, e.g., in the lungs (e.g., pathogen-induced lung injury). The composition (e.g., pharmaceutical composition) may further comprise the proteins and/or peptides in a pharmaceutically acceptable carrier.

### A. Pharmaceutical Compositions

A pharmaceutical composition comprising one or more of the proteins and peptides herein may be administered systemically or locally to inhibit cell apoptosis and for the treatment and prevention damage to tissues (e.g., the lungs). The pharmaceutical composition may be administered by at least one mode selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrathecal, intra-Ommaya, intravitreous, intraocular, intrasynovial, intrathoracic, intrauterine, intravesical, bolus, vaginal, rectal, buccal, sublingual, intranasal, or transdermal. For example, a dry powder formulation may be administered by installation into a subject (e.g., subcutaneous installation) or may be reconstituted in a liquid prior to injection. In some examples, the pharmaceutical composition may be delivered locally to the airway, such as administration of a nebulized formulation or a dry powder formulation using a dry powder inhaler. They may be administered alone or in combination with anti-fibrotic compounds.

In some embodiments, the proteins and/or peptides may be administered in combination, simultaneously or sequentially with at least one additional therapeutic agent (e.g., an agent used for treating lung fibrosis). An additional therapeutic agent may be comprised in the pharmaceutical composition comprising the one or more of the proteins and peptides. Alternatively or additionally, an additional therapeutic agent may be in another pharmaceutical composition different from the pharmaceutical composition comprising the one or more of the proteins and peptides.

Examples of the additional therapeutic agents include an NSAID, steroid, DMARD, immunosuppressive, biologic response modulators, bronchodilator or antifibrotic agent such as pirfenedone, an agent whose antifibrotic mechanism of action may be not fully understood but may involve blockade of TGF-beta, nintedanib, a broad tyrosine kinase blocker or any other antifibrotic agent. Examples of NSAIDS include the non-selective COX-inhibitors acetylsalicyclic acid, mesalazin, ibuprofen, naproxen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lornoxicam and nimesulide and the pharmaceutically acceptable salts thereof, the selective COX 2-inhibitors meloxicam, celecoxib and rofecoxib and the pharmaceutically acceptable salts thereof. Examples of steroids include prednisone, prednisolone, methylprednisolone, dexamethasone, budenoside, fluocortolone and triamcinolone. Suitable DMARDs may be sulfasalazine, olsalazine, chloroquin, gold derivatives (Auranofin), D-penicillamine and cytostatics such as methotrexate and cyclophosphamide. Examples of immunsuppressives include cyclosporine A and derivatives thereof, mycophenolatemofetil, FK 506, OKT-3, ATG, 15-desoxyspergualin, mizoribine, misoprostol, rapamycin, reflunomide and azathioprine. Examples of biologic response modifiers include interferon J, anti-TNF-α (Etanercept), IL-10, anti-CD3 or anti-CD25. Suitable bronchodilators may be ipratropiumbromide, oxytropiumbromide, tiotropiumbromide, epinephrinehydrochloride, salbutamole, terbutalinsulfate, fenoterolhydrobromide, salmeterole and formoterole. In some examples, in such combinations each active ingredient may be administered either in accordance with its usual dosage range or a dose below its usual dosage range. The dosage for the combined NSAIDs, steroids, DMARDs, immunosuppressives and biologic response modifiers may be appropriately 1/50 of the lowest dose normally recommended up to 1/1 of the normally recommended dosage, preferably 1/20 to ½ and more preferably 1/10 to ⅕. The normally recommended dose for the combined drug may be the dose disclosed for example in Rote Liste® 2002, Editio Cantor Verlag Aulendorf, Germany, or in Physician's Desk Reference.

Examples of the additional therapeutic agents further include those for treating the pathogen or pathogen-induced lung injury, such as, for example, chloroquine, hydroxychloroquine, type I interferon, Azithromycin, Tocilizumab, sarilumab, interferon beta, anti-virals, remdesivir, baricitinib, dexamethasone, monoclonal antibodies including bamlanivimab (LY-CoV555), etesevimab, Casirivimab and imdevimab, AZD7442, VIR-7831, bemnifosbuvir (AT-527), lenzilumab, leronlimab, favipiravir, lopinavir, nirmatrelvir, molnupiravir, ritonavir, and any combination thereof.

Hydroxychloroquine may be a chemical derivative of chloroquine (CQ) which features a hydroxyethyl group instead of an ethyl group. Hydroxychloroquine has been classified as an effective anti-malarial medication and has shown efficacy in treating systemic lupus erythematosus as well as rheumatoid arthritis and Sjögren's Syndrome. While hydroxychloroquine has been known for some time to increase lysosomal pH in antigen presenting cells, its mechanism of action in inflammatory conditions has been only recently elucidated and involves blocking the activation of toll-like receptors to on plasmacytoid dendritic cells (PDCs). Hydroxychloroquine has shown efficacy in treating RNA viruses, including hepatitis C. Hydroxychloroquine may be administered at a dose of 600 mg per day.

Human type I interferons (IFNs) are a large subgroup of interferon proteins that help regulate the activity of the immune system. The mammalian types are designated IFN-α (alpha), IFN-β (beta), IFN-κ (kappa), IFN-δ (delta), IFN—ε (epsilon), IFN-τ (tau), IFN-ω (omega), and IFN-ζ (zeta, also known as limitin). Type I interferons have shown efficacy against the replication of various viruses, included Zika virus, chikungunya virus, flaviviruses, and hepatitis C virus. “Interferon compounds” include interferon-alpha, interferon-alpha analogues, interferon-alpha derivatives, interferon-alpha conjugates, interferon beta, interferon-beta analogues, interferon-beta derivatives, interferon-beta conjugates and mixtures thereof. The whole protein or its fragments may be fused with other peptides and proteins such as immunoglobulins and other cytokines. Interferon-alpha and interferon-beta conjugates may represent, for example, a composition comprising interferon-beta coupled to a non-naturally occurring polymer comprising a polyalkylene glycol moiety. Preferred interferon compounds include Roferon®, Intron®, Alferon®, Infergen®, Omniferon®, Alfacon-1, interferon-alpha, interferon-alpha analogues, pegylated interferon-alpha, polymerized interferon-alpha, dimerized interferon-alpha, interferon-alpha conjugated to carriers, interferon-alpha as oral inhalant, interferon-alpha as injectable compositions, interferon-alpha as a topical composition, Roferon® analogues, Intron® analogues, Alferon® analogues, and Infergen® analogues, Omniferon® analogues, Alfacon-1 analogues, interferon beta, Avonex™, Betaseron™, Betaferon™, Rebif™, interferon-beta analogues, pegylated interferon-beta (Plegridy®), polymerized interferon-beta, dimerized interferon-beta, interferon-beta conjugated to carriers, interferon-beta as oral inhalant, interferon-beta as an injectable composition, interferon-beta as a topical composition, Avonex™ analogues, Betaseron™, Betaferon™ analogues, and Rebif™ analogues. Alternatively, agents that induce interferon-alpha or interferon-beta production or mimic the action of interferon-alpha or interferon-beta may also be employed. Interferon inducers include tilorone, poly(I)-poly(C), imiquimod, cridanimod, bropirimine.

Where clinical applications are contemplated, it may be necessary to prepare pharmaceutical compositions comprising proteins, antibodies, and drugs in a form appropriate for the intended application. Generally, pharmaceutical compositions may comprise an effective amount of one or more of the proteins and peptides according to the present disclosure, optionally with one or more additional agents, dissolved or dispersed in a pharmaceutically acceptable carrier. The phrases “pharmaceutical or pharmacologically acceptable” refers to molecular entities and compositions that do not produce an adverse, allergic, or other untoward reaction when administered to an animal, such as, for example, a human, as appropriate. The preparation of a pharmaceutical composition may be performed using methods exemplified by Remington's Pharmaceutical Sciences, 18th Ed., 1990, incorporated herein by reference.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed., 1990, incorporated herein by reference). Except insofar as any conventional carrier may be incompatible with the active ingredient, its use in the pharmaceutical compositions may be contemplated.

The pharmaceutical composition may be administered intravenously, intrathecally, intradermally, transdermally, intrathecally, intraarterially, intraperitoneally, intranasally, intravaginally, intrarectally, intramuscularly, subcutaneously, mucosally, orally, topically, locally, by inhalation (e.g., inhalation of a nebulized or dry powder formulation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art (see, for example, Remington's Pharmaceutical Sciences, 18th Ed., 1990, incorporated herein by reference). The choice of injection volume and needle size may be chosen by the person of ordinary skill in the art based on site of injection, syringeability and injectability, which includes considering the viscosity of the solution or suspension to be injected and drug concentration, pH, and osmolality. In some instances, the particle size of the active agent may be chosen in order to provide a desired rate of dissolution upon administration (e.g., by subcutaneous injection).

The one or more proteins and/or peptides may be formulated into a composition in a pharmaceutically acceptable free base, neutral, or salt form. Pharmaceutically acceptable salts include the acid addition salts, e.g., those formed with the free amino groups of a proteinaceous composition, or which may be formed with inorganic acids, such as, for example, hydrochloric or phosphoric acids, or such organic acids as acetic, oxalic, tartaric, or mandelic acid. Salts formed with the free carboxyl groups may also be derived from inorganic bases, such as, for example, sodium, potassium, ammonium, calcium, or ferric hydroxides; or such organic bases as isopropylamine, trimethylamine, histidine, or procaine. Upon formulation, solutions will be administered in a manner compatible with the dosage formulation and in such amount as may be therapeutically effective. The formulations may be administered in a variety of dosage forms, such as formulated for parenteral administrations, such as injectable solutions, or aerosols for delivery to the lungs, or formulated for alimentary administrations, such as drug release capsules and the like.

In some embodiments, the pharmaceutical composition may comprise a pharmaceutically acceptable carrier (e.g., with or without an inert diluent). Suitable pharmaceutical carrier may be chosen depending on whether it may be to be administered in solid, liquid, or aerosol form, and/or whether it needs to be sterile for the route of administration, such as injection. The carrier may be assimilable and examples of the carrier include liquid, semi-solid (e.g., pastes), or solid carriers. Except insofar as any conventional media, agent, diluent, or carrier may be detrimental to the recipient or to the therapeutic effectiveness of a composition contained therein, its use in administrable composition for use in practicing the methods may be appropriate. Examples of carriers or diluents include fats, oils, water, saline solutions, lipids, liposomes, resins, binders, fillers, and the like, or combinations thereof. The pharmaceutical composition may also comprise various antioxidants to retard oxidation of one or more component. Additionally, the prevention of the action of microorganisms may be brought about by preservatives, such as various antibacterial and antifungal agents, including but not limited to parabens (e.g., methylparabens, propylparabens), chlorobutanol, phenol, sorbic acid, thimerosal or combinations thereof.

Doses preferably include pharmaceutical dosage units comprising an effective amount of the therapeutic agent. Dosage unit form refers to physically discrete units suited as unitary dosages for a mammalian subject; each unit contains a predetermined quantity of active material calculated to produce the desired therapeutic effect, in association with the required pharmaceutical carrier.

Other pharmaceutically acceptable carriers the present composition are liposomes, pharmaceutical compositions in which the active protein is contained either dispersed or variously present in corpuscles consisting of aqueous concentric layers adherent to lipidic layers. The active protein is preferably present in the aqueous layer and in the lipidic layer, inside or outside, or, in any event, in the non-homogeneous system generally known as a liposomic suspension.

The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature.

In some embodiments, the components in the pharmaceutical composition may be combined with the carrier in any convenient and practical manner, e.g., by solution, suspension, emulsification, admixture, encapsulation, absorption, and the like. Such procedures may be routine for those skilled in the art. In some examples, the composition may be combined or mixed thoroughly with a semi-solid or solid carrier. The mixing may be carried out in any convenient manner, such as grinding. Stabilizing agents may be also added in the mixing process in order to protect the composition from loss of therapeutic activity, e.g., denaturation in the stomach. Examples of stabilizers for use in a composition include buffers, amino acids, such as glycine and lysine, carbohydrates or lyoprotectants, such as dextrose, mannose, galactose, fructose, lactose, sucrose, maltose, sorbitol, mannitol, etc.

In some embodiments, a pharmaceutical composition may comprise one or more surfactant. Surfactants used in accordance with the disclosed methods include ionic and non-ionic surfactants. Examples of non-ionic surfactants include polysorbates such as TWEEN®-20 and TWEEN-80® surfactants (ICI Americas Inc. of Bridgewater, N.J.); poloxamers (e.g., poloxamer 188); TRITON® surfactants (Sigma of St. Louis, Mo.); sodium dodecyl sulfate (SDS); sodium laurel sulfate; sodium octyl glycoside; lauryl-, myristyl-, linoleyl-, or stearyl-sulfobetaine; lauryl-myristyl-, linoleyl- or stearyl-sarcosine; linoleyl-, myristyl-, or cetyl-betaine; lauroamidopropyl-cocamidopropyl-, linoleamidopropyl-, myristamidopropyl-, palnidopropyl-, or (e.g., lauroamidopropyl); myristamidopropyl-, palmidopropyl-, or isostearamidopropyl-dimethylamine; sodium methyl cocoyl-, or disodium methyl oleyl-taurate; MONAQUAT™ surfactants (Mona Industries Inc. of Paterson, N.J.); polyethyl glycol; polypropyl glycol; block copolymers of ethylene and propylene glycol such as PLURONIC® surfactants (BASF of Mt. Olive, N.J.); oligo (ethylene oxide) alkyl ethers; alkyl (thio) glucosides, alkyl maltosides; and phospholipids. For example, the surfactant may be present in a formulation in an amount from about 0.01% to about 0.5% (weight of surfactant relative to total weight of other solid components of the formulation; “w/w”), from about 0.03% to about 0.5% (w/w), from about 0.05% to about 0.5% (w/w), or from about 0.1% to about 0.5% (w/w). In some examples, the pharmaceutical composition may be essentially free of non-ionic surfactants or essentially free of all surfactants.

The pharmaceutical composition may be administered in any suitable mode of administration, e.g., intramuscular, intravenous, intraperitoneal, intravesicular, intraarticular, intralesional, subcutaneous, or any other route sufficient to provide a dose adequate to treat the inflammation-related disorder. The pharmaceutical composition may be administered to the patient in a single dose or in multiple doses. When multiple doses are administered, the doses may be separated from one another by, for example, one hour, three hours, six hours, eight hours, one day, two days, one week, two weeks, or one month. For example, the pharmaceutical composition may be administered for, e.g., 1, 2, 3, 4, 5, 6, 7, 8, 10, 15, 20, or more weeks. It may be to be understood that, for any particular subject, specific dosage regimes may be adjusted over time according to the individual need and the professional judgment of the person administering or supervising the administration of the compositions. For example, the dosage of the pharmaceutical composition may be increased if the lower dose does not provide sufficient therapeutic activity.

Therapeutically effective amounts of the one or more proteins and peptides as disclosed herein or a mutant, variant, analog or derivative thereof may be provided at a dose of 0.0001, 0.01, 0.01, 0.1, 1, 5, 10, 25, 50, 100, 500, or 1,000 mg/kg or g/kg. Effective doses may be extrapolated from dose-response curves derived from in vitro or animal model test bioassays or systems.

Dosages for a particular patient or subject may be determined by one of ordinary skill in the art using conventional considerations, (e.g., by means of an appropriate, conventional pharmacological protocol). A physician may, for example, prescribe a relatively low dose at first, subsequently increasing the dose until an appropriate response may be obtained. The dose administered to a patient may be sufficient to effect a beneficial therapeutic response in the patient over time, or, e.g., to reduce symptoms, or other appropriate activity, depending on the application. The dose may be determined by the efficacy of the particular formulation, and the activity, stability or serum half-life of the one or more proteins and peptides as disclosed herein or a mutant, variant, analog or derivative thereof and the condition of the patient, as well as the body weight or surface area of the patient to be treated.

In some embodiments, a subject may be given a single dose, given once daily for treating a subject, preferably a mammal, more preferably human who his suffering from or susceptible to pulmonary fibrosis resulting therefrom may be between about 0.2 mg/kg and about 250 mg/kg, such as between about 10 mg/kg and about 50 mg/kg, for example, via instillation (by inhalation). Such a dose may be administered daily for anywhere from about 3 days to one or more weeks. Chronic administration may be also possible, though the dose may need to be adjusted downward as is well-understood in the art. The foregoing ranges are, however, suggestive, as the number of variables in an individual treatment regime may be large, and considerable excursions from these preferred values may be expected.

For continuous administration, e.g., by a pump system such as an osmotic pump, a total dosage for a time course of about 1-2 weeks may be preferably in the range of 1 mg/kg to 1 g/kg, preferably 20-300 mg/kg, more preferably 50-200 mg/kg. After such a continuous dosing regimen, the total concentration of the active compound may be in the range of about 0.5 to about 50 μM, preferably about 1 to about 1 μM. An effective concentration of the one or more proteins and peptides for inhibiting or preventing inhibiting apoptosis in vitro may be in the range of about 0.5 nM to about 100 nM, more preferably from about 2 nM to about 20 nM. Effective doses and optimal dose ranges may be determined in vitro using the methods described herein.

### B. Dry Powder and Inhalation Devices

In some embodiments, the pharmaceutical composition may be in a form of dry powder. In some examples, for effective inhalation and deposition of a powder into a subject (e.g., into the lungs), the particle size of the dry powder may have a mass median aerodynamic diameter of less than about 5 μm. The particle sizes of the formulations may be reduced by any suitable method, including but not limited to milling, grinding, thin film freezing, spray drying, or crushing. Milling may be performed by any method known in the art, such as by air jet mill, ball mill, wet mill, media mill, high pressure homogenization, or cryogenic mill. See WO2020/055824, which is incorporated by reference herein in its entirety.

The stability of the proteins and/or peptides following particle size reduction may be assessed using known techniques in the art, including size exclusion chromatography; electrophoretic techniques; HPLC; mass spectrometry; spectroscopic techniques such as UV spectroscopy and circular dichroism spectroscopy, and activity (measured in vitro or in vivo). To perform in vitro assays of protein stability, an aerosol composition may be collected and then distilled or absorbed onto a filter. To perform in vivo assays, or for pulmonary administration of a pharmaceutical composition to a subject, a device for dry powder dispersion may be adapted for inhalation by the subject. For example, protein stability may be assessed by determining the level of protein aggregation. Preferably, a dry powder composition of the invention may be substantially free of protein aggregates. The presence of soluble aggregates may be determined qualitatively using dynamic light scattering (DLS) (DynaPro-801TC, Protein Solutions Inc. of Charlottesville, Va.) and/or by UV spectrophotometry.

In some embodiments, the method may comprise modulating the drug release. In some embodiments, the pharmaceutical composition may be formulated for slow- or delayed-release. In some embodiments, the pharmaceutical composition may be formulated for fast-release. In further embodiments, the pharmaceutical composition may be formulated for both slow and fast release (e.g., dual release profile).

In some embodiments, the present disclosure provides methods for the administration of the inhalable pharmaceutical composition provided herein. Administration may be, but is not limited, to inhalation of the pharmaceutical composition using an inhaler. In some embodiments, an inhaler may be a passive dry powder inhaler (DPI), such as a Plastiape RSO1 monodose DPI. In a dry powder inhaler, dry powder may be stored in a reservoir and may be delivered to the lungs by inhalation without the use of propellants.

In some embodiments, an inhaler may be a single-dose DPI, such as a DoseOne™ Spinhaler, Rotohaler®, Aerolizer®, or Handihaler. In some embodiments, an inhaler may be a multidose DPI, such as a Plastiape RSO2, Turbuhaler®, Twisthaler™, Diskhaler®, Diskus®, or Ellipta™. In some embodiments, an inhaler may be a plurimonodose DPI for the concurrent delivery of single doses of multiple medications, such as a Plastiape RS04 plurimonodose DPI. In some examples, dry powder inhalers have medication stored in an internal reservoir, and medication may be delivered by inhalation with or without the use of propellants. Other types of dry powder inhalers may have medication in pre-divided doses stored in a capsule (e.g., cellulose or gelatin base) or foil pouch, each of which may be punctured by the device to release the dose to the patient. Dry powder inhalers may require an inspiratory flow rate greater than 30 L/min for effective delivery, such as between about 30-120 L/min. In some embodiments, efficient aerosolization of the pharmaceutical composition is independent of inspiratory force. In some embodiments, the dry powder inhaler has a flow resistance of between 0.01 kPa^{0.5} min/L and 0.05 kPa^{0.5} min/L, such as between 0.02 kPa^{0.5} min/L and 0.04 kPa^{0.5} min/L. The dry powder inhaler (e.g., high resistance, low resistance, passive, active) may be chosen based on the patient population and their inspiratory capabilities.

In some embodiments, the inhaler may be a metered dose inhaler. Metered dose inhalers deliver a defined amount of medication to the lungs in a short burst of aerosolized medicine aided by the use of propellants. Metered dose inhalers comprise three major parts: a canister, a metering valve, and an actuator, and may utilize a spacer device to de-accelerate the emitted particles and facilitate inhalation of the aerosolized cloud by the patient. The medication formulation, including propellants and any required excipients, may be stored in the canister. The metering valve allows a defined quantity of the medication formulation to be dispensed. The actuator of the metered dose inhaler, or mouthpiece, contains the mating discharge nozzle and typically includes a dust cap to prevent contamination. The required inspiratory flow rate required for the use of a metered dose inhaler may be less than 90 L/min, such as between about 15-90 L/min, preferably about 30 L/min. In some embodiments, efficient aerosolization of pharmaceutical composition may be independent of inspiratory force.

In some embodiments, an inhaler may be a nebulizer. A nebulizer may be used to deliver medication in the form of an aerosolized mist inhaled into the lungs. The medication formulation may be aerosolized by compressed gas, or by ultrasonic waves. A jet nebulizer may be connected to a compressor. The compressor emits compressed gas through a liquid medication formulation at a high velocity, causing the medication formulation to aerosolize. Aerosolized medication may be then inhaled by the patient. An ultrasonic wave nebulizer generates a high frequency ultrasonic wave, causing the vibration of an internal element in contact with a liquid reservoir of the medication formulation, which causes the medication formulation to aerosolize. Aerosolized medication may be then inhaled by the patient. A nebulizer may utilize a flow rate of between about 3-12 L/min, such as about 6 L/min. In some examples, the milled active may be suspended in a pharmaceutically acceptable liquid carrier vehicle and administered by nebulization (e.g., air jet nebulization). In further aspects, a composition of the embodiments may be administered by a vaporization method (e.g., rapid vaporization) such as by an e-cigarette device.

In some embodiments, the pharmaceutical composition may be administered on a routine schedule. As used herein, a routine schedule refers to a predetermined designated period of time. The routine schedule may encompass periods of time which may be identical or which differ in length, as long as the schedule may be predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In some embodiments, a one or more of the proteins and peptides may be administered once per day. In preferred embodiments, a protein or peptide may be administered less than once per day, such as every other day, every third day, or once per week. In some embodiments, a complete dose of pharmaceutical composition is between 1-100 mg, such as 20-100, 50-100, 10-20, 20-40, 50-70, or 80-90 mg.

In some embodiments, the one or more proteins and peptides may be provided in a unit dosage form (e.g., pre-divided dose), such as in a capsule, blister or a cartridge, wherein the unit dose comprises at least 1 mg of a protein or peptide, such as at least 5 mg, 10 mg, 15 mg or 20 mg of the one or more proteins and peptides per dose. In some aspects, the unit dose may be 1-10 mg (e.g., about 5 mg) of the one or more proteins and peptides. In particular aspects, the unit dosage form does not comprise the administration or addition of any excipient and may be merely used to hold the powder for inhalation (e.g., the capsule, blister, or cartridge may be not administered). In some aspects, more than one of the unit dose forms in administered to a subject. For example, in the case of a dry powder inhaler, proteins and peptides of the embodiments may be provided in unit dose capsules and more than one unit dose capsules (e.g., 3-4) may be administered to a subject by inhalation. In some embodiments, the one or more proteins and peptides may be administered in a high emitted dose, such as at least 10 mg, preferably at least 15 mg, even more preferably 20 mg. In some embodiments, administration of the one or more proteins and peptides may result in a high fine particle dose into the deep lung such as greater than 5 mg. Preferably, the fine particle dose into the deep lung may be at least 10 mg, even more preferably at least 15 mg. In some examples, the particle dose may be produced from 1, 2, 3, 4 or 5 or more capsules comprising doses of the one or more proteins and peptides. In some aspects, the fine particle dose may be at least, 50%, such as at least 60, 65, 70, 75, or 80% of the emitted dose.

In some embodiments, changes in inhalation pressure drop may result in a change in emitted dose. In some embodiments, changes in inhalation pressure of 3 kPa, such as from 4 kPa to 1 kPa, may result in a reduction of emitted dose of less than 25%, such as 24%, 23%, 22%, 21%, 20%, 19%, 18%, 17%, 16%, 15%, 14%, 13%, 12%, 11%, 10%, 9%, 8%, 7%, 6%, 5% or less. In some embodiments, changes in inhalation pressure may result in a change in fine particle dose. In some embodiments, changes in inhalation pressure of 3 kPa, such as from 4 kPa to 1 kPa may result in a reduction of fine particle dose of less than 15%, such as 14%, 13%, 12%, 11%, 10%, 9%, 8%, 7%, 6%, 5% or less.

### IV. Pulmonary Conditions for Treatment

The pharmaceutical composition the present disclosure may be used to treat a variety of pulmonary conditions. Pulmonary conditions for treatment may be acute or chronic. Acute pulmonary conditions may be acute lung injury, infection or chemical-induced. Chronic pulmonary conditions maybe the result of injury, infection or disease. The compositions, methods and uses of the present disclosure inhibit coronavirus and other pathogen growth, inhibit lung injury, inhibit alveolar epithelial cell (AEC) damage, inhibit lung and other organ remodeling and fibrosis, inhibit pro-fibrogenic phenotypes of lung fibroblasts, improve lung function and overall survival after infection.

### A. Lung Injuries

In some aspects, the method and composition herein may be used to a subject that has an acute lung injury (ALI) or infection or a chemical-induced lung injury. Viral ALI typically starts with formation of a transitional fibrinous matrix within the damaged alveoli that undergoes infiltration by fibroblasts and early scarification. In some embodiments, the subject may have plastic bronchitis, asthma, chronic obstructive airway/pulmonary (COPD), acute respiratory distress syndrome (ARDS), inhalational smoke induced acute lung injury (ISALI), bronchiectasis, inhalational toxin-induced airway disease (e.g., chlorine or other induced airways disease), exposure to mustard gas, exposure to particulate matter (e.g., silica dust), bronchiolitis obliterans, bronchiolitis obliterans organizing pneumonia, collagen vascular lung disease (e.g., from lupus, scleroderma or mixed connective tissue disease), interstitial lung disease (e.g., idiopathic pulmonary fibrosis or sarcoidosis), drug induced lung disease and accelerated pulmonary fibrosis (e.g., that occurs after acute lung injury including ARDS).

Acute lung injury, attempt at repair by fibroproliferation, and lung remodeling occur in COVID-19 disease, much like it does in other coronavirus infections. This leads to a potential increase in the risk of pulmonary fibrosis occurring as a sequela of COVID-19. Features of diffuse alveolar damage (DAD) with areas of consolidation by fibroblastic proliferation and deposition of ECM and fibrin in the alveolar spaces have been identified in humans after COVID-19 infection. The alveolar wall has three components: the alveolar epithelium with its basement membrane, capillary endothelium with a basement membrane, and an interstitium containing the fused basement membranes, fibroblasts, collagen fibrils, elastic fibers, and macrophages. Alveolar epithelial damage follows the application of the injurious stimulus, and this results in the release of damage-associated molecular patterns (DAMPs) from injured cells. DAMPs, in addition to pathogen-associated molecular patterns (PAMPs) from microbes, are recognized by alveolar macrophages, leading to a series of downstream transduction, and release of antimicrobial and proinflammatory cytokines, including IL-1 and TNF.

### B. Lung Diseases

Examples of lung diseases include chronic obstructive pulmonary disease, asthma, infections, as well as acute and chronic lung injury leading to fibrosis, which constitute the third leading cause of death world-wide (Murray et al., 1997; Rabe et al., 2007; Tsushima et al., 2009). Example of the disease include acute lung injury (ALI), which may be a serious medical problem amongst American military personnel. ALI during combat may result from very broad etiologies. Further examples of lung diseases include cystic fibrosis, chronic obstructive pulmonary disease (COPD), asthma, bronchiolitis obliterans, plastic bronchitis, and pulmonary infections, collagen vascular lung disease (e.g., from lupus, scleroderma or mixed connective tissue disease), interstitial lung disease (e.g., idiopathic pulmonary fibrosis or sarcoidosis), as well as acute and chronic lung injury leading to fibrosis (Murray et al., 1997; Rabe et al., 2007; Tsushima et al., 2009). These diseases constitute the third leading cause of death world-wide.

Cystic fibrosis is an inherited disease of the exocrine glands and exocrine sweat glands, which primarily affects the digestive and respiratory systems. This disease usually characterized by chronic respiratory infections, pancreatic insufficiency, abnormally viscid mucous secretions and premature death. Cystic fibrosis (CF) is characterized by progressive airflow obstruction. Subsets of individuals with CF also develop airway hyper-responsiveness to inhaled cholinergic agonists (Weinberger, 2002 and Mitchell et al., 1978) and reversibility of airflow limitation in response to bronchodilators (van Haren et al., 1991 and van Haren et al., 1992). The presence of bronchial hyper-responsiveness and airway obstruction suggest a possible shared etiology of disease between CF and other diseases of airway narrowing such as asthma or chronic obstructive pulmonary disease (COPD) where airway smooth muscle dysfunction is thought to contribute to the disease processes.

Chronic obstructive pulmonary disease (COPD) is a term used to classify two major airflow obstruction disorders: chronic bronchitis and emphysema. Approximately 16 million Americans have COPD, 80-90% of them were smokers throughout much of their lives. COPD is a leading cause of death in the U.S., accounting for 122,283 deaths in 2003. The cost to the USA for COPD was approximately $20.9 billion in direct health care expenditures in 2003. Chronic bronchitis is inflammation of the bronchial airways. The bronchial airways connect the trachea with the lungs. When inflamed, the bronchial tubes secrete mucus, causing a chronic cough.

In emphysema, the alveolar sacs may be overinflated as a result of damage to the elastin skeleton of the lung. Inflammatory cells in emphysematous lung release elastase enzymes, which degrade or damage elastin fibers within the lung matrix. Emphysema has a number of causes, including smoking, exposure to environmental pollutants, alpha-one antitrypsin deficiency, and aging.

### C. Lung Infection

In some embodiments, the method and composition herein may be used for treating lung infection (e.g., by a virus). A pulmonary infection may be a bacterial infection. The infectious bacteria may be Pseudomonas aeruginosa, Bacillus anthracis, Listeria monocytogenes, Staphylococcus aureus, Salmenellosis, Yersina pestis, Mycobacterium leprae, M. africanum, M. asiaticum, M. aviuin-intracellulaire, M. chelonei abscessus, M. fallax, M. fortuitum, M. kansasii, M. leprae, M. malmoense, M. shimoidei, M. simiae, M. szulgai, M. xenopi, M. tuberculosis, Brucella melitensis, Brucella suis, Brucella abortus, Brucella canis, Legionella pneumonophilia, Francisella tularensis, Pneurnocystis carinii, mycoplasma, or Burkholderia cepacia. The bacterial infection may result in pneumonia.

A pulmonary infection may be a viral infection. The infectious virus may be a coronavirus, a SARS-CoV-1 virus, a SARS-CoV-2, or a MERS-CoV. The viral infection may cause SARS, COVID-19, or MERS. The infectious virus may be an influenza virus including influenza A, B, C, or D, particularly an Influenza A virus, a respiratory syncytial virus (RSV), an enterovirus (Picornaviridae), a rhinovirus, parainfluenza, a metapneumovirus, a bocavirus, an adenovirus, a Coxsackie virus, or other lung-infecting virus. The viral infection may result in pneumonia.

Bronchiolitis is most commonly caused by viral lower respiratory tract infections, and primarily characterized by acute inflammation, edema, necrosis of epithelial cells lining small airways, and increased mucus production (Ralston et al., 2014). Signs and symptoms typically begin with rhinitis and cough, which may progress to tachypnea, wheezing, rales, use of accessory muscles, and/or nasal flaring.

Bronchiolitis obliterans is a progressive airflow reduction as a result of abnormal remodeling of the small airways in the lungs (Meyer et al., 2014). Bronchiolitis obliterans syndrome is a major complication of lung transplantations, and is often used to describe a delayed allograft dysfunction that results in persistent decline in forced expiratory volume and force that is not caused by other known causes (Meyer et al., 2014).

The term “asthma” may refer to acute asthma, chronic asthma, intermittent asthma, mild persistent asthma, moderate persistent asthma, severe persistent asthma, chronic persistent asthma, mild to moderate asthma, mild to moderate persistent asthma, mild to moderate chronic persistent asthma, allergic (extrinsic) asthma, non-allergic (intrinsic) asthma, nocturnal asthma, bronchial asthma, exercise induced asthma, occupational asthma, seasonal asthma, silent asthma, gastroesophageal asthma, idiopathic asthma and cough variant asthma. During asthma, the airways are persistently inflamed and may occasionally spasm.

The compositions and methods herein may be used to treat post-infection syndromes, such as the lung injuries, lung diseases, and lung infections that occur after SARS-CoV-2 infection. For example, the compositions and methods herein may be used for treating lung remodeling, fibrotic lung, and/or circulatory system issues that occur after SARS-CoV-2 infection, e.g., occur within 1 day, 2 days, 3 days, 4 days, 5 days, 6 days, 1 week, 2 weeks, 3 weeks, 1 month, 2 months, 3 months, 4 months, 5 months, 6 months, or a year after SARS-CoV-2 infection.

A pathogen-induced infection, for example, an infection by a coronavirus, a SARS-CoV-1 virus, a SARS-CoV-2, or a MERS-CoV, an influenza virus, a respiratory syncytial virus (RSV), an enterovirus (Picornaviridae), a rhinovirus, parainfluenza, a metapneumovirus, a bocavirus, an adenovirus, or a Coxsackie virus, may increase the expression of MEGF9 protein in the tissue of an infected subject relative to a non-infected subject. In some aspects, the tissue is pulmonary tissue, for example lung tissue. In some aspects, a MEGF9 protein or a biologically active fragment thereof is administered to a subject in a pharmaceutically effective amount sufficient to reduce MEGF9 protein expression in the subject having a pathogen-induced infection. In some aspects, a MEGF9 protein or a biologically active fragment thereof is administered to a subject in a pharmaceutically effective amount sufficient to reduce MEGF9 protein expression in the subject infected with an influenza virus.

A pathogen-induced infection, may increase tissue injury signaling (e.g., necroptotic pathway signaling) in the tissue of an infected subject relative to a non-infected subject. In some aspects, the tissue is pulmonary tissue, for example lung tissue. In some aspects, the signaling comprises increased activation of a ZBP1-MLKL cell injury pathway. In some aspects, a MEGF9 protein or a biologically active fragment thereof is administered to a subject in a pharmaceutically effective amount sufficient to reduce activation of the cell injury pathway in the subject with a pathogen-induced infection. In some aspects, a MEGF9 protein or a biologically active fragment thereof is administered to a subject in a pharmaceutically effective amount sufficient to reduce activation of the cell injury pathway in the subject infected with an influenza virus.

In some aspects, a MEGF9 protein or a biologically active fragment thereof is administered to a subject in a pharmaceutically effective amount sufficient to reduce the mortality and morbidity of the subject having a pathogen-induced infection. In some aspects, a MEGF9 protein or a biologically active fragment thereof is administered to a subject in a pharmaceutically effective amount sufficient to reduce pathogen-induced weight loss in the subject. In some aspects, the pathogen-induced weight loss in the subject is by an influenza virus infection.

In some aspects, the pharmaceutically effective amount of the MEGF9 protein or biologically active fragment of MEGF9, as described above, is a dose of about 0.0001 mg/kg to about 1,000 mg/kg, about 0.2 mg/kg to about 250 mg/kg, about 1 mg/kg to about 25 mg/kg, or about 1 mg/kg to about 2.5 mg/kg. In some aspects, a dose is administered daily. In some aspects, a dose is administered daily from about 3 days to one or more weeks.

Having now generally described the invention, the same will be more readily understood through reference to the following examples which are provided by way of illustration, and are not intended to be limiting of the present invention, unless specified. The examples are included to provide examples supporting the present disclosure and not limiting the scope of the invention.

### EXAMPLES

### Example 1

This example describes the effects of MEGF9, UNC5A, and ALG5 on the growth of SARS-CoV-2 in Vero E6 cells. The evaluation was performed by plaque reduction microneutralization (PRMNT) assay, which is adopted from Park et al., Rapid in vitro assays for screening neutralizing antibodies and antivirals against SARS-CoV-2. J Virol Methods. 2021 January; 287:113995, which is incorporated by reference in its entirety.

The antiviral activity was determined by assessing the effective concentration that inhibits virus replication in Vero E6 cells (96-well plates, about 4×10⁴ Vero E6 cells/well at confluency, triplicate) following viral incubation for 24 h.

In brief, 1×10⁴ Vero E6 cells were seeded in 96-well plate and infected with SARS-CoV-2 virus (about 100-200 PFU/well). After incubating the cells with the virus for 1 hour, the media were removed, and the protein or peptide was added to the 96-well plate to treat the infected cells for 24 hour. After the 24-hour incubation, the media were removed, and the cells in the plates were fixed and inactivated with 10% formalin solution. The fixed plates were developed with VECTASTAIN® ABC-HRP kit to visualize the level of virus growth.

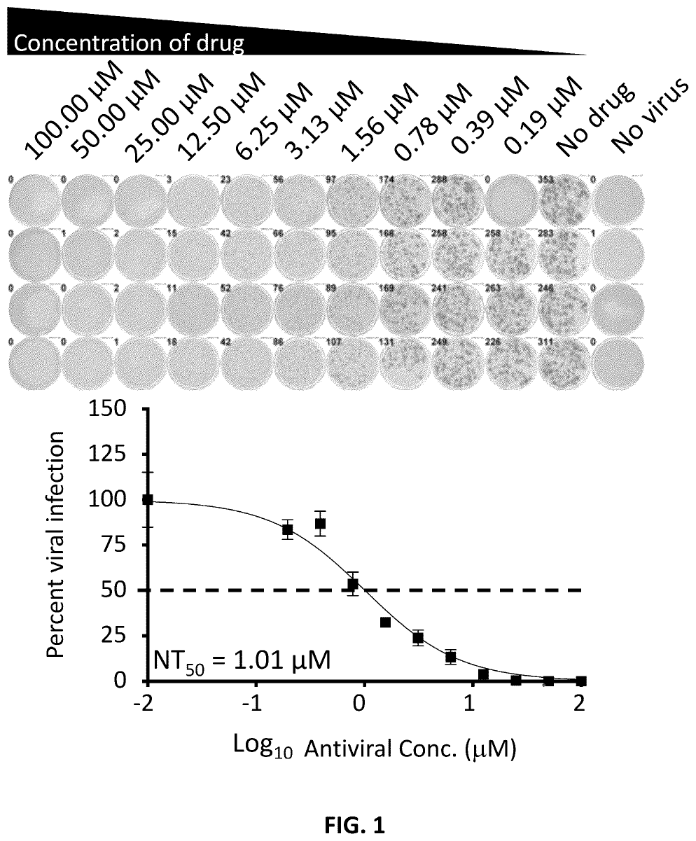

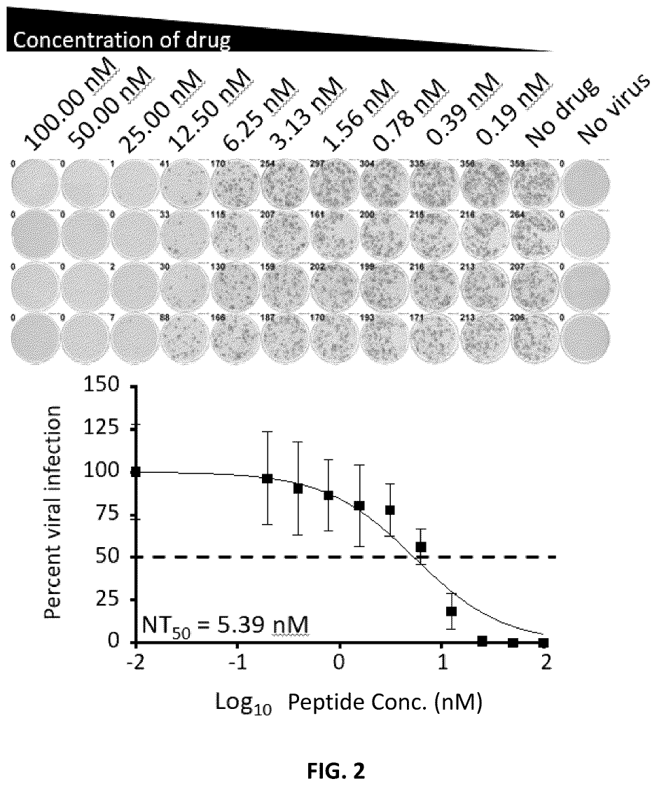

The virus neutralization was quantified. Specifically, 50% virus neutralization (NT50) was determined and results are shown in FIGS. 1-2. As a comparator, FIG. 1 shows the antivirus assay using various concentrations of the drug remdesivir, which has art-recognized anti-viral activity against SARS-CoV2 and has been approved around the world as a therapy for treatment of COVID-19. FIG. 2 shows the antivirus assay using various concentrations of a combination of MEGF9, UNC5A, and ALG5 (SEQ ID NOs: 1, 5, and 9) according to the present disclosure. The results demonstrated that MEGF9, UNC5A, and ALG5 have high anti-viral activity in the order of the art-recognized anti-viral therapy.

### Example 2

This example describes the effects of ECDs of MEGF9, UNC5A, and ALG5 on the growth of SARS-CoV-2 in Vero E6 cells. The evaluation was performed by plaque reduction microneutralization (PRMNT) assay as described in Example 1.

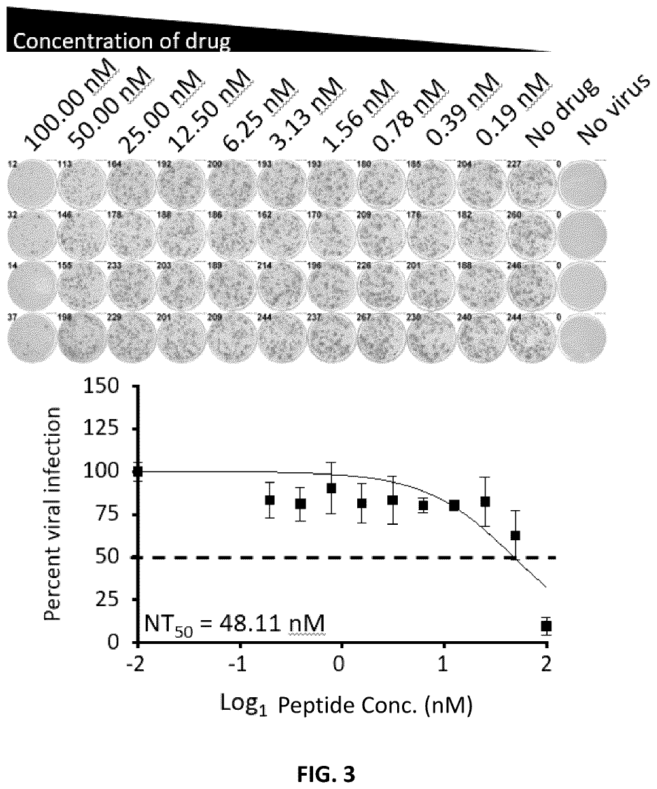

The virus neutralization was quantified. Specifically, 50% virus neutralization (NT50) was determined and results are shown in FIG. 3. FIG. 3 shows the antivirus assay using various concentrations of a combination of ECDs of MEGF9, UNC5A, and ALG5 (SEQ ID NOs: 102, 103, and 104, respectively) according to the present disclosure. The results demonstrated that ECDs of MEGF9, UNC5A, and ALG5 have high anti-viral activity.

### Example 3—Plaque Reduction Microneutralization Test (PRMNT)

Confluent monolayers of Vero E6 cells (5×10⁴ cells/well, 96-well plate format, quadruplicates) were infected with 100-200 plaque forming units (PFU)/well of SARS-CoV-2 USA/WA1/2020 (WA-1) and incubated at 37° C. in a 5% CO₂ incubator for 1 h. After virus adsorption, cells were washed with PBS and maintained at 37° C. in post-infection media [DMEM supplemented with 2% fetal bovine serum (FBS) and 1% penicillin-streptomycin-glutamine (PSG)] containing Avicel 1% and two-fold serial dilutions of the indicated antiviral compound. At 20 h post-infection, media were removed, and plates were fixed with 10% formalin solution for 24 h at 4° C. Fixed cells were immunostained (1 μg/mL) with a SARS-CoV-2 nucleocapsid (N) protein monoclonal antibody (1C7C7) and developed using a peroxidase-conjugated anti-mouse antibody (Vectastain). Viral plaques were visualized and counted using a CTL ImmunoSpot plate reader and a counting software (Cellular Technology Limited, Cleveland, OH, USA). The 50% inhibitory concentrations (IC50) were determined with a sigmoidal dose response curve (Graphpad Prism 9).

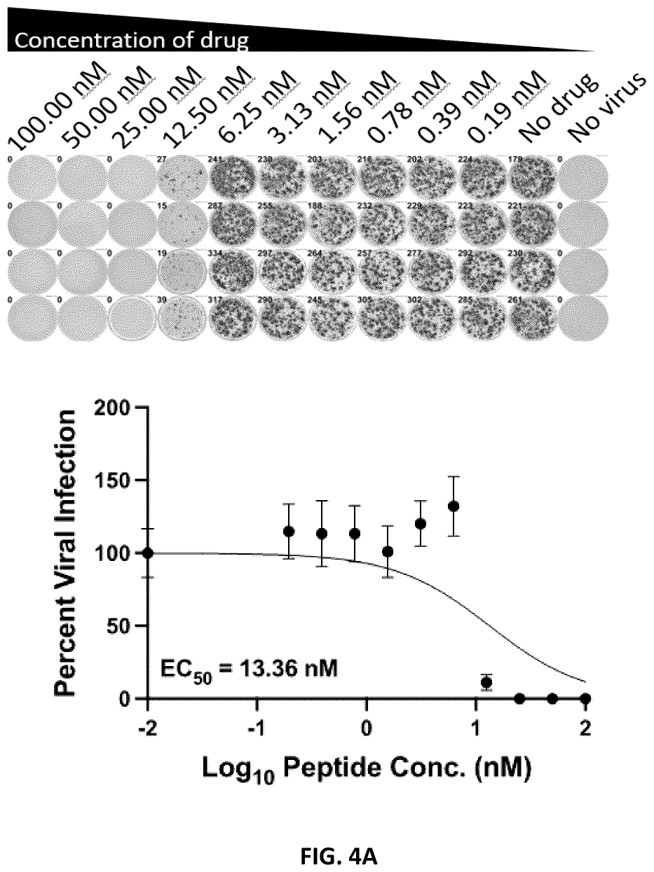

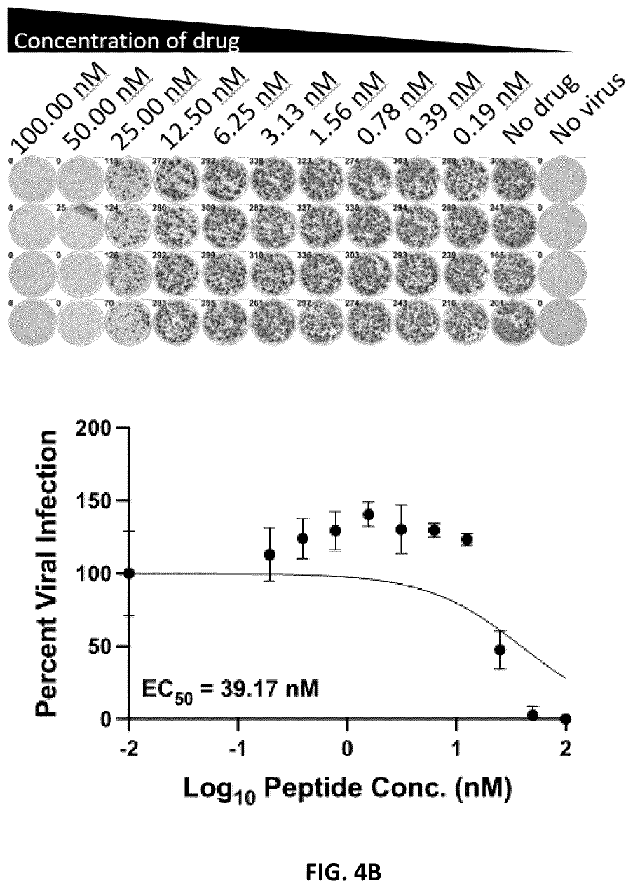

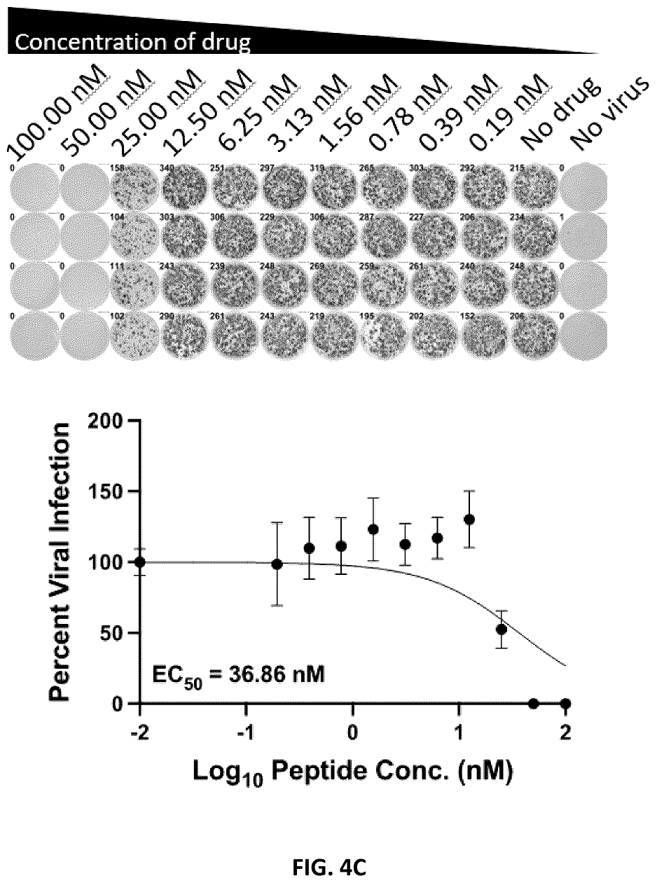

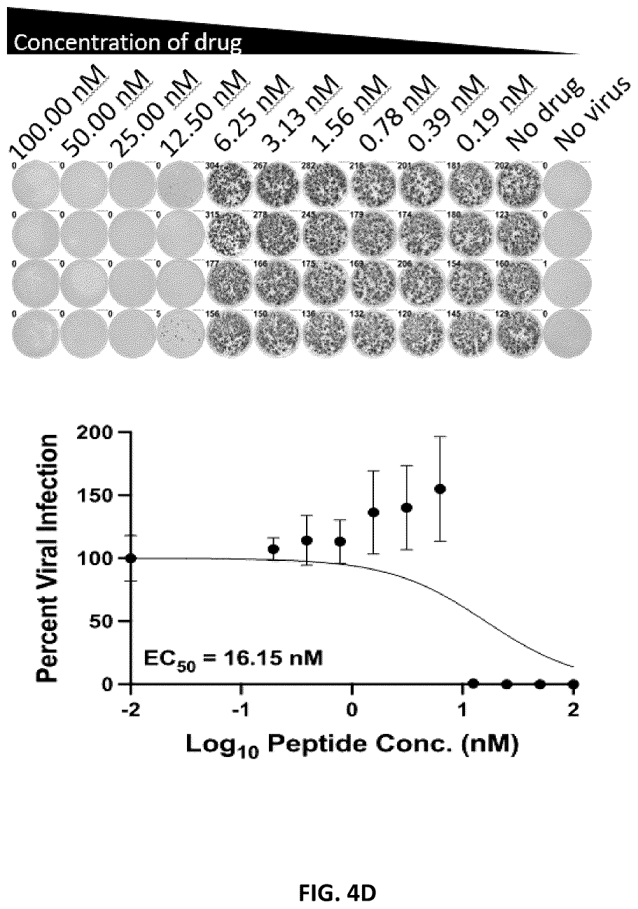

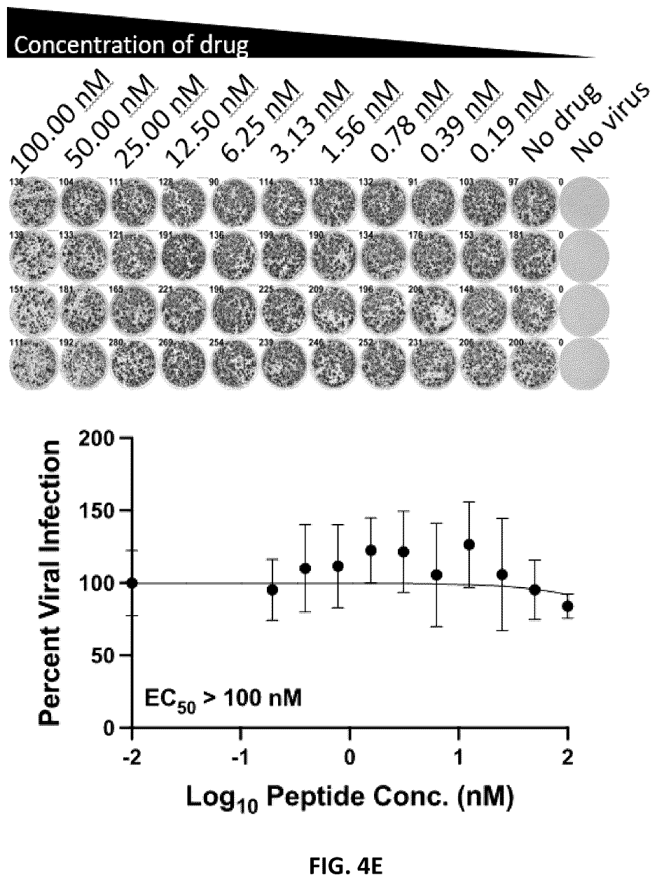

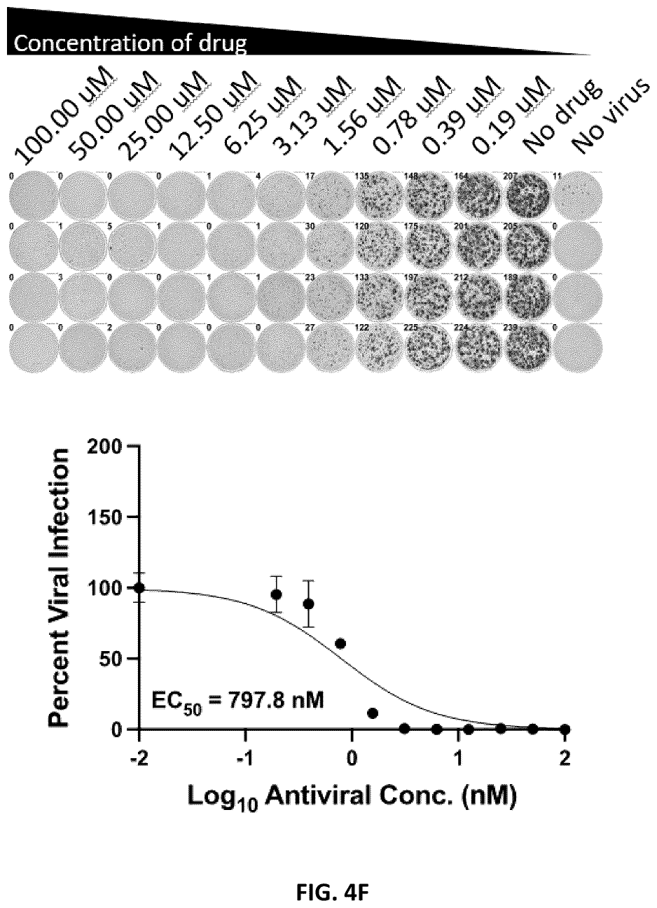

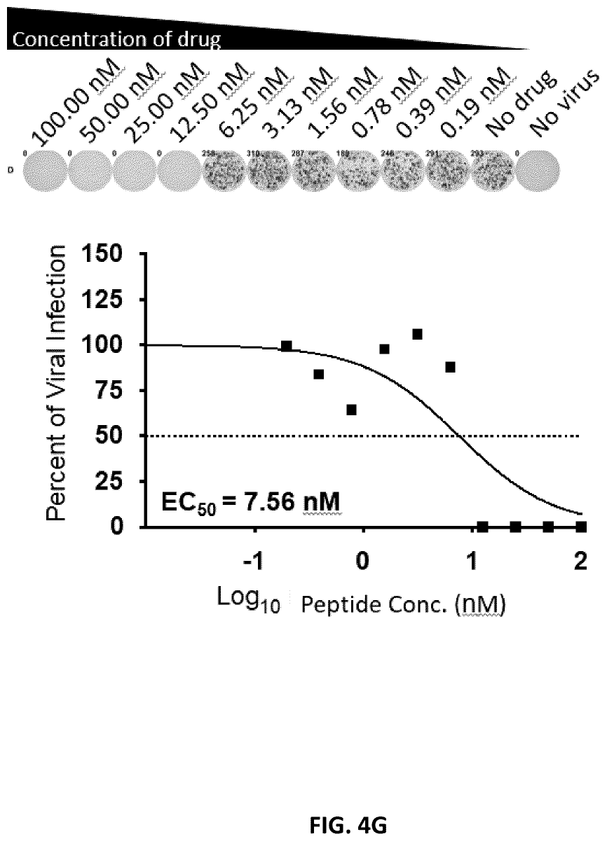

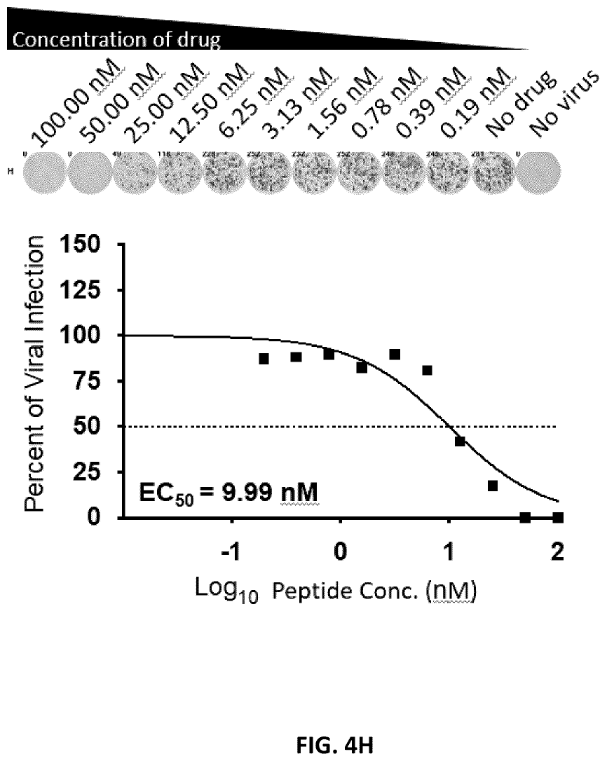

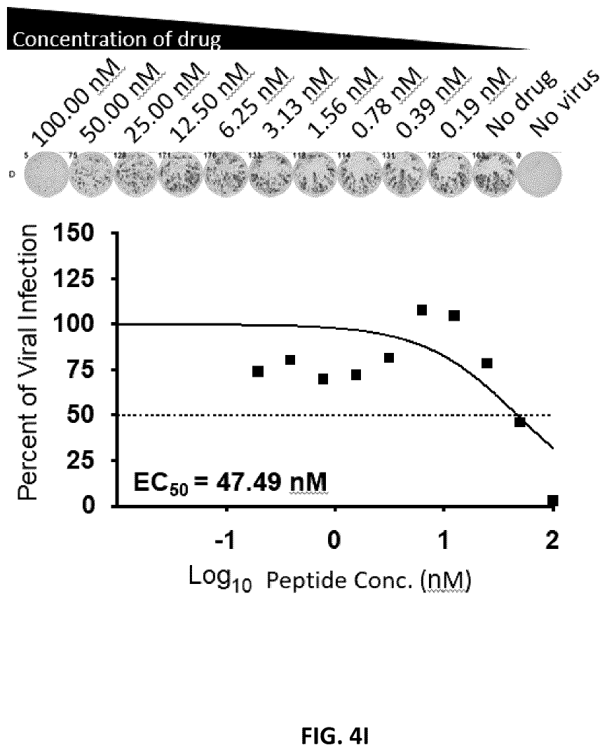

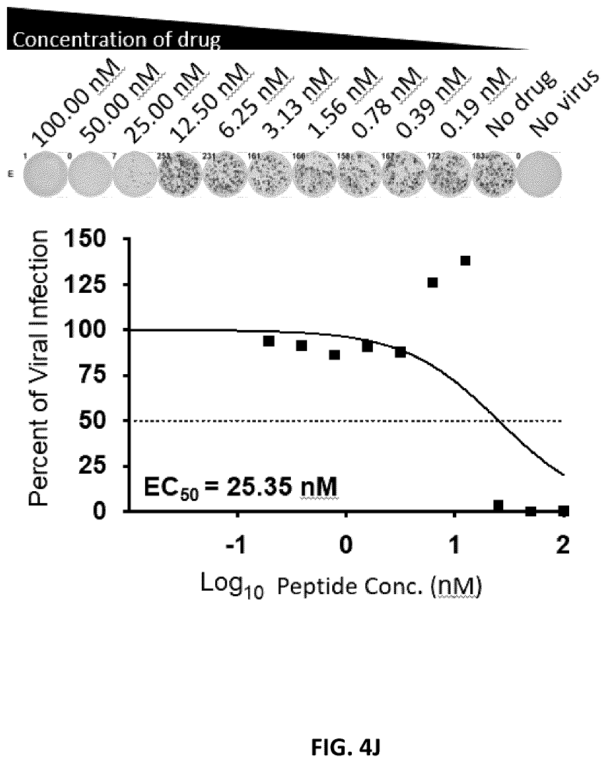

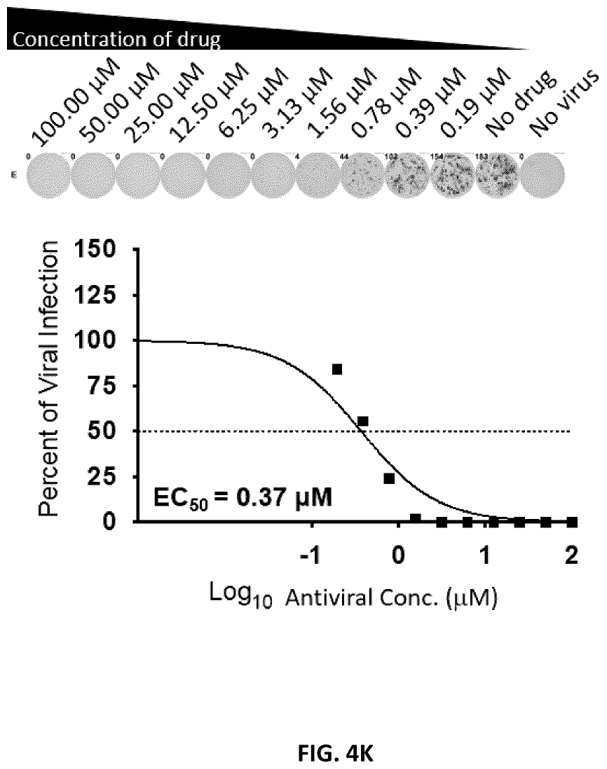

The results are shown in FIGS. 4A-4F. FIG. 4A shows the PRMNT results with the treatment of recombinant full length MEGF9 (M9^{FL}). FIG. 4B shows the PRMNT results with the treatment of recombinant full length UNC5a (U5a^{F}). FIG. 4C shows the PRMNT results with the treatment of recombinant full length ALG5 (A5FL). FIG. 4D shows the PRMNT results with the treatment of M9^{FL}, U5a^{FL}, and AS^{FL}. FIG. 4E shows the PRMNT results with the treatment of the vehicle (blank). FIG. 4F shows the PRMNT results with the treatment of Remdesivir. FIGS. 4G-4K show a repeated set of PRMNT assay results. FIG. 4G shows PRMNT results with use of recombinant full length MEGF9 (M9^{FL}). FIG. 4H shows PRMNT results with use of recombinant full length UNC5a (U5a^{FL}). FIG. 4I shows PRMNT results with the use of recombinant full length ALG5 (AS^{FL}). FIG. 4J shows PRMNT results with use of combined M9^{FL}, U5a^{FL}, and AS^{FL}. FIG. 4K shows PRMNT results with use of Remdesivir.

### Example 4

Four-to-six-week-old female K18 human angiotensin-converting enzyme 2 (hACE2) transgenic mice were purchased from The Jackson Laboratory and maintained in the animal biosafety laboratory level 3 (ABSL-3) at Texas Biomedical Research Institute. All mouse procedures were approved by Texas Biomedical Research Institute IACUC. To assess the in vivo antiviral efficacy of M9^{FL} or M9^{ECD}, K18 hACE2 transgenic mice were injected intraperitoneally (i.p.) with M9^{FL} or M9^{ECD} (2.5 mg/kg) or PBS once daily starting at 24 h prior to challenge with SARS-CoV-2 and until 4 days post-infection (dpi). For viral infections, mice were anesthetized and inoculated intranasally (i.n.) with a lethal dose (104 PFU) of a recombinant (r)SARS-CoV-2 expressing both mCherry and nanoluciferase (Nluc) on the backbone of WA-1 strain (rSARS-CoV-2/mCherry-Nluc) and monitored daily for morbidity and mortality as determined by changes in body weight and survival (n=5/group), respectively. Mice that lost greater than 20% of their initial weight were considered to have reached their experimental endpoint and were humanely euthanized. In vivo bioluminescence (Nluc) imaging of entire mouse was determined by an Ami HT in vivo imaging system, IVIS (Spectral Instruments). Briefly, K18 hACE2 mice infected with 10⁴ PFU of rSARS-CoV-2/mCherry-Nluc (n=4/group) were anesthetized and injected retro-orbitally with Nano-Glo luciferase substrate (Promega) before imaging in the Ami HT at 2 and 4 dpi. After imaging, mice were euthanized with a lethal dose of Fatal-Plus solution, and lungs and nasal turbinate were surgically extracted and washed in PBS before ex vivo fluorescence (mCherry) and bioluminescence (Nluc) imaging. Images were analyzed with Aura software to determine radiance with the region of interest (ROI), and fluorescence signal was normalized to background signal of lungs from mock-infected mice. Bright-field images of lungs were captured using an iPhone X camera. After imaging, lungs and nasal turbinate were homogenized in 1 mL of PBS using a Precellys tissue homogenizer (Bertin Instruments) and centrifuged at 21,500 g for 10 min to pellet cell debris. Clarified supernatants were collected and used to determine viral titers by standard plaque assay. All data are presented as mean values and SD for each group and were analyzed using Student t-test in Graphpad Prism 9 software.

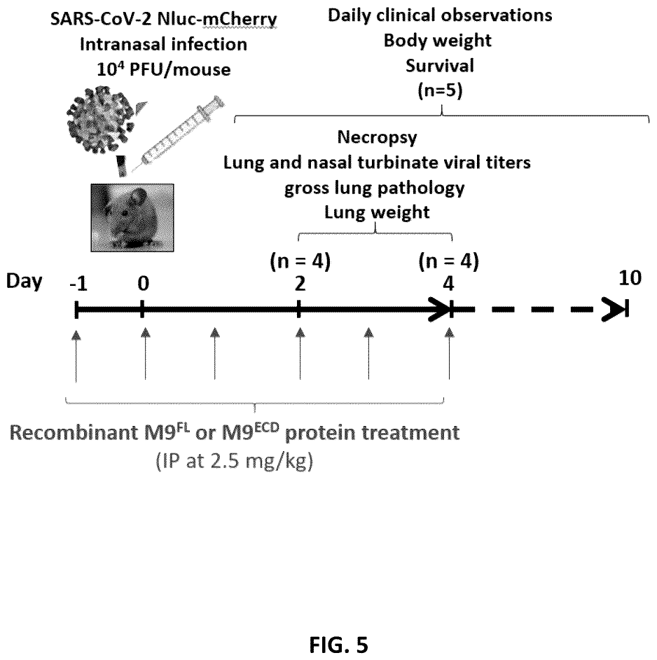

FIG. 5 shows the study design. Four groups of animals were used. Animals in Group 1 were mock treated and mock infected (n=7). Animals in Group 2 were mock treated, and SARS-CoV-2 infected (n=13). Animals in Group 3 were M9^{FL} treated and CoV2 infected (n=13). Animals in Group 4 were M9^{ECD} treated, SARS-CoV-2 infected (n=13).

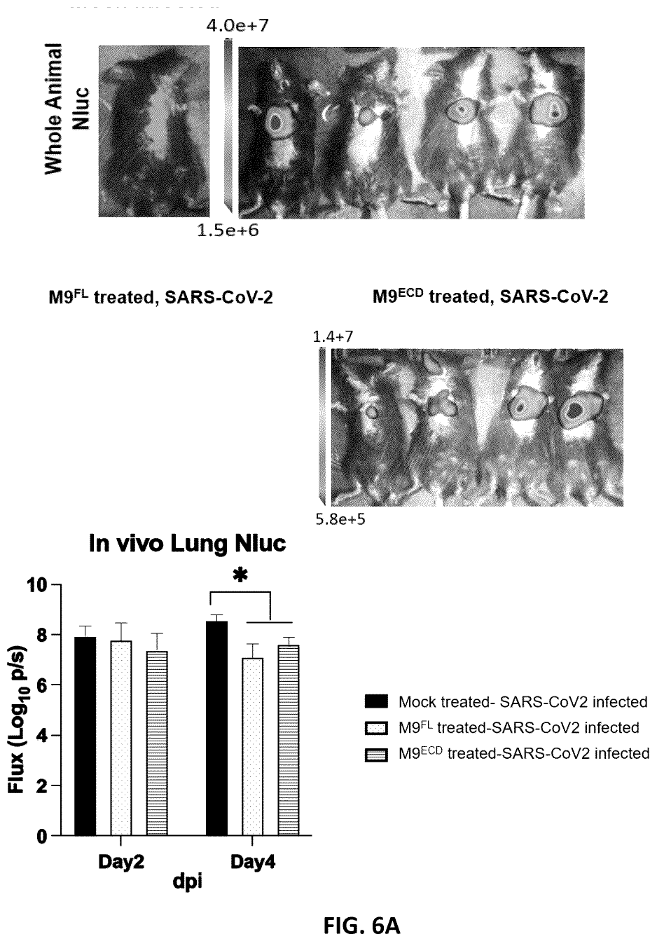

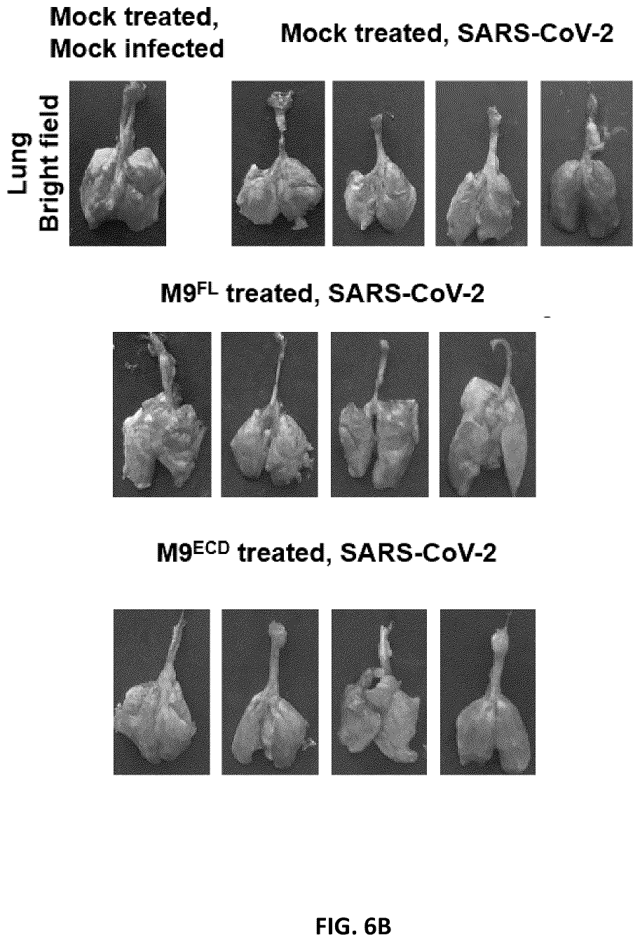

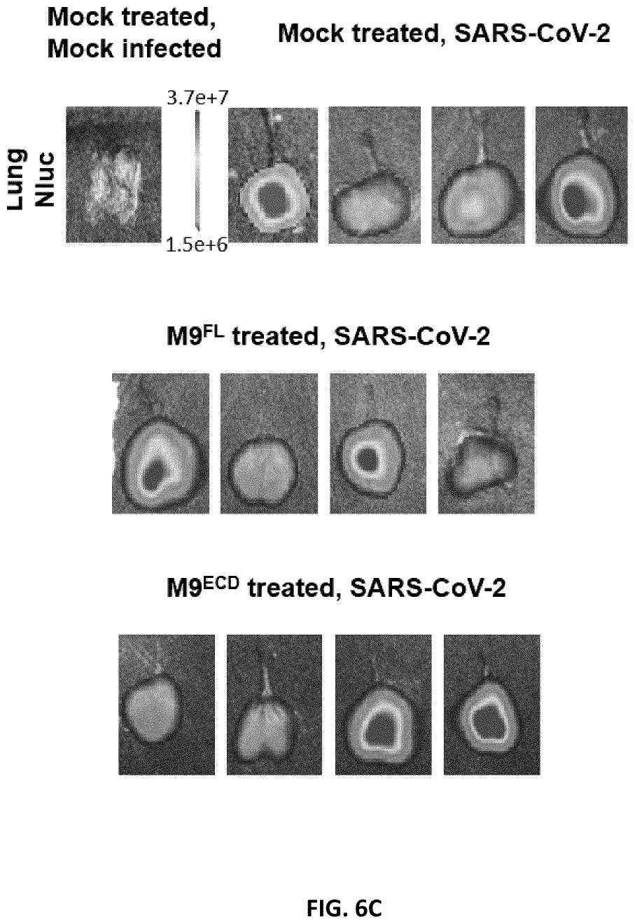

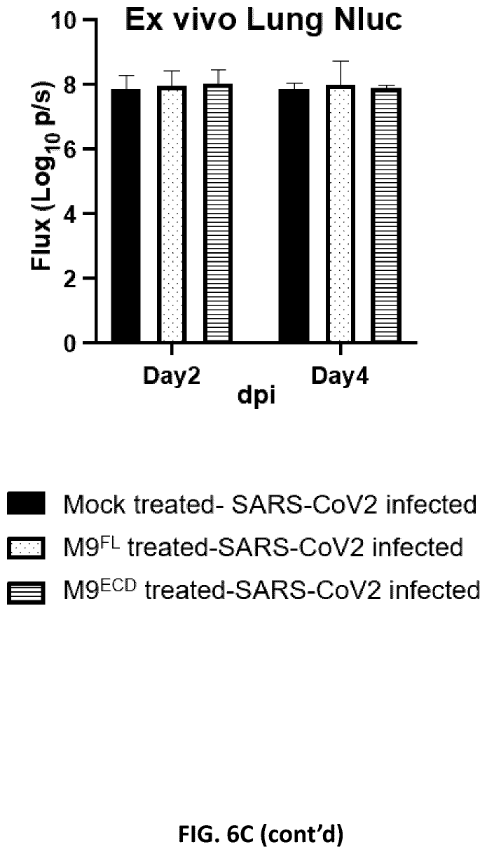

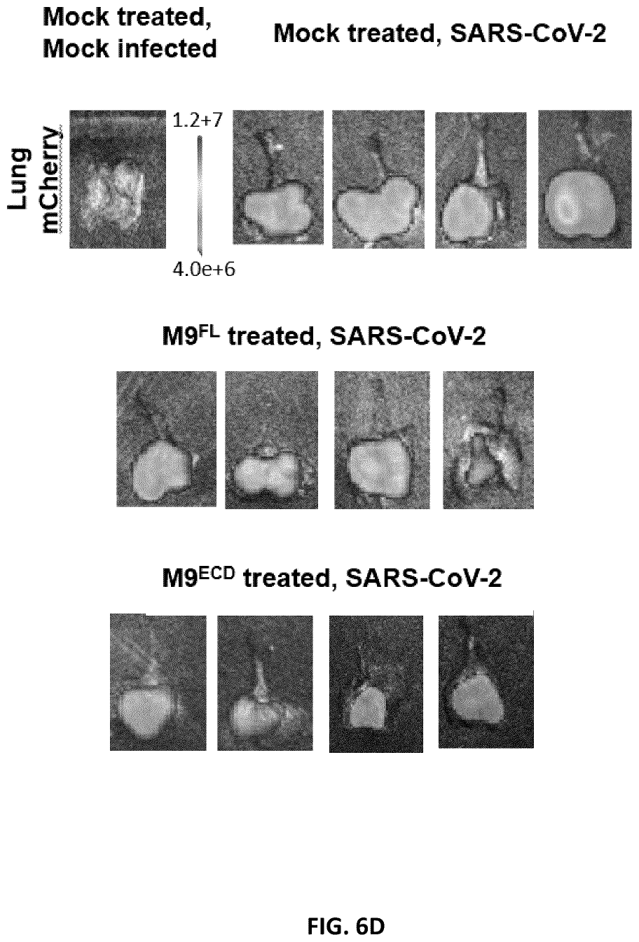

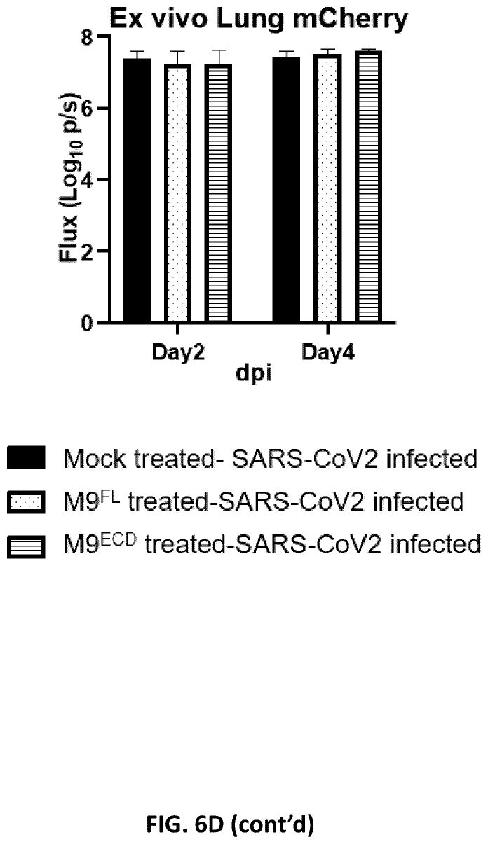

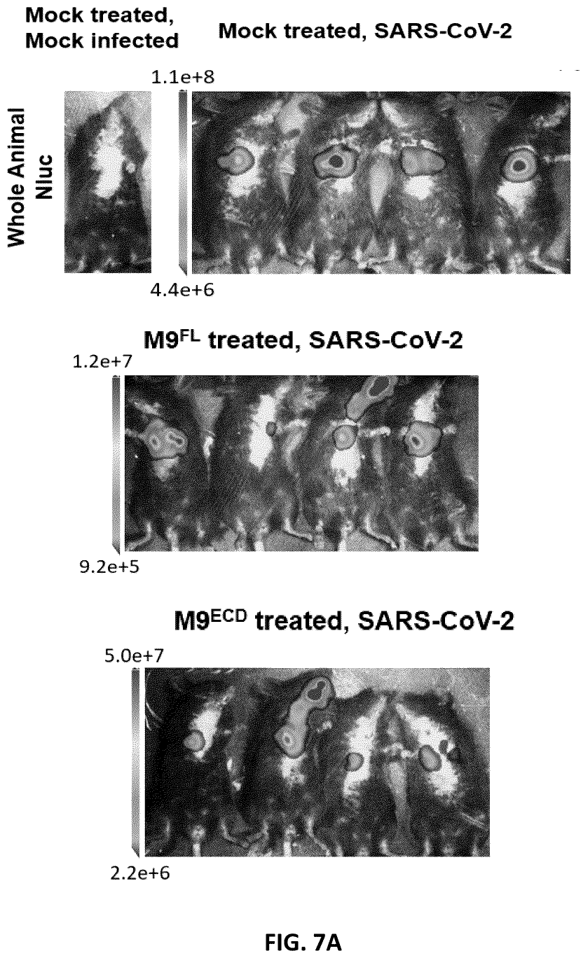

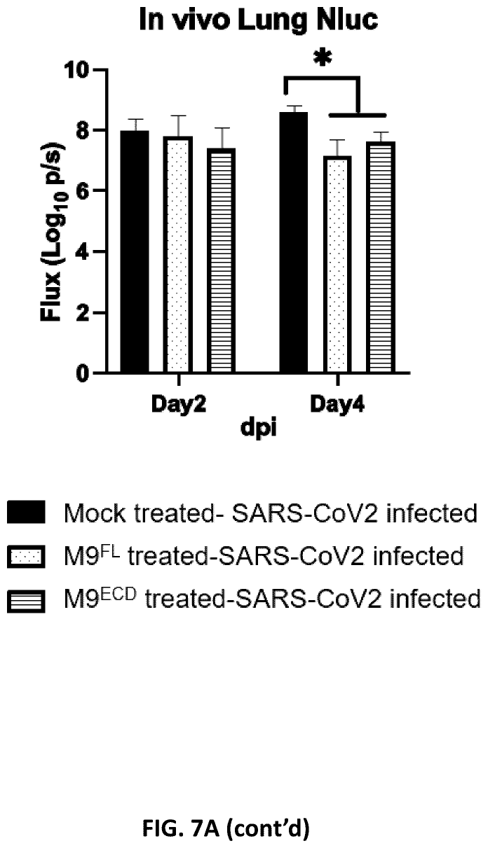

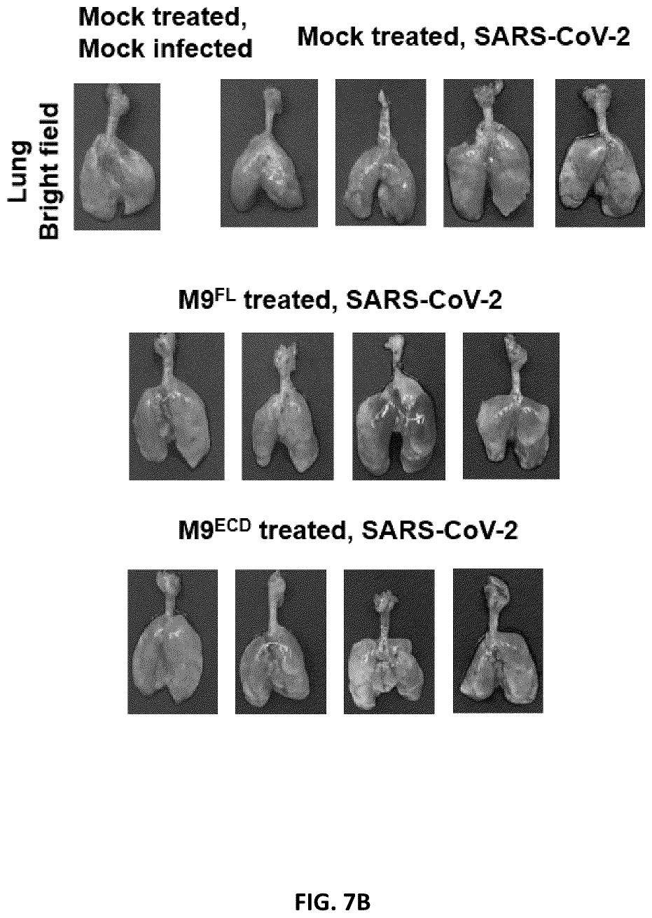

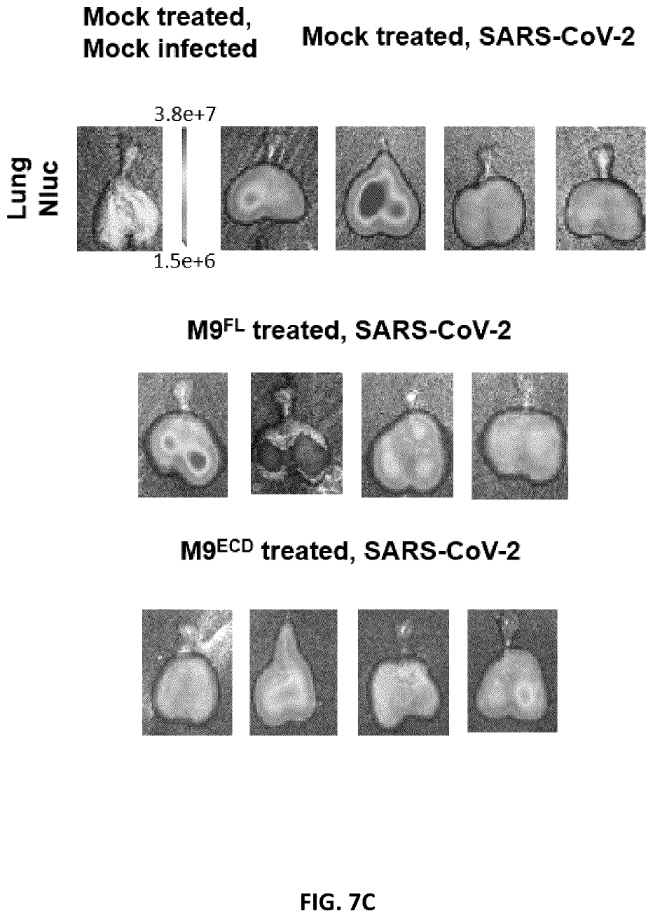

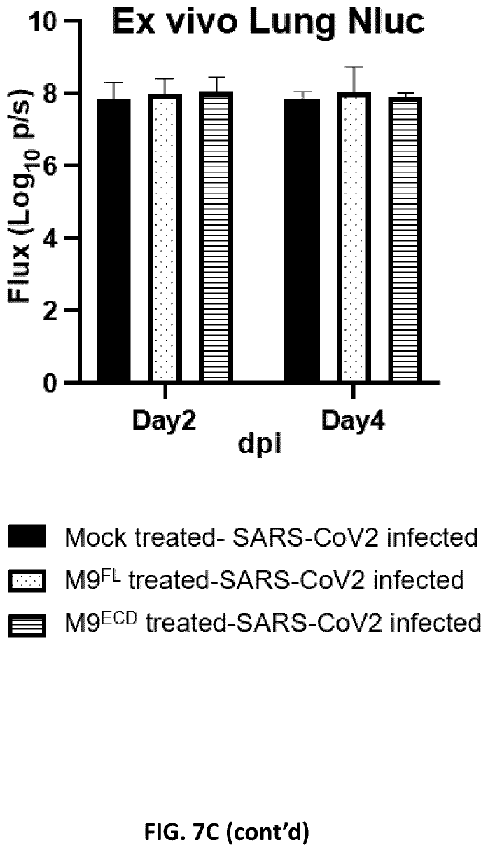

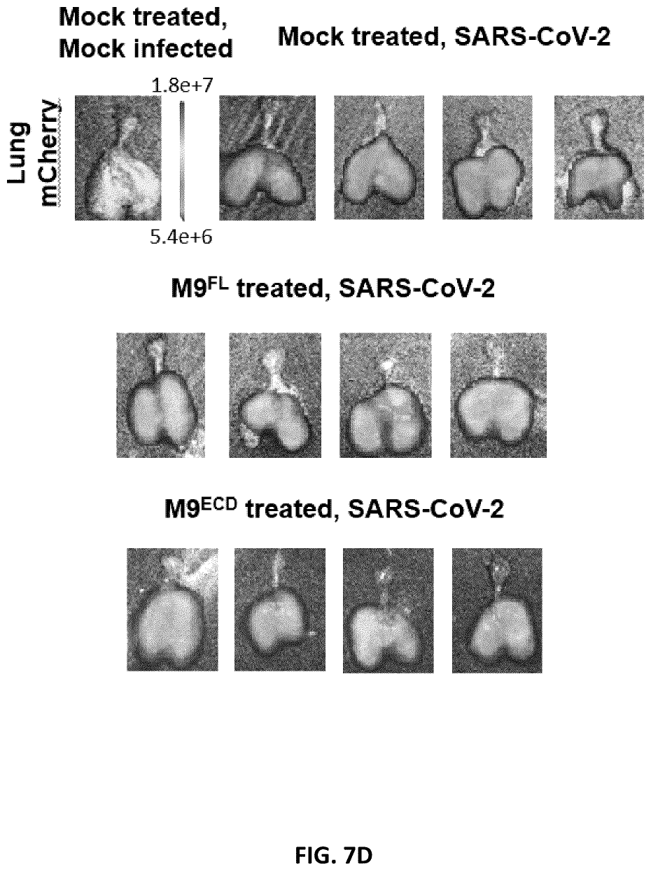

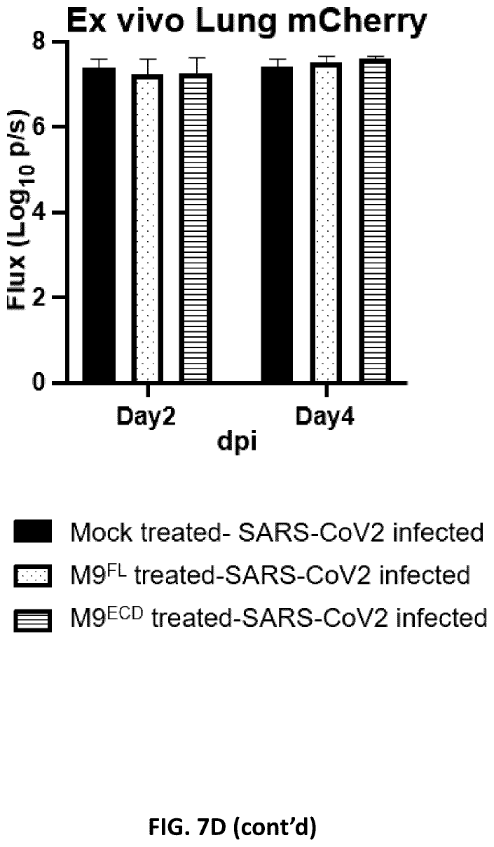

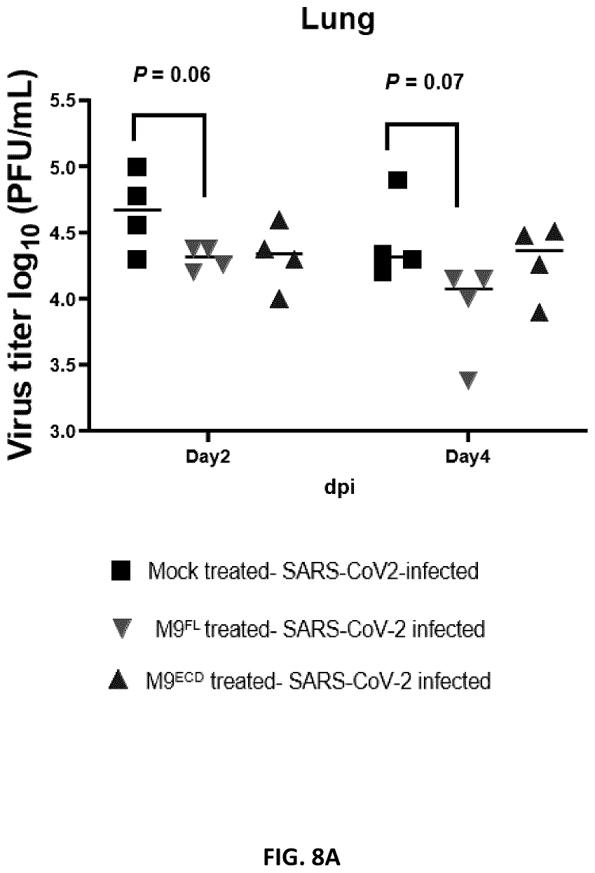

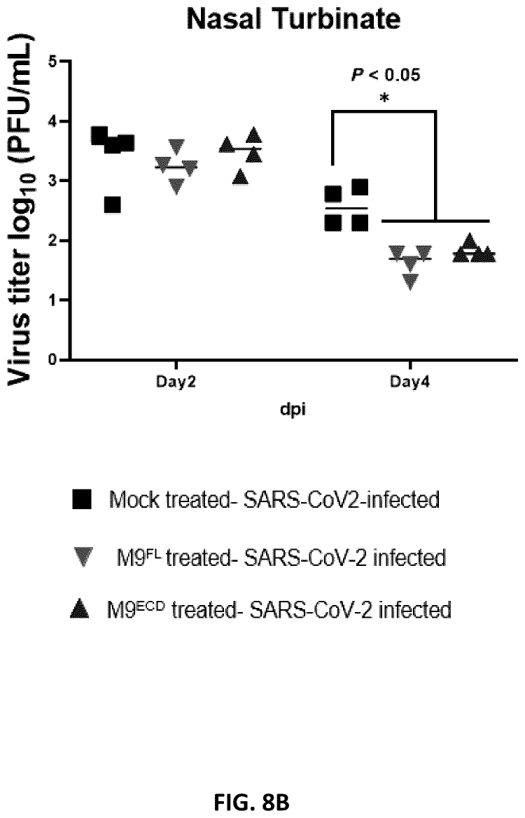

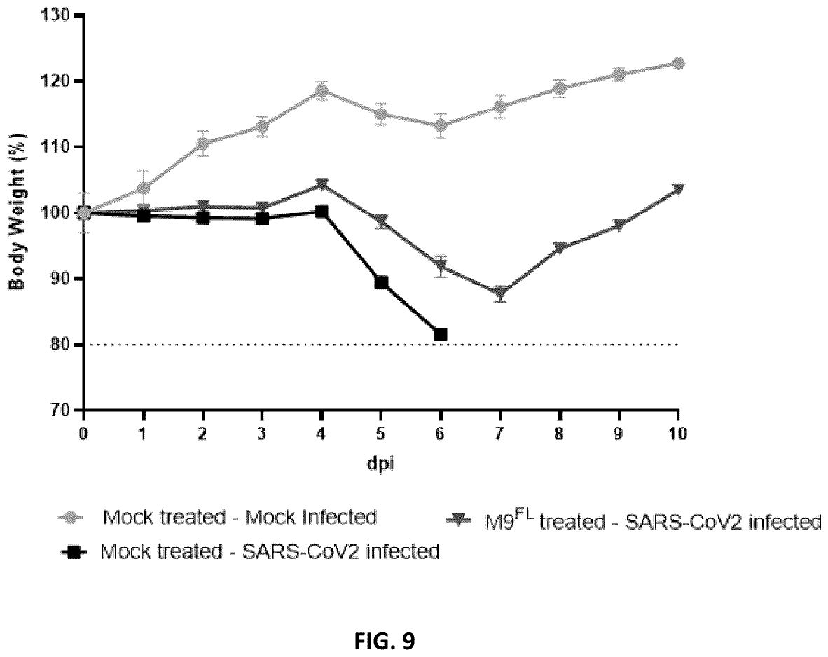

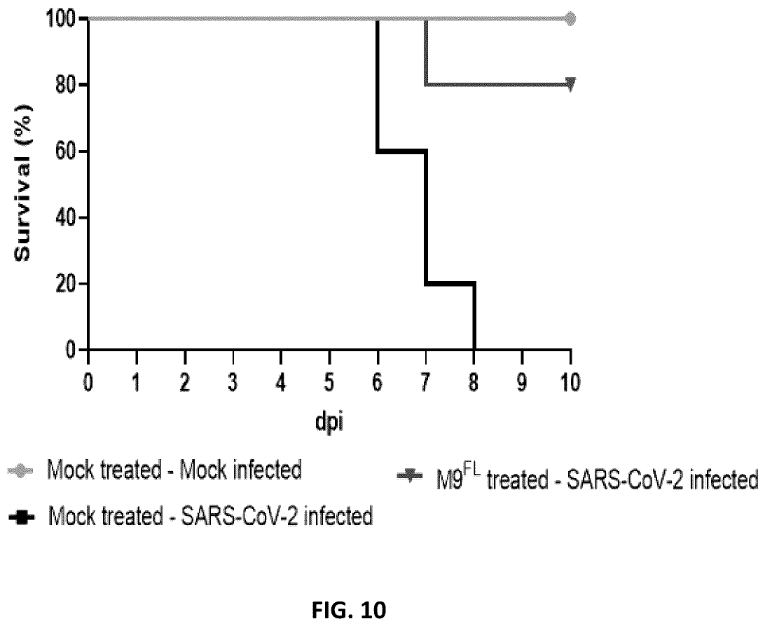

FIGS. 6A-6D show results on IVIS Day 2. FIG. 6A shows whole animal Nluc images. FIG. 6B shows lung bright field images. FIG. 6C shows lung Nluc images. FIG. 6D shows lung mCherry images. FIGS. 7A-7D show results on IVIS Day 4. FIG. 7A shows whole animal Nluc images. FIG. 7B shows lung bright field images. FIG. 7C shows lung Nluc images. FIG. 7D shows lung mCherry images. FIGS. 8A-8B show results of virus titrations. FIG. 8A shows virus titration in lungs. There is a clear trend of decreased viral titers in M9^{FL} or M9^{ECD} treated groups compared to the mock groups. FIG. 8B shows virus titration in nasal turbinates. FIG. 9 shows body weight change of the animals. The difference observed is highly statistically significant. The p value is <0.0001 on day 6 between Mock treated-SARS-CoV-2 infected group vs. M9^{FL} treated SARS-CoV-2 infected group. FIG. 10 shows survival of the animals. One out of five (20%) mice was euthanized when it lost 25% of its body weight. If no mice were euthanized due to body weight loss over 25%, the animals would have had 100% survival in the treatment arm. In conclusion, treating SARS-CoV2 infected mice with recombinant M9 protein or its ECD fragment reduced mortality by more than 80%. Further, the findings of PRMNT assay of Vero EC6 cells and viral titers of nasal turbinate and lung tissues further suggest that the anti-viral activity of recombinant M9 or its ECD fragment contributes to remarkable improvement in body weight and survival observed in ACE2 mice infected with SARS-CoV2.

### Example 5

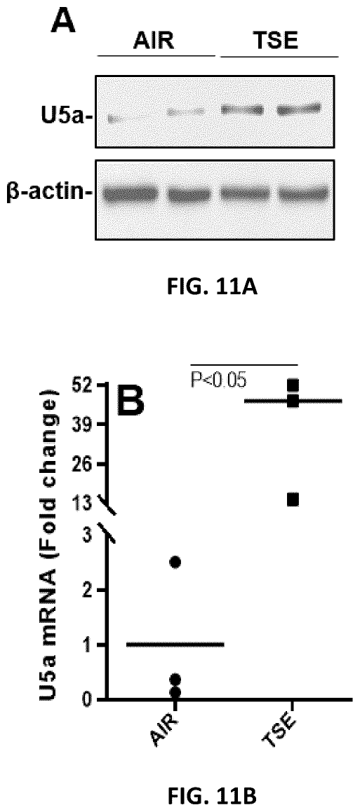

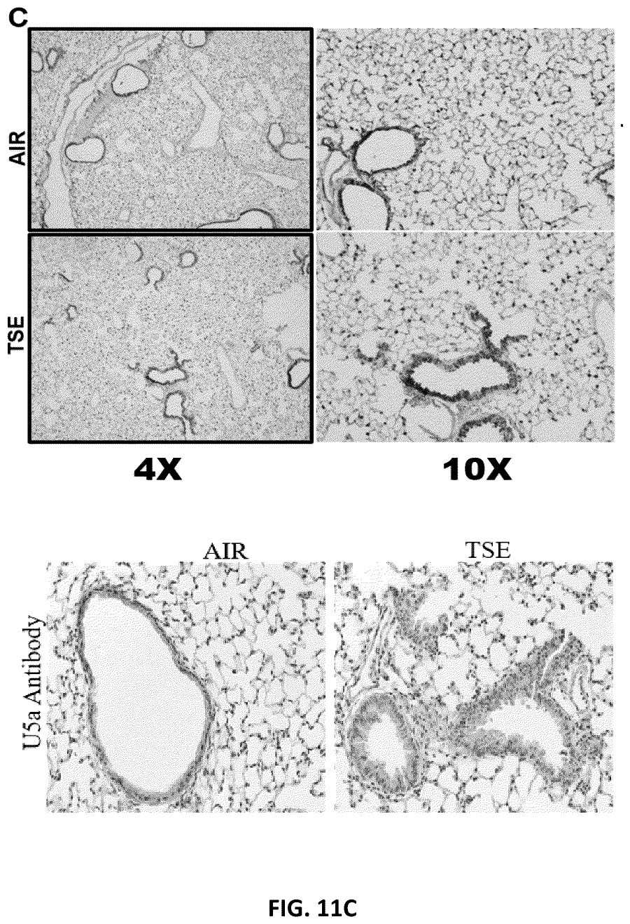

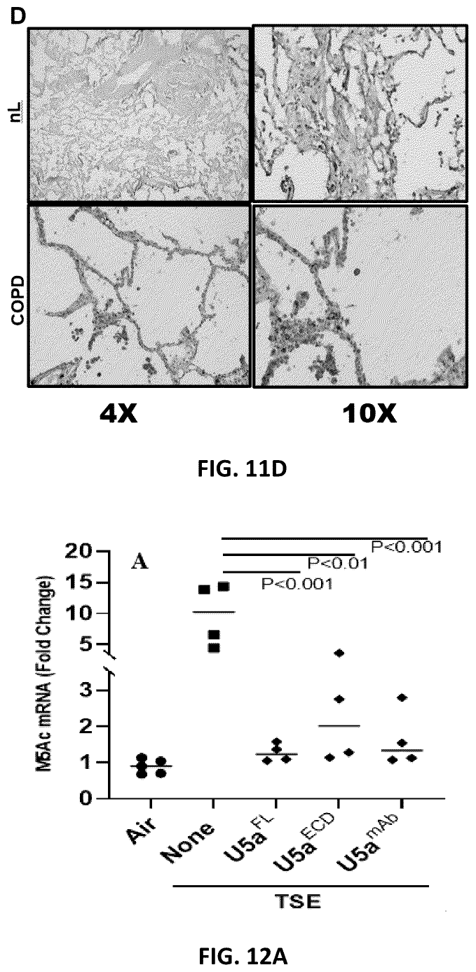

Induction of U5a expression in wild-type (WT) mice with tobacco smoke exposure (TSE)-induced lung injury. WT mice were kept in ambient AIR or exposed to tobacco smoke (TSE) for 4 h/day 5 days a week for 20 weeks. ATII cells were isolated and analyzed. FIGS. 11A-D show the results. The cell lysates were immunoblotted for U5a and 3-actin (loading control) protein. FIG. 11A shows the Western blot for the U5a levels of the control and TSE groups. The total RNA isolated from ATII cell lysates was analyzed for U5a mRNA by pPCR. FIG. 11B shows the mRNA levels of the control and TSE groups. Lung sections from WT mice kept in ambient AIR or exposed to TS for 20 weeks (FIG. 11C) or Human normal lung (nL) section from control donors or from patients with COPD (FIG. 11D) were subjected to immunohistochemical (IHC) analysis using anti-U5a antibody (PA5-103957 (Invitrogen)). Consistent with increase in ATII cell senescence and apoptosis, and mucous cell metaplasia and mucous hypersecretion by AECs, we found elevated expression of U5a in both ATII cells and AECs. Further, these findings link p53-mediated induction of U5a expression and reduced ATII cell viability and increased mucous cell metaplasia and mucus hypersecretion associated with COPD and TSE-LI.

### Example 6

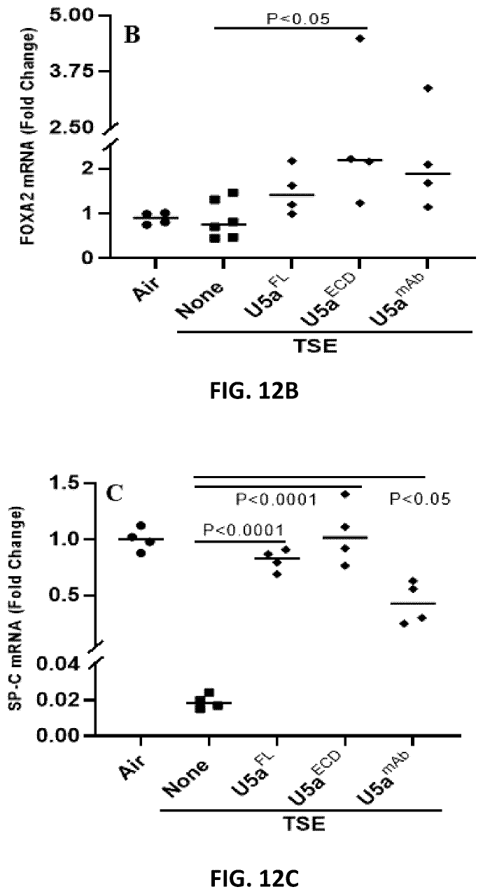

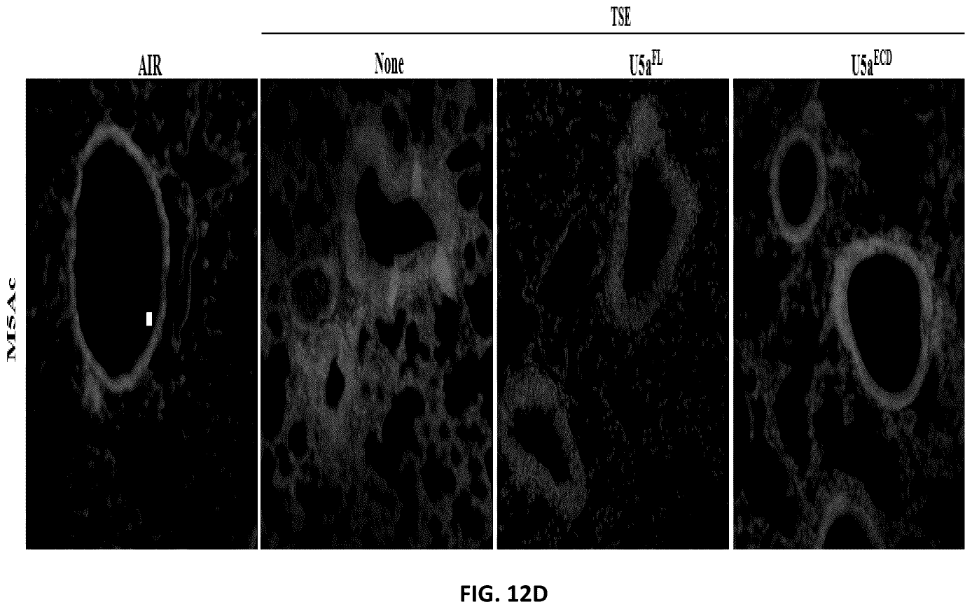

Recombinant full-length U5a protein (U5a^{FL}), extracellular domain of U5a^{F} (U5a^{ECD}) and U5a^{ECD}-specific monoclonal antibody (U5a^{mAb}, the 17C9-1 anti-U5a antibody comprising the sequences of SEQ ID NOs. 204 and 211) mitigated chronic TS exposure (TSE) induced lung injury (TSE-LI) in wild-type (WT) mice. WT mice were kept in ambient AIR or exposed to tobacco smoke (TSE) for 4 h/day 5 days a week. After 16 weeks, WT mice with TSE-LI were left untreated (None) or treated with 2.5 mg/kg of U5a^{FL}, U5a^{ECD} and U5amAb 5 days a week for 4 weeks by IP injection. Total lung RNA were analyzed for MUCSAC (M5Ac) (FIG. 12A), FOXA2 (FIG. 12B) and surfactant protein-C (SP—C) (FIG. 12C) mRNA by qPCR. Lung sections of mice treated as in FIG. 12A were subjected to IHC analysis for M5Ac (FIG. 12D). We found increased expression of U5a in AECs and ATII cells in lungs of patients with COPD and mice with TSE-LI. Further, targeting U5a signaling on TSE-LI induced signaling at the AEC and ATII cell surface using U5a^{FL}, U5a^{ECD} or monoclonal antibody developed against U5a significantly inhibits M5Ac by inducing FOXA2 in AECs suggesting inhibition of TSE-induced mucous cell metaplasia. In addition, increased surfactant protein-C (SP—C) indicates improved survival of ATII cells, which is otherwise reduced due to increased TSE-induced senescence and apoptosis of ATII cells seen in mice with TSE-LI left untreated.

### Example 7

Recombinant full-length multiple EGF-like-domains-9 (MEGF9; M9^{FL}) protein comprising the sequence of SEQ ID NO. 1 mitigated the effects of exposure to a lethal dose of Influenza A virus (IAV) in wild-type (WT) mice. WT mice weighing approximately 20 grams, between the ages of 7-8 weeks, were exposed to either 5×10⁶ IAV viral particles in saline or saline alone (mock infection control group) delivered via intranasal instillation. Three days post delivery of IAV, mice were either left untreated or treated with 2.5 mg/kg M9^{FL} daily for five days. The M9^{FL} treated group and non-treated group each totaled 7 animals (n=7). Mice were weighed daily, animals were euthanized by day 7-11 post-IAV infection, and lungs were collected for further analysis.

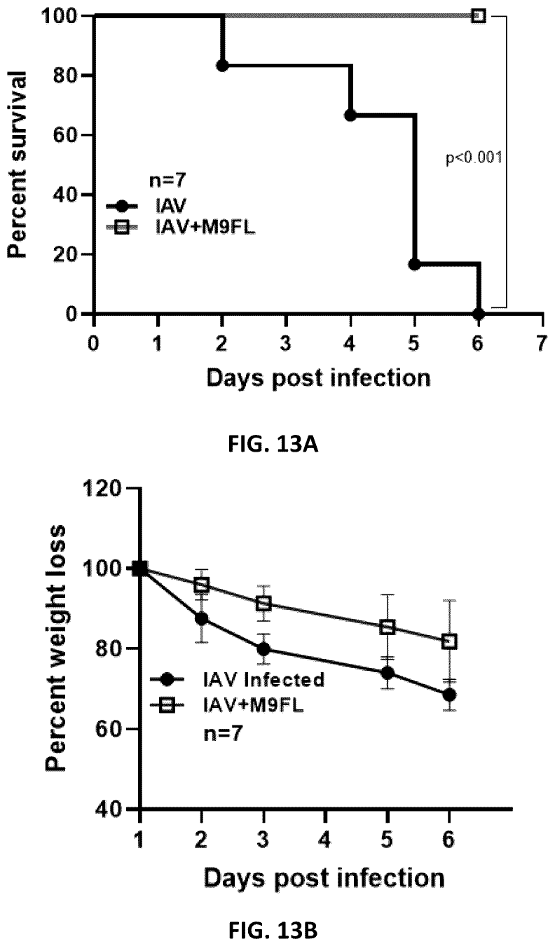

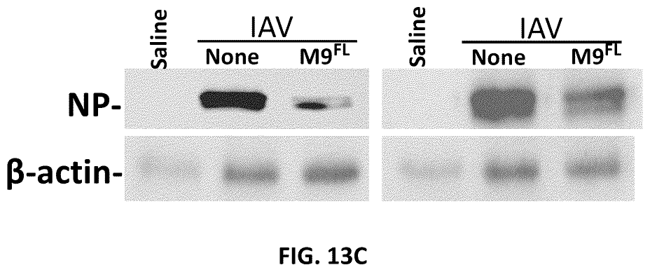

FIG. 13A shows survival of the animals infected with IAV with and without M9^{FL} treatment. The p value is <0.001 between the two groups on day 6, which is statistically significant. Animal weight loss for the animals infected with IAV with and without M9^{FL} treatment was measured starting at day 1 post IAV infection. As shown in FIG. 13B, animals treated with M9^{FL} exhibited lower weight loss compared to IAV infected animals that did not receive M9^{FL} treatment. FIG. 13C shows the Western blot for IAV Nucleoprotein (NP) levels to assess viral titer from whole lung homogenates of the saline treated control animals (mock infection) and the IAV infected animals (with M9^{FL} treatment and without “none”). NP was not detected via Western Blot analysis in the saline treated animals and the IAV infected animals had reduced NP in the M9^{FL} treatment group relative to non-treated animals.

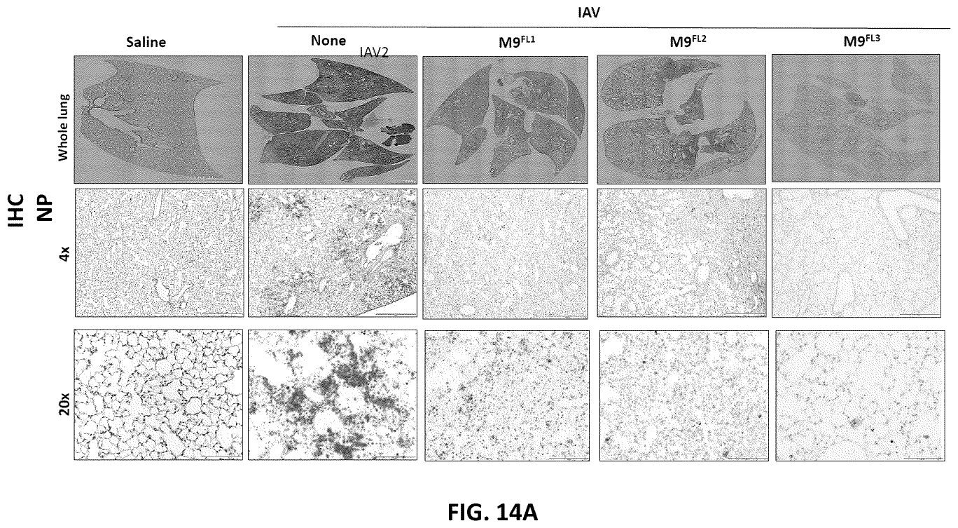

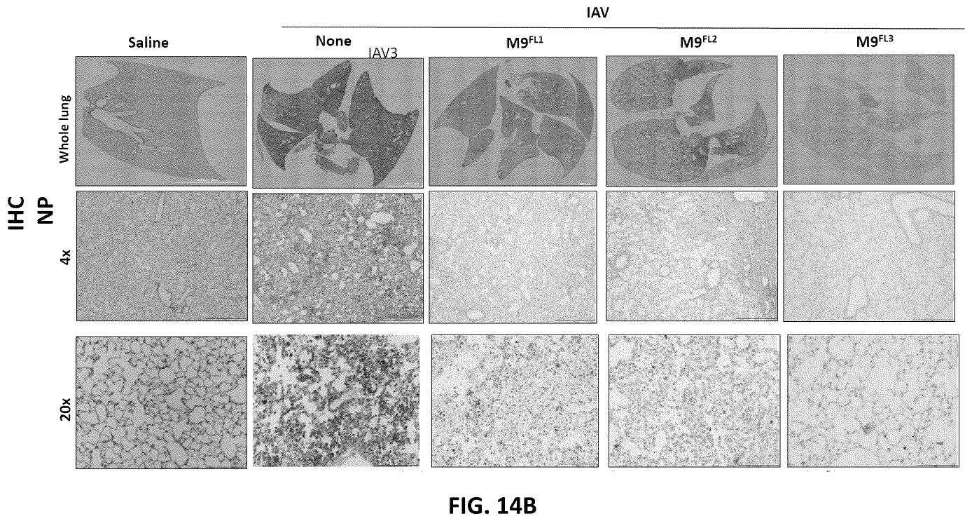

The presence of IAV antigen in lung tissue of the saline treated control animals and the IAV infected animals (with M9^{FL} treatment and without “none”) was assessed in situ using immunohistochemistry on formalin fixed and paraffin embedded lung sections (FIGS. 14A-14B). NP staining was reduced in the lung tissue of IAV infected animals treated with M9^{FL} compared to the lung tissue of IAV infected animals that were not treated with M9^{FL}

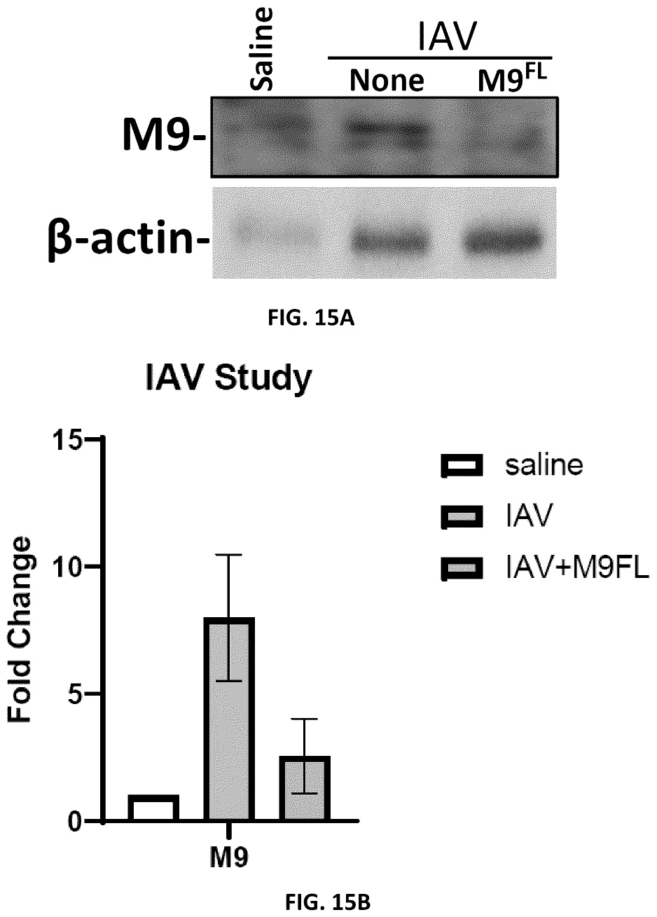

Additionally, M9 protein expression in lung tissue of the saline treated control animals and the IAV infected animals (with M9^{FL} treatment and without “none”) was assessed by subjecting total lung homogenates to Western blot analysis using an anti-M9 antibody (FIGS. 15A-15B). M9 protein expression increased in IAV infected animals compared to the saline treated control group. The M9 protein expression was reduced in IAV infected animals treated with 2.5 mg/kg M9^{FL}, as described above.

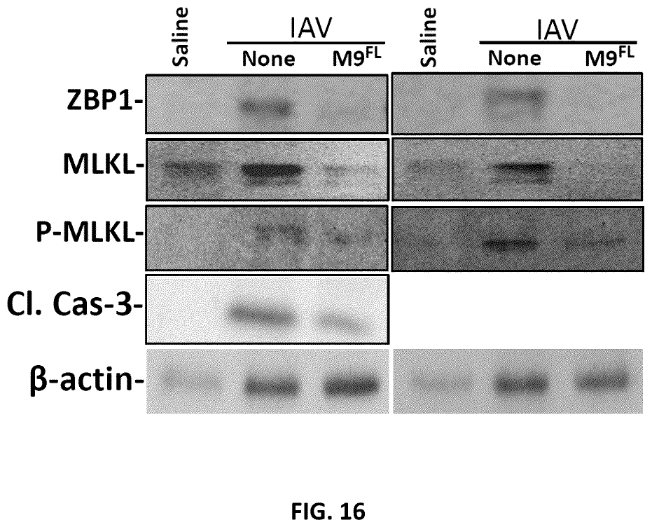

The effects of M9^{FL} treatment on IAV mediated induction of the ZBP1-MLKL pathway was assessed by Western blot analysis (FIG. 16). Total lung homogenates of WT mice from the saline treated control animals and the IAV infected animals (with M9^{FL} treatment and without “none”) was subjected to Western blotting using antibodies directed to ZBP1, MLKL, phospho-MLKL, and cleaved caspase-3 (Cl. Cas-3) to assess changes in molecular factors associated with lung injury. As shown in FIG. 16, the ZBP1-MLKL pathway was induced in IAV infected animals compared to the mock infection control group, but was reduced in M9^{FL} treated animals.

### Example 8

Recombinant multiple EGF-like-domains-9 biologically active fragment comprising a 12 amino acid (12-mer) peptide (MEGF9; M9^{P}) comprising the sequence of HGHVDPVKTPKI (SEQ ID NO. 350) mitigated the effects of exposure to a lethal dose of Influenza A virus (IAV) in wild-type (WT) mice. WT mice were exposed to either 5×10⁶ IAV viral particles in saline or saline alone (mock infection control group) delivered via intranasal instillation. Four days post delivery of IAV, mice were either left untreated or treated with 1.25 mg/kg M9^{P} daily for five days. Mice were weighed daily, animals were euthanized by day 7-11 post-IAV infection, and lungs were collected for further analysis.

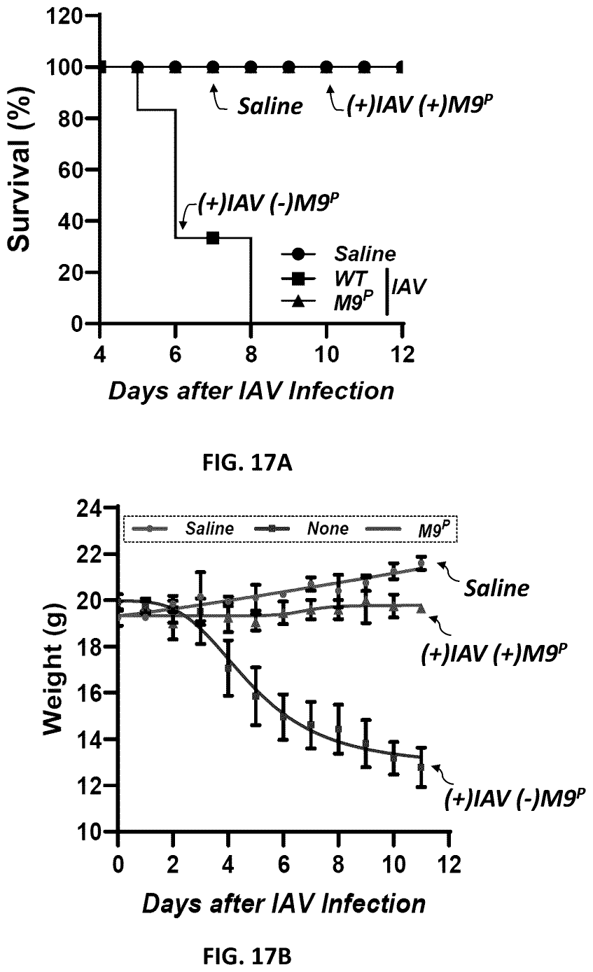

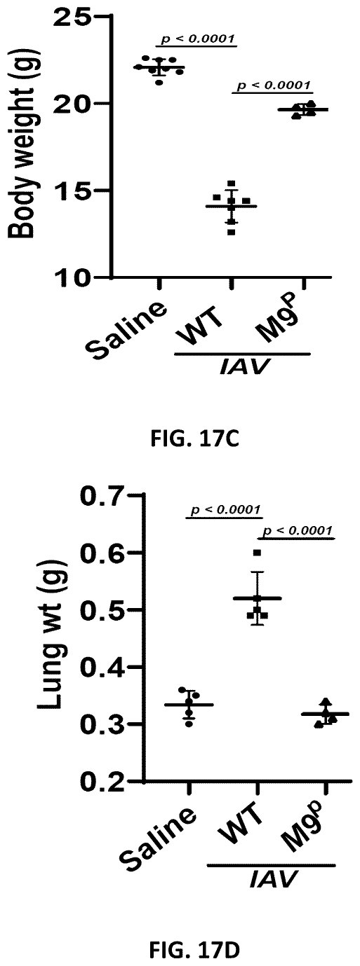

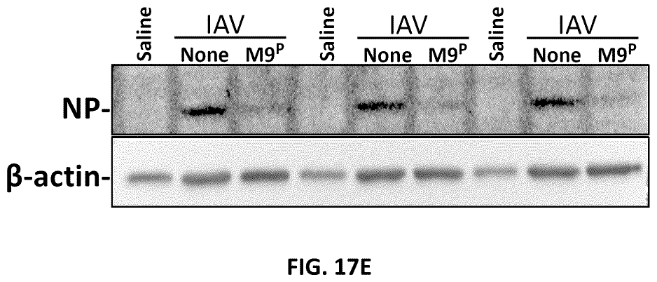

FIG. 17A shows survival of saline treated control animals (mock infection) and the IAV infected animals with M9^{P} treatment and without. Mice with >20% weight loss were considered moribund. Animals treated with M9^{P} exhibited lower total body weight loss over 11 days relative to saline treated animals compared to IAV infected animals that did not receive M9^{P} treatment (FIG. 17B). Changes in total body weight and lung weight for saline treated control animals and the IAV infected animals with M9^{P} treatment and without (“WT”) are shown in FIGS. 17C-17D. IAV infected animals treated with M9^{P} did not show the increased lung weight that was observed in IAV infected animals that were not treated with M9^{P}(FIG. 17D). FIG. 17E shows the Western blot for IAV Nucleoprotein (NP) levels to assess viral titer from whole lung homogenates of the saline treated control animals and the IAV infected animals (with M9^{P} treatment and without “none”). NP was not detected via Western Blot analysis in the saline treated mock infection animals and the IAV infected animals had reduced NP in the M9^{P} treatment group relative to animals that were not treated with M9^{P}.

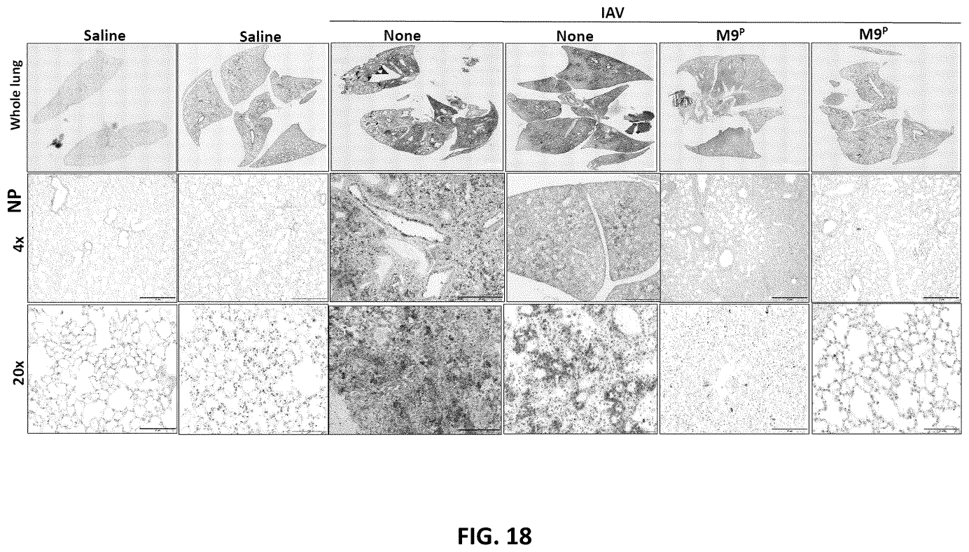

The presence of IAV antigen in lung tissue of the saline treated control animals and the IAV infected animals (with M9^{P} treatment and without “none”) was assessed in situ using immunohistochemistry formalin fixed and paraffin embedded lung sections (FIG. 18). NP staining was reduced in the lung tissue of IAV infected animals treated with M9^{P} compared to the lung tissue of IAV infected animals that were not treated with M9^{P}.

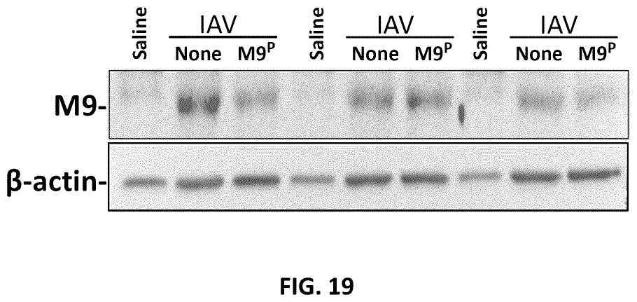

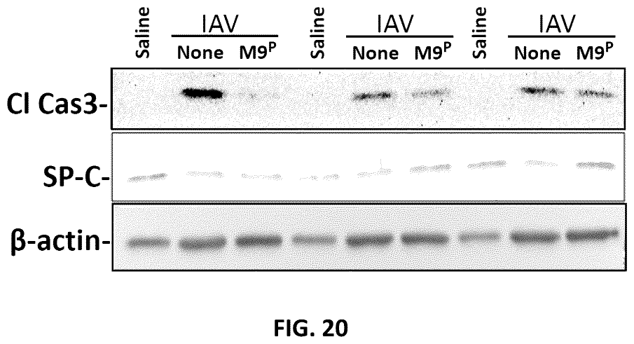

M9 protein expression in lung tissue of the saline treated control animals and the IAV infected animals (with M9^{P} treatment and without “none”) was assessed by subjecting total lung homogenates to Western blot analysis using an anti-M9 antibody (FIG. 19). M9 protein expression increased in IAV infected animals compared to the saline treated control group. The M9 protein expression was reduced in IAV infected animals treated with 1.25 mg/kg M9^{P}, as described above. Total lung homogenates of WT mice from the saline treated control animals and the IAV infected animals (with M9^{P} treatment and without “none”) were also subjected to Western blotting using antibodies directed to cleaved caspase-3 (Cl. Cas-3) and surfactant protein-C (SP—C) to assess changes in molecular factors associated with lung injury. As shown in FIG. 20, the cleaved caspase-3 was induced in IAV infected animals compared to the mock infection control group, but was reduced in IAV infected animals treated with M9^{P}.

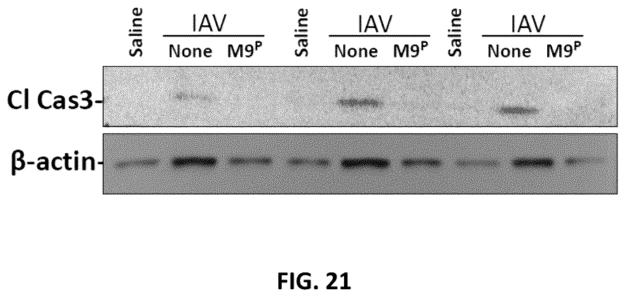

Alveolar macrophages (amQs) were also isolated from the saline treated control animals and the IAV infected animals (with M9^{P} treatment and without “none”) and subjected to Western blot analysis of cleaved caspase 3 levels as a molecular marker for lung injury (FIG. 21). The cleaved caspase-3 was induced in IAV infected animals, but was not detected in saline treated (mock infection) animals or IAV infected animals that were treated with 1.25 mg/kg M9^{P}.

### Example 9

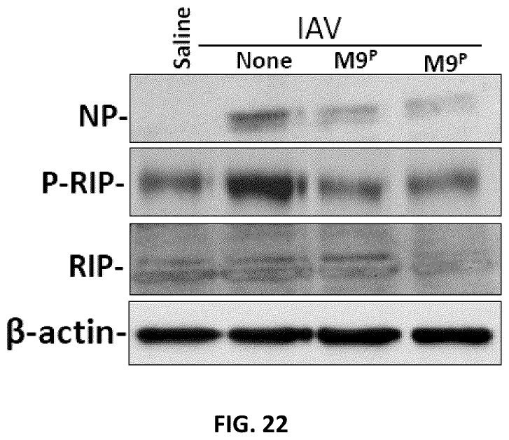

M9^{P} treatment inhibits necroptosis pathway markers in primary human alveolar epithelial cells (AECs). AECs were treated with M9^{P} (20 μM) two hours after IAV infection. Cellular lysates of the AECs were prepared 72 hours after infection. AEC lysates were subjected to Western blot analysis to assess viral titer using an antibody for NP. Induction of necroptosis pathway markers was assessed using antibodies for Receptor Interacting Protein (RIP) kinase and phosphorylated-RIP “p-RIP”). As shown in FIG. 22, NP was not detected via Western Blot analysis in the saline treated AECs and the IAV infected AECs had reduced NP in response to M9^{P} treatment relative to non-treated AECs (“none”). Increased phosphorylated RIP was observed in IAV infected AECs that were not treated with M9¹ (“none”) relative to AECs treated with 2 μM M9¹ two hours after IAV infection.

The references cited above are all incorporated by reference herein, whether specifically incorporated or not.

The present application also provides aspects and embodiments as set forth in the following numbered Statements:

Statement 1. A method of treating a subject in need of therapy for a pathogen-induced infection, symptom, disease, disorder, injury, or condition, comprising administering to the subject:
a) multiple EGF-like-domains-9 (MEGF9) or a biologically active fragment thereof,
b) uncoordinated receptor 5A (UNC5A) or a biologically active fragment thereof,
c) dolichyl-phosphate beta-glucosyltransferase (ALG5) or a biologically active fragment thereof,
d) a combination of two or three of a)-c);
e) an antibody specific for a);
f) an antibody specific for b);
g) an antibody specific for c);
h) a combination of two or three of e)-g); or
i) a combination of at least one of a)-c) and at least one of e)-g).

Statement 2. The method of Statement 1, comprising administering MEGF9 or a biologically active fragment thereof; and UNC5A or a biologically active fragment thereof.

Statement 3. The method of Statement 1, comprising administering UNC5A or a biologically active fragment thereof; and ALG5 or a biologically active fragment thereof.

Statement 4. The method of Statement 1, comprising administering MEGF9 or a biologically active fragment thereof; and ALG5 or a biologically active fragment thereof.

Statement 5. The method of Statement 1, comprising administering MEGF9 or a biologically active fragment thereof; UNC5A or a biologically active fragment thereof; and ALG5 or a biologically active fragment thereof.

Statement 6. The method of any one or combination of the Statements above, wherein the or biologically active fragment of MEGF9 comprises a sequence with at least 80%, at least 85%, at least 90%, or at least 95% sequence identity to SEQ ID NO: 1.

Statement 7. The method of any one or combination of the Statements above, wherein the UNC5A comprises a sequence with at least 80%, at least 85%, at least 90%, or at least 95% sequence identity to SEQ ID NO: 5.

Statement 8. The method of any one or combination of the Statements above, wherein the ALG5 comprises a sequence with at least 80%, at least 85%, at least 90%, or at least 95% sequence identity to SEQ ID NO: 9.

Statement 9. The method of any one or combination of the Statements above, wherein the ALG5 comprises a sequence with at least 80%, at least 85%, at least 90%, or at least 95% sequence identity to SEQ ID NO: 102.

Statement 10. The method of any one or combination of Statements 1-9, wherein the biologically active fragment of the ALG5 comprises a sequence of SEQ ID NO: 1 or a one-amino acid or two-amino acid modification thereof.

Statement 11. The method of any one or combination of Statements 1-9, wherein the biologically active fragment of the ALG5 comprises a sequence of SEQ ID NO: 5 or a one-amino acid or two-amino acid modification thereof.

Statement 12. The method of any one or combination of Statements 1-9, wherein the biologically active fragment of the ALG5 comprises a sequence of SEQ ID NO: 9 or a one-amino acid or two-amino acid modification thereof.

Statement 13. The method of any one or combination of Statements 1-9, wherein the biologically active fragment of the ALG5 comprises a sequence of SEQ ID NO: 102 or a one-amino acid or two-amino acid modification thereof.

Statement 14. The method of any one or combination of Statements 1-13, wherein the biologically active fragment of the MEGF9 comprises a sequence with at least 80%, at least 85%, at least 90%, or at least 95% sequence identity to SEQ ID NO: 3.

Statement 15. The method of any one or combination of Statements 1-13, wherein the biologically active fragment of the MEGF9 consists of a sequence with at least 80%, at least 85%, at least 90%, or at least 95% sequence identity to SEQ ID NO: 3.

Statement 16. The method of any one or combination of Statements 1-13, wherein the biologically active fragment of the MEGF9 consists of a sequence with at least 80%, at least 85%, at least 90%, or at least 95% sequence identity to SEQ ID NO: 104.

Statement 17. The method of any one or combination of Statements 1-13, wherein the biologically active fragment of the MEGF9 comprises a sequence of SEQ ID NO: 4 or a one-amino acid or two-amino acid modification thereof.

Statement 18. The method of any one or combination of Statements 1-13, wherein the biologically active fragment of the MEGF9 consists of a sequence of SEQ ID NO: 4 or a one-amino acid or two-amino acid modification thereof.

Statement 19. The method of any one or combination of Statements 1-13, wherein the biologically active fragment of the MEGF9 consists of a sequence of SEQ ID NO: 103 or a one-amino acid or two-amino acid modification thereof.

Statement 20. The method of any one or combination of Statements 1-9, wherein the biologically active fragment of the UNC5A comprises a sequence with at least 80%, at least 85%, at least 90%, or at least 95% sequence identity to SEQ ID NO: 7.

Statement 21. The method of any one or combination of Statements 1-20, wherein the biologically active fragment of the UNC5A consists of a sequence with at least 80%, at least 85%, at least 90%, or at least 95% sequence identity to SEQ ID NO: 7.

Statement 22. The method of any one or combination of Statements 1-20, wherein the biologically active fragment of the UNC5A comprises a sequence with at least 80%, at least 85%, at least 90%, or at least 95% sequence identity to SEQ ID NO: 8.

Statement 23. The method of any one or combination of Statements 1-20, wherein the biologically active fragment of the UNCSA consists of a sequence with at least 80%, at least 85%, at least 90%, or at least 95% sequence identity to SEQ ID NO: 8.

Statement 24. The method of any one or combination of Statements 1-20, wherein the biologically active fragment of the UNC5A consists of a sequence with at least 80%, at least 85%, at least 90%, or at least 95% sequence identity to SEQ ID NO: 104.

Statement 25. The method of any one or combination of Statements 1-20, wherein the biologically active fragment of the UNCSA consists of a sequence of SEQ ID NO: 7 or a one-amino acid or two-amino acid modification thereof.

Statement 26. The method of any one or combination of Statements 1-20, wherein the biologically active fragment of the UNC5A consists of a sequence of SEQ ID NO: 8 or a one-amino acid or two-amino acid modification thereof.

Statement 27. The method of any one or combination of the Statements above, further comprising administering a Cav1 scaffolding domain peptide (CSP).

Statement 28. The method of Statement 27, wherein the CSP comprises a sequence with at least at least 80%, at least 85%, at least 90%, or at least 95% sequence identity to SEQ ID NO: 10.

Statement 29. The method of Statement 27, wherein the biologically active fragment of the CSP comprises a sequence of SEQ ID NO: 11 or a one-amino acid modification thereof.

Statement 30. The method of Statement 27, wherein the biologically active fragment of the CSP consists of a sequence of SEQ ID NO: 11 or a one-amino acid modification thereof.

Statement 31. The method of any one or combination of the preceding Statements, wherein the pathogen-induced infection, symptom, disease, disorder, injury, or condition is caused by a viral infection.

Statement 32. The method of any one or combination of the preceding Statements, wherein the subject suffers from pathogen-induced acute lung injury (ALI), acute respiratory distress syndrome (ARDS), pulmonary fibrosis, COPD, asthma, pneumonia, a vascular lung disease, an interstitial lung disease, or a combination thereof.

Statement 33. The method of Statement 31, wherein the viral infection is an infection by an influenza virus, a respiratory syncytial virus (RSV), an enterovirus (Picornaviridae), a rhinovirus, parainfluenza, a metapneumovirus, a bocavirus, an adenovirus, or a Coxsackie virus.

Statement 34. The method of Statement 31, wherein the viral infection is a coronavirus infection.

Statement 35. The method of Statement 34, wherein the coronavirus is MERS-CoV, SARS-CoV-1, or SARS-CoV-2.

Statement 36. The method of Statement 34, wherein the coronavirus is MERS-Cov.

Statement 37. The method of Statement 34, wherein the subject has Middle East respiratory syndrome (MERS).

Statement 38. The method of Statement 34, wherein the coronavirus is SARS-CoV-1.

Statement 39. The method of Statement 34, wherein the coronavirus is SARS-CoV-2.

Statement 40. The method of any one or combination of the preceding Statements, wherein the subject has severe acute respiratory syndrome (SARS).

Statement 41. The method of any one or combination of the preceding Statements, wherein the subject has coronavirus disease 2019 (COVID-19).

Statement 42. The method of any one or combination of the preceding Statements, wherein the antibody is monoclonal antibody (mAb).

Statement 43. The method of any one or combination of the Statements above, further comprising administering a), b), c), d), e), f), g), h), or i) to the subject in the form of a pharmaceutical composition that further comprises a pharmaceutically acceptable vehicle or excipient.

Statement 44. The method of Statement 43, further comprising administering the pharmaceutical composition via a route selected from the group consisting of intrapulmonary, intravenous, intramuscular, subcutaneous, oral, or in any combination thereof.

Statement 45. The method of Statement 43, further comprising administering the pharmaceutical composition via an intrapulmonary route of administration.

Statement 46. The method of Statement 43, further comprising administering the pharmaceutical composition via inhalation.

Statement 47. The method of Statement 43, further comprising administering the pharmaceutical composition in the form of a dry powder formulation.

Statement 48. The method of Statement 47, wherein the dry powder comprises less than 10% or less than 1% (by weight) of water.

Statement 49. The method of Statement 43, wherein the pharmaceutical composition is formulated for lung delivery, inhalation, or pressurized metered dose inhalation.

Statement 50. The method of any one or combination of the preceding Statements, further comprising administering at least one additional therapeutic agent.

Statement 51. The method of Statement 50, wherein the at least one additional therapeutic agent is chloroquine, hydroxychloroquine, type I interferon, Azithromycin, Tocilizumab, sarilumab, interferon beta, anti-virals, remdesivir, baricitinib, dexamethasone, monoclonal antibodies including bamlanivimab (LY-CoV555), etesevimab, Casirivimab and imdevimab, AZD7442, VIR-7831, bemnifosbuvir (AT-527), lenzilumab, leronlimab, favipiravir, lopinavir, nirmatrelvir, molnupiravir, ritonavir, or a combination thereof.

Statement 52. A vector comprising a nucleic acid molecule comprising one or more sequences encoding
a) MEGF9 or a biologically active fragment thereof;
b) UNC5A or a biologically active fragment thereof;
c) ALG5 or a biologically active fragment thereof; or
d) a combination two or three of any of a-c.

Statement 53. A cell comprising the vector of Statement 52.

Statement 54. A method of producing a therapeutic protein, comprising:
culturing the cell of clam 53 in a culture medium under a condition sufficient to produce a therapeutic protein comprising the MEGF9, UNC5A, ALG5, and/or biological fragment thereof; and
recovering the therapeutic protein from the cell or the culture medium.

Statement 55. The method of Statement 54, further comprising isolating the therapeutic protein recovered from the cell or the culture medium.

Statement 56. The method of Statement 54 or 55, further comprising formulating the therapeutic protein into a pharmaceutical composition.

Statement 57. The method of Statement 56, wherein the therapeutic protein is formulated as dry powder.

Statement 58. The method of Statement 57, wherein the dry powder is produced by a milling process, spray-drying process, jet milling, ball milling, or wet milling.

Statement 59. The method of Statement 57, wherein the dry powder is produced by thin film freezing.

Statement 60. The method of any one or combination of Statements 54-59, wherein the subject is a human.

Statement 61. Use of an antibody specific for any one or combination of SEQ ID NOs:1-9 and 102-104 for the treatment of an infection by a coronavirus, an influenza virus, a respiratory syncytial virus (RSV), an enterovirus (Picornaviridae), a rhinovirus, parainfluenza, a metapneumovirus, a bocavirus, an adenovirus, or a Coxsackie virus in a subject.

Statement 62. Use of an antibody specific for any one of SEQ ID NOs:1-9 and 102-104 for the manufacture of a medicament for treatment of an infection by a coronavirus, an influenza virus, a respiratory syncytial virus (RSV), an enterovirus (Picornaviridae), a rhinovirus, parainfluenza, a metapneumovirus, a bocavirus, an adenovirus, or a Coxsackie virus in a subject.

Statement 63. The use of Statement 62, wherein the medicament further comprises peptide CSP7.

Statement 64. The use of any one of Statements 61-63, wherein the antibody is specific for SEQ ID NO: 1.

Statement 65. The use of any one of Statements 61-63, wherein the antibody is specific for SEQ ID NO: 2.

Statement 66. The use of any one of Statements 61-63, wherein the antibody is specific for SEQ ID NO: 3.

Statement 67. The use of any one of Statements 61-63, wherein the antibody is specific for SEQ ID NO: 4.

Statement 68. The use of any one of Statements 61-63, wherein the antibody is specific for SEQ ID NO: 5.

Statement 69. The use of any one of Statements 61-63, wherein the antibody is specific for SEQ ID NO: 6.

Statement 70. The use of any one of Statements 61-63, wherein the antibody is specific for SEQ ID NO: 7.

Statement 71. The use of any one of Statements 61-63, wherein the antibody is specific for SEQ ID NO: 8.

Statement 72. The use of any one of Statements 61-63, wherein the antibody is specific for SEQ ID NO: 9.

Statement 73. The use of any one of Statements 61-63, wherein the antibody is specific for SEQ ID NO: 102.

Statement 74. The use of any one of Statements 61-63, wherein the antibody is specific for SEQ ID NO: 103.

Statement 75. The use of any one of Statements 61-63, wherein the antibody is specific for SEQ ID NO: 104.

Statement 76. The use of any one of Statements 61-63, wherein the antibody is a mAb.

Statement 77. An M9-binding protein comprising:
a heavy chain variable region comprising
   a variable heavy chain complementarity determining region 1 (V_{H} CDR1) comprising SEQ ID NO: 108,
   a variable heavy chain complementarity determining region 2 (V_{H} CDR2) comprising SEQ ID NO: 109, and
   a variable heavy chain complementarity determining region 3 (V_{H} CDR3) comprising SEQ ID NO: 110; and
   a light chain variable region comprising a variable light chain complementarity determining region 1 (V_{L} CDR1) comprising SEQ ID NO: 115,
   a variable light chain complementarity determining region 2 (V_{L} CDR2) comprising SEQ ID NO: 116, and
   a variable light chain complementarity determining region 3 (V_{L} CDR3) comprising a SEQ ID NO: 117.

Statement 78. The M9-binding protein of Statement 77, wherein the heavy chain variable region comprises a sequence at least 80%, 85%, 90%, 95%, 99%, or 100% identical to SEQ ID NO: 107, and the light chain variable region comprises a sequence at least 80%, 85%, 90%, 95%, 99%, or 100% identical to SEQ ID NO: 114.

Statement 79. An M9-binding protein comprising:
a heavy chain variable region comprising
   a V_{H} CDR1 comprising SEQ ID NO: 122,
   a V_{H} CDR2 comprising SEQ ID NO: 123, and
   a V_{H} CDR3 comprising SEQ ID NO: 124; and
a light chain variable region comprising
   a V_{L} CDR1 comprising SEQ ID NO: 129,
   a V_{L} CDR2 comprising SEQ ID NO: 130, and
   a V_{L} CDR3 comprising a SEQ ID NO: 131.

Statement 80. The M9-binding protein of Statement 79, wherein the heavy chain variable region comprises a sequence at least 80%, 85%, 90%, 95%, 99%, or 100% identical to SEQ ID NO: 121, and the light chain variable region comprises a sequence at least 80%, 85%, 90%, 95%, 99%, or 100% identical to SEQ ID NO: 128.

Statement 81. An M9-binding protein comprising:
a heavy chain variable region comprising
   a V_{H} CDR1 comprising SEQ ID NO: 136,
   a V_{H} CDR2 comprising SEQ ID NO: 137, and
   a V_{H} CDR3 comprising SEQ ID NO: 138; and
a light chain variable region comprising
   a V_{L} CDR1 comprising SEQ ID NO: 143,
   a V_{L} CDR2 comprising SEQ ID NO: 144, and
   a V_{L} CDR3 comprising a SEQ ID NO: 145.

Statement 82. The M9-binding protein of Statement 81, wherein the heavy chain variable region comprises a sequence at least 80%, 85%, 90%, 95%, 99%, or 100% identical to SEQ ID NO: 135, and the light chain variable region comprises a sequence at least 80%, 85%, 90%, 95%, 99%, or 100% identical to SEQ ID NO: 142.

Statement 83. An M9-binding protein comprising:
a heavy chain variable region comprising
   a V_{H} CDR1 comprising SEQ ID NO: 150,
   a V_{H} CDR2 comprising SEQ ID NO: 151, and
   a V_{H} CDR3 comprising SEQ ID NO: 152; and
a light chain variable region comprising
   a V_{L} CDR1 comprising SEQ ID NO: 157,
   a V_{L} CDR2 comprising SEQ ID NO: 158, and
   a V_{L} CDR3 comprising a SEQ ID NO: 159.

Statement 84. The M9-binding protein of Statement 83, wherein the heavy chain variable region comprises a sequence at least 80%, 85%, 90%, 95%, 99%, or 100% identical to SEQ ID NO: 149, and the light chain variable region comprises a sequence at least 80%, 85%, 90%, 95%, 99%, or 100% identical to SEQ ID NO: 156.

Statement 85. An M9-binding protein comprising:
a heavy chain variable region comprising
   a V_{H} CDR1 comprising SEQ ID NO: 164,
   a V_{H} CDR2 comprising SEQ ID NO: 165, and
   a V_{H} CDR3 comprising SEQ ID NO: 166; and
a light chain variable region comprising
   a V_{L} CDR1 comprising SEQ ID NO: 171,
   a V_{L} CDR2 comprising SEQ ID NO: 172, and
   a V_{L} CDR3 comprising a SEQ ID NO: 173.

Statement 86. The M9-binding protein of Statement 85, wherein the heavy chain variable region comprises a sequence at least 80%, 85%, 90%, 95%, 99%, or 100% identical to SEQ ID NO: 163, and the light chain variable region comprises a sequence at least 80%, 85%, 90%, 95%, 99%, or 100% identical to SEQ ID NO: 170.

Statement 87. An M9-binding protein comprising:
a heavy chain variable region comprising
   a V_{H} CDR1 comprising SEQ ID NO: 178,
   a V_{H} CDR2 comprising SEQ ID NO: 179, and
   a CDR3 comprising SEQ ID NO: 180; and
a light chain variable region comprising
   a V_{L} CDR1 comprising SEQ ID NO: 185,
   a V_{L} CDR2 comprising SEQ ID NO: 186, and
   a V_{L} CDR3 comprising a SEQ ID NO: 187.

Statement 88. The M9-binding protein of Statement 87, wherein the heavy chain variable region comprises a sequence at least 80%, 85%, 90%, 95%, 99%, or 100% identical to SEQ ID NO: 177, and the light chain variable region comprises a sequence at least 80%, 85%, 90%, 95%, 99%, or 100% identical to SEQ ID NO: 184.

Statement 89. An U5A-binding protein comprising:
a heavy chain variable region comprising
   a V_{H} CDR1 comprising SEQ ID NO: 192,
   a V_{H} CDR2 comprising SEQ ID NO: 193, and
   a V_{H} CDR3 comprising SEQ ID NO: 194; and
a light chain variable region comprising
   a V_{L} CDR1 comprising SEQ ID NO: 199,
   a V_{L} CDR2 comprising SEQ ID NO: 200, and
   a V_{L} CDR3 comprising a SEQ ID NO: 201.

Statement 90. The U5A-binding protein of Statement 89, wherein the heavy chain variable region comprises a sequence at least 80%, 85%, 90%, 95%, 99%, or 100% identical to SEQ ID NO: 191, and the light chain variable region comprises a sequence at least 80%, 85%, 90%, 95%, 99%, or 100% identical to SEQ ID NO: 198.

Statement 91. An U5A-binding protein comprising:
a heavy chain variable region comprising
   a V_{H} CDR1 comprising SEQ ID NO: 206,
   a V_{H} CDR2 comprising SEQ ID NO: 207, and
   a V_{H} CDR3 comprising SEQ ID NO: 208; and
a light chain variable region comprising
   a V_{L} CDR1 comprising SEQ ID NO: 213,
   a V_{L} CDR2 comprising SEQ ID NO: 214, and
   a V_{L} CDR3 comprising a SEQ ID NO: 215.

Statement 92. The U5A-binding protein of Statement 91, wherein the heavy chain variable region comprises a sequence at least 80%, 85%, 90%, 95%, 99%, or 100% identical to SEQ ID NO: 205, and the light chain variable region comprises a sequence at least 80%, 85%, 90%, 95%, 99%, or 100% identical to SEQ ID NO: 212.

Statement 93. An U5A-binding protein comprising:
a heavy chain variable region comprising
   a V_{H} CDR1 comprising SEQ ID NO: 220,
   a V_{H} CDR2 comprising SEQ ID NO: 221, and
   a V_{H} CDR3 comprising SEQ ID NO: 222; and
a light chain variable region comprising
   a V_{L} CDR1 comprising SEQ ID NO: 227,
   a V_{L} CDR2 comprising SEQ ID NO: 228, and
   a V_{L} CDR3 comprising a SEQ ID NO: 229.

Statement 94. The U5A-binding protein of Statement 93, wherein the heavy chain variable region comprises a sequence at least 80%, 85%, 90%, 95%, 99%, or 100% identical to SEQ ID NO: 219, and the light chain variable region comprises a sequence at least 80%, 85%, 90%, 95%, 99%, or 100% identical to SEQ ID NO: 226.

Statement 95. An U5A-binding protein comprising:
a heavy chain variable region comprising
   a V_{H} CDR1 comprising SEQ ID NO: 234,
   a V_{H} CDR2 comprising SEQ ID NO: 235, and
   a V_{H} CDR3 comprising SEQ ID NO: 236; and
a light chain variable region comprising
   a V_{L} CDR1 comprising SEQ ID NO: 241,
   a V_{L} CDR2 comprising SEQ ID NO: 242, and
   a V_{L} CDR3 comprising a SEQ ID NO: 243.

Statement 96. The U5A-binding protein of Statement 95, wherein the heavy chain variable region comprises a sequence at least 80%, 85%, 90%, 95%, 99%, or 100% identical to SEQ ID NO: 233, and the light chain variable region comprises a sequence at least 80%, 85%, 90%, 95%, 99%, or 100% identical to SEQ ID NO: 240.

Statement 97. A U5A-binding protein comprising:
a heavy chain variable region comprising
   a V_{H} CDR1 comprising SEQ ID NO: 248,
   a V_{H} CDR2 comprising SEQ ID NO: 249, and
   a V_{H} CDR3 comprising SEQ ID NO: 250; and
a light chain variable region comprising
   a V_{L} CDR1 comprising SEQ ID NO: 255,
   a V_{L} CDR2 comprising SEQ ID NO: 256, and
   a V_{L} CDR3 comprising a SEQ ID NO: 257.

Statement 98. The U5A-binding protein of Statement 97, wherein the heavy chain variable region comprises a sequence at least 80%, 85%, 90%, 95%, 99%, or 100% identical to SEQ ID NO: 247, and the light chain variable region comprises a sequence at least 80%, 85%, 90%, 95%, 99%, or 100% identical to SEQ ID NO: 254.

Statement 99. An ALG5-binding protein comprising:
a heavy chain variable region comprising
   a V_{H} CDR1 comprising SEQ ID NO: 262,
   a V_{H} CDR2 comprising SEQ ID NO: 263, and
   a V_{H} CDR3 comprising SEQ ID NO: 264; and
a light chain variable region comprising
   a V_{L} CDR1 comprising SEQ ID NO: 269,
   a V_{L} CDR2 comprising SEQ ID NO: 270, and
   a V_{L} CDR3 comprising a SEQ ID NO: 271.

Statement 100. The ALG5-binding protein of Statement 99, wherein the heavy chain variable region comprises a sequence at least 80%, 85%, 90%, 95%, 99%, or 100% identical to SEQ ID NO: 261, and the light chain variable region comprises a sequence at least 80%, 85%, 90%, 95%, 99%, or 100% identical to SEQ ID NO: 268.

Statement 101. An ALG5-binding protein comprising:
a heavy chain variable region comprising
   a V_{H} CDR1 comprising SEQ ID NO: 276,
   a V_{H} CDR2 comprising SEQ ID NO: 277, and
   a V_{H} CDR3 comprising SEQ ID NO: 278; and
a light chain variable region comprising
   a V_{L} CDR1 comprising SEQ ID NO: 283,
   a V_{L} CDR2 comprising SEQ ID NO: 284, and
   a V_{L} CDR3 comprising a SEQ ID NO: 285.

Statement 102. The ALG5-binding protein of Statement 101, wherein the heavy chain variable region comprises a sequence at least 80%, 85%, 90%, 95%, 99%, or 100% identical to SEQ ID NO: 275, and the light chain variable region comprises a sequence at least 80%, 85%, 90%, 95%, 99%, or 100% identical to SEQ ID NO: 282.

Statement 103. An ALG5-binding protein comprising:
a heavy chain variable region comprising
   a V_{H} CDR1 comprising SEQ ID NO: 290,
   a V_{H} CDR2 comprising SEQ ID NO: 291, and
   a V_{H} CDR3 comprising SEQ ID NO: 292; and
a light chain variable region comprising
   a V_{L} CDR1 comprising SEQ ID NO: 297,
   a V_{L} CDR2 comprising SEQ ID NO: 298, and
   a V_{L} CDR3 comprising a SEQ ID NO: 299.

Statement 104. The ALG5-binding protein of Statement 103, wherein the heavy chain variable region comprises a sequence at least 80%, 85%, 90%, 95%, 99%, or 100% identical to SEQ ID NO: 289, and the light chain variable region comprises a sequence at least 80%, 85%, 90%, 95%, 99%, or 100% identical to SEQ ID NO: 296.

Statement 105. An ALG5-binding protein comprising:
a heavy chain variable region comprising
   a V_{H} CDR1 comprising SEQ ID NO: 304,
   a V_{H} CDR2 comprising SEQ ID NO: 305, and
   a V_{H} CDR3 comprising SEQ ID NO: 306; and
a light chain variable region comprising
   a V_{L} CDR1 comprising SEQ ID NO: 311,
   a V_{L} CDR2 comprising SEQ ID NO: 312, and
   a V_{L} CDR3 comprising a SEQ ID NO: 313.

Statement 106. The ALG5-binding protein of Statement 105, wherein the heavy chain variable region comprises a sequence at least 80%, 85%, 90%, 95%, 99%, or 100% identical to SEQ ID NO: 303, and the light chain variable region comprises a sequence at least 80%, 85%, 90%, 95%, 99%, or 100% identical to SEQ ID NO: 310.

Statement 107. An ALG5-binding protein comprising:
a heavy chain variable region comprising
   a V_{H} CDR1 comprising SEQ ID NO: 318,
   a V_{H} CDR2 comprising SEQ ID NO: 319, and
   a V_{H} CDR3 comprising SEQ ID NO: 320; and
a light chain variable region comprising
   a V_{L} CDR1 comprising SEQ ID NO: 325,
   a V_{L} CDR2 comprising SEQ ID NO: 326, and
   a V_{L} CDR3 comprising a SEQ ID NO: 327.

Statement 108. The ALG5-binding protein of Statement 107, wherein the heavy chain variable region comprises a sequence at least 80%, 85%, 90%, 95%, 99%, or 100% identical to SEQ ID NO: 317, and the light chain variable region comprises a sequence at least 80%, 85%, 90%, 95%, 99%, or 100% identical to SEQ ID NO: 324.

Statement 109. An ALG5-binding protein comprising:
a heavy chain variable region comprising
   a V_{H} CDR1 comprising SEQ ID NO: 332,
   a V_{H} CDR2 comprising SEQ ID NO: 333, and
   a V_{H} CDR3 comprising SEQ ID NO: 334; and
a light chain variable region comprising
   a V_{L} CDR1 comprising SEQ ID NO: 339,
   a V_{L} CDR2 comprising SEQ ID NO: 340, and
   a V_{L} CDR3 comprising a SEQ ID NO: 341.

Statement 110. The ALG5-binding protein of Statement 109, wherein the heavy chain variable region comprises a sequence at least 80%, 85%, 90%, 95%, 99%, or 100% identical to SEQ ID NO: 331, and the light chain variable region comprises a sequence at least 80%, 85%, 90%, 95%, 99%, or 100% identical to SEQ ID NO: 338.

Statement 111. The method of any one or combination of Statements 1-60, comprising administering a M9-binding protein of any one of Statements 77-88.

Statement 112. The method of any one or combination of Statements 1-60, comprising administering a U5A-binding protein of any one of Statements 89-100.

Statement 113. The method of any one or combination of Statements 1-60, comprising administering an ALG-binding protein of any one of Statements 101-110.

Statement 114. Use of a M9-binding protein of any one of Statements 77-88 for the manufacture of a medicament.

Statement 115. Use of a U5A-binding protein of any one of Statements 89-100 for the manufacture of a medicament.

Statement 116. Use of an ALG5-binding protein of any one of Statements 101-110 for the manufacture of a medicament.

Statement 117. The use of any one of Statements 114-116, wherein the medicament is for treatment of an infection by a coronavirus, an influenza virus, a respiratory syncytial virus (RSV), an enterovirus (Picornaviridae), a rhinovirus, parainfluenza, a metapneumovirus, a bocavirus, an adenovirus, or a Coxsackie virus in a subject.

Statement 118. The method of statement 1, wherein the MEGF9 protein or biologically active fragment of MEGF9 comprises a sequence with at least 80%, at least 85%, at least 90%, or at least 95% sequence identity to a sequence selected from SEQ ID NOs: 1-4 or 102.

Statement 119. The method of statement 1, wherein the biologically active fragment of MEGF9 comprises a sequence with at least 80%, at least 85%, at least 90%, or at least 95% sequence identity to HGHVDPVKTPKI (SEQ ID NO: 350).

Statement 120. The method of statement 118, wherein the MEGF9 protein or biologically active fragment of MEGF9 comprises a sequence according to SEQ ID NOs: 1-4 or 102.

Statement 121. The method of statement 119, wherein the biologically active fragment of MEGF9 is a sequence consisting of HGHVDPVKTPKI (SEQ ID NO: 350).

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

TABLE 2
Exemplary sequences

| SEQ | | |
|---|---|---|
| ID | | |
| NO | Notes | Sequences |
| 1 | Human | MNGGAERAMR SLPSLGGLAL LCCAAAAAAA AVASAASAGN |
| | MEGF9 | VTGGGGAAGQ VDASPGPGLR GEPSHPFPRA TAPTAQAPRT |
| | full-length | GPPRATVHRP LAATSPAQSP ETTPLWATAG PSSTTFQAPL |
| | with | GPSPTTPPAA ERTSTTSQAP TRPAPTTLST TTGPAPTTPV |
| | signal | ATTVPAPTTP RTPTPDLPSS SNSSVLPTPP ATEAPSSPPP |
| | peptide | EYVCNCSVVG SLNVNRCNQT TGQCECRPGY QGLHCETCKE |
| | (amino | GFYLNYTSGL CQPCDCSPHG ALSIPCNSSG KCQCKVGVIG |
| | acid 1-30) | SICDRCQDGY YGFSKNGCLP CQCNNRSASC DALTGACLNC |
| | | QENSKGNHCE ECKEGFYQSP DATKECLRCP CSAVTSTGSC |
| | | SIKSSELEPE CDQCKDGYIG PNCNKCENGY YNFDSICRKC |
| | | QCHGHVDPVK TPKICKPESG ECINCLHNTT GFWCENCLEG |
| | | YVHDLEGNCI KKEVILPTPE GSTILVSNAS LTTSVPTPVI |
| | | NSTFTPTTLQ TIFSVSTSEN STSALADVSW TQFNIIILTV |
| | | IIIVVVLLMG FVGAVYMYRE YQNRKLNAPF WTIELKEDNI |
| | | SFSSYHDSIP NADVSGLLED DGNEVAPNGQ LTLTTPIHNY |
| | | KA |
| 2 | Mature | AVASAASAGN VTGGGGAAGQ VDASPGPGLR GEPSHPFPRA |
| | human | TAPTAQAPRT GPPRATVHRP LAATSPAQSP ETTPLWATAG |
| | MEGF9 | PSSTTFQAPL GPSPTTPPAA ERTSTTSQAP TRPAPTTLST |
| | (amino | TTGPAPTTPV ATTVPAPTTP RTPTPDLPSS SNSSVLPTPP |
| | acids 31- | ATEAPSSPPP EYVCNCSVVG SLNVNRCNQT TGQCECRPGY |
| | 602 of | QGLHCETCKE GFYLNYTSGL CQPCDCSPHG ALSIPCNSSG |
| | SEQ ID | KCQCKVGVIG SICDRCQDGY YGFSKNGCLP CQCNNRSASC |
| | NO: 1) | DALTGACLNC QENSKGNHCE ECKEGFYQSP DATKECLRCP |
| | | CSAVTSTGSC SIKSSELEPE CDQCKDGYIG PNCNKCENGY |
| | | YNFDSICRKC QCHGHVDPVK TPKICKPESG ECINCLHNTT |
| | | GFWCENCLEG YVHDLEGNCI KKEVILPTPE GSTILVSNAS |
| | | LTTSVPTPVI NSTFTPTTLQ TIFSVSTSEN STSALADVSW |
| | | TQFNIIILTV IIIVVVLLMG FVGAVYMYRE YQNRKLNAPF |
| | | WTIELKEDNI SFSSYHDSIP NADVSGLLED DGNEVAPNGQ |
| | | LTLTTPIHNY KA |
| 3 | ECD of | AVASAASAGN VTGGGGAAGQ VDASPGPGLR GEPSHPFPRA |
| | MEGF9 | TAPTAQAPRT GPPRATVHRP LAATSPAQSP ETTPLWATAG |
| | (amino | PSSTTFQAPL GPSPTTPPAA ERTSTTSQAP TRPAPTTLST |
| | acids 31- | TTGPAPTTPV ATTVPAPTTP RTPTPDLPSS SNSSVLPTPP |
| | 514 of | ATEAPSSPPP EYVCNCSVVG SLNVNRCNQT TGQCECRPGY |
| | SEQ ID | QGLHCETCKE GFYLNYTSGL CQPCDCSPHG ALSIPCNSSG |
| | NO: 1) | KCQCKVGVIG SICDRCQDGY YGFSKNGCLP CQCNNRSASC |
| | | DALTGACLNC QENSKGNHCE ECKEGFYQSP DATKECLRCP |
| | | CSAVTSTGSC SIKSSELEPE CDQCKDGYIG PNCNKCENGY |
| | | YNFDSICRKC QCHGHVDPVK TPKICKPESG ECINCLHNTT |
| | | GFWCENCLEG YVHDLEGNCI KKEVILPTPE GSTILVSNAS |
| | | LTTSVPTPVI NSTFTPTTLQ TIFSVSTSEN STSALADVSW |
| | | TQFN |
| 4 | A peptide | HGHVDPVKTP K |
| | in the | |
| | ECD of | |
| | MEGF9 | |
| | (amino | |
| | acids 403- | |
| | 413 of | |
| | SEQ ID | |
| | NO: 1) | |
| 350 | A peptide | HGHVDPVKTP KI |
| | in the | |
| | ECD of | |
| | MEGF9 | |
| | (amino | |
| | acids 403- | |
| | 414 of | |
| | SEQ ID | |
| | NO: 1) | |
| 5 | Human | MAVRPGLWPA LLGIVLAAWL RGSGAQQSAT VANPVPGANP |
| | UNC5A | DLLPHFLVEP EDVYIVKNKP VLLVCKAVPA TQIFFKCNGE |
| | full-length | WVRQVDHVIE RSTDGSSGLP TMEVRINVSR QQVEKVFGLE |
| | with | EYWCQCVAWS SSGTTKSQKA YIRIAYLRKN FEQEPLAKEV |
| | signal | SLEQGIVLPC RPPEGIPPAE VEWLRNEDLV DPSLDPNVYI |
| | peptide | TREHSLVVRQ ARLADTANYT CVAKNIVARR RSASAAVIVY |
| | (amino | VDGSWSPWSK WSACGLDCTH WRSRECSDPA PRNGGEECQG |
| | acid 1-30) | TDLDTRNCTS DLCVHTASGP EDVALYVGLI AVAVCLVLLL |
| | | LVLILVYCRK KEGLDSDVAD SSILTSGFQP VSIKPSKADN |
| | | PHLLTIQPDL STTTTTYQGS LCPRQDGPSP KFQLTNGHLL |
| | | SPLGGGRHTL HHSSPTSEAE EFVSRLSTQN YFRSLPRGTS |
| | | NMTYGTFNFL GGRLMIPNTG ISLLIPPDAI PRGKIYEIYL |
| | | TLHKPEDVRL PLAGCQTLLS PIVSCGPPGV LLTRPVILAM |
| | | DHCGEPSPDS WSLRLKKQSC EGSWEDVLHL GEEAPSHLYY |
| | | CQLEASACYV FTEQLGRFAL VGEALSVAAA KRLKLLLFAP |
| | | VACTSLEYNI RVYCLHDTHD ALKEVVQLEK QLGGQLIQEP |
| | | RVLHFKDSYH NLRLSIHDVP SSLWKSKLLV SYQEIPFYHI |
| | | WNGTQRYLHC TFTLERVSPS TSDLACKLWV WQVEGDGQSF |
| | | SINFNITKDT RFAELLALES EAGVPALVGP SAFKIPFLIR |
| | | QKIISSLDPP CRRGADWRTL AQKLHLDSHL SFFASKPSPT |
| | | AMILNLWEAR HFPNGNLSQL AAAVAGLGQP DAGLFTVSEA |
| | | EC |
| 6 | Mature | QQSAT VANPVPGANP DLLPHFLVEP EDVYIVKNKP |
| | human | VLLVCKAVPA TQIFFKCNGE WVRQVDHVIE RSTDGSSGLP |
| | UNC5A | TMEVRINVSR QQVEKVFGLE EYWCQCVAWS SSGTTKSQKA |
| | (amino | YIRIAYLRKN FEQEPLAKEV SLEQGIVLPC RPPEGIPPAE |
| | acids 26- | VEWLRNEDLV DPSLDPNVYI TREHSLVVRQ ARLADTANYT |
| | 842 of | CVAKNIVARR RSASAAVIVY VDGSWSPWSK WSACGLDCTH |
| | SEQ ID | WRSRECSDPA PRNGGEECQG TDLDTRNCTS DLCVHTASGP |
| | NO: 5) | EDVALYVGLI AVAVCLVLLL LVLILVYCRK KEGLDSDVAD |
| | | SSILTSGFQP VSIKPSKADN PHLLTIQPDL STTTTTYQGS |
| | | LCPRQDGPSP KFQLTNGHLL SPLGGGRHTL HHSSPTSEAE |
| | | EFVSRLSTQN YFRSLPRGTS NMTYGTFNFL GGRLMIPNTG |
| | | ISLLIPPDAI PRGKIYEIYL TLHKPEDVRL PLAGCQTLLS |
| | | PIVSCGPPGV LLTRPVILAM DHCGEPSPDS WSLRLKKQSC |
| | | EGSWEDVLHL GEEAPSHLYY CQLEASACYV FTEQLGRFAL |
| | | VGEALSVAAA KRLKLLLFAP VACTSLEYNI RVYCLHDTHD |
| | | ALKEVVQLEK QLGGQLIQEP RVLHFKDSYH NLRLSIHDVP |
| | | SSLWKSKLLV SYQEIPFYHI WNGTQRYLHC TFTLERVSPS |
| | | TSDLACKLWV WQVEGDGQSF SINFNITKDT RFAELLALES |
| | | EAGVPALVGP SAFKIPFLIR QKIISSLDPP CRRGADWRTL |
| | | AQKLHLDSHL SFFASKPSPT AMILNLWEAR HFPNGNLSQL |
| | | AAAVAGLGQP DAGLFTVSEA EC |
| 7 | An | IVARRRSASA AVIVYVDGSW SPWSKWSACG LDCTHWRSRE |
| | exemplary | CSDPAPRNGG EECQGTDLDT RNCTSDLCVH TASGPEDVAL |
| | biologically | YVGLIAVAVC LVLLLLVLIL VYCRKKEGLD SDVADSSILT |
| | active | SGFQPVSIKP SKADNPHLLT IQPDLSTTTT TYQGSLCPRQ |
| | fragment | DGPSPKFQLT NGHLLSPLGG GRHTLHHSSP TSEAEEFVSR |
| | of | LSTQNYFRSL PRGTSNMTYG TFNFLGGRLM IPNTGISLLI |
| | UNC5A | PPDAIPRGKI YEIYLTLHKP EDVRLPLAGC QTLLSPIVS |
| | (amino | |
| | acids 226- | |
| | 504 of | |
| | SEQ ID | |
| | NO: 5) | |
| 8 | An | VYCRKKEGLD SDVADSSILT SGFQPVSIKP SKADNPHLLT |
| | exemplary | IQPDLSTTTT |
| | biologically | |
| | active | |
| | fragment | |
| | of | |
| | UNC5A | |
| | (amino | |
| | acids 326- | |
| | 375 of | |
| | SEQ ID | |
| | NO: 5) | |
| 9 | Human | MAPLLLQLAV LGAALAAAAL VLISIVAFTT ATKMPALHRH |
| | ALG5 | EEEKFFLNAK GQKETLPSIW DSPTKQLSVV VPSYNEEKRL |
| | full-length | PVMMDEALSY LEKRQKRDPA FTYEVIVVDD GSKDQTSKVA |
| | | FKYCQKYGSD KVRVITLVKN RGKGGAIRMG IFSSRGEKIL |
| | | MADADGATKF PDVEKLEKGL NDLQPWPNQM AIACGSRAHL |
| | | EKESIAQRSY FRTLLMYGFH FLVWFLCVKG IRDTQCGFKL |
| | | FTREAASRTF SSLHVERWAF DVELLYIAQF FKIPIAEIAV |
| | | NWTEIEGSKL VPFWSWLQMG KDLLFIRLRY LTGAWRLEQT |
| | | RKMN |
| 10 | CSP | DGIWKASFTTFTVTKYWFYR |
| 11 | CSP7 | FTTFTVT |
| 102 | ECD of | MGWSCIILFLVATATGVHSAAVASAASAGNVTGGGGAAGQVDAS |
| | hMEGF9 | PGPGLRGEPSHPFPRATAPTAQAPRTGPPRATVHRPLAATSPAQS |
| | (555 | PETTPLWATAGPSSTTFQAPLGPSPTTPPAAERTSTTSQAPTRPA |
| | amino | PTTLSTTTGPAPTTPVATTVPAPTTPRTPTPDLPSSSNSSVLPTP |
| | acid | PATEAPSSPPPGHQWPVAKMPQKYLGKYACESNLKSKYLPLTQPV |
| | sequence) | MNLRVSEAVKTEYVCNCSVVGSLNVNRCNQTTGQCECRPGYQGLH |
| | | CETCKEGFYLNYTSGLCQPCDCSPHGALSIPCNSSGKCQCKVGVI |
| | | GSICDRCQDGYYGFSKNGCLPCQCNNRSASCDALTGACLNCQENS |
| | | KGNHCEECKEGFYQSPDATKECLRCPCSAVTSTGSCSIKSSELEP |
| | | ECDQCKDGYIGPNCNKCENGYYNFDSICRKCQCHGHVDPVKTPKI |
| | | CKPESGECINCLHNTTGFWCENCLEGYVHDLEGNCIKKEVILPTP |
| | | EGSTILVSNASLTTSVPTPVINSTFTPTTLQTIFSVSTSENSTSA |
| | | LADVSWTQFN |
| 103 | ECD of | MAVRPGLWPALLGIVLAAWLRGSGAQQSATVANPVPGANPDLLP |
| | hUNC5a | HFLVEPEDVYIVKNKPVLLVCKAVPATQIFFKCNGEWVRQVDHVI |
| | (312 | ERSTDGSSGLPTMEVRINVSRQQVEKVFGLEEYWCQCVAWSSSGT |
| | amino | TKSQKAYIRIAYLRKNFEQEPLAKEVSLEQGIVLPCRPPEGIPPA |
| | acid | EVEWLRNEDLVDPSLDPNVYITREHSLVVRQAALADTANYTCVAK |
| | sequence) | NIVARRRSASAAVIVYVDGSWSPWSKWSACGLDCTHWRSRECSDP |
| | | APRNGGEECQGTDLDTRNCTSDLCVHTASGPEDVALY |
| 104 | ECD of | MTTATKMPALHRHEEEKFFLNAKGQKETLPSIWDSPTKQLSVVVP |
| | hALG5 | SYNEEKRLPVMMDEALSYLEKRQKADPAFTYEVIVVDDGSKDQTS |
| | (303 | KVAFKYCQKYGSDKVRVITLVKNRGKGGAIRMGIFSSRGEKILM |
| | amino | ADADGATKFPDVEKLEKGLNDLQPWPNQMAIACGSRAHLEKESI |
| | acid | AQRSYFRTLLMYGFHFLVWFLCVKGIRDTQCGFKLFTREAASRT |
| | sequence) | FSSLHVERWAFDVELLYIAQFFKIPIAEIAVNWTEIEGSKLVPF |
| | | WSWLQMGKDLLFIRLRYLTGAWRLEQTRKMN |
| 105 | M9 mAb | MNFGLSLIFLVLILKGVQSEVKLVESGGGLVKPGGSLKLSCAASG |
| | 8C2-1 | FIFSRYGMSWVRQTPEKRLEWVATISSGGGYTSYPDSVKGRFTMS |
| | heavy | RDNANNNLFLQMSSLRSEDTALYYCARQGGTYYRHDDAPMDY |
| | chain | WGQGTSVTVSSAKTTPPSVYPLAPGSAAQTNSMVTLGCLVKGYF |
| | | PEPVTVTWNSGSLSSGVHTFPAVLQSDLYTLSSSVTVPSSTWPSET |
| | | VTCNVAHPASSTKVDKKIVPRDCGCKPCICTVPEVSSVFIFPPKPK |
| | | DVLTITLTPKVTCVVVDISKDDPEVQFSWFVDDVEVHTAQTQPRE |
| | | EQFNSTFRSVSELPIMHQDWLNGKEFKCRVNSAAFPAPIEKTISKT |
| | | KGRPKAPQVYTIPPPKEQMAKDKVSLTCMITDFFPEDITVEWQWN |
| | | GQPAENYKNTQPIMDTDGSYFVYSKLNVQKSNWEAGNTFTCSVL |
| | | HEGLHNHHTEKSLSHSPGK |
| | | Note: the sequence include signal peptide (italicized)- |
| | | FR1 (underlined)-CDR1 (bolded)-FR2 (underlined)- |
| | | CDR2 (bolded)-FR3 (underlined)- |
| | | CDR3 (bolded)-FR4 (underlined)-constant region |
| 106 | M9 mAb | EVKLVESGGGLVKPGGSLKLSCAASGFIFSRYGMSWVRQTPEKR |
| | 8C2-1 | EVKLVESGGGLVKPGGSLKLSCAASGFIFSRYGMSWVRQTPEKR |
| | heavy | LEWVATISSGGGYTSYPDSVKGRFTMSRDNANNNLFLQMSSLRS |
| | chain | EDTALYYCARQGGTYYRHDDAPMDYWGQGTSVTVSSAKTTPPS |
| | protein | VYPLAPGSAAQTNSMVTLGCLVKGYFPEPVTVTWNSGSLSSGVH |
| | w/o signal | TFPAVLQSDLYTLSSSVTVPSSTWPSETVTCNVAHPASSTKVDKKI |
| | peptide | VPRDCGCKPCICTVPEVSSVFIFPPKPKDVLTITLTPKVTCVVVDIS |
| | | KDDPEVQFSWFVDDVEVHTAQTQPREEQFNSTFRSVSELPIMHQD |
| | | WLNGKEFKCRVNSAAFPAPIEKTISKTKGRPKAPQVYTIPPPKEQM |
| | | AKDKVSLTCMITDFFPEDITVEWQWNGQPAENYKNTQPIMDTDG |
| | | SYFVYSKLNVQKSNWEAGNTFTCSVLHEGLHNHHTEKSLSHSPG |
| | | K |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 107 | M9 mAb | EVKLVESGGGLVKPGGSLKLSCAASGFIFSRYGMSWVRQTPEKR |
| | 8C2-1 | LEWVATISSGGGYTSYPDSVKGRFTMSRDNANNNLFLQMSSLRS |
| | heavy | EDTALYYCARQGGTYYRHDDAPMDYWGQGTSVTVSS |
| | chain | Note: |
| | variable | the sequence include FR1 (underlined)-CDR1 (bolded)-FR2 |
| | region | (underlined)-CDR2 (bolded)-FR3 (underlined)-CDR3 (bolded)-FR4 |
| | | (underlined) |
| 108 | M9 mAb | RYGMS |
| | 8C2-1 | |
| | heavy | |
| | chain | |
| | CDR1 | |
| 109 | M9 mAb | TISSGGGYTSYPDSVKG |
| | 8C2-1 | |
| | heavy | |
| | chain | |
| | CDR2 | |
| 110 | M9 mAb | QGGTYYRHDDAPMDY |
| | 8C2-1 | |
| | heavy | |
| | chain | |
| | CDR3 | |
| 111 | M9 mAb | ATGAACTTCGGACTCAGCTTGATTTTCCTTGTCCTAATTTTAAAA |
| | 8C2-1 | GGTGTCCAGTCTGAAGTAAAGCTGGTGGAGTCTGGGGGAGGCTTA |
| | heavy | GTGAAGCCTGGAGGGTCCCTGAAACTCTCCTGTGCAGCCTCTGG |
| | chain | ATTCATTTTCAGTAGGTATGGCATGTCTTGGGTTCGCCAGACT |
| | nucleic | CCGGAGAAGAGGCTGGAGTGGGTCGCAACCATTAGTAGTGGT |
| | acid | GGTGGTTACACCTCCTATCCAGACAGTGTGAAGGGGCGATT |
| | | CACCATGTCCAGAGACAATGCCAACAACAACCTGTTCCTGCAA |
| | | ATGAGCAGTCTGAGGTCTGAGGACACGGCCTTATATTACTGTG |
| | | CAAGACAGGGGGGGACCTACTATAGGCACGACGACGCTCC |
| | | TATGGACTACTGGGGTCAAGGAACCTCAGTCACCGTCTCCTCA |
| | | GCCAAAACGACACCCCCATCTGTCTATCCACTGGCCCCTGGAT |
| | | CTGCTGCCCAAACTAACTCCATGGTGACCCTGGGATGCCTGGT |
| | | CAAGGGCTATTTCCCTGAGCCAGTGACAGTGACCTGGAACTCT |
| | | GGATCCCTGTCCAGCGGTGTGCACACCTTCCCAGCTGTCCTGCA |
| | | GTCTGACCTCTACACTCTGAGCAGCTCAGTGACTGTCCCCTCCA |
| | | GCACCTGGCCCAGCGAGACCGTCACCTGCAACGTTGCCCACCC |
| | | GGCCAGCAGCACCAAGGTGGACAAGAAAATTGTGCCCAGGGA |
| | | TTGTGGTTGTAAGCCTTGCATATGTACAGTCCCAGAAGTATCAT |
| | | CTGTCTTCATCTTCCCCCCAAAGCCCAAGGATGTGCTCACCATT |
| | | ACTCTGACTCCTAAGGTCACGTGTGTTGTGGTAGACATCAGCA |
| | | AGGATGATCCCGAGGTCCAGTTCAGCTGGTTTGTAGATGATGT |
| | | GGAGGTGCACACAGCTCAGACGCAACCCCGGGAGGAGCAGTT |
| | | CAACAGCACTTTCCGCTCAGTCAGTGAACTTCCCATCATGCACC |
| | | AGGACTGGCTCAATGGCAAGGAGTTCAAATGCAGGGTCAACA |
| | | GTGCAGCTTTCCCTGCCCCCATCGAGAAAACCATCTCCAAAAC |
| | | CAAAGGCAGACCGAAGGCTCCACAGGTGTACACCATTCCACCT |
| | | CCCAAGGAGCAGATGGCCAAGGATAAAGTCAGTCTGACCTGC |
| | | ATGATAACAGACTTCTTCCCTGAAGACATTACTGTGGAGTGGC |
| | | AGTGGAATGGGCAGCCAGCGGAGAACTACAAGAACACTCAGC |
| | | CCATCATGGACACAGATGGCTCTTACTTCGTCTACAGCAAGCT |
| | | CAATGTGCAGAAGAGCAACTGGGAGGCAGGAAATACTTTCAC |
| | | CTGCTCTGTGTTACATGAGGGCCTGCACAACCACCATACTGAG |
| | | AAGAGCCTCTCCCACTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 112 | M9 mAb | MESDTLLLWVLLLWVPGSTGDIILSQSPASVAVSLGQRATISCRAS |
| | 8C2-1 | ESVEYYGRSLMQWYQQKPGQPPKLLIYVASNVESGVPARFSGSGS |
| | light chain | GTDFSLNIHPVEEDDVAMYFCQQSRKVPWTFGGGTKLEIKRADAA |
| | | PTVSIFPPSSEQLTSGGASVVCFLNNFYPKDINVKWKIDGSERQN |
| | | GVLNSWTDQDSKDSTYSMSSTLTLTKDEYERHNSYTCEATHKTS |
| | | TSPIVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 113 | M9 mAb | DIILSQSPASVAVSLGQRATISCRASESVEYYGRSLMQWYQQKPG |
| | 8C2-1 | QPPKLLIYVASNVESGVPARFSGSGSGTDFSLNIHPVEEDDVAMYF |
| | light chain | CQQSRKVPWTFGGGTKLEIKRADAAPTVSIFPPSSEQLTSGGASV |
| | w/o signal | VCFLNNFYPKDINVKWKIDGSERQNGVLNSWTDQDSKDSTYSMS |
| | peptide | STLTLTKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 114 | M9 mAb | DIILSQSPASVAVSLGQRATISCRASESVEYYGRSLMQWYQQKPG |
| | 8C2-1 | QPPKLLIYVASNVESGVPARFSGSGSGTDFSLNIHPVEEDDVAMYF |
| | light chain | CQQSRKVPWTFGGGTKLEIK |
| | variable | |
| | region | |
| 115 | M9 mAb | RASESVEYYGRSLMQ |
| | 8C2-1 | |
| | light chain | |
| | CDR1 | |
| 116 | M9 mAb | VASNVES |
| | 8C2-1 | |
| | light chain | |
| | CDR2 | |
| 117 | M9 mAb | QQSRKVPWT |
| | 8C2-1 | |
| | light chain | |
| | CDR3 | |
| 118 | M9 mAb | ATGGAGTCAGACACACTCCTGCTATGGGTGCTGCTGCTCTGGGTT |
| | 8C2-1 | CCAGGCTCCACTGGTGACATTATACTCTCCCAATCTCCAGCTTC |
| | light chain | TGTGGCTGTGTCTCTGGGGCAGAGAGCCACCATCTCCTGCAGA |
| | nucleic | GCCAGTGAAAGTGTTGAATATTATGGCAGAAGTTTAATGC |
| | acid | AGTGGTACCAACAGAAACCAGGACAGCCACCCAAACTCCTCA |
| | | TCTATGTTGCATCCAACGTAGAATCTGGGGTCCCTGCCAGGT |
| | | TTAGTGGCAGTGGGTCTGGGACAGACTTCAGCCTCAACATCCA |
| | | TCCTGTGGAGGAGGATGATGTTGCAATGTATTTCTGTCAGCAA |
| | | AGTAGGAAGGTTCCTTGGACGTTCGGTGGAGGCACCAAGCT |
| | | GGAAATCAAACGGGCTGATGCTGCACCAACTGTATCCATCTTC |
| | | CCACCATCCAGTGAGCAGTTAACATCTGGAGGTGCCTCAGTCG |
| | | TGTGCTTCTTGAACAACTTCTACCCCAAAGACATCAATGTCAA |
| | | GTGGAAGATTGATGGCAGTGAACGACAAAATGGCGTCCTGAA |
| | | CAGTTGGACTGATCAGGACAGCAAAGACAGCACCTACAGCAT |
| | | GAGCAGCACCCTCACGTTGACCAAGGACGAGTATGAACGACA |
| | | TAACAGCTATACCTGTGAGGCCACTCACAAGACATCAACTTCA |
| | | CCCATTGTCAAGAGCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 119 | M9 mAb | MEWTWVFLFLLSVTAGVHSQFQLQQSGGELMKPGASVKISCKATG |
| | 4E1-1 | YTFSNYWIEWMKQRPGHGLEWIGEILPGSGITKYNEKFKGKAT |
| | heavy | FTADPSSNTAYMQLSSLTSEDSAVYYCSRGGWDGYWGQGTTLV |
| | chain | ASAKTTPPSVYPLAPGSAAQTNSMVTLGCLVKGYFPEPVTVTWNS |
| | | GSLSSGVHTFPAVLQSDLYTLSSSVTVPSSTWPSETVTCNVAHPAS |
| | | STKVDKKIVPRDCGCKPCICTVPEVSSVFIFPPKPKDVLTITLTPK |
| | | VTCVVVDISKDDPEVQFSWFVDDVEVHTAQTQPREEQFNSTFRSVS |
| | | ELPIMHQDWLNGKEFKCRVNSAAFPAPIEKTISKTKGRPKAPQVY |
| | | TIPPPKEQMAKDKVSLTCMITDFFPEDITVEWQWNGQPAENYKNT |
| | | QPIMDTDGSYFVYSKLNVQKSNWEAGNTFTCSVLHEGLHNHHTE |
| | | KSLSHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 120 | M9 mAb | QFQLQQSGGELMKPGASVKISCKATGYTFSNYWIEWMKQRPGH |
| | 4E1-1 | GLEWIGEILPGSGITKYNEKFKGKATFTADPSSNTAYMQLSSLTS |
| | heavy | EDSAVYYCSRGGWDGYWGQGTTLVASAKTTPPSVYPLAPGSAA |
| | chain | QTNSMVTLGCLVKGYFPEPVTVTWNSGSLSSGVHTFPAVLQSDL |
| | protein | YTLSSSVTVPSSTWPSETVTCNVAHPASSTKVDKKIVPRDCGCKPC |
| | w/o signal | ICTVPEVSSVFIFPPKPKDVLTITLTPKVTCVVVDISKDDPEVQFSW |
| | peptide | FVDDVEVHTAQTQPREEQFNSTFRSVSELPIMHQDWLNGKEFKCR |
| | | VNSAAFPAPIEKTISKTKGRPKAPQVYTIPPPKEQMAKDKVSLTCM |
| | | ITDFFPEDITVEWQWNGQPAENYKNTQPIMDTDGSYFVYSKLNVQ |
| | | KSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 121 | M9 mAb | QFQLQQSGGELMKPGASVKISCKATGYTFSNYWIEWMKQRPGH |
| | 4E1-1 | GLEWIGEILPGSGITKYNEKFKGKATFTADPSSNTAYMQLSSLTS |
| | heavy | EDSAVYYCSRGGWDGYWGQGTTLVAS |
| | chain | |
| | variable | |
| | region | |
| 122 | M9 mAb | NYWIE |
| | 4E1-1 | |
| | heavy | |
| | chain | |
| | CDR1 | |
| 123 | M9 mAb | EILPGSGITKYNEKFKG |
| | 4E1-1 | |
| | heavy | |
| | chain | |
| | CDR2 | |
| 124 | M9 mAb | GGWDGY |
| | 4E1-1 | |
| | heavy | |
| | chain | |
| | CDR3 | |
| 125 | M9 mAb | ATGGAATGGACCTGGGTCTTTCTCTTCCTCCTGTCAGTAACTGCA |
| | 4E1-1 | GGTGTCCACTCCCAGTTTCAGCTGCAGCAGTCTGGAGGTGAGCT |
| | heavy | GATGAAGCCTGGGGCCTCAGTGAAGATATCCTGCAAGGCTAC |
| | chain | TGGCTACACATTCAGTAACTACTGGATAGAGTGGATGAAGCA |
| | nucleic | GAGGCCTGGACATGGCCTTGAGTGGATTGGAGAGATTTTACC |
| | acid | TGGAAGTGGTATTACTAAATACAATGAGAAGTTCAAGGGC |
| | | AAGGCCACATTCACTGCAGATCCATCCTCCAACACAGCCTACA |
| | | TGCAACTCAGTAGCCTGACATCTGAGGACTCTGCCGTCTATTA |
| | | CTGTTCAAGAGGGGGCTGGGACGGCTACTGGGGCCAAGGCA |
| | | CCACTCTCACAGTCGCCTCAGCCAAAACGACACCCCCATCTGT |
| | | CTATCCACTGGCCCCTGGATCTGCTGCCCAAACTAACTCCATG |
| | | GTGACCCTGGGATGCCTGGTCAAGGGCTATTTCCCTGAGCCAG |
| | | TGACAGTGACCTGGAACTCTGGATCCCTGTCCAGCGGTGTGCA |
| | | CACCTTCCCAGCTGTCCTGCAGTCTGACCTCTACACTCTGAGC |
| | | AGCTCAGTGACTGTCCCCTCCAGCACCTGGCCCAGCGAGACCG |
| | | TCACCTGCAACGTTGCCCACCCGGCCAGCAGCACCAAGGTGG |
| | | ACAAGAAAATTGTGCCCAGGGATTGTGGTTGTAAGCCTTGCAT |
| | | ATGTACAGTCCCAGAAGTATCATCTGTCTTCATCTTCCCCCCA |
| | | AAGCCCAAGGATGTGCTCACCATTACTCTGACTCCTAAGGTCA |
| | | CGTGTGTTGTGGTAGACATCAGCAAGGATGATCCCGAGGTCCA |
| | | GTTCAGCTGGTTTGTAGATGATGTGGAGGTGCACACAGCTCAG |
| | | ACGCAACCCCGGGAGGAGCAGTTCAACAGCACTTTCCGCTCA |
| | | GTCAGTGAACTTCCCATCATGCACCAGGACTGGCTCAATGGCA |
| | | AGGAGTTCAAATGCAGGGTCAACAGTGCAGCTTTCCCTGCCCC |
| | | CATCGAGAAAACCATCTCCAAAACCAAAGGCAGACCGAAGGC |
| | | TCCACAGGTGTACACCATTCCACCTCCCAAGGAGCAGATGGCC |
| | | AAGGATAAAGTCAGTCTGACCTGCATGATAACAGACTTCTTCC |
| | | CTGAAGACATTACTGTGGAGTGGCAGTGGAATGGGCAGCCAG |
| | | CGGAGAACTACAAGAACACTCAGCCCATCATGGACACAGATG |
| | | GCTCTTACTTCGTCTACAGCAAGCTCAATGTGCAGAAGAGCAA |
| | | CTGGGAGGCAGGAAATACTTTCACCTGCTCTGTGTTACATGAG |
| | | GGCCTGCACAACCACCATACTGAGAAGAGCCTCTCCCACTCTC |
| | | CTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 126 | M9 mAb | MVLAQFLAFLLLWFPGVRCDILMTQSPSSMSVSLGDTVSITCHANQ |
| | 4E1-1 | DISSNIGWLQQKPGKSFQGLIYHGTNLEDGVSSRFSGSGSGTDYS |
| | light chain | LTISSLESEDFADYYCVQYAQFPYTFGGGTKLEIKRADAAPTVSIF |
| | | PPSSEQLTSGGASVVCFLNNFYPKDINVKWKIDGSERQNGVLNSW |
| | | TDQDSKDSTYSMSSTLTLTKDEYERHNSYTCEATHKTSTSPIVKSF |
| | | NRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 127 | M9 mAb | DILMTQSPSSMSVSLGDTVSITCHANQDISSNIGWLQQKPGKSFQ |
| | 4E1-1 | GLIYHGTNLEDGVSSRFSGSGSGTDYSLTISSLESEDFADYYCVQY |
| | light chain | AQFPYTFGGGTKLEIKRADAAPTVSIFPPSSEQLTSGGASVVCFLN |
| | w/o signal | NFYPKDINVKWKIDGSERQNGVLNSWTDQDSKDSTYSMSSTLTL |
| | peptide | TKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 128 | M9 mAb | DILMTQSPSSMSVSLGDTVSITCHANQDISSNIGWLQQKPGKSFQ |
| | 4E1-1 | GLIYHGTNLEDGVSSRFSGSGSGTDYSLTISSLESEDFADYYCVQY |
| | light chain | AQFPYTFGGGTKLEIK |
| | variable | |
| | region | |
| 129 | M9 mAb | HANQDISSNIG |
| | 4E1-1 | |
| | light chain | |
| | CDR1 | |
| 130 | M9 mAb | HGTNLED |
| | 4E1-1 | |
| | light chain | |
| | CDR2 | |
| 131 | M9 mAb | VQYAQFPYT |
| | 4E1-1 | |
| | light chain | |
| | CDR3 | |
| 132 | M9 mAb | ATGGTCCTTGCTCAGTTTCTTGCATTCTTGTTGCTTTGGTTTCCAGG |
| | 4E1-1 | TGTAAGATGTGACATCCTGATGACCCAATCTCCTTCCTCCATGT |
| | light chain | CTGTATCTCTGGGAGACACAGTCAGCATCACTTGCCATGCAA |
| | nucleic | ATCAGGACATTAGTAGTAATATAGGGTGGTTGCAGCAGAAA |
| | acid | CCAGGGAAATCATTTCAGGGCCTGATCTATCATGGAACCAAC |
| | | TTGGAAGATGGAGTTTCATCAAGGTTCAGTGGCAGTGGATCT |
| | | GGAACAGATTATTCTCTCACCATCAGCAGTCTGGAATCTGAAG |
| | | ATTTTGCAGACTATTATTGTGTACAGTATGCTCAGTTTCCGT |
| | | ACACGTTCGGAGGGGGGACCAAGTTGGAAATAAAACGGGCTG |
| | | ATGCTGCACCAACTGTATCCATCTTCCCACCATCCAGTGAGCA |
| | | GTTAACATCTGGAGGTGCCTCAGTCGTGTGCTTCTTGAACAAC |
| | | TTCTACCCCAAAGACATCAATGTCAAGTGGAAGATTGATGGCA |
| | | GTGAACGACAAAATGGCGTCCTGAACAGTTGGACTGATCAGG |
| | | ACAGCAAAGACAGCACCTACAGCATGAGCAGCACCCTCACGT |
| | | TGACCAAGGACGAGTATGAACGACATAACAGCTATACCTGTG |
| | | AGGCCACTCACAAGACATCAACTTCACCCATTGTCAAGAGCTT |
| | | CAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 133 | M9 mAb | MDWLWNLLFLMAAAQSAQAQIQLVQSGPELKKPGETVKISCKAS |
| | 5F6-1 | GYTFTKFGLNWVKQAPGEGLKWMGWLNTYTGEPTYAVDFRGR |
| | heavy | FAFSLETSASTAYLQIDNLKNEDMATYFCATGGTTVVANPFAYW |
| | chain | GQGTLVTVSAAKTTPPSVYPLAPGSAAQTNSMVTLGCLVKGYFPE |
| | | PVTVTWNSGSLSSGVHTFPAVLQSDLYTLSSSVTVPSSTWPSETVT |
| | | CNVAHPASSTKVDKKIVPRDCGCKPCICTVPEVSSVFIFPPKPKDV |
| | | LTITLTPKVTCVVVDISKDDPEVQFSWFVDDVEVHTAQTQPREEQ |
| | | FNSTFRSVSELPIMHQDWLNGKEFKCRVNSAAFPAPIEKTISKTKG |
| | | RPKAPQVYTIPPPKEQMAKDKVSLTCMITDFFPEDITVEWQWNGQ |
| | | PAENYKNTQPIMDTDGSYFVYSKLNVQKSNWEAGNTFTCSVLHE |
| | | GLHNHHTEKSLSHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 134 | M9 mAb | QIQLVQSGPELKKPGETVKISCKASGYTFTKFGLNWVKQAPGEGL |
| | 5F6-1 | KWMGWLNTYTGEPTYAVDFRGRFAFSLETSASTAYLQIDNLKN |
| | heavy | EDMATYFCATGGTTVVANPFAYWGQGTLVTVSAAKTTPPSVYP |
| | chain | LAPGSAAQTNSMVTLGCLVKGYFPEPVTVTWNSGSLSSGVHTFPA |
| | protein | VLQSDLYTLSSSVTVPSSTWPSETVTCNVAHPASSTKVDKKIVPRD |
| | w/o signal | CGCKPCICTVPEVSSVFIFPPKPKDVLTITLTPKVTCVVVDISKDDP |
| | peptide | EVQFSWFVDDVEVHTAQTQPREEQFNSTFRSVSELPIMHQDWLN |
| | | GKEFKCRVNSAAFPAPIEKTISKTKGRPKAPQVYTIPPPKEQMAKD |
| | | KVSLTCMITDFFPEDITVEWQWNGQPAENYKNTQPIMDTDGSYFV |
| | | YSKLNVQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 135 | M9 mAb | QIQLVQSGPELKKPGETVKISCKASGYTFTKFGLNWVKQAPGEGL |
| | 5F6-1 | KWMGWLNTYTGEPTYAVDFRGRFAFSLETSASTAYLQIDNLKN |
| | heavy | EDMATYFCATGGTTVVANPFAYWGQGTLVTVSAA |
| | chain | |
| | variable | |
| | region | |
| 136 | M9 mAb | KFGLN |
| | 5F6-1 | |
| | heavy | |
| | chain | |
| | CDR1 | |
| 137 | M9 mAb | WLNTYTGEPTYAVDFRG |
| | 5F6-1 | |
| | heavy | |
| | chain | |
| | CDR2 | |
| 138 | M9 mAb | GGTTVVANPFAY |
| | 5F6-1 | |
| | heavy | |
| | chain | |
| | CDR3 | |
| 139 | M9 mAb | ATGGATTGGCTGTGGAACTTGCTATTCCTGATGGCAGCTGCCCA |
| | 5F6-1 | AAGTGCCCAAGCACAGATCCAGTTGGTGCAGTCTGGACCTGAAC |
| | heavy | TGAAGAAGCCTGGAGAGACAGTCAAGATCTCCTGCAAGGCTTC |
| | chain | TGGGTATACCTTCACAAAGTTTGGACTGAACTGGGTGAAGCA |
| | nucleic | GGCTCCAGGAGAGGGTTTAAAGTGGATGGGCTGGCTAAACAC |
| | acid | CTACACTGGAGAGCCAACATATGCTGTTGACTTCAGGGGA |
| | | CGATTTGCCTTCTCTTTGGAAACCTCTGCCAGCACTGCCTATTT |
| | | GCAGATCGACAACCTCAAAAATGAGGACATGGCTACATATTT |
| | | CTGTGCGACCGGAGGAACTACGGTAGTAGCCAATCCGTTTG |
| | | CTTACTGGGGCCAAGGGACTCTGGTCACTGTCTCTGCAGCCAA |
| | | AACGACACCCCCATCTGTCTATCCACTGGCCCCTGGATCTGCT |
| | | GCCCAAACTAACTCCATGGTGACCCTGGGATGCCTGGTCAAGG |
| | | GCTATTTCCCTGAGCCAGTGACAGTGACCTGGAACTCTGGATC |
| | | CCTGTCCAGCGGTGTGCACACCTTCCCAGCTGTCCTGCAGTCT |
| | | GACCTCTACACTCTGAGCAGCTCAGTGACTGTCCCCTCCAGCA |
| | | CCTGGCCCAGCGAGACCGTCACCTGCAACGTTGCCCACCCGGC |
| | | CAGCAGCACCAAGGTGGACAAGAAAATTGTGCCCAGGGATTG |
| | | TGGTTGTAAGCCTTGCATATGTACAGTCCCAGAAGTATCATCT |
| | | GTCTTCATCTTCCCCCCAAAGCCCAAGGATGTGCTCACCATTA |
| | | CTCTGACTCCTAAGGTCACGTGTGTTGTGGTAGACATCAGCAA |
| | | GGATGATCCCGAGGTCCAGTTCAGCTGGTTTGTAGATGATGTG |
| | | GAGGTGCACACAGCTCAGACGCAACCCCGGGAGGAGCAGTTC |
| | | AACAGCACTTTCCGCTCAGTCAGTGAACTTCCCATCATGCACC |
| | | AGGACTGGCTCAATGGCAAGGAGTTCAAATGCAGGGTCAACA |
| | | GTGCAGCTTTCCCTGCCCCCATCGAGAAAACCATCTCCAAAAC |
| | | CAAAGGCAGACCGAAGGCTCCACAGGTGTACACCATTCCACC |
| | | TCCCAAGGAGCAGATGGCCAAGGATAAAGTCAGTCTGACCTG |
| | | CATGATAACAGACTTCTTCCCTGAAGACATTACTGTGGAGTGG |
| | | CAGTGGAATGGGCAGCCAGCGGAGAACTACAAGAACACTCAG |
| | | CCCATCATGGACACAGATGGCTCTTACTTCGTCTACAGCAAGC |
| | | TCAATGTGCAGAAGAGCAACTGGGAGGCAGGAAATACTTTCA |
| | | CCTGCTCTGTGTTACATGAGGGCCTGCACAACCACCATACTGA |
| | | GAAGAGCCTCTCCCACTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 140 | M9 mAb | MRFQVQVLGLLLLWISGAQCDVQITQSPSSLAASPGETITINCRASK |
| | 5F6-1 | SISKYLAWYQEKPGKTNKLLIYSGSTLRSGVPSRFSGSGSGTDFT |
| | light chain | LTISSLEPEDFAMYYCQQHDEYPITFGAGTILELKRADAAPTVSIF |
| | | PPSSEQLTSGGASVVCFLNNFYPKDINVKWKIDGSERQNGVLNSW |
| | | TDQDSKDSTYSMSSTLTLTKDEYERHNSYTCEATHKTSTSPIVKSF |
| | | NRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 141 | M9 mAb | DVQITQSPSSLAASPGETITINCRASKSISKYLAWYQEKPGKTNKL |
| | 5F6-1 | LIYSGSTLRSGVPSRFSGSGSGTDFTLTISSLEPEDFAMYYCQQHD |
| | light chain | EYPITFGAGTILELKRADAAPTVSIFPPSSEQLTSGGASVVCFLNNF |
| | w/o signal | YPKDINVKWKIDGSERQNGVLNSWTDQDSKDSTYSMSSTLTLTK |
| | peptide | DEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 142 | M9 mAb | DVQITQSPSSLAASPGETITINCRASKSISKYLAWYQEKPGKTNKL |
| | 5F6-1 | LIYSGSTLRSGVPSRFSGSGSGTDFTLTISSLEPEDFAMYYCQQHD |
| | light chain | EYPITFGAGTILELK |
| | variable | |
| | region | |
| 143 | M9 mAb | RASKSISKYLA |
| | 5F6-1 | |
| | light chain | |
| | CDR1 | |
| 144 | M9 mAb | SGSTLRS |
| | 5F6-1 | |
| | light chain | |
| | CDR2 | |
| 145 | M9 mAb | QQHDEYPIT |
| | 5F6-1 | |
| | light chain | |
| | CDR3 | |
| 146 | M9 mAb | ATGAGGTTCCAGGTTCAGGTTCTGGGGCTCCTTCTGCTCTGGATA |
| | 5F6-1 | TCAGGTGCCCAGTGTGATGTCCAGATAACCCAGTCTCCATCTT |
| | light chain | CTCTTGCTGCATCTCCTGGAGAAACCATTACTATTAATTGCAG |
| | nucleic | GGCAAGTAAGAGCATTAGCAAATATTTAGCCTGGTATCAA |
| | acid | GAGAAACCTGGGAAAACTAATAAACTTCTTATCTACTCTGGA |
| | | TCCACTTTGCGATCTGGAGTTCCATCAAGGTTCAGTGGCAGT |
| | | GGATCTGGTACAGATTTCACTCTCACCATCAGTAGCCTGGAG |
| | | CCTGAAGATTTTGCAATGTATTACTGTCAACAGCATGATGAA |
| | | TACCCGATCACGTTCGGTGCTGGGACCATTCTGGAGCTGAA |
| | | ACGGGCTGATGCTGCACCAACTGTATCCATCTTCCCACCATCC |
| | | AGTGAGCAGTTAACATCTGGAGGTGCCTCAGTCGTGTGCTTC |
| | | TTGAACAACTTCTACCCCAAAGACATCAATGTCAAGTGGAAG |
| | | ATTGATGGCAGTGAACGACAAAATGGCGTCCTGAACAGTTGG |
| | | ACTGATCAGGACAGCAAAGACAGCACCTACAGCATGAGCAG |
| | | CACCCTCACGTTGACCAAGGACGAGTATGAACGACATAACAG |
| | | CTATACCTGTGAGGCCACTCACAAGACATCAACTTCACCCAT |
| | | TGTCAAGAGCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 147 | M9 mAb | MEWSWVFLFFLSVTTGVHSQVQLQQSDAELVKPGASVKISCKASG |
| | 4D5-1 | YTFTDHAIHWVKQKPEQGLEWIGYISPGNGDIKYNEKFEGKATL |
| | heavy | TADKSSSTAYIQLNSLTSEDCAVYFCIEFYMDYWGQGTSVTVSSA |
| | chain | KTTPPSVYPLAPGCGDTTGSSVTLGCLVKGYFPESVTVTWNSGSL |
| | | SSSVHTFPALLQSGLYTMSSSVTVPSSTWPSQTVTCSVAHPASSTT |
| | | VDKKLEPSGPISTINPCPPCKECHKCPAPNLEGGPSVFIFPPNIKDVL |
| | | MISLTPKVTCVVVDVSEDDPDVQISWFVNNVEVHTAQTQTHRED |
| | | YNSTIRVVSTLPIQHQDWMSGKEFKCKVNNKDLPSPIERTISKIKG |
| | | LVRAPQVYILPPPAEQLSRKDVSLTCLVVGFNPGDISVEWTSNGHT |
| | | EENYKDTAPVLDSDGSYFIYSKLNMKTSKWEKTDSFSCNVRHEG |
| | | LKNYYLKKTISRSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 148 | M9 mAb | QVQLQQSDAELVKPGASVKISCKASGYTFTDHAIHWVKQKPEQG |
| | 5F6-1 | LEWIGYISPGNGDIKYNEKFEGKATLTADKSSSTAYIQLNSLTSE |
| | heavy | DCAVYFCIEFYMDYWGQGTSVTVSSAKTTPPSVYPLAPGCGDTT |
| | chain | GSSVTLGCLVKGYFPESVTVTWNSGSLSSSVHTFPALLQSGLYTM |
| | protein | SSSVTVPSSTWPSQTVTCSVAHPASSTTVDKKLEPSGPISTINPCPP |
| | w/o signal | CKECHKCPAPNLEGGPSVFIFPPNIKDVLMISLTPKVTCVVVDVSE |
| | peptide | DDPDVQISWFVNNVEVHTAQTQTHREDYNSTIRVVSTLPIQHQD |
| | | WMSGKEFKCKVNNKDLPSPIERTISKIKGLVRAPQVYILPPPAEQL |
| | | SRKDVSLTCLVVGFNPGDISVEWTSNGHTEENYKDTAPVLDSDGS |
| | | YFIYSKLNMKTSKWEKTDSFSCNVRHEGLKNYYLKKTISRSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 149 | M9 mAb | QVQLQQSDAELVKPGASVKISCKASGYTFTDHAIHWVKQKPEQG |
| | 4D5-1 | LEWIGYISPGNGDIKYNEKFEGKATLTADKSSSTAYIQLNSLTSE |
| | heavy | DCAVYFCIEFYMDYWGQGTSVTVSS |
| | chain | |
| | variable | |
| | region | |
| 150 | M9 mAb | DHAIH |
| | 4D5-1 | |
| | heavy | |
| | chain | |
| | CDR1 | |
| 151 | M9 mAb | YISPGNGDIKYNEKFEG |
| | 4D5-1 | |
| | heavy | |
| | chain | |
| | CDR2 | |
| 152 | M9 mAb | FYMDY |
| | 4D5-1 | |
| | heavy | |
| | chain | |
| | CDR3 | |
| 153 | M9 mAb | ATGGAATGGAGCTGGGTCTTTCTCTTCTTCCTGTCAGTAACTAC |
| | 4D5-1 | AGGTGTCCACTCCCAGGTTCAGCTGCAGCAGTCTGACGCTGAGT |
| | heavy | TGGTGAAACCTGGGGCTTCAGTGAAGATATCCTGCAAGGCTTC |
| | chain | TGGCTACACCTTCACTGACCATGCTATCCACTGGGTAAAGCA |
| | nucleic | GAAGCCTGAACAGGGCCTGGAATGGATTGGATACATTTCTCC |
| | acid | CGGAAATGGTGATATTAAGTATAATGAGAAGTTCGAGGGC |
| | | AAGGCCACACTGACTGCAGACAAATCCTCCAGCACTGCCTAC |
| | | ATACAGCTCAACAGCCTGACATCTGAGGATTGTGCAGTGTATT |
| | | TCTGTATTGAATTTTATATGGACTACTGGGGTCAAGGAACCT |
| | | CAGTCACCGTCTCCTCAGCCAAAACAACACCCCCATCAGTCTA |
| | | TCCACTGGCCCCTGGGTGTGGAGATACAACTGGTTCCTCCGTG |
| | | ACTCTGGGATGCCTGGTCAAGGGCTACTTCCCTGAGTCAGTGA |
| | | CTGTGACTTGGAACTCTGGATCCCTGTCCAGCAGTGTGCACAC |
| | | CTTCCCAGCTCTCCTGCAGTCTGGACTCTACACTATGAGCAGC |
| | | TCAGTGACTGTCCCCTCCAGCACCTGGCCAAGTCAGACCGTCA |
| | | CCTGCAGCGTTGCTCACCCAGCCAGCAGCACCACGGTGGACA |
| | | AAAAACTTGAGCCCAGCGGGCCCATTTCAACAATCAACCCCT |
| | | GTCCTCCATGCAAGGAGTGTCACAAATGCCCAGCTCCTAACCT |
| | | CGAGGGTGGACCATCCGTCTTCATCTTCCCTCCAAATATCAAG |
| | | GATGTACTCATGATCTCCCTGACACCCAAGGTCACGTGTGTGG |
| | | TGGTGGATGTGAGCGAGGATGACCCAGACGTCCAGATCAGCT |
| | | GGTTTGTGAACAACGTGGAAGTACACACAGCTCAGACACAAA |
| | | CCCATAGAGAGGATTACAACAGTACTATCCGGGTGGTCAGCA |
| | | CCCTCCCCATCCAGCACCAGGACTGGATGAGTGGCAAGGAGT |
| | | TCAAATGCAAGGTCAACAACAAAGACCTCCCATCACCCATCG |
| | | AGAGAACCATCTCAAAAATTAAAGGGCTAGTCAGAGCTCCAC |
| | | AAGTATACATCTTGCCGCCACCAGCAGAGCAGTTGTCCAGGA |
| | | AAGATGTCAGTCTCACTTGCCTGGTCGTGGGCTTCAACCCTGG |
| | | AGACATCAGTGTGGAGTGGACCAGCAATGGGCATACAGAGG |
| | | AGAACTACAAGGACACCGCACCAGTCCTGGACTCTGACGGTT |
| | | CTTACTTCATATATAGCAAGCTCAATATGAAAACAAGCAAGT |
| | | GGGAGAAAACAGATTCCTTCTCATGCAACGTGAGACACGAGG |
| | | GTCTGAAAAATTACTACCTGAAGAAGACCATCTCCCGGTCTCC |
| | | GGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 154 | M9 mAb | MSPAQFLFLLVLWIRETNGDVVMTQIPLTLSVTIGQPASISCKSS |
| | 4D5-1 | QSLLYSDGQTYLNWVLQRPGQSPKRLIYVVSKVDSGVPDRFTGSGS |
| | light chain | GTDFTLKISRVEAEDLGVYYCWQGTHFPFTFGSGTKLEIKRADAAP |
| | | TVSIFPPSSEQLTSGGASVVCFLNNFYPKDINVKWKIDGSERQNGV |
| | | LNSWTDQDSKDSTYSMSSTLTLTKDEYERHNSYTCEATHKTSTSP |
| | | IVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 155 | M9 mAb | DVVMTQIPLTLSVTIGQPASISCKSSQSLLYSDGQTYLNWVLQRP |
| | 4D5-1 | GQSPKRLIYVVSKVDSGVPDRFTGSGSGTDFTLKISRVEAEDLGV |
| | light chain | YYCWQGTHFPFTFGSGTKLEIKRADAAPTVSIFPPSSEQLTSGGA |
| | w/o signal | SVVCFLNNFYPKDINVKWKIDGSERQNGVLNSWTDQDSKDSTYSM |
| | peptide | SSTLTLTKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 156 | M9 mAb | DVVMTQIPLTLSVTIGQPASISCKSSQSLLYSDGQTYLNWVLQRP |
| | 4D5-1 | GQSPKRLIYVVSKVDSGVPDRFTGSGSGTDFTLKISRVEAEDLGV |
| | light chain | YYCWQGTHFPFTFGSGTKLEIK |
| | variable | |
| | region | |
| 157 | M9 mAb | KSSQSLLYSDGQTYLN |
| | 4D5-1 | |
| | light chain | |
| | CDR1 | |
| 158 | M9 mAb | VVSKVDS |
| | 4D5-1 | |
| | light chain | |
| | CDR2 | |
| 159 | M9 mAb | WQGTHFPFT |
| | 4D5-1 | |
| | light chain | |
| | CDR3 | |
| 160 | M9 mAb | ATGAGTCCTGCCCAGTTCCTGTTTCTGTTAGTGCTCTGGATTCGG |
| | 4D5-1 | GAAACCAACGGTGATGTTGTGATGACCCAGATTCCACTCACTT |
| | light chain | TGTCGGTTACCATTGGACAACCAGCCTCCATCTCTTGCAAGT |
| | nucleic | CAAGTCAGAGCCTCTTATATAGTGATGGTCAGACATATTT |
| | acid | GAACTGGGTGTTACAGAGGCCAGGCCAGTCTCCAAAGCGCCT |
| | | AATCTATGTGGTGTCTAAAGTGGACTCTGGAGTCCCTGACA |
| | | GGTTCACTGGCAGTGGATCAGGGACAGATTTCACACTGAAAA |
| | | TCAGCAGAGTGGAGGCTGAGGATTTGGGAGTTTATTATTGCT |
| | | GGCAAGGTACACATTTTCCATTCACGTTCGGCTCGGGGACA |
| | | AAGTTGGAAATAAAACGGGCTGATGCTGCACCAACTGTATCC |
| | | ATCTTCCCACCATCCAGTGAGCAGTTAACATCTGGAGGTGCC |
| | | TCAGTCGTGTGCTTCTTGAACAACTTCTACCCCAAAGACATCA |
| | | ATGTCAAGTGGAAGATTGATGGCAGTGAACGACAAAATGGC |
| | | GTCCTGAACAGTTGGACTGATCAGGACAGCAAAGACAGCACC |
| | | TACAGCATGAGCAGCACCCTCACGTTGACCAAGGACGAGTAT |
| | | GAACGACATAACAGCTATACCTGTGAGGCCACTCACAAGACA |
| | | TCAACTTCACCCATTGTCAAGAGCTTCAACAGGAATGAGTGT |
| | | TAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 161 | M9 mAb | MRVLILLCLFTAFPGILSVELQESGPDLVKPSQSLSLTCTVTGWSI |
| | 15H5-1 | ASGYSWHWIRQFPGNRLEWMAYIHYSGSINYNPSLKSRISITRDTS |
| | heavy | KNQFFLQLNSVTTEDTATYYCARNYDYPYFFDYWGQGTTLTVSSA |
| | chain | KTTPPSVYPLAPGSAAQTNSMVTLGCLVKGYFPEPVTVTWNSGSL |
| | | SSGVHTFPAVLQSDLYTLSSSVTVPSSTWPSETVTCNVAHPASSTK |
| | | VDKKIVPRDCGCKPCICTVPEVSSVFIFPPKPKDVLTITLTPKVTCV |
| | | VVDISKDDPEVQFSWFVDDVEVHTAQTQPREEQFNSTFRSVSELPI |
| | | MHQDWLNGKEFKCRVNSAAFPAPIEKTISKTKGRPKAPQVYTIPP |
| | | PKEQMAKDKVSLTCMITDFFPEDITVEWQWNGQPAENYKNTQPI |
| | | MDTDGSYFVYSKLNVQKSNWEAGNTFTCSVLHEGLHNHHTEKSL |
| | | SHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 162 | M9 mAb | VELQESGPDLVKPSQSLSLTCTVTGWSIASGYSWHWIRQFPGNRL |
| | 15H5-1 | EWMAYIHYSGSINYNPSLKSRISITRDTSKNQFFLQLNSVTTEDTA |
| | heavy | TYYCARNYDYPYFFDYWGQGTTLTVSSAKTTPPSVYPLAPGSAA |
| | chain | QTNSMVTLGCLVKGYFPEPVTVTWNSGSLSSGVHTFPAVLQSDL |
| | protein | YTLSSSVTVPSSTWPSETVTCNVAHPASSTKVDKKIVPRDCGCKPC |
| | w/o signal | ICTVPEVSSVFIFPPKPKDVLTITLTPKVTCVVVDISKDDPEVQFSW |
| | peptide | FVDDVEVHTAQTQPREEQFNSTFRSVSELPIMHQDWLNGKEFKCR |
| | | VNSAAFPAPIEKTISKTKGRPKAPQVYTIPPPKEQMAKDKVSLTCM |
| | | ITDFFPEDITVEWQWNGQPAENYKNTQPIMDTDGSYFVYSKLNVQ |
| | | KSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 163 | M9 mAb | VELQESGPDLVKPSQSLSLTCTVTGWSIASGYSWHWIRQFPGNRL |
| | 15H5-1 | EWMAYIHYSGSINYNPSLKSRISITRDTSKNQFFLQLNSVTTEDTA |
| | heavy | TYYCARNYDYPYFFDYWGQGTTLTVSS |
| | chain | |
| | variable | |
| | region | |
| 164 | M9 mAb | SGYSWH |
| | 15H5-1 | |
| | heavy | |
| | chain | |
| | CDR1 | |
| 165 | M9 mAb | YIHYSGSINYNPSLKS |
| | 15H5-1 | |
| | heavy | |
| | chain | |
| | CDR2 | |
| 166 | M9 mAb | NYDYPYFFDY |
| | 15H5-1 | |
| | heavy | |
| | chain | |
| | CDR3 | |
| 167 | M9 mAb | ATGAGAGTGCTGATTCTTTTGTGCCTGTTCACAGCCTTTCCTGGTA |
| | 15H5-1 | TCCTGTCTTATGTGGAACTTCAGGAGTCAGGACCTGACCTGGT |
| | heavy | GAAACCTTCTCAGTCACTTTCACTCACCTGCACTGTCACTGGCT |
| | chain | GGTCCATCGCCAGTGGTTATAGCTGGCACTGGATCCGGCAGT |
| | nucleic | TTCCAGGAAACAGACTGGAATGGATGGCCTACATACACTACA |
| | acid | GTGGTAGTATTAATTACAACCCATCTCTCAAAAGTCGCATC |
| | | TCTATCACTCGAGACACATCCAAGAACCAGTTCTTCCTGCAAT |
| | | TGAATTCTGTGACTACTGAGGACACAGCCACATATTACTGTGC |
| | | ACGAAACTATGATTACCCGTACTTCTTTGACTACTGGGGCC |
| | | AAGGCACCACTCTCACAGTCTCCTCAGCCAAAACGACACCCCC |
| | | ATCTGTCTATCCACTGGCCCCTGGATCTGCTGCCCAAACTAAC |
| | | TCCATGGTGACCCTGGGATGCCTGGTCAAGGGCTATTTCCCTG |
| | | AGCCAGTGACAGTGACCTGGAACTCTGGATCCCTGTCCAGCGG |
| | | TGTGCACACCTTCCCAGCTGTCCTGCAGTCTGACCTCTACACTC |
| | | TGAGCAGCTCAGTGACTGTCCCCTCCAGCACCTGGCCCAGCGA |
| | | GACCGTCACCTGCAACGTTGCCCACCCGGCCAGCAGCACCAA |
| | | GGTGGACAAGAAAATTGTGCCCAGGGATTGTGGTTGTAAGCC |
| | | TTGCATATGTACAGTCCCAGAAGTATCATCTGTCTTCATCTTCC |
| | | CCCCAAAGCCCAAGGATGTGCTCACCATTACTCTGACTCCTAA |
| | | GGTCACGTGTGTTGTGGTAGACATCAGCAAGGATGATCCCGA |
| | | GGTCCAGTTCAGCTGGTTTGTAGATGATGTGGAGGTGCACACA |
| | | GCTCAGACGCAACCCCGGGAGGAGCAGTTCAACAGCACTTTC |
| | | CGCTCAGTCAGTGAACTTCCCATCATGCACCAGGACTGGCTCA |
| | | ATGGCAAGGAGTTCAAATGCAGGGTCAACAGTGCAGCTTTCC |
| | | CTGCCCCCATCGAGAAAACCATCTCCAAAACCAAAGGCAGAC |
| | | CGAAGGCTCCACAGGTGTACACCATTCCACCTCCCAAGGAGC |
| | | AGATGGCCAAGGATAAAGTCAGTCTGACCTGCATGATAACAG |
| | | ACTTCTTCCCTGAAGACATTACTGTGGAGTGGCAGTGGAATGG |
| | | GCAGCCAGCGGAGAACTACAAGAACACTCAGCCCATCATGGA |
| | | CACAGATGGCTCTTACTTCGTCTACAGCAAGCTCAATGTGCAG |
| | | AAGAGCAACTGGGAGGCAGGAAATACTTTCACCTGCTCTGTGT |
| | | TACATGAGGGCCTGCACAACCACCATACTGAGAAGAGCCTCT |
| | | CCCACTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 168 | M9 mAb | MESQIQVFVFVFLWLSGVDGDIVMTQSHKFMSTSEGDRVSLTCRA |
| | 15H5-1 | SQNVNNAVAWYQQKPGQSPKLLIYSTSYRYSGVPDRFTGSGSGTD |
| | light chain | FTFTISSVQAEDLAVYYCQQHYNYPLTFGAGTKLELKRADAAPTV |
| | | SIFPPSSEQLTSGGASVVCFLNNFYPKDINVKWKIDGSERQNGVL |
| | | NSWTDQDSKDSTYSMSSTLTLTKDEYERHNSYTCEATHKTSTSPI |
| | | VKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 169 | M9 mAb | DIVMTQSHKFMSTSEGDRVSLTCRASQNVNNAVAWYQQKPGQSP |
| | 15H5-1 | KLLIYSTSYRYSGVPDRFTGSGSGTDFTFTISSVQAEDLAVYYCQ |
| | light chain | QHYNYPLTFGAGTKLELKRADAAPTVSIFPPSSEQLTSGGASVVC |
| | w/o signal | FLNNFYPKDINVKWKIDGSERQNGVLNSWTDQDSKDSTYSMSST |
| | peptide | LTLTKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 170 | M9 mAb | DIVMTQSHKFMSTSEGDRVSLTCRASQNVNNAVAWYQQKPGQSP |
| | 15H5-1 | KLLIYSTSYRYSGVPDRFTGSGSGTDFTFTISSVQAEDLAVYYC |
| | light chain | QQHYNYPLTFGAGTKLELK |
| | variable | |
| | region | |
| 171 | M9 mAb | RASQNVNNAVA |
| | 15H5-1 | |
| | light chain | |
| | CDR1 | |
| 172 | M9 mAb | STSYRYS |
| | 15H5-1 | |
| | light chain | |
| | CDR2 | |
| 173 | M9 mAb | QQHYNYPLT |
| | 15H5-1 | |
| | light chain | |
| | CDR3 | |
| 174 | M9 mAb | ATGGAGTCACAGATTCAGGTCTTTGTATTCGTGTTTCTCTGGTT |
| | 15H5-1 | GTCTGGTGTTGACGGAGACATTGTGATGACCCAGTCTCACAAAT |
| | light chain | TCATGTCCACTTCAGAAGGAGACAGGGTCAGCCTCACCTGCAG |
| | nucleic | GGCCAGTCAAAATGTGAATAATGCTGTAGCCTGGTATCAA |
| | acid | CAGAAACCAGGACAATCTCCTAAATTGCTGATTTATTCGACA |
| | | TCCTACCGGTACTCTGGAGTCCCTGATCGCTTCACTGGCAGT |
| | | GGATCTGGGACGGATTTCACTTTCACCATCAGCAGTGTGCAG |
| | | GCTGAAGACCTGGCAGTCTATTACTGTCAGCAACATTATAAT |
| | | TATCCGCTCACGTTCGGTGCTGGGACCAAGCTGGAGCTGAA |
| | | ACGGGCTGATGCTGCACCAACTGTATCCATCTTCCCACCATCC |
| | | AGTGAGCAGTTAACATCTGGAGGTGCCTCAGTCGTGTGCTTC |
| | | TTGAACAACTTCTACCCCAAAGACATCAATGTCAAGTGGAAG |
| | | ATTGATGGCAGTGAACGACAAAATGGCGTCCTGAACAGTTGG |
| | | ACTGATCAGGACAGCAAAGACAGCACCTACAGCATGAGCAG |
| | | CACCCTCACGTTGACCAAGGACGAGTATGAACGACATAACAG |
| | | CTATACCTGTGAGGCCACTCACAAGACATCAACTTCACCCAT |
| | | TGTCAAGAGCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 175 | M9 mAb | MEWTWVFLFLLSVTAGVHSQFQLQQSGGELMKPGASVKISCKATG |
| | 4E1-2 | YTFSNYWIEWMKQRPGHGLEWIGEILPGSGITKYNEKFKGKAT |
| | heavy | FTADPSSNTAYMQLSSLTSEDSAVYYCSRGGWDGYWGQGTTLT |
| | chain | VASAKTTPPSVYPLAPGSAAQTNSMVTLGCLVKGYFPEPVTVTW |
| | | NSGSLSSGVHTFPAVLQSDLYTLSSSVTVPSSTWPSETVTCNVAHP |
| | | ASSTKVDKKIVPRDCGCKPCICTVPEVSSVFIFPPKPKDVLTITLTP |
| | | KVTCVVVDISKDDPEVQFSWFVDDVEVHTAQTQPREEQFNSTFRS |
| | | VSELPIMHQDWLNGKEFKCRVNSAAFPAPIEKTISKTKGRPKAPQ |
| | | VYTIPPPKEQMAKDKVSLTCMITDFFPEDITVEWQWNGQPAENYK |
| | | NTQPIMDTDGSYFVYSKLNVQKSNWEAGNTFTCSVLHEGLHNHH |
| | | TEKSLSHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 176 | M9 mAb | QFQLQQSGGELMKPGASVKISCKATGYTFSNYWIEWMKQRPGH |
| | 4E1-2 | GLEWIGEILPGSGITKYNEKFKGKATFTADPSSNTAYMQLSSLT |
| | heavy | SEDSAVYYCSRGGWDGYWGQGTTLTVASAKTTPPSVYPLAPGSA |
| | chain | AQTNSMVTLGCLVKGYFPEPVTVTWNSGSLSSGVHTFPAVLQSDL |
| | protein | YTLSSSVTVPSSTWPSETVTCNVAHPASSTKVDKKIVPRDCGCKP |
| | w/o signal | CICTVPEVSSVFIFPPKPKDVLTITLTPKVTCVVVDISKDDPEVQ |
| | peptide | FSWFVDDVEVHTAQTQPREEQFNSTFRSVSELPIMHQDWLNGKEF |
| | | KCRVNSAAFPAPIEKTISKTKGRPKAPQVYTIPPPKEQMAKDKVS |
| | | LTCMITDFFPEDITVEWQWNGQPAENYKNTQPIMDTDGSYFVYSK |
| | | LNVQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 177 | M9 mAb | QFQLQQSGGELMKPGASVKISCKATGYTFSNYWIEWMKQRPGH |
| | 4E1-2 | GLEWIGEILPGSGITKYNEKFKGKATFTADPSSNTAYMQLSSLTS |
| | heavy | EDSAVYYCSRGGWDGYWGQGTTLTVAS |
| | chain | |
| | variable | |
| | region | |
| 178 | M9 mAb | NYWIE |
| | 4E1-2 | |
| | heavy | |
| | chain | |
| | CDR1 | |
| 179 | M9 mAb | EILPGSGITKYNEKFKG |
| | 4E1-2 | |
| | heavy | |
| | chain | |
| | CDR2 | |
| 180 | M9 mAb | GGWDGY |
| | 4E1-2 | |
| | heavy | |
| | chain | |
| | CDR3 | |
| 181 | M9 mAb | ATGGAATGGACCTGGGTCTTTCTCTTCCTCCTGTCAGTAACTGC |
| | 4E1-2 | AGGTGTCCACTCCCAGTTTCAGCTGCAGCAGTCTGGAGGTGAGC |
| | heavy | TGATGAAGCCTGGGGCCTCAGTGAAGATATCCTGCAAGGCTAC |
| | chain | TGGCTACACATTCAGTAACTACTGGATAGAGTGGATGAAGCA |
| | nucleic | GAGGCCTGGACATGGCCTTGAGTGGATTGGAGAGATTTTACC |
| | acid | TGGAAGTGGTATTACTAAATACAATGAGAAGTTCAAGGGC |
| | | AAGGCCACATTCACTGCAGATCCATCCTCCAACACAGCCTACA |
| | | TGCAACTCAGTAGCCTGACATCTGAGGACTCTGCCGTCTATTA |
| | | CTGTTCAAGAGGGGGCTGGGACGGCTACTGGGGCCAAGGCA |
| | | CCACTCTCACAGTCGCCTCAGCCAAAACGACACCCCCATCTGT |
| | | CTATCCACTGGCCCCTGGATCTGCTGCCCAAACTAACTCCATG |
| | | GTGACCCTGGGATGCCTGGTCAAGGGCTATTTCCCTGAGCCAG |
| | | TGACAGTGACCTGGAACTCTGGATCCCTGTCCAGCGGTGTGCA |
| | | CACCTTCCCAGCTGTCCTGCAGTCTGACCTCTACACTCTGAGC |
| | | AGCTCAGTGACTGTCCCCTCCAGCACCTGGCCCAGCGAGACCG |
| | | TCACCTGCAACGTTGCCCACCCGGCCAGCAGCACCAAGGTGG |
| | | ACAAGAAAATTGTGCCCAGGGATTGTGGTTGTAAGCCTTGCAT |
| | | ATGTACAGTCCCAGAAGTATCATCTGTCTTCATCTTCCCCCCA |
| | | AAGCCCAAGGATGTGCTCACCATTACTCTGACTCCTAAGGTCA |
| | | CGTGTGTTGTGGTAGACATCAGCAAGGATGATCCCGAGGTCCA |
| | | GTTCAGCTGGTTTGTAGATGATGTGGAGGTGCACACAGCTCAG |
| | | ACGCAACCCCGGGAGGAGCAGTTCAACAGCACTTTCCGCTCA |
| | | GTCAGTGAACTTCCCATCATGCACCAGGACTGGCTCAATGGCA |
| | | AGGAGTTCAAATGCAGGGTCAACAGTGCAGCTTTCCCTGCCCC |
| | | CATCGAGAAAACCATCTCCAAAACCAAAGGCAGACCGAAGGC |
| | | TCCACAGGTGTACACCATTCCACCTCCCAAGGAGCAGATGGCC |
| | | AAGGATAAAGTCAGTCTGACCTGCATGATAACAGACTTCTTCC |
| | | CTGAAGACATTACTGTGGAGTGGCAGTGGAATGGGCAGCCAG |
| | | CGGAGAACTACAAGAACACTCAGCCCATCATGGACACAGATG |
| | | GCTCTTACTTCGTCTACAGCAAGCTCAATGTGCAGAAGAGCAA |
| | | CTGGGAGGCAGGAAATACTTTCACCTGCTCTGTGTTACATGAG |
| | | GGCCTGCACAACCACCATACTGAGAAGAGCCTCTCCCACTCTC |
| | | CTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 182 | M9 mAb | MVLAQFLAFLLLWFPGVRCDILMTQSPSSMSVSLGDTVSITCHANQ |
| | 4E1-2 | DISSNIGWLQQKPGKSFQGLIYHGTNLEDGVSSRFSGSGSGTDYS |
| | light chain | LTISSLESEDFADYYCVQYAQFPYTFGGGTKLEIKRADAAPTVSIF |
| | | PPSSEQLTSGGASVVCFLNNFYPKDINVKWKIDGSERQNGVLNSW |
| | | TDQDSKDSTYSMSSTLTLTKDEYERHNSYTCEATHKTSTSPIVKSF |
| | | NRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 183 | M9 mAb | DILMTQSPSSMSVSLGDTVSITCHANQDISSNIGWLQQKPGKSFQ |
| | 4E1-2 | GLIYHGTNLEDGVSSRFSGSGSGTDYSLTISSLESEDFADYYCVQY |
| | light chain | AQFPYTFGGGTKLEIKRADAAPTVSIFPPSSEQLTSGGASVVCFLN |
| | w/o signal | NFYPKDINVKWKIDGSERQNGVLNSWTDQDSKDSTYSMSSTLTL |
| | peptide | TKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 184 | M9 mAb | DILMTQSPSSMSVSLGDTVSITCHANQDISSNIGWLQQKPGKSFQ |
| | 4E1-2 | GLIYHGTNLEDGVSSRFSGSGSGTDYSLTISSLESEDFADYYCVQY |
| | light chain | AQFPYTFGGGTKLEIK |
| | variable | |
| | region | |
| 185 | M9 mAb | HANQDISSNI |
| | 4E1-2 | |
| | light chain | |
| | CDR1 | |
| 186 | M9 mAb | HGTNLED |
| | 4E1-2 | |
| | light chain | |
| | CDR2 | |
| 187 | M9 mAb | VQYAQFPYT |
| | 4E1-2 | |
| | light chain | |
| | CDR3 | |
| 188 | M9 mAb | ATGGTCCTTGCTCAGTTTCTTGCATTCTTGTTGCTTTGGTTTCCAGG |
| | 4E1-2 | TGTAAGATGTGACATCCTGATGACCCAATCTCCTTCCTCCATGT |
| | light chain | CTGTATCTCTGGGAGACACAGTCAGCATCACTTGCCATGCAA |
| | nucleic | ATCAGGACATTAGTAGTAATATAGGGTGGTTGCAGCAGAAA |
| | acid | CCAGGGAAATCATTTCAGGGCCTGATCTATCATGGAACCAAC |
| | | TTGGAAGATGGAGTTTCATCAAGGTTCAGTGGCAGTGGATCT |
| | | GGAACAGATTATTCTCTCACCATCAGCAGTCTGGAATCTGAAG |
| | | ATTTTGCAGACTATTATTGTGTACAGTATGCTCAGTTTCCGT |
| | | ACACGTTCGGAGGGGGGACCAAGTTGGAAATAAAACGGGCTG |
| | | ATGCTGCACCAACTGTATCCATCTTCCCACCATCCAGTGAGCA |
| | | GTTAACATCTGGAGGTGCCTCAGTCGTGTGCTTCTTGAACAAC |
| | | TTCTACCCCAAAGACATCAATGTCAAGTGGAAGATTGATGGCA |
| | | GTGAACGACAAAATGGCGTCCTGAACAGTTGGACTGATCAGG |
| | | ACAGCAAAGACAGCACCTACAGCATGAGCAGCACCCTCACGT |
| | | TGACCAAGGACGAGTATGAACGACATAACAGCTATACCTGTG |
| | | AGGCCACTCACAAGACATCAACTTCACCCATTGTCAAGAGCTT |
| | | CAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 189 | U5A mAb | MEWSWVSLFFLSVTTGVHSQVQLQQSDAELVKPGASVKISCKVSGY |
| | 17C1_1 | TFSDHTLHWMKQRPEQGLEWIGYIYLRNGSVNYNERFKGKATL |
| | heavy | TADRSSTTAYMQLNSLTSEDSAVFFCARGNFAVWGTGTTVTVSS |
| | chain | AKTTPPSVYPLAPGSAAQTNSMVTLGCLVKGYFPEPVTVTWNSGS |
| | | LSSGVHTFPAVLQSDLYTLSSSVTVPSSTWPSQTVTCNVAHPASST |
| | | KVDKKIVPRDCGCKPCICTVPEVSSVFIFPPKPKDVLTITLTPKVTC |
| | | VVVDISKDDPEVQFSWFVDDVEVHTAQTKPREEQINSTFRSVSELP |
| | | IMHQDWLNGKEFKCRVNSAAFPAPIEKTISKTKGRPKAPQVYTIPP |
| | | PKEQMAKDKVSLTCMITNFFPEDITVEWQWNGQPAENYKNTQPI |
| | | MDTDGSYFVYSKLNVQKSNWEAGNTFTCSVLHEGLHNHHTEKSL |
| | | SHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 190 | U5A mAb | QVQLQQSDAELVKPGASVKISCKVSGYTFSDHTLHWMKQRPEQG |
| | 17C1_1 | LEWIGYIYLRNGSVNYNERFKGKATLTADRSSTTAYMQLNSLTS |
| | heavy | EDSAVFFCARGNFAVWGTGTTVTVSSAKTTPPSVYPLAPGSAAQ |
| | chain | TNSMVTLGCLVKGYFPEPVTVTWNSGSLSSGVHTFPAVLQSDLYT |
| | protein | LSSSVTVPSSTWPSQTVTCNVAHPASSTKVDKKIVPRDCGCKPCI |
| | w/o signal | CTVPEVSSVFIFPPKPKDVLTITLTPKVTCVVVDISKDDPEVQFS |
| | peptide | WFVDDVEVHTAQTKPREEQINSTFRSVSELPIMHQDWLNGKEFKC |
| | | SRVNAAFPAPIEKTISKTKGRPKAPQVYTIPPPKEQMAKDKVSLT |
| | | CMITNFFPEDITVEWQWNGQPAENYKNTQPIMDTDGSYFVYSKLN |
| | | VQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 191 | U5A mAb | QVQLQQSDAELVKPGASVKISCKVSGYTFSDHTLHWMKQRP |
| | 17C1_1 | EQGLEWIGYIYLRNGSVNYNERFKGKATLTADRSSTTAYM |
| | heavy | QLNSLTSEDSAVFFCARGNFAVWGTGTTVTVSS |
| | chain | |
| | variable | |
| | region | |
| 192 | U5A mAb | DHTLH |
| | 17C1_1 | |
| | heavy | |
| | chain | |
| | CDR1 | |
| 193 | U5A mAb | YIYLRNGSVNYNERFKG |
| | 17C1_1 | |
| | heavy | |
| | chain | |
| | CDR2 | |
| 194 | U5A mAb | GNFAV |
| | 17C1_1 | |
| | heavy | |
| | chain | |
| | CDR3 | |
| 195 | U5A mAb | ATGGAATGGAGCTGGGTCTCTCTCTTCTTCCTGTCAGTAACTACAG |
| | 17C1_1 | GTGTCCACTCCCAGGTTCAGCTGCAACAGTCAGACGCTGAGTTG |
| | heavy | GTGAAACCTGGAGCTTCAGTGAAGATATCCTGCAAGGTTTCTG |
| | chain | GCTACACCTTCTCTGACCATACTCTTCACTGGATGAAGCAGAG |
| | nucleic | GCCTGAACAGGGCCTGGAATGGATTGGATATATTTATCTTAG |
| | acid | AAATGGTAGTGTTAACTACAATGAGAGGTTCAAGGGCAAGG |
| | | CCACGTTGACTGCAGACAGATCCTCCACCACAGCCTACATGCA |
| | | GCTCAACAGCCTGACATCTGAGGACTCTGCAGTCTTTTTCTGTG |
| | | CAAGAGGGAACTTCGCTGTCTGGGGCACAGGGACCACGGTC |
| | | ACCGTCTCCTCAGCCAAAACGACACCCCCATCTGTCTATCCACT |
| | | GGCCCCTGGATCTGCTGCCCAAACTAACTCCATGGTGACCCTG |
| | | GGATGCCTGGTCAAGGGCTATTTCCCTGAGCCAGTGACAGTGA |
| | | CCTGGAACTCTGGATCCCTGTCCAGCGGTGTGCACACCTTCCCA |
| | | GCTGTCCTGCAGTCTGACCTCTACACTCTGAGCAGCTCAGTGAC |
| | | TGTCCCCTCCAGCACCTGGCCCAGCCAGACCGTCACCTGCAAC |
| | | GTTGCCCACCCGGCCAGCAGCACCAAGGTGGACAAGAAAATT |
| | | GTGCCCAGGGATTGTGGTTGTAAGCCTTGCATATGTACAGTCC |
| | | CAGAAGTATCATCTGTCTTCATCTTCCCCCCAAAGCCCAAGGAT |
| | | GTGCTCACCATTACTCTGACTCCTAAGGTCACGTGTGTTGTGGT |
| | | AGACATCAGCAAGGATGATCCCGAGGTCCAGTTCAGCTGGTTT |
| | | GTAGATGATGTGGAGGTGCACACAGCTCAGACGAAACCCCGG |
| | | GAGGAGCAGATCAACAGCACTTTCCGTTCAGTCAGTGAACTTC |
| | | CCATCATGCACCAGGACTGGCTCAATGGCAAGGAGTTCAAATG |
| | | CAGGGTCAACAGTGCAGCTTTCCCTGCCCCCATCGAGAAAACC |
| | | ATCTCCAAAACCAAAGGCAGACCGAAGGCTCCACAGGTGTAC |
| | | ACCATTCCACCTCCCAAGGAGCAGATGGCCAAGGATAAAGTCA |
| | | GTCTGACCTGCATGATAACAAACTTCTTCCCTGAAGACATTACT |
| | | GTGGAGTGGCAGTGGAATGGGCAGCCAGCGGAGAACTACAAG |
| | | AACACTCAGCCCATCATGGACACAGATGGCTCTTACTTCGTCT |
| | | ACAGCAAGCTCAATGTGCAGAAGAGCAACTGGGAGGCAGGAA |
| | | ATACTTTCACCTGCTCTGTGTTACATGAGGGCCTGCACAACCAC |
| | | CATACTGAGAAGAGCCTCTCCCACTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 196 | U5A mAb | MKLPVRLLVLMFWIPASSSDVVMTQIPLSLPVSLGDQASISCRSSQS |
| | 17C1_1 | LVHSNGNTYLHWYLQKPGQSPKLLIYKVSNRFSGVPDRFSGSGS |
| | light chain | GTDFTLKISRVEAEDLGVYFCSQSTHVPFTFGSGTKLEIKRADAAP |
| | | TVSIFPPSSEQLTSGGASVVCFLNNFYPKDINVKWKIDGSERQNGV |
| | | LNSWTDQDSKDSTYSMSSTLTLTKDEYERHNSYTCEATHKTSTSP |
| | | IVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 197 | U5A mAb | DVVMTQIPLSLPVSLGDQASISCRSSQSLVHSNGNTYLHWYLQKPG |
| | 17C1_1 | QSPKLLIYKVSNRFSGVPDRFSGSGSGTDFTLKISRVEAEDLGVYF |
| | light chain | CSQSTHVPFTFGSGTKLEIKRADAAPTVSIFPPSSEQLTSGGASVV |
| | w/o signal | CFLNNFYPKDINVKWKIDGSERQNGVLNSWTDQDSKDSTYSMSS |
| | peptide | TLTLTKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 198 | U5A mAb | DVVMTQIPLSLPVSLGDQASISCRSSQSLVHSNGNTYLHWYLQKPG |
| | 17C1_1 | QSPKLLIYKVSNRFSGVPDRFSGSGSGTDFTLKISRVEAEDLGVYF |
| | light chain | CSQSTHVPFTFGSGTKLEIK |
| | variable | |
| | region | |
| 199 | U5A mAb | RSSQSLVHSNGNTYLH |
| | 17C1_1 | |
| | light chain | |
| | CDR1 | |
| 200 | U5A mAb | KVSNRFS |
| | 17C1_1 | |
| | light chain | |
| | CDR2 | |
| 201 | U5A mAb | SQSTHVPFT |
| | 17C1_1 | |
| | light chain | |
| | CDR3 | |
| 202 | U5A mAb | ATGAAGTTGCCTGTTAGGCTGTTGGTGCTGATGTTCTGGATTCCTG |
| | 17C1_1 | CTTCCAGCAGTGATGTTGTGATGACCCAAATTCCACTCTCCCTG |
| | light chain | CCTGTCAGTCTTGGAGATCAAGCCTCCATCTCTTGCAGATCTA |
| | nucleic | GTCAGAGCCTTGTACACAGTAATGGAAACACCTATTTACAT |
| | acid | TGGTACCTGCAGAAGCCAGGCCAGTCTCCAAAGCTCCTGATCT |
| | | ATAAAGTTTCCAACCGATTTTCTGGGGTCCCAGACAGGTTCA |
| | | GTGGCAGTGGATCAGGGACAGATTTCACACTCAAGATCAGCAG |
| | | AGTGGAGGCTGAGGATCTGGGAGTTTATTTCTGCTCTCAAAGT |
| | | ACACATGTTCCATTCACGTTCGGCTCGGGGACAAAGTTGGAA |
| | | ATAAAACGGGCTGATGCTGCACCAACTGTATCCATCTTCCCAC |
| | | CATCCAGTGAGCAGTTAACATCTGGAGGTGCCTCAGTCGTGTG |
| | | CTTCTTGAACAACTTCTACCCCAAAGACATCAATGTCAAGTGG |
| | | AAGATTGATGGCAGTGAACGACAAAATGGCGTCCTGAACAGTT |
| | | GGACTGATCAGGACAGCAAAGACAGCACCTACAGCATGAGCA |
| | | GCACCCTCACGTTGACCAAGGACGAGTATGAACGACATAACAG |
| | | CTATACCTGTGAGGCCACTCACAAGACATCAACTTCACCCATT |
| | | GTCAAGAGCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 203 | U5A mAb | MGWSCIILILVAAATGVHSQVQLQQPGAELVKPGASVKMSCKASGY |
| | 17C9_1 | TFTSYWITWVKQRPGQGLEWIGDIYPGSGSTTYNEKFKSKATLT |
| | heavy | VDTSSSTAYMQLSSLTSEDSAVYYCARRHYGSSSAWFAYWGQG |
| | chain | TLVTVSAAKTTPPSVYPLAPGSAAQTNSMVTLGCLVKGYFPEPVT |
| | | VTWNSGSLSSGVHTFPAVLQSDLYTLSSSVTVPSSTWPSQTVTCN |
| | | VAHPASSTKVDKKIVPRDCGCKPCICTVPEVSSVFIFPPKPKDVLTI |
| | | TLTPKVTCVVVDISKDDPEVQFSWFVDDVEVHTAQTKPREEQINS |
| | | TFRSVSELPIMHQDWLNGKEFKCRVNSAAFPAPIEKTISKTKGRPK |
| | | APQVYTIPPPKEQMAKDKVSLTCMITNFFPEDITVEWQWNGQPAE |
| | | NYKNTQPIMDTDGSYFVYSKLNVQKSNWEAGNTFTCSVLHEGLH |
| | | NHHTEKSLSHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 204 | U5A mAb | QVQLQQPGAELVKPGASVKMSCKASGYTFTSYWITWVKQRPGQ |
| | 17C9_1 | GLEWIGDIYPGSGSTTYNEKFKSKATLTVDTSSSTAYMQLSSLTS |
| | heavy | EDSAVYYCARRHYGSSSAWFAYWGQGTLVTVSAAKTTPPSVYP |
| | chain | LAPGSAAQTNSMVTLGCLVKGYFPEPVTVTWNSGSLSSGVHTFPA |
| | protein | VLQSDLYTLSSSVTVPSSTWPSQTVTCNVAHPASSTKVDKKIVPR |
| | w/o signal | DCGCKPCICTVPEVSSVFIFPPKPKDVLTITLTPKVTCVVVDISKDD |
| | peptide | PEVQFSWFVDDVEVHTAQTKPREEQINSTFRSVSELPIMHQDWLN |
| | | GKEFKCRVNSAAFPAPIEKTISKTKGRPKAPQVYTIPPPKEQMAKD |
| | | KVSLTCMITNFFPEDITVEWQWNGQPAENYKNTQPIMDTDGSYFV |
| | | YSKLNVQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 205 | U5A mAb | QVQLQQPGAELVKPGASVKMSCKASGYTFTSYWITWVKQRPGQG |
| | 17C9_1 | LEWIGDIYPGSGSTTYNEKFKSKATLTVDTSSSTAYMQLSSLTS |
| | heavy | EDSAVYYCARRHYGSSSAWFAYWGQGTLVTVSA |
| | chain | |
| | variable | |
| | region | |
| 206 | U5A mAb | SYWIT |
| | 17C9_1 | |
| | heavy | |
| | chain | |
| | CDR1 | |
| 207 | U5A mAb | DIYPGSGSTTYNEKFKS |
| | 17C9_1 | |
| | heavy | |
| | chain | |
| | CDR2 | |
| 208 | U5A mAb | RHYGSSSAWFAY |
| | 17C9_1 | |
| | heavy | |
| | chain | |
| | CDR3 | |
| 209 | U5A mAb | ATGGGATGGAGCTGTATCATCCTCATTTTGGTAGCAGCAGCTACAG |
| | 17C9_1 | GTGTCCACTCCCAGGTCCAACTGCAGCAGCCTGGGGCTGAGCTT |
| | heavy | GTGAAGCCTGGGGCTTCAGTGAAGATGTCCTGCAAGGCTTCTG |
| | chain | GCTACACCTTCACCAGCTACTGGATAACCTGGGTGAAGCAGA |
| | nucleic | GGCCTGGACAAGGCCTTGAGTGGATTGGAGATATTTATCCTG |
| | acid | GTAGTGGTAGTACTACCTACAATGAGAAGTTCAAGAGCAAG |
| | | GCCACACTGACTGTTGACACATCCTCCAGCACAGCCTACATGC |
| | | AGCTCAGCAGCCTGACATCTGAGGACTCTGCGGTCTATTACTG |
| | | TGCAAGAAGGCACTACGGTAGTAGCTCGGCCTGGTTTGCTT |
| | | ACTGGGGCCAAGGGACTCTGGTCACTGTCTCTGCAGCCAAAAC |
| | | GACACCCCCATCTGTCTATCCACTGGCCCCTGGATCTGCTGCCC |
| | | AAACTAACTCCATGGTGACCCTGGGATGCCTGGTCAAGGGCTA |
| | | TTTCCCTGAGCCAGTGACAGTGACCTGGAACTCTGGATCCCTGT |
| | | CCAGCGGTGTGCACACCTTCCCAGCTGTCCTGCAGTCTGACCTC |
| | | TACACTCTGAGCAGCTCAGTGACTGTCCCCTCCAGCACCTGGC |
| | | CCAGCCAGACCGTCACCTGCAACGTTGCCCACCCGGCCAGCAG |
| | | CACCAAGGTGGACAAGAAAATTGTGCCCAGGGATTGTGGTTGT |
| | | AAGCCTTGCATATGTACAGTCCCAGAAGTATCATCTGTCTTCAT |
| | | CTTCCCCCCAAAGCCCAAGGATGTGCTCACCATTACTCTGACTC |
| | | CTAAGGTCACGTGTGTTGTGGTAGACATCAGCAAGGATGATCC |
| | | CGAGGTCCAGTTCAGCTGGTTTGTAGATGATGTGGAGGTGCAC |
| | | ACAGCTCAGACGAAACCCCGGGAGGAGCAGATCAACAGCACT |
| | | TTCCGTTCAGTCAGTGAACTTCCCATCATGCACCAGGACTGGCT |
| | | CAATGGCAAGGAGTTCAAATGCAGGGTCAACAGTGCAGCTTTC |
| | | CCTGCCCCCATCGAGAAAACCATCTCCAAAACCAAAGGCAGAC |
| | | CGAAGGCTCCACAGGTGTACACCATTCCACCTCCCAAGGAGCA |
| | | GATGGCCAAGGATAAAGTCAGTCTGACCTGCATGATAACAAAC |
| | | TTCTTCCCTGAAGACATTACTGTGGAGTGGCAGTGGAATGGGC |
| | | AGCCAGCGGAGAACTACAAGAACACTCAGCCCATCATGGACA |
| | | CAGATGGCTCTTACTTCGTCTACAGCAAGCTCAATGTGCAGAA |
| | | GAGCAACTGGGAGGCAGGAAATACTTTCACCTGCTCTGTGTTA |
| | | CATGAGGGCCTGCACAACCACCATACTGAGAAGAGCCTCTCCC |
| | | ACTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 210 | U5A mAb | MSSAQFLGLLLLCFQGTRCDIQMTQTTSSLSASLGDRVTISCSASQG |
| | 17C9_1 | IRNYLNWYQQKPDGTVKLLIYYTSSLHSGVPSRFSGSGSGTDYSL |
| | light chain | TISNLEPEDIATYYCQQYSKLPFTFGSGTKLEIKRADAAPTVSIFPP |
| | | SSEQLTSGGASVVCFLNNFYPKDINVKWKIDGSERQNGVLNSWT |
| | | DQDSKDSTYSMSSTLTLTKDEYERHNSYTCEATHKTSTSPIVKSFN |
| | | RNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 211 | U5A mAb | DIQMTQTTSSLSASLGDRVTISCSASQGIRNYLNWYQQKPDGTVK |
| | 17C9_1 | LLIYYTSSLHSGVPSRFSGSGSGTDYSLTISNLEPEDIATYYCQQYS |
| | light chain | KLPFTFGSGTKLEIKRADAAPTVSIFPPSSEQLTSGGASVVCFLNNF |
| | w/o signal | YPKDINVKWKIDGSERQNGVLNSWTDQDSKDSTYSMSSTLTLTK |
| | peptide | DEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 212 | U5A mAb | DIQMTQTTSSLSASLGDRVTISCSASQGIRNYLNWYQQKPDGTVK |
| | 17C9_1 | LLIYYTSSLHSGVPSRFSGSGSGTDYSLTISNLEPEDIATYYCQQYS |
| | light chain | KLPFTFGSGTKLEIK |
| | variable | |
| | region | |
| 213 | U5A mAb | SASQGIRNYLN |
| | 17C9_1 | |
| | light chain | |
| | CDR1 | |
| 214 | U5A mAb | YTSSLHS |
| | 17C9_1 | |
| | light chain | |
| | CDR2 | |
| 215 | U5A mAb | QQYSKLPFT |
| | 17C9_1 | |
| | light chain | |
| | CDR3 | |
| 216 | U5A mAb | ATGTCCTCTGCTCAGTTCCTTGGTCTCCTGTTGCTCTGTTTTCAAG |
| | 17C9_1 | GTACCAGATGTGATATCCAGATGACACAGACTACATCCTCCCTG |
| | light chain | TCTGCCTCTCTGGGAGACAGAGTCACCATCAGTTGCAGTGCA |
| | nucleic | AGTCAGGGCATTAGGAATTATTTAAACTGGTATCAGCAGAA |
| | acid | ACCAGATGGAACTGTTAAACTCCTGATCTATTACACATCAAG |
| | | TTTACACTCAGGAGTCCCATCAAGGTTCAGTGGCAGTGGGTC |
| | | TGGGACAGATTATTCTCTCACCATCAGCAACCTGGAACCTGAA |
| | | GATATTGCCACTTACTATTGTCAGCAGTATAGTAAGCTTCCA |
| | | TTCACGTTCGGCTCGGGGACAAAGTTGGAAATAAAACGGGCT |
| | | GATGCTGCACCAACTGTATCCATCTTCCCACCATCCAGTGAGC |
| | | AGTTAACATCTGGAGGTGCCTCAGTCGTGTGCTTCTTGAACAA |
| | | CTTCTACCCCAAAGACATCAATGTCAAGTGGAAGATTGATGGC |
| | | AGTGAACGACAAAATGGCGTCCTGAACAGTTGGACTGATCAG |
| | | GACAGCAAAGACAGCACCTACAGCATGAGCAGCACCCTCACG |
| | | TTGACCAAGGACGAGTATGAACGACATAACAGCTATACCTGT |
| | | GAGGCCACTCACAAGACATCAACTTCACCCATTGTCAAGAGCT |
| | | TCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 217 | U5A mAb | MGWSCIILILVAAATGVHSQVQLQQPGAELVKPGASVKMSCKASGY |
| | 2E6_1 | TFTSYWITWVKQRPGQGLEWIGDIYPGSGSTTYNEKFKSKATLT |
| | heavy | VDTSSSTAYMQFSSLTSEDSAVYYCARRHYGSTSAWFAYWGQG |
| | chain | TLVTVSAAKTTPPSVYPLAPGSAAQTNSMVTLGCLVKGYFPEPVT |
| | | VTWNSGSLSSGVHTFPAVLQSDLYTLSSSVTVPSSTWPSQTVTCN |
| | | VAHPASSTKVDKKIVPRDCGCKPCICTVPEVSSVFIFPPKPKDVLTI |
| | | TLTPKVTCVVVDISKDDPEVQFSWFVDDVEVHTAQTKPREEQINS |
| | | TFRSVSELPIMHQDWLNGKEFKCRVNSAAFPAPIEKTISKTKGRPK |
| | | APQVYTIPPPKEQMAKDKVSLTCMITNFFPEDITVEWQWNGQPAE |
| | | NYKNTQPIMDTDGSYFVYSKLNVQKSNWEAGNTFTCSVLHEGLH |
| | | NHHTEKSLSHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 218 | U5A mAb | QVQLQQPGAELVKPGASVKMSCKASGYTFTSYWITWVKQRPGQ |
| | 2E6_1 | GLEWIGDIYPGSGSTTYNEKFKSKATLTVDTSSSTAYMQFSSLT |
| | heavy | SEDSAVYYCARRHYGSTSAWFAYWGQGTLVTVSAAKTTPPSVYP |
| | chain | LAPGSAAQTNSMVTLGCLVKGYFPEPVTVTWNSGSLSSGVHTFPA |
| | protein | VLQSDLYTLSSSVTVPSSTWPSQTVTCNVAHPASSTKVDKKIVPR |
| | w/o signal | DCGCKPCICTVPEVSSVFIFPPKPKDVLTITLTPKVTCVVVDISKDD |
| | peptide | PEVQFSWFVDDVEVHTAQTKPREEQINSTFRSVSELPIMHQDWLN |
| | | GKEFKCRVNSAAFPAPIEKTISKTKGRPKAPQVYTIPPPKEQMAKD |
| | | KVSLTCMITNFFPEDITVEWQWNGQPAENYKNTQPIMDTDGSYFV |
| | | YSKLNVQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 219 | U5A mAb | QVQLQQPGAELVKPGASVKMSCKASGYTFTSYWITWVKQRPGQ |
| | 2E6_1 | GLEWIGDIYPGSGSTTYNEKFKSKATLTVDTSSSTAYMQFSSLTS |
| | heavy | EDSAVYYCARRHYGSTSAWFAYWGQGTLVTVSA |
| | chain | |
| | variable | |
| | region | |
| 220 | U5A mAb | SYWIT |
| | 2E6_1 | |
| | heavy | |
| | chain | |
| | CDR1 | |
| 221 | U5A mAb | DIYPGSGSTTYNEKFKS |
| | 2E6_1 | |
| | heavy | |
| | chain | |
| | CDR2 | |
| 222 | U5A mAb | RHYGSTSAWFAY |
| | 2E6_1 | |
| | heavy | |
| | chain | |
| | CDR3 | |
| 223 | U5A mAb | ATGGGATGGAGCTGTATCATCCTCATTTTGGTAGCAGCAGCTA |
| | 2E6_1 | CAGGTGTCCACTCCCAGGTCCAACTGCAGCAGCCTGGGGCTGA |
| | heavy | GCTTGTGAAGCCTGGGGCTTCAGTGAAGATGTCCTGCAAGGCT |
| | chain | TCTGGCTACACCTTCACCAGCTACTGGATAACCTGGGTGAAGC |
| | nucleic | AGAGGCCTGGACAAGGCCTTGAGTGGATTGGAGATATTTATCCT |
| | acid | GGTAGTGGTAGTACTACCTACAATGAGAAGTTCAAGAGCAAGGCC |
| | | ACACTGACTGTTGACACATCCTCCAGCACAGCCTACATGCAGT |
| | | TCAGCAGCCTGACATCTGAGGACTCTGCGGTCTATTACTGTGC |
| | | AAGAAGGCATTACGGTAGTACCTCGGCCTGGTTTGCTTACT |
| | | GGGGCCAGGGGACTCTGGTCACTGTCTCTGCAGCCAAAACGAC |
| | | ACCCCCATCTGTCTATCCACTGGCCCCTGGATCTGCTGCCCAAA |
| | | CTAACTCCATGGTGACCCTGGGATGCCTGGTCAAGGGCTATTT |
| | | CCCTGAGCCAGTGACAGTGACCTGGAACTCTGGATCCCTGTCC |
| | | AGCGGTGTGCACACCTTCCCAGCTGTCCTGCAGTCTGACCTCTA |
| | | CACTCTGAGCAGCTCAGTGACTGTCCCCTCCAGCACCTGGCCC |
| | | AGCCAGACCGTCACCTGCAACGTTGCCCACCCGGCCAGCAGCA |
| | | CCAAGGTGGACAAGAAAATTGTGCCCAGGGATTGTGGTTGTAA |
| | | GCCTTGCATATGTACAGTCCCAGAAGTATCATCTGTCTTCATCT |
| | | TCCCCCCAAAGCCCAAGGATGTGCTCACCATTACTCTGACTCCT |
| | | AAGGTCACGTGTGTTGTGGTAGACATCAGCAAGGATGATCCCG |
| | | AGGTCCAGTTCAGCTGGTTTGTAGATGATGTGGAGGTGCACAC |
| | | AGCTCAGACGAAACCCCGGGAGGAGCAGATCAACAGCACTTT |
| | | CCGTTCAGTCAGTGAACTTCCCATCATGCACCAGGACTGGCTC |
| | | AATGGCAAGGAGTTCAAATGCAGGGTCAACAGTGCAGCTTTCC |
| | | CTGCCCCCATCGAGAAAACCATCTCCAAAACCAAAGGCAGACC |
| | | GAAGGCTCCACAGGTGTACACCATTCCACCTCCCAAGGAGCAG |
| | | ATGGCCAAGGATAAAGTCAGTCTGACCTGCATGATAACAAACT |
| | | TCTTCCCTGAAGACATTACTGTGGAGTGGCAGTGGAATGGGCA |
| | | GCCAGCGGAGAACTACAAGAACACTCAGCCCATCATGGACAC |
| | | AGATGGCTCTTACTTCGTCTACAGCAAGCTCAATGTGCAGAAG |
| | | AGCAACTGGGAGGCAGGAAATACTTTCACCTGCTCTGTGTTAC |
| | | ATGAGGGCCTGCACAACCACCATACTGAGAAGAGCCTCTCCCA |
| | | CTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 224 | U5A mAb | MSSAQFLGLLLLCFQGTRCDIQMTQTTSSLSASLGDRVTISCSASQG |
| | 2E6_1 | IRNYLNWYQQKPDGTVKLLIYYTSSLHSGVPSRFSGSGSGTDYSL |
| | light chain | TISNLEPEDIATYFCQQYSKLPFTFGSGTQLEIKRADAAPTVSIFPP |
| | | SSEQLTSGGASVVCFLNNFYPKDINVKWKIDGSERQNGVLNSWT |
| | | DQDSKDSTYSMSSTLTLTKDEYERHNSYTCEATHKTSTSPIVKSFN |
| | | RNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 225 | U5A mAb | DIQMTQTTSSLSASLGDRVTISCSASQGIRNYLNWYQQKPDGTVK |
| | 2E6_1 | LLIYYTSSLHSGVPSRFSGSGSGTDYSLTISNLEPEDIATYFCQQYS |
| | light chain | KLPFTFGSGTQLEIKRADAAPTVSIFPPSSEQLTSGGASVVCFLNNF |
| | w/o signal | YPKDINVKWKIDGSERQNGVLNSWTDQDSKDSTYSMSSTLTLTK |
| | peptide | DEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 226 | U5A mAb | DIQMTQTTSSLSASLGDRVTISCSASQGIRNYLNWYQQKPDGTVK |
| | 2E6_1 | LLIYYTSSLHSGVPSRFSGSGSGTDYSLTISNLEPEDIATYFCQQYS |
| | light chain | KLPFTFGSGTQLEIK |
| | variable | |
| | region | |
| 227 | U5A mAb | SASQGIRNYLN |
| | 2E6_1 | |
| | light chain | |
| | CDR1 | |
| 228 | U5A mAb | YTSSLHS |
| | 2E6_1 | |
| | light chain | |
| | CDR2 | |
| 229 | U5A mAb | QQYSKLPFT |
| | 2E6_1 | |
| | light chain | |
| | CDR3 | |
| 230 | U5A mAb | ATGTCCTCTGCTCAGTTCCTTGGTCTCCTGTTGCTCTGTTTTCAAG |
| | 2E6_1 | GTACCAGATGTGATATCCAGATGACACAGACTACATCCTCCCTG |
| | light chain | TCTGCCTCTCTGGGAGACAGAGTCACCATCAGTTGCAGTGCA |
| | nucleic | AGTCAGGGCATTAGGAATTATTTAAACTGGTATCAGCAGAA |
| | acid | ACCAGATGGAACTGTTAAACTCCTGATCTATTACACATCAAG |
| | | TTTACACTCAGGAGTCCCATCAAGGTTCAGTGGCAGTGGGTC |
| | | TGGGACAGATTATTCTCTCACCATCAGCAACCTGGAACCTGAA |
| | | GATATTGCCACTTACTTTTGTCAGCAATATAGTAAGCTTCCA |
| | | TTCACGTTCGGCTCGGGGACACAGTTGGAAATAAAACGGGCT |
| | | GATGCTGCACCAACTGTATCCATCTTCCCACCATCCAGTGAGC |
| | | AGTTAACATCTGGAGGTGCCTCAGTCGTGTGCTTCTTGAACAA |
| | | CTTCTACCCCAAAGACATCAATGTCAAGTGGAAGATTGATGGC |
| | | AGTGAACGACAAAATGGCGTCCTGAACAGTTGGACTGATCAG |
| | | GACAGCAAAGACAGCACCTACAGCATGAGCAGCACCCTCACG |
| | | TTGACCAAGGACGAGTATGAACGACATAACAGCTATACCTGT |
| | | GAGGCCACTCACAAGACATCAACTTCACCCATTGTCAAGAGCT |
| | | TCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 231 | U5A mAb | MEWSWVSLFFLSLTTGVHSQVQLQQSDAELVKPGASVKISCKVSGY |
| | 3B3_1 | TFTDHTIHWMKQWPEQGLEWIGYIYPRDGGTKYSEKFKGKATL |
| | heavy | TADKSSSTASMQLNRLTSEDSAVYFCARGQLDYWGQGTTLTVSS |
| | chain | AKTTPPSVYPLAPGSAAQTNSMVTLGCLVKGYFPEPVTVTWNSGS |
| | | LSSGVHTFPAVLQSDLYTLSSSVTVPSSTWPSQTVTCNVAHPASST |
| | | KVDKKIVPRDCGCKPCICTVPEVSSVFIFPPKPKDVLTITLTPKVTC |
| | | VVVDISKDDPEVQFSWFVDDVEVHTAQTKPREEQINSTFRSVSELP |
| | | IMHQDWLNGKEFKCRVNSAAFPAPIEKTISKTKGRPKAPQVYTIPP |
| | | PKEQMAKDKVSLTCMITNFFPEDITVEWQWNGQPAENYKNTQPI |
| | | MDTDGSYFVYSKLNVQKSNWEAGNTFTCSVLHEGLHNHHTEKSL |
| | | SHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 232 | U5A mAb | QVQLQQSDAELVKPGASVKISCKVSGYTFTDHTIHWMKQWPEQ |
| | 3B3_1 | GLEWIGYIYPRDGGTKYSEKFKGKATLTADKSSSTASMQLNRLT |
| | heavy | SEDSAVYFCARGQLDYWGQGTTLTVSSAKTTPPSVYPLAPGSAA |
| | chain | QTNSMVTLGCLVKGYFPEPVTVTWNSGSLSSGVHTFPAVLQSDL |
| | protein | YTLSSSVTVPSSTWPSQTVTCNVAHPASSTKVDKKIVPRDCGCKP |
| | w/o signal | CICTVPEVSSVFIFPPKPKDVLTITLTPKVTCVVVDISKDDPEVQFS |
| | peptide | WFVDDVEVHTAQTKPREEQINSTFRSVSELPIMHQDWLNGKEFKC |
| | | RVNSAAFPAPIEKTISKTKGRPKAPQVYTIPPPKEQMAKDKVSLTC |
| | | MITNFFPEDITVEWQWNGQPAENYKNTQPIMDTDGSYFVYSKLN |
| | | VQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 233 | U5A mAb | QVQLQQSDAELVKPGASVKISCKVSGYTFTDHTIHWMKQWPEQ |
| | 3B3_1 | GLEWIGYIYPRDGGTKYSEKFKGKATLTADKSSSTASMQLNRLT |
| | heavy | SEDSAVYFCARGQLDYWGQGTTLTVSS |
| | chain | |
| | variable | |
| | region | |
| 234 | U5A mAb | DHTIH |
| | 3B3_1 | |
| | heavy | |
| | chain | |
| | CDR1 | |
| 235 | U5A mAb | YIYPRDGGTKYSEKFKG |
| | 3B3_1 | |
| | heavy | |
| | chain | |
| | CDR2 | |
| 236 | U5A mAb | GQLDY |
| | 3B3_1 | |
| | heavy | |
| | chain | |
| | CDR3 | |
| 237 | U5A mAb | ATGGAATGGAGCTGGGTCTCTCTCTTCTTCCTGTCACTAACTACAG |
| | 3B3_1 | GTGTCCACTCCCAGGTTCAGCTGCAACAGTCTGACGCTGAGTT |
| | heavy | GGTGAAACCTGGAGCTTCAGTGAAGATATCCTGCAAGGTTTCT |
| | chain | GGCTACACCTTCACTGACCATACTATTCACTGGATGAAACAG |
| | nucleic | TGGCCTGAACAGGGCCTGGAATGGATTGGATATATTTATCCT |
| | acid | AGAGATGGTGGTACTAAGTACAGTGAGAAGTTCAAGGGCA |
| | | AGGCCACATTGACTGCAGACAAATCCTCCAGCACAGCCTCCAT |
| | | GCAGCTCAACAGGCTGACATCTGAGGACTCTGCAGTCTATTTC |
| | | TGTGCAAGAGGTCAACTAGACTACTGGGGCCAAGGCACCAC |
| | | TCTCACAGTCTCCTCAGCCAAAACGACACCCCCATCTGTCTAT |
| | | CCACTGGCCCCTGGATCTGCTGCCCAAACTAACTCCATGGTGA |
| | | CCCTGGGATGCCTGGTCAAGGGCTATTTCCCTGAGCCAGTGAC |
| | | AGTGACCTGGAACTCTGGATCCCTGTCCAGCGGTGTGCACACC |
| | | TTCCCAGCTGTCCTGCAGTCTGACCTCTACACTCTGAGCAGCT |
| | | CAGTGACTGTCCCCTCCAGCACCTGGCCCAGCCAGACCGTCAC |
| | | CTGCAACGTTGCCCACCCGGCCAGCAGCACCAAGGTGGACAA |
| | | GAAAATTGTGCCCAGGGATTGTGGTTGTAAGCCTTGCATATGT |
| | | ACAGTCCCAGAAGTATCATCTGTCTTCATCTTCCCCCCAAAGC |
| | | CCAAGGATGTGCTCACCATTACTCTGACTCCTAAGGTCACGTG |
| | | TGTTGTGGTAGACATCAGCAAGGATGATCCCGAGGTCCAGTTC |
| | | AGCTGGTTTGTAGATGATGTGGAGGTGCACACAGCTCAGACG |
| | | AAACCCCGGGAGGAGCAGATCAACAGCACTTTCCGTTCAGTC |
| | | AGTGAACTTCCCATCATGCACCAGGACTGGCTCAATGGCAAG |
| | | GAGTTCAAATGCAGGGTCAACAGTGCAGCTTTCCCTGCCCCCA |
| | | TCGAGAAAACCATCTCCAAAACCAAAGGCAGACCGAAGGCTC |
| | | CACAGGTGTACACCATTCCACCTCCCAAGGAGCAGATGGCCA |
| | | AGGATAAAGTCAGTCTGACCTGCATGATAACAAACTTCTTCCC |
| | | TGAAGACATTACTGTGGAGTGGCAGTGGAATGGGCAGCCAGC |
| | | GGAGAACTACAAGAACACTCAGCCCATCATGGACACAGATGG |
| | | CTCTTACTTCGTCTACAGCAAGCTCAATGTGCAGAAGAGCAAC |
| | | TGGGAGGCAGGAAATACTTTCACCTGCTCTGTGTTACATGAGG |
| | | GCCTGCACAACCACCATACTGAGAAGAGCCTCTCCCACTCTCC |
| | | TGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 238 | U5A mAb | MKLPVRLLVLMFWIPASSSDVVMTQTPLSLPASLGDQASISCRSSQS |
| | 3B3_1 | LVHSSGNTYLYWYLQKPGQSPKLLIYRVSNRFSGVPDRFSGSGS |
| | light chain | GTDFTLKISRVEADDLGVYFCSQSTHVPFTFGGGTKLEIKRADAA |
| | | PTVSIFPPSSEQLTSGGASVVCFLNNFYPKDINVKWKIDGSERQNG |
| | | VLNSWTDQDSKDSTYSMSSTLTLTKDEYERHNSYTCEATHKTSTS |
| | | PIVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 239 | U5A mAb | DVVMTQTPLSLPASLGDQASISCRSSQSLVHSSGNTYLYWYLQK |
| | 3B3_1 | PGQSPKLLIYRVSNRFSGVPDRFSGSGSGTDFTLKISRVEADDLGV |
| | light chain | YFCSQSTHVPFTFGGGTKLEIKRADAAPTVSIFPPSSEQLTSGGAS |
| | w/o signal | VVCFLNNFYPKDINVKWKIDGSERQNGVLNSWTDQDSKDSTYSM |
| | peptide | SSTLTLTKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 240 | U5A mAb | DVVMTQTPLSLPASLGDQASISCRSSQSLVHSSGNTYLYWYLQK |
| | 3B3_1 | PGQSPKLLIYRVSNRFSGVPDRFSGSGSGTDFTLKISRVEADDLGV |
| | light chain | YFCSQSTHVPFTFGGGTKLEIK |
| | variable | |
| | region | |
| 241 | U5A mAb | RSSQSLVHSSGNTYLY |
| | 3B3_1 | |
| | light chain | |
| | CDR1 | |
| 242 | U5A mAb | RVSNRFS |
| | 3B3_1 | |
| | light chain | |
| | CDR2 | |
| 243 | U5A mAb | SQSTHVPFT |
| | 3B3_1 | |
| | light chain | |
| | CDR3 | |
| 244 | U5A mAb | ATGAAGTTGCCTGTTAGGCTGTTGGTGCTGATGTTCTGGATTCCTG |
| | 3B3_1 | CTTCCAGCAGTGATGTTGTGATGACCCAAACTCCACTCTCCCTG |
| | light chain | CCTGCCAGTCTTGGAGATCAAGCCTCCATCTCTTGCAGATCTA |
| | nucleic | GTCAGAGCCTTGTACACAGTAGTGGAAACACCTATTTATA |
| | acid | TTGGTATCTGCAGAAGCCAGGCCAGTCTCCAAAGCTCCTGATC |
| | | TACAGAGTTTCCAACCGATTTTCTGGGGTCCCAGACAGGTTC |
| | | AGTGGCAGTGGATCAGGGACAGATTTCACACTCAAGATCAGC |
| | | AGAGTGGAGGCTGACGATCTGGGAGTTTATTTCTGCTCTCAA |
| | | AGTACACATGTTCCGTTCACGTTCGGAGGGGGGACCAAGCT |
| | | GGAAATAAAACGGGCTGATGCTGCACCAACTGTATCCATCTT |
| | | CCCACCATCCAGTGAGCAGTTAACATCTGGAGGTGCCTCAGTC |
| | | GTGTGCTTCTTGAACAACTTCTACCCCAAAGACATCAATGTCA |
| | | AGTGGAAGATTGATGGCAGTGAACGACAAAATGGCGTCCTGA |
| | | ACAGTTGGACTGATCAGGACAGCAAAGACAGCACCTACAGCA |
| | | TGAGCAGCACCCTCACGTTGACCAAGGACGAGTATGAACGAC |
| | | ATAACAGCTATACCTGTGAGGCCACTCACAAGACATCAACTT |
| | | CACCCATTGTCAAGAGCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 245 | U5A mAb | MELCWVFLFLLSVTAGVHSQVQLQQSGAELVKPGASVKLSCKSSG |
| | 16G1_1 | NSFNEYIINWVKQRPGQGLEWIGWFYPGSGTKKCNEKFKAKAT |
| | heavy | LTADKSSSTVYMELSGLTSEDSAVYFCTRHEYYNGSSLGFAYWG |
| | chain | QGTLVSVSAAKTTPPSVYPLAPGSAAQTNSMVTLGCLVKGYFPEP |
| | | VTVTWNSGSLSSGVHTFPAVLQSDLYTLSSSVTVPSSTWPSQTVT |
| | | CNVAHPASSTKVDKKIVPRDCGCKPCICTVPEVSSVFIFPPKPKDV |
| | | LTITLTPKVTCVVVDISKDDPEVQFSWFVDDVEVHTAQTKPREEQI |
| | | NSTFRSVSELPIMHQDWLNGKEFKCRVNSAAFPAPIEKTISKTKGR |
| | | PKAPQVYTIPPPKEQMAKDKVSLTCMITNFFPEDITVEWQWNGQP |
| | | AENYKNTQPIMDTDGSYFVYSKLNVQKSNWEAGNTFTCSVLHEG |
| | | LHNHHTEKSLSHSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 246 | U5A mAb | QVQLQQSGAELVKPGASVKLSCKSSGNSFNEYIINWVKQRPGQG |
| | 16G1_1 | LEWIGWFYPGSGTKKCNEKFKAKATLTADKSSSTVYMELSGLT |
| | heavy | SEDSAVYFCTRHEYYNGSSLGFAYWGQGTLVSVSAAKTTPPSVY |
| | chain | PLAPGSAAQTNSMVTLGCLVKGYFPEPVTVTWNSGSLSSGVHTFP |
| | protein | AVLQSDLYTLSSSVTVPSSTWPSQTVTCNVAHPASSTKVDKKIVP |
| | w/o signal | RDCGCKPCICTVPEVSSVFIFPPKPKDVLTITLTPKVTCVVVDISKD |
| | peptide | DPEVQFSWFVDDVEVHTAQTKPREEQINSTFRSVSELPIMHQDWL |
| | | NGKEFKCRVNSAAFPAPIEKTISKTKGRPKAPQVYTIPPPKEQMAK |
| | | DKVSLTCMITNFFPEDITVEWQWNGQPAENYKNTQPIMDTDGSYF |
| | | VYSKLNVQKSNWEAGNTFTCSVLHEGLHNHHTEKSLSHSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 247 | U5A mAb | QVQLQQSGAELVKPGASVKLSCKSSGNSFNEYIINWVKQRPGQG |
| | 16G1_1 | LEWIGWFYPGSGTKKCNEKFKAKATLTADKSSSTVYMELSGLT |
| | heavy | SEDSAVYFCTRHEYYNGSSLGFAYWGQGTLVSVSA |
| | chain | |
| | variable | |
| | region | |
| 248 | U5A mAb | EYIIN |
| | 16G1_1 | |
| | heavy | |
| | chain | |
| | CDR1 | |
| 249 | U5A mAb | WFYPGSGTKKCNEKFKA |
| | 16G1_1 | |
| | heavy | |
| | chain | |
| | CDR2 | |
| 250 | U5A mAb | HEYYNGSSLGFAY |
| | 16G1_1 | |
| | heavy | |
| | chain | |
| | CDR3 | |
| 251 | U5A mAb | ATGGAATTGTGCTGGGTCTTTCTCTTCCTCCTGTCAGTAACTGCAG |
| | 16G1_1 | GTGTCCACTCCCAGGTCCAGCTTCAGCAGTCTGGAGCTGAACT |
| | heavy | GGTGAAACCCGGGGCATCAGTGAAGCTGTCCTGCAAGTCTTCT |
| | chain | GGCAACTCCTTCAATGAGTATATTATAAATTGGGTTAAACAG |
| | nucleic | AGGCCTGGACAGGGTCTTGAGTGGATCGGGTGGTTTTACCCT |
| | acid | GGAAGCGGTACTAAAAAGTGCAATGAGAAATTTAAGGCCA |
| | | AGGCCACATTGACTGCGGACAAATCCTCCAGTACAGTCTACAT |
| | | GGAACTAAGTGGATTGACATCTGAAGACTCTGCGGTCTATTTC |
| | | TGTACAAGACACGAATATTATAACGGAAGTAGTCTAGGGTT |
| | | TGCTTACTGGGGCCAAGGGACACTGGTCAGTGTCTCTGCAGC |
| | | CAAAACGACACCCCCATCTGTCTATCCACTGGCCCCTGGATCT |
| | | GCTGCCCAAACTAACTCCATGGTGACCCTGGGATGCCTGGTCA |
| | | AGGGCTATTTCCCTGAGCCAGTGACAGTGACCTGGAACTCTGG |
| | | ATCCCTGTCCAGCGGTGTGCACACCTTCCCAGCTGTCCTGCAG |
| | | TCTGACCTCTACACTCTGAGCAGCTCAGTGACTGTCCCCTCCA |
| | | GCACCTGGCCCAGCCAGACCGTCACCTGCAACGTTGCCCACCC |
| | | GGCCAGCAGCACCAAGGTGGACAAGAAAATTGTGCCCAGGGA |
| | | TTGTGGTTGTAAGCCTTGCATATGTACAGTCCCAGAAGTATCA |
| | | TCTGTCTTCATCTTCCCCCCAAAGCCCAAGGATGTGCTCACCA |
| | | TTACTCTGACTCCTAAGGTCACGTGTGTTGTGGTAGACATCAG |
| | | CAAGGATGATCCCGAGGTCCAGTTCAGCTGGTTTGTAGATGAT |
| | | GTGGAGGTGCACACAGCTCAGACGAAACCCCGGGAGGAGCAG |
| | | ATCAACAGCACTTTCCGTTCAGTCAGTGAACTTCCCATCATGC |
| | | ACCAGGACTGGCTCAATGGCAAGGAGTTCAAATGCAGGGTCA |
| | | ACAGTGCAGCTTTCCCTGCCCCCATCGAGAAAACCATCTCCAA |
| | | AACCAAAGGCAGACCGAAGGCTCCACAGGTGTACACCATTCC |
| | | ACCTCCCAAGGAGCAGATGGCCAAGGATAAAGTCAGTCTGAC |
| | | CTGCATGATAACAAACTTCTTCCCTGAAGACATTACTGTGGAG |
| | | TGGCAGTGGAATGGGCAGCCAGCGGAGAACTACAAGAACACT |
| | | CAGCCCATCATGGACACAGATGGCTCTTACTTCGTCTACAGCA |
| | | AGCTCAATGTGCAGAAGAGCAACTGGGAGGCAGGAAATACTT |
| | | TCACCTGCTCTGTGTTACATGAGGGCCTGCACAACCACCATAC |
| | | TGAGAAGAGCCTCTCCCACTCTCCTGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 252 | U5A mAb | MRCLAEFLGLLVLWIPGAIGDIVMTQAAPSVTVTPGESVTISCRSSK |
| | 16G1_1 | SLLHSYGSTYLYWFLQRPGQSPQLLIYRMSNLASGVPDRFSGSGS |
| | light chain | GTAFTLRISRVEAEDVGVYYCMQHLEYPLTFGAGTKLELKRADA |
| | | APTVSIFPPSSEQLTSGGASVVCFLNNFYPKDINVKWKIDGSERQN |
| | | GVLNSWTDQDSKDSTYSMSSTLTLTKDEYERHNSYTCEATHKTS |
| | | TSPIVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 253 | U5A mAb | DIVMTQAAPSVTVTPGESVTISCRSSKSLLHSYGSTYLYWFLQRP |
| | 16G1_1 | GQSPQLLIYRMSNLASGVPDRFSGSGSGTAFTLRISRVEAEDVGV |
| | light chain | YYCMQHLEYPLTFGAGTKLELKRADAAPTVSIFPPSSEQLTSGGA |
| | w/o signal | SVVCFLNNFYPKDINVKWKIDGSERQNGVLNSWTDQDSKDSTYS |
| | peptide | MSSTLTLTKDEYERHNSYTCEATHKTSTSPIVKSFNRNEC |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 254 | U5A mAb | DIVMTQAAPSVTVTPGESVTISCRSSKSLLHSYGSTYLYWFLQRP |
| | 16G1_1 | GQSPQLLIYRMSNLASGVPDRFSGSGSGTAFTLRISRVEAEDVGV |
| | light chain | YYCMQHLEYPLTFGAGTKLELK |
| | variable | |
| | region | |
| 255 | U5A mAb | RSSKSLLHSYGSTYLY |
| | 16G1_1 | |
| | light chain | |
| | CDR1 | |
| 256 | U5A mAb | RMSNLAS |
| | 16G1_1 | |
| | light chain | |
| | CDR2 | |
| 257 | U5A mAb | MQHLEYPLT |
| | 16G1_1 | |
| | light chain | |
| | CDR3 | |
| 258 | U5A mAb | ATGAGGTGCCTAGCTGAGTTCCTGGGGCTGCTTGTGCTCTGGATC |
| | 16G1_1 | CCTGGAGCCATTGGGGATATTGTGATGACTCAGGCTGCACCCT |
| | light chain | CTGTAACTGTCACTCCTGGAGAGTCAGTAACCATCTCCTGCA |
| | nucleic | GGTCTAGTAAGAGTCTCCTCCATAGTTATGGCAGCACTTA |
| | acid | CTTGTATTGGTTCCTACAGAGGCCAGGCCAGTCTCCTCAACT |
| | | CCTGATATATCGGATGTCCAACCTTGCCTCAGGAGTCCCAG |
| | | ACAGGTTCAGTGGCAGTGGGTCAGGAACTGCTTTCACACTGA |
| | | GAATCAGCAGAGTGGAGGCTGAGGATGTGGGTGTTTATTACT |
| | | GTATGCAACATCTAGAATATCCGCTCACGTTCGGTGCTGGG |
| | | ACCAAGCTGGAGTTGAAACGGGCTGATGCTGCACCAACTGTA |
| | | TCCATCTTCCCACCATCCAGTGAGCAGTTAACATCTGGAGGT |
| | | GCCTCAGTCGTGTGCTTCTTGAACAACTTCTACCCCAAAGACA |
| | | TCAATGTCAAGTGGAAGATTGATGGCAGTGAACGACAAAATG |
| | | GCGTCCTGAACAGTTGGACTGATCAGGACAGCAAAGACAGC |
| | | ACCTACAGCATGAGCAGCACCCTCACGTTGACCAAGGACGAG |
| | | TATGAACGACATAACAGCTATACCTGTGAGGCCACTCACAAG |
| | | ACATCAACTTCACCCATTGTCAAGAGCTTCAACAGGAATGAG |
| | | TGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 259 | ALG5 | MYFRLSSVFLVLILKGVQCEVKLVESEGGLVQPGSSMKLSCTASGFT |
| | mAb | FSDYYMAWVRQVPEKGLEWVANINYDGSSTYYLDSLKSRFIISR |
| | 6A7_1 | DNAKNILYLQMSSLKSEDTATYYCARDSYYYGRSYGYFDVWGT |
| | heavy | GTTVTVSSAKTTPPSVYPLAPGCGDTTGSSVTLGCLVKGYFPESVT |
| | chain | VTWNSGSLSSSVHTFPALLQSGLYTMSSSVTVPSSTWPSQTVTCSV |
| | | AHPASSTTVDKKLEPSGPISTINPCPPCKECHKCPAPNLEGGPSVFIF |
| | | PPNIKDVLMISLTPKVTCVVVDVSEDDPDVRISWFVNNVEVHTAQ |
| | | TQTHREDYNSTIRVVSALPIQHQDWMSGKEFKCKVNNKDLPSPIE |
| | | RTISKIKGLVRAPQVYILPPPAEQLSRKDVSLTCLVVGFNPGDISVE |
| | | WTSNGHTEENYKDTAPVLDSDGSYFIYSKLDIKTSKWEKTDSFSC |
| | | NVRHEGLKNYYLKKTISRSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 260 | ALG5 | EVKLVESEGGLVQPGSSMKLSCTASGFTFSDYYMAWVRQVPEKG |
| | mAb | LEWVANINYDGSSTYYLDSLKSRFIISRDNAKNILYLQMSSLKSE |
| | 6A7_1 | DTATYYCARDSYYYGRSYGYFDVWGTGTTVTVSSAKTTPPSVYP |
| | heavy | LAPGCGDTTGSSVTLGCLVKGYFPESVTVTWNSGSLSSSVHTFPA |
| | chain | LLQSGLYTMSSSVTVPSSTWPSQTVTCSVAHPASSTTVDKKLEPSG |
| | protein | PISTINPCPPCKECHKCPAPNLEGGPSVFIFPPNIKDVLMISLTPKVT |
| | w/o signal | CVVVDVSEDDPDVRISWFVNNVEVHTAQTQTHREDYNSTIRVVS |
| | peptide | ALPIQHQDWMSGKEFKCKVNNKDLPSPIERTISKIKGLVRAPQVYI |
| | | LPPPAEQLSRKDVSLTCLVVGFNPGDISVEWTSNGHTEENYKDTA |
| | | PVLDSDGSYFIYSKLDIKTSKWEKTDSFSCNVRHEGLKNYYLK |
| | | KTISRSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 261 | ALG5 | EVKLVESEGGLVQPGSSMKLSCTASGFTFSDYYMAWVRQVPEKG |
| | mAb | LEWVANINYDGSSTYYLDSLKSRFIISRDNAKNILYLQMSSLKSE |
| | 6A7_1 | DTATYYCARDSYYYGRSYGYFDVWGTGTTVTVSS |
| | heavy | |
| | chain | |
| | variable | |
| | region | |
| 262 | ALG5 | DYYMA |
| | mAb | |
| | 6A7_1 | |
| | heavy | |
| | chain | |
| | CDR1 | |
| 263 | ALG5 | NINYDGSSTYYLDSLKS |
| | mAb | |
| | 6A7_1 | |
| | heavy | |
| | chain | |
| | CDR2 | |
| 264 | ALG5 | DSYYYGRSYGYFDV |
| | mAb | |
| | 6A7_1 | |
| | heavy | |
| | chain | |
| | CDR3 | |
| 265 | ALG5 | ATGTACTTCAGGCTCAGCTCAGTTTTTCTTGTTCTTATTTTAAAAGGA |
| | mAb | GTCCAGTGTGAAGTGAAGCTGGTGGAGTCTGAGGGAGGCTTAG |
| | 6A7_1 | TGCAGCCTGGAAGTTCCATGAAACTCTCCTGCACAGCCTCTGG |
| | heavy | ATTCACTTTCAGTGACTATTACATGGCTTGGGTCCGCCAGGTT |
| | chain | CCAGAAAAGGGTCTTGAATGGGTTGCAAACATTAATTATGAT |
| | nucleic | GGTAGTAGTACCTACTATCTGGACTCCTTGAAGAGCCGTTT |
| | acid | CATCATCTCGAGAGACAATGCAAAGAACATTCTATACCTGCAA |
| | | ATGAGCAGTCTGAAGTCTGAGGACACAGCCACGTATTACTGTG |
| | | CAAGAGATTCCTATTACTACGGTCGTAGCTACGGGTACTTC |
| | | GATGTCTGGGGCACAGGGACCACGGTCACCGTCTCCTCAGCCA |
| | | AAACAACACCCCCATCAGTCTATCCACTGGCCCCTGGGTGTGG |
| | | AGATACAACTGGTTCCTCTGTGACTCTGGGATGCCTGGTCAAG |
| | | GGCTACTTCCCTGAGTCAGTGACTGTGACTTGGAACTCTGGATC |
| | | CCTGTCCAGCAGTGTGCACACCTTCCCAGCTCTCCTGCAGTCTG |
| | | GACTCTACACTATGAGCAGCTCAGTGACTGTCCCCTCCAGCAC |
| | | CTGGCCAAGTCAGACCGTCACCTGCAGCGTTGCTCACCCAGCC |
| | | AGCAGCACCACGGTGGACAAAAAACTTGAGCCCAGCGGGCCC |
| | | ATTTCAACAATCAACCCCTGTCCTCCATGCAAGGAGTGTCACA |
| | | AATGCCCAGCTCCTAACCTCGAGGGTGGACCATCCGTCTTCAT |
| | | CTTCCCTCCAAATATCAAGGATGTACTCATGATCTCCCTGACAC |
| | | CCAAGGTCACGTGTGTGGTGGTGGATGTGAGCGAGGATGACCC |
| | | AGACGTCCGGATCAGCTGGTTTGTGAACAACGTGGAAGTACAC |
| | | ACAGCTCAGACACAAACCCATAGAGAGGATTACAACAGTACT |
| | | ATCCGGGTGGTCAGTGCCCTCCCCATCCAGCACCAGGACTGGA |
| | | TGAGTGGCAAGGAGTTCAAATGCAAGGTCAACAACAAAGACC |
| | | TCCCATCACCCATCGAGAGAACCATCTCAAAAATTAAAGGGCT |
| | | AGTCAGAGCTCCACAAGTATACATCTTGCCGCCACCAGCAGAG |
| | | CAGTTGTCCAGGAAAGATGTCAGTCTCACTTGCCTGGTCGTGG |
| | | GCTTCAACCCTGGAGACATCAGTGTGGAGTGGACCAGCAATGG |
| | | GCATACAGAGGAGAACTACAAGGACACCGCACCAGTCCTGGA |
| | | CTCTGACGGTTCTTACTTCATATACAGCAAGCTCGATATAAAA |
| | | ACAAGCAAGTGGGAGAAAACAGATTCCTTCTCATGCAACGTGA |
| | | GACACGAGGGTCTGAAAAATTACTACCTGAAGAAGACCATCTC |
| | | CCGGTCTCCGGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 266 | ALG5 | MDSQAQVLMLLLLWVSGTCGDIVMSQSPSSLAVSVGEKVTMSCKS |
| | mAb | SQRLLYSSNQKNYLAWYQQKPGQSPKLLIYWASTRESGVPDRFT |
| | 6A7_1 | GSGSGTDFTLTISSVKAEDLAVYYCQQYYSYPPTFGAGTKLELKR |
| | light chain | ADAAPTVSIFPPSSEQLTSGGASVVCFLNNFYPKDINVKWKIDGSE |
| | | RQNGVLNSWTDQDSKDSTYSMSSTLTLTKDEYERHNSYTCETHK |
| | | TSTSPIVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 267 | ALG5 | DIVMSQSPSSLAVSVGEKVTMSCKSSQRLLYSSNQKNYLAWYQ |
| | mAb | QKPGQSPKLLIYWASTRESGVPDRFTGSGSGTDFTLTISSVKAEDL |
| | 6A7_1 | AVYYCQQYYSYPPTFGAGTKLELKRADAAPTVSIFPPSSEQLTSG |
| | light chain | GASVVCFLNNFYPKDINVKWKIDGSERQNGVLNSWTDQDSKDST |
| | w/o signal | YSMSSTLTLTKDEYERHNSYTCETHKTSTSPIVKSFNRNEC |
| | peptide | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 268 | ALG5 | DIVMSQSPSSLAVSVGEKVTMSCKSSQRLLYSSNQKNYLAWYQ |
| | mAb | QKPGQSPKLLIYWASTRESGVPDRFTGSGSGTDFTLTISSVKAEDL |
| | 6A7_1 | AVYYCQQYYSYPPTFGAGTKLELK |
| | light chain | |
| | variable | |
| | region | |
| 269 | ALG5 | KSSQRLLYSSNQKNYLA |
| | mAb | |
| | 6A7_1 | |
| | light chain | |
| | CDR1 | |
| 270 | ALG5 | WASTRES |
| | mAb | |
| | 6A7_1 | |
| | light chain | |
| | CDR2 | |
| 271 | ALG5 | QQYYSYPPT |
| | mAb | |
| | 6A7_1 | |
| | light chain | |
| | CDR3 | |
| 272 | ALG5 | ATGGATTCACAGGCCCAGGTTCTTATGTTACTGCTGCTATGGGTAT |
| | mAb | CTGGTACCTGTGGGGACATTGTGATGTCACAGTCTCCATCCTCC |
| | 6A7_1 | CTAGCTGTGTCAGTTGGAGAGAAGGTTACTATGAGCTGCAAG |
| | light chain | TCCAGTCAGAGACTTTTATATAGTAGCAATCAAAAGAACTA |
| | nucleic | CTTGGCCTGGTACCAGCAGAAACCAGGGCAGTCTCCTAAACT |
| | acid | GCTGATTTACTGGGCATCCACTAGGGAATCTGGGGTCCCTG |
| | | ATCGCTTCACAGGCAGTGGATCTGGGACAGATTTCACTCTCAC |
| | | CATCAGCAGTGTGAAGGCTGAAGACCTGGCAGTTTATTACTGT |
| | | CAGCAATATTATAGCTATCCTCCCACGTTCGGTGCTGGGAC |
| | | CAAGCTGGAGCTGAAACGGGCTGATGCTGCACCAACTGTATC |
| | | CATCTTCCCACCATCCAGTGAGCAGTTAACATCTGGAGGTGCC |
| | | TCAGTCGTGTGCTTCTTGAACAACTTCTACCCCAAAGACATCA |
| | | ATGTCAAGTGGAAGATTGATGGCAGTGAACGACAAAATGGCG |
| | | TCCTGAACAGTTGGACTGATCAGGACAGCAAAGACAGCACCT |
| | | ACAGCATGAGCAGCACCCTCACGTTGACCAAGGACGAGTATG |
| | | AACGACATAACAGCTATACCTGTGAGGCCACTCACAAGACAT |
| | | CAACTTCACCCATTGTCAAGAGCTTCAACAGGAATGAGTGTTA |
| | | G |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 273 | ALG5 | MYFRLSSVFLVLILKGVQCEVKLVESEGGLVQPGSSMKLSCTASGFT |
| | mAb | FSDYYMAWVRQVPEKGLEWVANINYDGSSTYYLDSLKSRFIISR |
| | 1D5_1 | DNAKNILYLQMSSLKSDDTATYYCARDSYYYGRFYGYFDVWGT |
| | heavy | GTTVTVSSAKTTAPSVYPLAPVCGGTTGSSVTLGCLVKGYFPEPV |
| | chain | TLTWNSGSLSSGVHTFPALLQSGLYTLSSSVTVTSNTWPSQTITCN |
| | | VAHPASSTKVDKKIEPRVPITQNPCPPLKECPPCAAPDLLGGPSVFI |
| | | FPPKIKDVLMISLSPMVTCVVVDVSEDDPDVQISWFVNNVEVHTA |
| | | QTQTHREDYNSTLRVVSALPIQHQDWMSGKEFKCKVNNRALPSPI |
| | | EKTISKPRGPVRAPQVYVLPPPAEEMTKKEFSLTCMITGFLPAEIA |
| | | VDWTSNGRTEQNYKNTATVLDSDGSYFMYSKLRVQKSTWERGS |
| | | LFACSVVHEGLHNHLTTKTISRSLGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 274 | ALG5 | EVKLVESEGGLVQPGSSMKLSCTASGFTFSDYYMAWVRQVPEKG |
| | mAb | LEWVANINYDGSSTYYLDSLKSRFIISRDNAKNILYLQMSSLKSD |
| | 1D5_1 | DTATYYCARDSYYYGRFYGYFDVWGTGTTVTVSSAKTTAPSVY |
| | heavy | PLAPVCGGTTGSSVTLGCLVKGYFPEPVTLTWNSGSLSSGVHTFP |
| | chain | ALLQSGLYTLSSSVTVTSNTWPSQTITCNVAHPASSTKVDKKIEPR |
| | protein | VPITQNPCPPLKECPPCAAPDLLGGPSVFIFPPKIKDVLMISLSPMVT |
| | w/o signal | CVVVDVSEDDPDVQISWFVNNVEVHTAQTQTHREDYNSTLRVVS |
| | peptide | ALPIQHQDWMSGKEFKCKVNNRALPSPIEKTISKPRGPVRAPQVY |
| | | VLPPPAEEMTKKEFSLTCMITGFLPAEIAVDWTSNGRTEQNYKNT |
| | | ATVLDSDGSYFMYSKLRVQKSTWERGSLFACSVVHEGLHNHLTT |
| | | KTISRSLGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 275 | ALG5 | EVKLVESEGGLVQPGSSMKLSCTASGFTFSDYYMAWVRQVPEKG |
| | mAb | LEWVANINYDGSSTYYLDSLKSRFIISRDNAKNILYLQMSSLKSD |
| | 1D5_1 | DTATYYCARDSYYYGRFYGYFDVWGTGTTVTVSS |
| | heavy | |
| | chain | |
| | variable | |
| | region | |
| 276 | ALG5 | DYYMA |
| | mAb | |
| | 1D5_1 | |
| | heavy | |
| | chain | |
| | CDR1 | |
| 277 | ALG5 | NINYDGSSTYYLDSLKS |
| | mAb | |
| | 1D5_1 | |
| | heavy | |
| | chain | |
| | CDR2 | |
| 278 | ALG5 | DSYYYGRFYGYFDV |
| | mAb | |
| | 1D5_1 | |
| | heavy | |
| | chain | |
| | CDR3 | |
| 279 | ALG5 | ATGTACTTCAGGCTCAGCTCAGTTTTTCTTGTTCTTATTTTAAAAG |
| | mAb | GAGTTCAGTGTGAAGTGAAGCTGGTGGAGTCTGAGGGAGGCTTAG |
| | 1D5_1 | TGCAGCCTGGAAGTTCCATGAAACTCTCCTGCACAGCCTCTGG |
| | heavy | ATTCACTTTCAGTGACTATTACATGGCTTGGGTCCGCCAGGTT |
| | chain | CCAGAAAAGGGTCTTGAGTGGGTTGCAAACATTAATTATGAT |
| | nucleic | GGTAGTAGCACCTACTATCTGGACTCCTTGAAGAGCCGTTT |
| | acid | CATCATCTCGAGAGACAATGCAAAGAACATTCTATACCTGCAA |
| | | ATGAGCAGTCTGAAGTCTGACGACACAGCCACGTATTACTGTG |
| | | CACGAGATTCCTATTACTACGGTCGTTTCTACGGGTACTTC |
| | | GATGTCTGGGGCACAGGGACCACGGTCACCGTCTCCTCAGCCA |
| | | AAACAACAGCCCCATCGGTCTATCCACTGGCCCCTGTGTGTGG |
| | | AGGTACAACTGGCTCCTCGGTGACTCTAGGATGCCTGGTCAAG |
| | | GGTTATTTCCCTGAGCCAGTGACCTTGACCTGGAACTCTGGATC |
| | | CCTGTCCAGTGGTGTGCACACCTTCCCAGCTCTCCTGCAGTCTG |
| | | GCCTCTACACCCTCAGCAGCTCAGTGACTGTAACCTCGAACAC |
| | | CTGGCCCAGCCAGACCATCACCTGCAATGTGGCCCACCCGGCA |
| | | AGCAGCACCAAAGTGGACAAGAAAATTGAGCCCAGAGTGCCC |
| | | ATAACACAGAACCCCTGTCCTCCACTCAAAGAGTGTCCCCCAT |
| | | GCGCAGCTCCAGACCTCTTGGGTGGACCATCCGTCTTCATCTTC |
| | | CCTCCAAAGATCAAGGATGTACTCATGATCTCCCTGAGCCCCA |
| | | TGGTCACATGTGTGGTGGTGGATGTGAGCGAGGATGACCCAGA |
| | | CGTCCAGATCAGCTGGTTTGTGAACAACGTGGAAGTACACACA |
| | | GCTCAGACACAAACCCATAGAGAGGATTACAACAGTACTCTCC |
| | | GGGTGGTCAGTGCCCTCCCCATCCAGCACCAGGACTGGATGAG |
| | | TGGCAAGGAGTTCAAATGCAAGGTCAACAACAGAGCCCTCCCA |
| | | TCCCCCATCGAGAAAACCATCTCAAAACCCAGAGGGCCAGTAA |
| | | GAGCTCCACAGGTATATGTCTTGCCTCCACCAGCAGAAGAGAT |
| | | GACTAAGAAAGAGTTCAGTCTGACCTGCATGATCACAGGCTTC |
| | | TTACCTGCCGAAATTGCTGTGGACTGGACCAGCAATGGGCGTA |
| | | CAGAGCAAAACTACAAGAACACCGCAACAGTCCTGGACTCTG |
| | | ATGGTTCTTACTTCATGTACAGCAAGCTCAGAGTACAAAAGAG |
| | | CACTTGGGAAAGAGGAAGTCTTTTCGCCTGCTCAGTGGTCCAC |
| | | GAGGGTCTGCACAATCACCTTACGACTAAGACCATCTCCCGGT |
| | | CTCTGGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 280 | ALG5 | MDSQAQVLMLLLLWVSGTCGDIVMSQSPSSLAVSVGEKVTMSCKS |
| | mAb | SQRLLYSTSQKNYLAWYQQKPGQSPKLLIYWASTRESGVPDRFT |
| | 1D5_1 | GSGSGTDFTLTISSVKAEDLAVYYCQQYYSYPPTFGAGTKLELKR |
| | light chain | ADAAPTVSIFPPSSEQLTSGGASVVCFLNNFYPKDINVKWKIDGSE |
| | | RQNGVLNSWTDQDSKDSTYSMSSTLTLTKDEYERHNSYTCEATH |
| | | KTSTSPIVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 281 | ALG5 | DIVMSQSPSSLAVSVGEKVTMSCKSSQRLLYSTSQKNYLAWYQQ |
| | mAb | KPGQSPKLLIYWASTRESGVPDRFTGSGSGTDFTLTISSVKAEDLA |
| | 1D5_1 | VYYCQQYYSYPPTFGAGTKLELKRADAAPTVSIFPPSSEQLTSGG |
| | light chain | ASVVCFLNNFYPKDINVKWKIDGSERQNGVLNSWTDQDSKDSTY |
| | w/o signal | SMSSTLTLTKDEYERHNSYTCEATHKTSTSPIVKSFNRNEC |
| | peptide | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 282 | ALG5 | DIVMSQSPSSLAVSVGEKVTMSCKSSQRLLYSTSQKNYLAWYQQ |
| | mAb | KPGQSPKLLIYWASTRESGVPDRFTGSGSGTDFTLTISSVKAEDLA |
| | 1D5_1 | VYYCQQYYSYPPTFGAGTKLELK |
| | light chain | |
| | variable | |
| | region | |
| 283 | ALG5 | KSSQRLLYSTSQKNYLA |
| | mAb | |
| | 1D5_1 | |
| | light chain | |
| | CDR1 | |
| 284 | ALG5 | WASTRES |
| | mAb | |
| | 1D5_1 | |
| | light chain | |
| | CDR2 | |
| 285 | ALG5 | QQYYSYPPT |
| | mAb | |
| | 1D5_1 | |
| | light chain | |
| | CDR3 | |
| 286 | ALG5 | ATGGATTCACAGGCCCAGGTTCTTATGTTACTGCTGCTATGGGTATC |
| | mAb | TGGTACCTGTGGGGACATTGTGATGTCACAGTCTCCATCCTCCC |
| | 1D5_1 | TAGCTGTGTCAGTTGGAGAGAAGGTTACTATGAGCTGCAAGTC |
| | light chain | CAGTCAGAGACTTTTATATAGTACCAGTCAAAAGAACTACT |
| | nucleic | TGGCCTGGTACCAGCAGAAGCCAGGGCAGTCTCCTAAACTGCT |
| | acid | GATTTACTGGGCATCCACTAGGGAATCTGGGGTCCCTGATCG |
| | | CTTCACAGGCAGTGGATCTGGGACAGATTTCACTCTCACCATC |
| | | AGCAGTGTGAAGGCTGAAGACCTGGCAGTTTATTACTGTCAGC |
| | | AATATTATAGCTATCCTCCCACGTTCGGTGCTGGGACCAAGC |
| | | TGGAGCTGAAACGGGCTGATGCTGCACCAACTGTATCCATCTT |
| | | CCCACCATCCAGTGAGCAGTTAACATCTGGAGGTGCCTCAGTC |
| | | GTGTGCTTCTTGAACAACTTCTACCCCAAAGACATCAATGTCA |
| | | AGTGGAAGATTGATGGCAGTGAACGACAAAATGGCGTCCTGA |
| | | ACAGTTGGACTGATCAGGACAGCAAAGACAGCACCTACAGCA |
| | | TGAGCAGCACCCTCACGTTGACCAAGGACGAGTATGAACGACA |
| | | TAACAGCTATACCTGTGAGGCCACTCACAAGACATCAACTTCA |
| | | CCCATTGTCAAGAGCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 287 | ALG5 | MGWSWIFLFLLSETAGVLSEVQLQQSGPELVKPGASVKMSCKAS |
| | mAb | GYTFTDSNMHWVKQSLGKSLEWIGYINTNNGGTTYNQKFKGTAT |
| | 2A11-1 | LTVNKSSSTAYMELRSLTSEDSAVYYCARADGYYWGQGTTLSVS |
| | heavy | SAKTTPPSVYPLAPGCGDTTGSSVTLGCLVKGYFPESVTVTWNSG |
| | chain | SLSSSVHTFPALLQSGLYTMSSSVTVPSSTWPSQTVTCSVAHPASS |
| | | TTVDKKLEPSGPISTINPCPPCKECHKCPAPNLEGGPSVFIFPPNI |
| | | KDVLMISLTPKVTCVVVDVSEDDPDVRISWFVNNVEVHTAQTQTHR |
| | | EDYNSTIRVVSALPIQHQDWMSGKEFKCKVNNKDLPSPIERTISKI |
| | | KGLVRAPQVYILPPPAEQLSRKDVSLTCLVVGFNPGDISVEWTSN |
| | | GHTEENYKDTAPVLDSDGSYFIYSKLDIKTSKWEKTDSFSCNVRH |
| | | EGLKNYYLKKTISRSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 288 | ALG5 | EVQLQQSGPELVKPGASVKMSCKASGYTFTDSNMHWVKQSLGK |
| | mAb | SLEWIGYINTNNGGTTYNQKFKGTATLTVNKSSSTAYMELRSLT |
| | 2A11-1 | SEDSAVYYCARADGYYWGQGTTLSVSSAKTTPPSVYPLAPGCGD |
| | heavy | TTGSSVTLGCLVKGYFPESVTVTWNSGSLSSSVHTFPALLQSGLYT |
| | chain | MSSSVTVPSSTWPSQTVTCSVAHPASSTTVDKKLEPSGPISTINPCP |
| | protein | PCKECHKCPAPNLEGGPSVFIFPPNIKDVLMISLTPKVTCVVVDVS |
| | w/o signal | EDDPDVRISWFVNNVEVHTAQTQTHREDYNSTIRVVSALPIQHQD |
| | peptide | WMSGKEFKCKVNNKDLPSPIERTISKIKGLVRAPQVYILPPPAEQL |
| | | SRKDVSLTCLVVGFNPGDISVEWTSNGHTEENYKDTAPVLDSDGS |
| | | YFIYSKLDIKTSKWEKTDSFSCNVRHEGLKNYYLKKTISRSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 289 | ALG5 | EVQLQQSGPELVKPGASVKMSCKASGYTFTDSNMHWVKQSLGK |
| | mAb | SLEWIGYINTNNGGTTYNQKFKGTATLTVNKSSSTAYMELRSLT |
| | 2A11-1 | SEDSAVYYCARADGYYWGQGTTLSVSS |
| | heavy | |
| | chain | |
| | variable | |
| | region | |
| 290 | ALG5 | DSNMH |
| | mAb | |
| | 2A11-1 | |
| | heavy | |
| | chain | |
| | CDR1 | |
| 291 | ALG5 | YINTNNGGTTYNQKFKG |
| | mAb | |
| | 2A11-1 | |
| | heavy | |
| | chain | |
| | CDR2 | |
| 292 | ALG5 | ADGYY |
| | mAb | |
| | 2A11-1 | |
| | heavy | |
| | chain | |
| | CDR3 | |
| 293 | ALG5 | ATGGGATGGAGCTGGATCTTTCTCTTTCTCCTGTCAGAAACTGCAG |
| | mAb | GTGTCCTCTCTGAGGTCCAGCTGCAACAGTCTGGACCTGAGCTG |
| | 2A11-1 | GTGAAGCCTGGGGCTTCAGTGAAGATGTCCTGCAAGGCTTCTG |
| | heavy | GATACACATTCACTGACTCCAACATGCACTGGGTGAAACAGA |
| | chain | GCCTTGGAAAGAGCCTTGAGTGGATTGGTTATATTAACACTAA |
| | nucleic | CAACGGTGGTACTACCTACAACCAGAAGTTCAAGGGCACGG |
| | acid | CCACATTGACTGTAAACAAGTCCTCCAGCACAGCCTACATGGA |
| | | GCTCCGCAGCCTGACATCGGAGGATTCTGCAGTCTATTACTGT |
| | | GCAAGAGCCGATGGTTACTACTGGGGCCAAGGCACCACTCTC |
| | | TCAGTCTCCTCAGCCAAAACAACACCCCCATCAGTCTATCCACT |
| | | GGCCCCTGGGTGTGGAGATACAACTGGTTCCTCTGTGACTCTG |
| | | GGATGCCTGGTCAAGGGCTACTTCCCTGAGTCAGTGACTGTGA |
| | | CTTGGAACTCTGGATCCCTGTCCAGCAGTGTGCACACCTTCCCA |
| | | GCTCTCCTGCAGTCTGGACTCTACACTATGAGCAGCTCAGTGA |
| | | CTGTCCCCTCCAGCACCTGGCCAAGTCAGACCGTCACCTGCAG |
| | | CGTTGCTCACCCAGCCAGCAGCACCACGGTGGACAAAAAACTT |
| | | GAGCCCAGCGGGCCCATTTCAACAATCAACCCCTGTCCTCCAT |
| | | GCAAGGAGTGTCACAAATGCCCAGCTCCTAACCTCGAGGGTGG |
| | | ACCATCCGTCTTCATCTTCCCTCCAAATATCAAGGATGTACTCA |
| | | TGATCTCCCTGACACCCAAGGTCACGTGTGTGGTGGTGGATGT |
| | | GAGCGAGGATGACCCAGACGTCCGGATCAGCTGGTTTGTGAAC |
| | | AACGTGGAAGTACACACAGCTCAGACACAAACCCATAGAGAG |
| | | GATTACAACAGTACTATCCGGGTGGTCAGTGCCCTCCCCATCC |
| | | AGCACCAGGACTGGATGAGTGGCAAGGAGTTCAAATGCAAGG |
| | | TCAACAACAAAGACCTCCCATCACCCATCGAGAGAACCATCTC |
| | | AAAAATTAAAGGGCTAGTCAGAGCTCCACAAGTATACATCTTG |
| | | CCGCCACCAGCAGAGCAGTTGTCCAGGAAAGATGTCAGTCTCA |
| | | CTTGCCTGGTCGTGGGCTTCAACCCTGGAGACATCAGTGTGGA |
| | | GTGGACCAGCAATGGGCATACAGAGGAGAACTACAAGGACAC |
| | | CGCACCAGTCCTGGACTCTGACGGTTCTTACTTCATATACAGCA |
| | | AGCTCGATATAAAAACAAGCAAGTGGGAGAAAACAGATTCCT |
| | | TCTCATGCAACGTGAGACACGAGGGTCTGAAAAATTACTACCT |
| | | GAAGAAGACCATCTCCCGGTCTCCGGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 294 | ALG5 | MESQTQVFVYMLLWLSGVDGDIVMTQSQKFMSTSVGDRVSVTCKA |
| | mAb | SQNVGTNVAWYQQKPGQSPKALIYSASSRYSGVPDRLTGSGSGT |
| | 2A11-1 | DFTLTISNVQSEDLAEYFCQQYNSFPLTFGAGTKLELKRADAAPT |
| | light chain | VSIFPPSSEQLTSGGASVVCFLNNFYPKDINVKWKIDGSERQNGVL |
| | | NSWTDQDSKDSTYSMSSTLTLTKDEYERHNSYTCEATHKTSTSPI |
| | | VKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 295 | ALG5 | DIVMTQSQKFMSTSVGDRVSVTCKASQNVGTNVAWYQQKPGQS |
| | mAb | PKALIYSASSRYSGVPDRLTGSGSGTDFTLTISNVQSEDLAEYFCQ |
| | 2A11-1 | QYNSFPLTFGAGTKLELKRADAAPTVSIFPPSSEQLTSGGASVVCF |
| | light chain | LNNFYPKDINVKWKIDGSERQNGVLNSWTDQDSKDSTYSMSSTL |
| | w/o signal | TLTKDEYERHNSYTCEATHKTSTSPIVKSFNRNEC |
| | peptide | |
| 296 | ALG5 | DIVMTQSQKFMSTSVGDRVSVTCKASQNVGTNVAWYQQKPGQSP |
| | mAb | KALIYSASSRYSGVPDRLTGSGSGTDFTLTISNVQSEDLAEYFC |
| | 2A11-1 | QQYNSFPLTFGAGTKLELK |
| | light chain | |
| | variable | |
| | region | |
| 297 | ALG5 | KASQNVGTNVA |
| | mAb | |
| | 2A11-1 | |
| | light chain | |
| | CDR1 | |
| 298 | ALG5 | SASSRYS |
| | mAb | |
| | 2A11-1 | |
| | light chain | |
| | CDR2 | |
| 299 | ALG5 | QQYNSFPLT |
| | mAb | |
| | 2A11-1 | |
| | light chain | |
| | CDR3 | |
| 300 | ALG5 | ATGGAGTCACAGACTCAGGTCTTTGTATACATGTTGCTGTGGTTG |
| | mAb | TCTGGTGTTGATGGAGACATTGTGATGACCCAGTCTCAAAAATTC |
| | 2A11-1 | ATGTCCACATCAGTAGGAGACAGGGTCAGCGTCACCTGCAAGG |
| | light chain | CCAGTCAGAATGTGGGTACTAATGTAGCCTGGTATCAACAG |
| | nucleic | AAACCAGGGCAATCTCCTAAAGCACTGATTTACTCGGCATCCT |
| | acid | CCCGGTACAGTGGAGTCCCTGATCGCCTCACAGGCAGTGGAT |
| | | CTGGGACAGATTTCACTCTCACCATCAGCAATGTGCAGTCTGA |
| | | GGACTTGGCAGAGTATTTCTGTCAGCAATATAACAGCTTTCC |
| | | TCTCACGTTCGGTGCTGGGACCAAGCTGGAGCTGAAACGGGC |
| | | TGATGCTGCACCAACTGTATCCATCTTCCCACCATCCAGTGAGC |
| | | AGTTAACATCTGGAGGTGCCTCAGTCGTGTGCTTCTTGAACAA |
| | | CTTCTACCCCAAAGACATCAATGTCAAGTGGAAGATTGATGGC |
| | | AGTGAACGACAAAATGGCGTCCTGAACAGTTGGACTGATCAGG |
| | | ACAGCAAAGACAGCACCTACAGCATGAGCAGCACCCTCACGTT |
| | | GACCAAGGACGAGTATGAACGACATAACAGCTATACCTGTGA |
| | | GGCCACTCACAAGACATCAACTTCACCCATTGTCAAGAGCTTC |
| | | AACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 301 | ALG5 | MGWSWIFLFLLSETAGVLSEVQLQQSGPELVKPGASVKMSCKASG |
| | mAb | YTFTDYSFHWVKQSLGKSLEWIGYINPNNGGSSYNQKFKGTATL |
| | 9H3_1 | TVNKSSSTAYMELRSLTSEDSAVYYCARADGYYWGQGTILTVSS |
| | heavy | AKTTPPSVYPLAPGCGDTTGSSVTLGCLVKGYFPESVTVTWNSGS |
| | chain | LSSSVHTFPALLQSGLYTMSSSVTVPSSTWPSQTVTCSVAHPASST |
| | | TVDKKLEPSGPISTINPCPPCKECHKCPAPNLEGGPSVFIFPPNIKDV |
| | | LMISLTPKVTCVVVDVSEDDPDVRISWFVNNVEVHTAQTQTHRE |
| | | DYNSTIRVVSALPIQHQDWMSGKEFKCKVNNKDLPSPIERTISKIK |
| | | GLVRAPQVYILPPPAEQLSRKDVSLTCLVVGFNPGDISVEWTSNG |
| | | HTEENYKDTAPVLDSDGSYFIYSKLDIKTSKWEKTDSFSCNVRHE |
| | | GLKNYYLKKTISRSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 302 | ALG5 | EVQLQQSGPELVKPGASVKMSCKASGYTFTDYSFHWVKQSLGKS |
| | mAb | LEWIGYINPNNGGSSYNQKFKGTATLTVNKSSSTAYMELRSLTSE |
| | 9H3_1 | DSAVYYCARADGYYWGQGTILTVSSAKTTPPSVYPLAPGCGDTT |
| | heavy | GSSVTLGCLVKGYFPESVTVTWNSGSLSSSVHTFPALLQSGLYTM |
| | chain | SSSVTVPSSTWPSQTVTCSVAHPASSTTVDKKLEPSGPISTINPCPP |
| | protein | CKECHKCPAPNLEGGPSVFIFPPNIKDVLMISLTPKVTCVVVDVSE |
| | w/o signal | DDPDVRISWFVNNVEVHTAQTQTHREDYNSTIRVVSALPIQHQD |
| | peptide | WMSGKEFKCKVNNKDLPSPIERTISKIKGLVRAPQVYILPPPAEQL |
| | | SRKDVSLTCLVVGFNPGDISVEWTSNGHTEENYKDTAPVLDSDGS |
| | | YFIYSKLDIKTSKWEKTDSFSCNVRHEGLKNYYLKKTISRSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 303 | ALG5 | EVQLQQSGPELVKPGASVKMSCKASGYTFTDYSFHWVKQSLGKS |
| | mAb | LEWIGYINPNNGGSSYNQKFKGTATLTVNKSSSTAYMELRSLTSE |
| | 9H3_1 | DSAVYYCARADGYYWGQGTILTVSS |
| | heavy | |
| | chain | |
| | variable | |
| | region | |
| 304 | ALG5 | DYSFH |
| | mAb | |
| | 9H3_1 | |
| | heavy | |
| | chain | |
| | CDR1 | |
| 305 | ALG5 | YINPNNGGSSYNQKFKG |
| | mAb | |
| | 9H3_1 | |
| | heavy | |
| | chain | |
| | CDR2 | |
| 306 | ALG5 | ADGYY |
| | mAb | |
| | 9H3_1 | |
| | heavy | |
| | chain | |
| | CDR3 | |
| 307 | ALG5 | ATGGGATGGAGCTGGATCTTTCTCTTTCTCCTGTCAGAAACTGCA |
| | mAb | GGTGTCCTCTCTGAGGTCCAGCTGCAACAGTCTGGACCTGAGCTG |
| | 9H3_1 | GTGAAGCCTGGGGCTTCAGTGAAGATGTCCTGCAAGGCTTCTG |
| | heavy | GATACACATTCACTGACTACAGCTTCCACTGGGTGAAGCAGA |
| | chain | GCCTTGGAAAGAGCCTTGAGTGGATTGGATATATTAACCCTA |
| | nucleic | ATAATGGTGGTTCTTCCTACAACCAGAAGTTCAAGGGCACG |
| | acid | GCCACATTGACTGTAAACAAGTCCTCCAGCACAGCCTACATGG |
| | | AACTCCGCAGCCTGACATCGGAGGATTCTGCAGTCTATTACTG |
| | | TGCAAGAGCCGATGGTTATTACTGGGGCCAAGGCACCATTCT |
| | | CACAGTCTCCTCAGCCAAAACAACACCCCCATCAGTCTATCCA |
| | | CTGGCCCCTGGGTGTGGAGATACAACTGGTTCCTCTGTGACTCT |
| | | GGGATGCCTGGTCAAGGGCTACTTCCCTGAGTCAGTGACTGTG |
| | | ACTTGGAACTCTGGATCCCTGTCCAGCAGTGTGCACACCTTCCC |
| | | AGCTCTCCTGCAGTCTGGACTCTACACTATGAGCAGCTCAGTG |
| | | ACTGTCCCCTCCAGCACCTGGCCAAGTCAGACCGTCACCTGCA |
| | | GCGTTGCTCACCCAGCCAGCAGCACCACGGTGGACAAAAAACT |
| | | TGAGCCCAGCGGGCCCATTTCAACAATCAACCCCTGTCCTCCA |
| | | TGCAAGGAGTGTCACAAATGCCCAGCTCCTAACCTCGAGGGTG |
| | | GACCATCCGTCTTCATCTTCCCTCCAAATATCAAGGATGTACTC |
| | | ATGATCTCCCTGACACCCAAGGTCACGTGTGTGGTGGTGGATG |
| | | TGAGCGAGGATGACCCAGACGTCCGGATCAGCTGGTTTGTGAA |
| | | CAACGTGGAAGTACACACAGCTCAGACACAAACCCATAGAGA |
| | | GGATTACAACAGTACTATCCGGGTGGTCAGTGCCCTCCCCATC |
| | | CAGCACCAGGACTGGATGAGTGGCAAGGAGTTCAAATGCAAG |
| | | GTCAACAACAAAGACCTCCCATCACCCATCGAGAGAACCATCT |
| | | CAAAAATTAAAGGGCTAGTCAGAGCTCCACAAGTATACATCTT |
| | | GCCGCCACCAGCAGAGCAGTTGTCCAGGAAAGATGTCAGTCTC |
| | | ACTTGCCTGGTCGTGGGCTTCAACCCTGGAGACATCAGTGTGG |
| | | AGTGGACCAGCAATGGGCATACAGAGGAGAACTACAAGGACA |
| | | CCGCACCAGTCCTGGACTCTGACGGTTCTTACTTCATATACAGC |
| | | AAGCTCGATATAAAAACAAGCAAGTGGGAGAAAACAGATTCC |
| | | TTCTCATGCAACGTGAGACACGAGGGTCTGAAAAATTACTACC |
| | | TGAAGAAGACCATCTCCCGGTCTCCGGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 308 | ALG5 | MESQTQVFVYMLLWLSGVDGDIVMTQSQKFMSTSVGDRVSVTCKA |
| | mAb | SHNVGTNVAWYQQKPGQSPKALIYSASSRFSGVPDRFAGSGSGT |
| | 9H3_1 | DFTLTISSVQSEDLAEYFCQQYNNFPLTFGAGTNLELKRADAAPT |
| | light chain | VSIFPPSSEQLTSGGASVVCFLNNFYPKDINVKWKIDGSERQNGVL |
| | | NSWTDQDSKDSTYSMSSTLTLTKDEYERHNSYTCEATHKTSTSPI |
| | | VKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 309 | ALG5 | DIVMTQSQKFMSTSVGDRVSVTCKASHNVGTNVAWYQQKPGQS |
| | mAb | PKALIYSASSRFSGVPDRFAGSGSGTDFTLTISSVQSEDLAEYFCQ |
| | 9H3_1 | QYNNFPLTFGAGTNLELKRADAAPTVSIFPPSSEQLTSGGASVVCF |
| | light chain | LNNFYPKDINVKWKIDGSERQNGVLNSWTDQDSKDSTYSMSSTL |
| | w/o signal | TLTKDEYERHNSYTCEATHKTSTSPIVKSFNRNEC |
| | peptide | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 310 | ALG5 | DIVMTQSQKFMSTSVGDRVSVTCKASHNVGTNVAWYQQKPGQS |
| | mAb | PKALIYSASSRFSGVPDRFAGSGSGTDFTLTISSVQSEDLAEYFCQ |
| | 9H3_1 | QYNNFPLTFGAGTNLELK |
| | light chain | |
| | variable | |
| | region | |
| 311 | ALG5 | KASHNVGTNVA |
| | mAb | |
| | 9H3_1 | |
| | light chain | |
| | CDR1 | |
| 312 | ALG5 | SASSRFS |
| | mAb | |
| | 9H3_1 | |
| | light chain | |
| | CDR2 | |
| 313 | ALG5 | QQYNNFPLT |
| | mAb | |
| | 9H3_1 | |
| | light chain | |
| | CDR3 | |
| 314 | ALG5 | ATGGAGTCACAGACTCAGGTCTTTGTATACATGTTGCTGTGGTTG |
| | mAb | TCTGGTGTTGATGGAGACATTGTGATGACCCAGTCTCAAAAATTC |
| | 9H3_1 | ATGTCCACATCAGTAGGAGACAGGGTCAGCGTCACCTGCAAGG |
| | light chain | CCAGTCACAATGTGGGTACTAATGTAGCCTGGTATCAACAG |
| | nucleic | AAGCCAGGGCAATCTCCTAAAGCACTGATTTACTCGGCTTCCT |
| | acid | CCCGGTTCAGTGGAGTCCCTGATCGCTTCGCAGGCAGTGGAT |
| | | CTGGGACAGATTTCACTCTCACCATCAGCAGTGTGCAGTCTGA |
| | | AGACTTGGCAGAGTATTTCTGTCAACAATATAACAACTTTCCT |
| | | CTCACGTTCGGTGCTGGGACCAACCTGGAGCTGAAGCGGGCT |
| | | GATGCTGCACCAACTGTATCCATCTTCCCACCATCCAGTGAGC |
| | | AGTTAACATCTGGAGGTGCCTCAGTCGTGTGCTTCTTGAACAA |
| | | CTTCTACCCCAAAGACATCAATGTCAAGTGGAAGATTGATGGC |
| | | AGTGAACGACAAAATGGCGTCCTGAACAGTTGGACTGATCAGG |
| | | ACAGCAAAGACAGCACCTACAGCATGAGCAGCACCCTCACGTT |
| | | GACCAAGGACGAGTATGAACGACATAACAGCTATACCTGTGA |
| | | GGCCACTCACAAGACATCAACTTCACCCATTGTCAAGAGCTTC |
| | | AACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 315 | ALG5 | MGWSCIILILVAAASGVHSQVQLQQPGTELVKPGASVSLSCKTSGYT |
| | mAb | FTNYWMHWVKQRPGQGLEWIGNINPSNGRSNYIEKFKRKATLT |
| | 1H1_1 | VDTSSSTAYMQLSSLTSEDSAVYFCTRGDYDSDSSWFDYWGQGT |
| | heavy | LVTVSAAKTTPPSVYPLAPGCGDTTGSSVTLGCLVKGYFPESVTV |
| | chain | TWNSGSLSSSVHTFPALLQSGLYTMSSSVTVPSSTWPSQTVTCSVA |
| | | HPASSTTVDKKLEPSGPISTINPCPPCKECHKCPAPNLEGGPSVFIFP |
| | | PNIKDVLMISLTPKVTCVVVDVSEDDPDVRISWFVNNVEVHTAQT |
| | | QTHREDYNSTIRVVSALPIQHQDWMSGKEFKCKVNNKDLPSPIER |
| | | TISKIKGLVRAPQVYILPPPAEQLSRKDVSLTCLVVGFNPGDISVE |
| | | WTSNGHTEENYKDTAPVLDSDGSYFIYSKLDIKTSKWEKTDSFSC |
| | | NVRHEGLKNYYLKKTISRSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 316 | ALG5 | QVQLQQPGTELVKPGASVSLSCKTSGYTFTNYWMHWVKQRPGQ |
| | mAb | GLEWIGNINPSNGRSNYIEKFKRKATLTVDTSSSTAYMQLSSLTS |
| | 1H1_1 | EDSAVYFCTRGDYDSDSSWFDYWGQGTLVTVSAAKTTPPSVYPL |
| | heavy | APGCGDTTGSSVTLGCLVKGYFPESVTVTWNSGSLSSSVHTFPAL |
| | chain | LQSGLYTMSSSVTVPSSTWPSQTVTCSVAHPASSTTVDKKLEPSGP |
| | protein | ISTINPCPPCKECHKCPAPNLEGGPSVFIFPPNIKDVLMISLTPKVTC |
| | w/o signal | VVVDVSEDDPDVRISWFVNNVEVHTAQTQTHREDYNSTIRVVSA |
| | peptide | LPIQHQDWMSGKEFKCKVNNKDLPSPIERTISKIKGLVRAPQVYIL |
| | | PPPAEQLSRKDVSLTCLVVGFNPGDISVEWTSNGHTEENYKDTAP |
| | | VLDSDGSYFIYSKLDIKTSKWEKTDSFSCNVRHEGLKNYYLKKTIS |
| | | RSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 317 | ALG5 | QVQLQQPGTELVKPGASVSLSCKTSGYTFTNYWMHWVKQRPGQ |
| | mAb | GLEWIGNINPSNGRSNYIEKFKRKATLTVDTSSSTAYMQLSSLTS |
| | 1H1_1 | EDSAVYFCTRGDYDSDSSWFDYWGQGTLVTVSA |
| | heavy | |
| | chain | |
| | variable | |
| | region | |
| 318 | ALG5 | NYWMH |
| | mAb | |
| | 1H1_1 | |
| | heavy | |
| | chain | |
| | CDR1 | |
| 319 | ALG5 | NINPSNGRSNYIEKFKR |
| | mAb | |
| | 1H1_1 | |
| | heavy | |
| | chain | |
| | CDR2 | |
| 320 | ALG5 | GDYDSDSSWFDY |
| | mAb | |
| | 1H1_1 | |
| | heavy | |
| | chain | |
| | CDR3 | |
| 321 | ALG5 | ATGGGATGGAGCTGTATCATCCTCATTTTGGTAGCAGCAGCTTC |
| | mAb | AGGTGTCCACTCCCAGGTCCAACTGCAGCAGCCTGGGACTGAAC |
| | 1H1_1 | TGGTGAAGCCTGGGGCTTCAGTGAGCCTGTCCTGTAAGACTTCT |
| | heavy | GGCTACACCTTCACCAACTACTGGATGCACTGGGTGAAACAG |
| | chain | AGGCCTGGACAGGGCCTTGAGTGGATTGGAAATATTAATCCA |
| | nucleic | AGCAATGGTCGTAGTAACTACATAGAGAAGTTCAAGAGAA |
| | acid | AGGCCACACTGACTGTCGACACATCCTCCAGCACAGCCTACAT |
| | | GCAGCTCAGCAGTCTGACATCTGAGGACTCTGCGGTCTATTTT |
| | | TGTACACGAGGGGACTACGATAGTGACTCCTCCTGGTTTGA |
| | | TTACTGGGGCCAAGGGACTCTGGTCACTGTCTCTGCAGCCAA |
| | | AACAACACCCCCATCAGTCTATCCACTGGCCCCTGGGTGTGGA |
| | | GATACAACTGGTTCCTCTGTGACTCTGGGATGCCTGGTCAAGG |
| | | GCTACTTCCCTGAGTCAGTGACTGTGACTTGGAACTCTGGATC |
| | | CCTGTCCAGCAGTGTGCACACCTTCCCAGCTCTCCTGCAGTCT |
| | | GGACTCTACACTATGAGCAGCTCAGTGACTGTCCCCTCCAGCA |
| | | CCTGGCCAAGTCAGACCGTCACCTGCAGCGTTGCTCACCCAGC |
| | | CAGCAGCACCACGGTGGACAAAAAACTTGAGCCCAGCGGGCC |
| | | CATTTCAACAATCAACCCCTGTCCTCCATGCAAGGAGTGTCAC |
| | | AAATGCCCAGCTCCTAACCTCGAGGGTGGACCATCCGTCTTCA |
| | | TCTTCCCTCCAAATATCAAGGATGTACTCATGATCTCCCTGAC |
| | | ACCCAAGGTCACGTGTGTGGTGGTGGATGTGAGCGAGGATGA |
| | | CCCAGACGTCCGGATCAGCTGGTTTGTGAACAACGTGGAAGT |
| | | ACACACAGCTCAGACACAAACCCATAGAGAGGATTACAACAG |
| | | TACTATCCGGGTGGTCAGTGCCCTCCCCATCCAGCACCAGGAC |
| | | TGGATGAGTGGCAAGGAGTTCAAATGCAAGGTCAACAACAAA |
| | | GACCTCCCATCACCCATCGAGAGAACCATCTCAAAAATTAAA |
| | | GGGCTAGTCAGAGCTCCACAAGTATACATCTTGCCGCCACCAG |
| | | CAGAGCAGTTGTCCAGGAAAGATGTCAGTCTCACTTGCCTGGT |
| | | CGTGGGCTTCAACCCTGGAGACATCAGTGTGGAGTGGACCAG |
| | | CAATGGGCATACAGAGGAGAACTACAAGGACACCGCACCAGT |
| | | CCTGGACTCTGACGGTTCTTACTTCATATACAGCAAGCTCGAT |
| | | ATAAAAACAAGCAAGTGGGAGAAAACAGATTCCTTCTCATGC |
| | | AACGTGAGACACGAGGGTCTGAAAAATTACTACCTGAAGAAG |
| | | ACCATCTCCCGGTCTCCGGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 322 | ALG5 | MDFHVQIFSFMLISVTVMLSSGEFVLTQSPAVLAAFPGEKVTITCSV |
| | mAb | NAFLSSSDLHWYQQRSEASPKPLIYGTSNLASGVPVRFSGSGSGT |
| | 1H1_1 | SFSLTISSMEAEDAATYYCQQWSTYPLTFGAGTKLELKRADAAP |
| | light chain | TVSIFSPSSEQLTSGGASVVCFLNNFYPKDINVKWKIDGSERQNGV |
| | | LNSWTDQDSKDSTYSMSSTLTLTKDEYERHNSYTCEATHKTSTSP |
| | | IVKSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 323 | ALG5 | EFVLTQSPAVLAAFPGEKVTITCSVNAFLSSSDLHWYQQRSEASP |
| | mAb | KPLIYGTSNLASGVPVRFSGSGSGTSFSLTISSMEAEDAATYYCQQ |
| | 1H1_1 | WSTYPLTFGAGTKLELKRADAAPTVSIFSPSSEQLTSGGASVVCFL |
| | light chain | NNFYPKDINVKWKIDGSERQNGVLNSWTDQDSKDSTYSMSSTLT |
| | w/o signal | LTKDEYERHNSYTCEATHKTSTSPIVKSFNRNEC |
| | peptide | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 324 | ALG5 | EFVLTQSPAVLAAFPGEKVTITCSVNAFLSSSDLHWYQQRSEASP |
| | mAb | KPLIYGTSNLASGVPVRFSGSGSGTSFSLTISSMEAEDAATYYCQQ |
| | 1H1_1 | WSTYPLTFGAGTKLELK |
| | light chain | |
| | variable | |
| | region | |
| 325 | ALG5 | SVNAFLSSSDLH |
| | mAb | |
| | 1H1_1 | |
| | light chain | |
| | CDR1 | |
| 326 | ALG5 | GTSNLAS |
| | mAb | |
| | 1H1_1 | |
| | light chain | |
| | CDR2 | |
| 327 | ALG5 | QQWSTYPLT |
| | mAb | |
| | 1H1_1 | |
| | light chain | |
| | CDR3 | |
| 328 | ALG5 | ATGGATTTTCATGTGCAGATTTTCAGCTTCATGCTAATCAGTGTC |
| | mAb | ACAGTCATGTTGTCCAGTGGAGAATTTGTGCTCACCCAGTCTCCA |
| | 1H1_1 | GCAGTCTTGGCTGCATTTCCGGGGGAGAAGGTCACCATCACCTGC |
| | light chain | AGTGTCAACGCATTTCTAAGTTCCAGCGACTTGCACTGGTA |
| | nucleic | CCAGCAGAGGTCAGAAGCCTCCCCCAAACCCTTGATTTATGGC |
| | acid | ACATCCAACCTGGCTTCTGGAGTCCCTGTTCGCTTCAGTGGCA |
| | | GTGGATCTGGGACCTCTTTCTCTCTCACAATCAGCAGCATGGA |
| | | GGCGGAAGATGCTGCCACTTATTACTGTCAACAGTGGAGTAC |
| | | TTACCCGCTCACGTTCGGTGCTGGGACCAAGCTGGAGCTGAA |
| | | ACGGGCTGATGCTGCACCAACTGTATCCATCTTCTCACCATCCA |
| | | GTGAGCAGTTAACATCTGGAGGTGCCTCAGTCGTGTGCTTCTTG |
| | | AACAACTTCTACCCCAAAGACATCAATGTCAAGTGGAAGATTG |
| | | ATGGCAGTGAACGACAAAATGGCGTCCTGAACAGTTGGACTGA |
| | | TCAGGACAGCAAAGACAGCACCTACAGCATGAGCAGCACCCT |
| | | CACGTTGACCAAGGACGAGTATGAACGACATAACAGCTATACC |
| | | TGTGAGGCCACTCACAAGACATCAACTTCACCCATTGTCAAGA |
| | | GCTTCAACAGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |
| 329 | ALG5 | MKCSWIIFFLMAVVTGVNSEVQLQQSGAELVKPGASVKLSCTSSGF |
| | mAb | NIKDYYIHWVKQRTESGLEWIGRIDPEDVATKYAPKFQGKATM |
| | 11F5-1 | TADTSSNTAYLQLSSLTSEDAAVYYCTFSHYGNYGWGQGTLVTV |
| | heavy | SAAKTTPPSVYPLAPGCGDTTGSSVTLGCLVKGYFPESVTVTWNS |
| | chain | GSLSSSVHTFPALLQSGLYTMSSSVTVPSSTWPSQTVTCSVAHPAS |
| | | STTVDKKLEPSGPISTINPCPPCKECHKCPAPNLEGGPSVFIFPPNIK |
| | | DVLMISLTPKVTCVVVDVSEDDPDVRISWFVNNVEVHTAQTQTH |
| | | REDYNSTIRVVSALPIQHQDWMSGKEFKCKVNNKDLPSPIERTISK |
| | | IKGLVRAPQVYILPPPAEQLSRKDVSLTCLVVGFNPGDISVEWTSN |
| | | GHTEENYKDTAPVLDSDGSYFIYSKLDIKTSKWEKTDSFSCNVRH |
| | | EGLKNYYLKKTISRSPGK |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 330 | ALG5 | EVQLQQSGAELVKPGASVKLSCTSSGFNIKDYYIHWVKQRTESGL |
| | mAb | EWIGRIDPEDVATKYAPKFQGKATMTADTSSNTAYLQLSSLTSE |
| | 11F5-1 | DAAVYYCTFSHYGNYGWGQGTLVTVSAAKTTPPSVYPLAPGCG |
| | heavy | DTTGSSVTLGCLVKGYFPESVTVTWNSGSLSSSVHTFPALLQSGL |
| | chain | YTMSSSVTVPSSTWPSQTVTCSVAHPASSTTVDKKLEPSGPISTINP |
| | protein | CPPCKECHKCPAPNLEGGPSVFIFPPNIKDVLMISLTPKVTCVVVD |
| | w/o signal | VSEDDPDVRISWFVNNVEVHTAQTQTHREDYNSTIRVVSALPIQH |
| | peptide | QDWMSGKEFKCKVNNKDLPSPIERTISKIKGLVRAPQVYILPPPAE |
| | | QLSRKDVSLTCLVVGFNPGDISVEWTSNGHTEENYKDTAPVLDSD |
| | | GSYFIYSKLDIKTSKWEKTDSFSCNVRHEGLKNYYLKKTISRSPGK |
| | | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 331 | ALG5 | EVQLQQSGAELVKPGASVKLSCTSSGFNIKDYYIHWVKQRTESGL |
| | mAb | EWIGRIDPEDVATKYAPKFQGKATMTADTSSNTAYLQLSSLTSE |
| | 11F5-1 | DAAVYYCTFSHYGNYGWGQGTLVTVSA |
| | heavy | |
| | chain | |
| | variable | |
| | region | |
| 332 | ALG5 | DYYIH |
| | mAb | |
| | 11F5-1 | |
| | heavy | |
| | chain | |
| | CDR1 | |
| 333 | ALG5 | RIDPEDVATKYAPKFQG |
| | mAb | |
| | 11F5-1 | |
| | heavy | |
| | chain | |
| | CDR2 | |
| 334 | ALG5 | SHYGNYG |
| | mAb | |
| | 11F5-1 | |
| | heavy | |
| | chain | |
| | CDR3 | |
| 335 | ALG5 | ATGAAATGCAGCTGGATCATCTTCTTCCTGATGGCAGTGGTTACAG |
| | mAb | GGGTCAATTCAGAGGTTCAGCTGCAGCAATCTGGGGCAGAACTC |
| | 11F5-1 | GTGAAGCCGGGGGCCTCAGTCAAGTTGTCCTGCACATCTTCTG |
| | heavy | GCTTCAACATTAAAGACTACTATATACACTGGGTGAAGCAGA |
| | chain | GGACTGAATCGGGCCTGGAATGGATTGGAAGGATTGATCCTG |
| | nucleic | AAGATGTTGCAACTAAATATGCCCCGAAATTCCAGGGCAAG |
| | acid | GCCACTATGACAGCAGACACATCCTCCAACACAGCCTACCTCC |
| | | AGCTCAGCAGCCTGACATCTGAGGACGCTGCCGTCTATTATTG |
| | | TACTTTTAGTCATTATGGTAACTACGGGTGGGGCCAGGGGAC |
| | | TCTGGTCACTGTCTCTGCAGCCAAAACAACACCCCCATCAGTCT |
| | | ATCCACTGGCCCCTGGGTGTGGAGATACAACTGGTTCCTCTGT |
| | | GACTCTGGGATGCCTGGTCAAGGGCTACTTCCCTGAGTCAGTG |
| | | ACTGTGACTTGGAACTCTGGATCCCTGTCCAGCAGTGTGCACA |
| | | CCTTCCCAGCTCTCCTGCAGTCTGGACTCTACACTATGAGCAGC |
| | | TCAGTGACTGTCCCCTCCAGCACCTGGCCAAGTCAGACCGTCA |
| | | CCTGCAGCGTTGCTCACCCAGCCAGCAGCACCACGGTGGACAA |
| | | AAAACTTGAGCCCAGCGGGCCCATTTCAACAATCAACCCCTGT |
| | | CCTCCATGCAAGGAGTGTCACAAATGCCCAGCTCCTAACCTCG |
| | | AGGGTGGACCATCCGTCTTCATCTTCCCTCCAAATATCAAGGAT |
| | | GTACTCATGATCTCCCTGACACCCAAGGTCACGTGTGTGGTGG |
| | | TGGATGTGAGCGAGGATGACCCAGACGTCCGGATCAGCTGGTT |
| | | TGTGAACAACGTGGAAGTACACACAGCTCAGACACAAACCCAT |
| | | AGAGAGGATTACAACAGTACTATCCGGGTGGTCAGTGCCCTCC |
| | | CCATCCAGCACCAGGACTGGATGAGTGGCAAGGAGTTCAAATG |
| | | CAAGGTCAACAACAAAGACCTCCCATCACCCATCGAGAGAACC |
| | | ATCTCAAAAATTAAAGGGCTAGTCAGAGCTCCACAAGTATACA |
| | | TCTTGCCGCCACCAGCAGAGCAGTTGTCCAGGAAAGATGTCAG |
| | | TCTCACTTGCCTGGTCGTGGGCTTCAACCCTGGAGACATCAGTG |
| | | TGGAGTGGACCAGCAATGGGCATACAGAGGAGAACTACAAGG |
| | | ACACCGCACCAGTCCTGGACTCTGACGGTTCTTACTTCATATAC |
| | | AGCAAGCTCGATATAAAAACAAGCAAGTGGGAGAAAACAGAT |
| | | TCCTTCTCATGCAACGTGAGACACGAGGGTCTGAAAAATTACT |
| | | ACCTGAAGAAGACCATCTCCCGGTCTCCGGGTAAATGA |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TGA) |
| 336 | ALG5 | MESQTQVFVYMLLWLSGVDGDTVMTQSQKFMSSSIGDRVSVTCKA |
| | mAb | SKNVGISVAWYQQKPGQSPKALIYSASYRYSGVPDRFTGSGSGTD |
| | 11F5-1 | FTLTINNVQSEDLAEYFCQQYNGYPFTFGSGTKLEIKRADAAPTV |
| | light chain | SIFPPSSEQLTSGGASVVCFLNNFYPKDINVKWKIDGSERQNGVLN |
| | | SWTDQDSKDSTYSMSSTLTLTKDEYERHNSYTCEATHKTSTSPIV |
| | | KSFNRNEC |
| | | Note: the sequence include signal peptide (italicized)-FR1 (underlined)- |
| | | CDR1 (bolded)-FR2 (underlined)-CDR2 (bolded)-FR3 (underlined)- |
| | | CDR3 (bolded)-FR4 (underlined)-constant region |
| 337 | ALG5 | DTVMTQSQKFMSSSIGDRVSVTCKASKNVGISVAWYQQKPGQSP |
| | mAb | KALIYSASYRYSGVPDRFTGSGSGTDFTLTINNVQSEDLAEYFC |
| | 11F5-1 | QQYNGYPFTFGSGTKLEIKRADAAPTVSIFPPSSEQLTSGGASV |
| | light chain | VCFLNNFYPKDINVKWKIDGSERQNGVLNSWTDQDSKDSTYSMS |
| | w/o signal | STLTLTKDEYERHNSYTCEATHKTSTSPIVKSFNRNEC |
| | peptide | Note: the sequence include FR1 (underlined)-CDR1 (bolded)-FR2 |
| | | (underlined)-CDR2 (bolded)-FR3 (underlined)-CDR3 (bolded)-FR4 |
| | | (underlined)-constant region |
| 338 | ALG5 | DTVMTQSQKFMSSSIGDRVSVTCKASKNVGISVAWYQQKPGQSP |
| | mAb | KALIYSASYRYSGVPDRFTGSGSGTDFTLTINNVQSEDLAEYFC |
| | 11F5-1 | QQYNGYPFTFGSGTKLEIK |
| | light chain | |
| | variable | |
| | region | |
| 339 | ALG5 | KASKNVGISVA |
| | mAb | |
| | 11F5-1 | |
| | light chain | |
| | CDR1 | |
| 340 | ALG5 | SASYRYS |
| | mAb | |
| | 11F5-1 | |
| | light chain | |
| | CDR2 | |
| 341 | ALG5 | QQYNGYPFT |
| | mAb | |
| | 11F5-1 | |
| | light chain | |
| | CDR3 | |
| 342 | ALG5 | ATGGAGTCACAGACTCAGGTCTTTGTATACATGTTGCTGTGGTTG |
| | mAb | TCTGGTGTTGATGGAGACACTGTGATGACCCAGTCTCAAAAATTC |
| | 11F5-1 | ATGTCCTCATCAATAGGAGACAGGGTCAGCGTCACCTGCAAGGC |
| | light chain | CAGTAAGAATGTGGGTATTAGTGTCGCCTGGTATCAGCAGA |
| | nucleic | AACCAGGGCAATCTCCTAAAGCATTGATTTACTCGGCATCCTA |
| | acid | CCGGTACAGTGGAGTCCCTGATCGCTTCACAGGCAGTGGATCT |
| | | GGGACAGATTTCACTCTCACCATCAACAATGTGCAGTCTGAAG |
| | | ACTTGGCAGAATATTTCTGTCAGCAATATAACGGCTATCCAT |
| | | TCACGTTCGGCTCGGGGACAAAGTTGGAAATAAAACGGGCTG |
| | | ATGCTGCACCAACTGTATCCATCTTCCCACCATCCAGTGAGCA |
| | | GTTAACATCTGGAGGTGCCTCAGTCGTGTGCTTCTTGAACAACT |
| | | TCTACCCCAAAGACATCAATGTCAAGTGGAAGATTGATGGCAG |
| | | TGAACGACAAAATGGCGTCCTGAACAGTTGGACTGATCAGGAC |
| | | AGCAAAGACAGCACCTACAGCATGAGCAGCACCCTCACGTTGA |
| | | CCAAGGACGAGTATGAACGACATAACAGCTATACCTGTGAGGC |
| | | CACTCACAAGACATCAACTTCACCCATTGTCAAGAGCTTCAAC |
| | | AGGAATGAGTGTTAG |
| | | Note: the sequence include coding sequences for signal peptide |
| | | (italicized)-FR1 (underlined)-CDR1 (bolded)-FR2 (underlined)-CDR2 |
| | | (bolded)-FR3 (underlined)-CDR3 (bolded)-FR4 (underlined)-constant |
| | | region-stop codon (last three nucleotides TAG) |

What is claimed is:

1. A method of treating a subject in need of therapy for a pathogen-induced infection, comprising administering to the subject a multiple EGF-like-domains-9 (MEGF9) protein or a biologically active fragment thereof,
wherein the pathogen-induced infection is a coronavirus, a SARS-CoV-1 virus, a SARS-CoV-2, or a MERS-CoV, an influenza virus, a respiratory syncytial virus (RSV), an enterovirus (Picornaviridae), a rhinovirus, parainfluenza, a metapneumovirus, a bocavirus, an adenovirus, or a Coxsackie virus infection.

2. The method of claim 1, wherein the MEGF9 protein or biologically active fragment of MEGF9 comprises a sequence with at least 80%, at least 85%, at least 90%, or at least 95% sequence identity to a sequence selected from SEQ ID NOs: 1-4 or 102.

3. The method of claim 1, wherein the biologically active fragment of MEGF9 comprises a sequence with at least 80%, at least 85%, at least 90%, or at least 95% sequence identity to HGHVDPVKTPKI (SEQ ID NO: 350).

4. The method of claim 2, wherein the MEGF9 protein or biologically active fragment of MEGF9 comprises a sequence according to SEQ ID NOs: 1-4 or 102.

5. The method of claim 3, wherein the biologically active fragment of MEGF9 is a sequence consisting of HGHVDPVKTPKI (SEQ ID NO: 350).

6. The method of claim 1, comprising treating the influenza virus pathogen in the subject by administering to the subject a sequence comprising:
(SEQ ID NO: 2)
AVASAASAGNVTGGGGAAGQVDASPGPGLRGEPSHPFPRATAPTAQAPRTG
PPRATVHRPLAATSPAQSPETTPLWATAGPSSTTFQAPLGPSPTTPPAAER
TSTTSQAPTRPAPTTLSTTTGPAPTTPVATTVPAPTTPRTPTPDLPSSSNS
SVLPTPPATEAPSSPPPEYVCNCSVVGSLNVNRCNQTTGQCECRPGYQGLH
CETCKEGFYLNYTSGLCQPCDCSPHGALSIPCNSSGKCQCKVGVIGSICDR
CQDGYYGFSKNGCLPCQCNNRSASCDALTGACLNCQENSKGNHCEECKEGF
YQSPDATKECLRCPCSAVTSTGSCSIKSSELEPECDQCKDGYIGPNCNKCE
NGYYNFDSICRKCQCHGHVDPVKTPKICKPESGECINCLHNTTGFWCENCL
EGYVHDLEGNCIKKEVILPTPEGSTILVSNASLTTSVPTPVINSTFTPTTL
QTIFSVSTSENSTSALADVSWTQFNIIILTVIIIVVVLLMGFVGAVYMYRE
YQNRKLNAPFWTIELKEDNISFSSYHDSIPNADVSGLLEDDGNEVAPNGQL
TLTTPIHNYKA.

7. The method of claim 1, comprising treating the influenza virus pathogen in the subject by administering to the subject a sequence consisting of: HGHVDPVKTPKI (SEQ ID NO: 350).

8. The method of claim 1, wherein the MEGF9 protein or biologically active fragment of MEGF9 is administered at a dose of about 0.2 mg/kg to about 250 mg/kg.

9. The method of claim 8, wherein the MEGF9 protein or biologically active fragment of MEGF9 is administered at a dose of about 1 mg/kg to about 25 mg/kg.

10. The method of claim 9, wherein the MEGF9 protein or biologically active fragment of MEGF9 is administered at a dose of about 1 mg/kg to about 2.5 mg/kg.

11. The method of claim 1, wherein the pathogen-induced infection is a pulmonary viral infection.

12. The method of claim 1, wherein the infection is by an influenza virus.

13. The method of claim 12, wherein the influenza virus is selected from influenza A, B, C, or D.

14. The method of claim 13, wherein the influenza virus is influenza A virus.

15. The method of claim 1, wherein the MEGF9 protein or a biologically active fragment thereof is administered in a pharmaceutical composition that further comprises a pharmaceutically acceptable vehicle or excipient.

16. The method of claim 15, further comprising administering the pharmaceutical composition via a route selected from the group consisting of intrapulmonary, intravenous, intramuscular, subcutaneous, oral, or in any combination thereof.

17. The method of claim 1, further comprising administering at least one additional therapeutic agent.

18. The method of claim 17, wherein the at least one additional therapeutic agent is chloroquine, hydroxychloroquine, type I interferon, Azithromycin, Tocilizumab, sarilumab, interferon beta, anti-virals, remdesivir, baricitinib, dexamethasone, monoclonal antibodies including bamlanivimab (LY-CoV555), etesevimab, Casirivimab and imdevimab, AZD7442, VIR-7831, bemnifosbuvir (AT-527), lenzilumab, leronlimab, favipiravir, lopinavir, nirmatrelvir, molnupiravir, ritonavir, or a combination thereof.

19. The method of claim 1, wherein the subject is a human.

20. A method of treating a subject in need of therapy for a pathogen-induced lung injury, comprising administering to the subject a multiple EGF-like-domains-9 (MEGF9) protein or a biologically active fragment thereof,
wherein the pathogen-induced lung injury is caused by a coronavirus, a SARS-CoV-1 virus, a SARS-CoV-2, or a MERS-CoV, an influenza virus, a respiratory syncytial virus (RSV), an enterovirus (Picornaviridae), a rhinovirus, parainfluenza, a metapneumovirus, a bocavirus, an adenovirus, or a Coxsackie virus.

21. The method of claim 20, wherein the pathogen-induced lung injury comprises alveolar epithelial cell (AEC) damage.

22. The method of claim 20, wherein the pathogen-induced lung injury comprises lung tissue remodeling.

23. The method of claim 20, comprising treating the influenza virus pathogen-induced lung injury in the subject by administering to the subject a sequence comprising:
(SEQ ID NO: 2)
AVASAASAGNVTGGGGAAGQVDASPGPGLRGEPSHPFPRATAPTAQAPRT
GPPRATVHRPLAATSPAQSPETTPLWATAGPSSTTFQAPLGPSPTTPPAA
ERTSTTSQAPTRPAPTTLSTTTGPAPTTPVATTVPAPTTPRTPTPDLPSS
SNSSVLPTPPATEAPSSPPPEYVCNCSVVGSLNVNRCNQTTGQCECRPGY
QGLHCETCKEGFYLNYTSGLCQPCDCSPHGALSIPCNSSGKCQCKVGVIG
SICDRCQDGYYGFSKNGCLPCQCNNRSASCDALTGACLNCQENSKGNHCE
ECKEGFYQSPDATKECLRCPCSAVTSTGSCSIKSSELEPECDQCKDGYIG
PNCNKCENGYYNFDSICRKCQCHGHVDPVKTPKICKPESGECINCLHNTT
GFWCENCLEGYVHDLEGNCIKKEVILPTPEGSTILVSNASLTTSVPTPVI
NSTFTPTTLQTIFSVSTSENSTSALADVSWTQFNIIILTVIIIVVVLLMG
FVGAVYMYREYQNRKLNAPFWTIELKEDNISFSSYHDSIPNADVSGLLED
DGNEVAPNGQLTLTTPIHNYKA.

24. The method of claim 20, comprising treating the influenza virus pathogen-induced lung injury in the subject by administering to the subject a sequence consisting of: HGHVDPVKTPKI (SEQ ID NO: 350).

25. The method of claim 20, wherein the MEGF9 protein or biologically active fragment of MEGF9 is administered at a dose of about 0.2 mg/kg to about 250 mg/kg.
